(19) United States
(12) Patent Application Publication
(10) Pub. No.: US 2022/0354967 A1
(43) Pub. Date: Nov. 10, 2022

(54) COMPOSITIONS AND METHODS FOR TRANSGENE EXPRESSION FROM AN ALBUMIN LOCUS

(71) Applicants: Intellia Therapeutics, Inc., Cambridge, MA (US); Regeneron Pharmaceuticals, Inc., Tarrytown, NY (US)

(72) Inventors: Jonathan Douglas Finn, Melrose, MA (US); Hon-Ren Huang, Cambridge, MA (US); Moitri Roy, Cambridge, MA (US); KehDih Lai, Tarrytown, NY (US); Rachel Sattler, Tarrytown, NY (US); Christos Kyratsous, Tarrytown, NY (US); Cheng Wang, Tarrytown, NY (US)

(21) Appl. No.: 17/233,373

(22) Filed: Apr. 16, 2021

Related U.S. Application Data
(63) Continuation of application No. PCT/US19/57086, filed on Oct. 18, 2019.
(60) Provisional application No. 62/840,346, filed on Apr. 29, 2019.
(60) Provisional application No. 62/747,402, filed on Oct. 18, 2018.

(51) Int. Cl. A61K 48/00; C12N 15/11; C12N 9/22

(52) U.S. Cl. CPC A61K 48/0041; A61K 48/005; C12N 15/11; C12N 2750/14143; C12N 2310/20; C12N 2800/80; C12N 9/22

(57) ABSTRACT

The present disclosure is directed to methods and compositions useful for inserting and expressing a heterologous (exogenous) gene within a genomic locus, such as a safe harbor site, of a host cell.

This application claims the benefit of priority from U.S. Provisional Application No. 62/747,402, filed on Oct. 18, 2018 and U.S. Provisional Application No. 62/840,346, filed on Apr. 29, 2019. The specifications of each of the foregoing applications are incorporated herein by reference in their entirety.

The instant application contains a Sequence Listing which has been submitted electronically in ASCII format and is hereby incorporated by reference in its entirety. Said ASCII copy, created on Jun. 23, 2021, is named ILH-02102_SL.txt and is 197,814 bytes in size.

Genome editing in gene therapy approaches arises from the idea that the exogenous introduction of the missing or otherwise compromised genetic material can correct a genetic disease. Gene therapy has long been recognized for its enormous potential in how practitioners approach and treat human diseases. Instead of relying on drugs or surgery, patients with underlying genetic factors can be treated by directly targeting the underlying cause. Furthermore, by targeting the underlying genetic cause, gene therapy can provide the potential to effectively cure patients. However, clinical applications of gene therapy approaches still require improvement in several aspects.

Provided herein are compositions and methods useful for inserting and expressing a heterologous (exogenous) gene within a genomic locus, such as a safe harbor site, of a host cell. Several safe harbor loci have been described, including CCR5, HPRT, AAVS1, Rosa and albumin. As described herein, targeting and inserting an exogenous gene at the albumin locus (e.g., at intron 1) allows the use of albumin's endogenous promoter to drive robust expression of the exogenous gene. The present disclosure is based, in part, on the identification of guide RNAs that specifically target sites within the albumin gene, e.g., intron 1 of the albumin gene, and which provide efficient insertion and/or expression of an exogenous gene. The following embodiments are provided.

In one aspect, the present disclosure provides a method of inserting a nucleic acid encoding a heterologous polypeptide into an albumin locus of a host cell or cell population, comprising administering: i) a gRNA that comprises a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; g) a sequence that is complementary to 15 consecutive nucleotides +/−10 nucleotides of the genomic coordinates listed for SEQ ID NOs: 2-33; h) a sequence that is at least 95%, 90%, 85%, 80%, or 75% identical to a sequence selected from the group consisting of SEQ ID NOs: 98-119; i) at least 17, 18, 19, or 20 contiguous nucleotides of a sequence selected from the group consisting of SEQ ID NOs: 98-119; and j) a sequence selected from the group consisting of SEQ ID NOs: 120-163; ii) an RNA-guided DNA binding agent; and iii) a construct comprising a nucleic acid encoding the heterologous polypeptide, thereby inserting the nucleic acid encoding the heterologous polypeptide into an albumin locus of the host cell or cell population.

In another aspect, the present disclosure provides a method of expressing a heterologous polypeptide from an albumin locus of a host cell or cell population, comprising administering: i) a gRNA that comprises a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; 0 a sequence selected from the group consisting of SEQ ID NOs: 34-97; and g) a sequence that comprises 15 consecutive nucleotides +/−10 nucleotides of the genomic coordinates listed for SEQ ID NOs: 2-33; ii) an RNA-guided DNA binding agent; and iii) a construct comprising a coding sequence for the heterologous polypeptide, thereby expressing the heterologous polypeptide in the host cell or cell population.

In another aspect, the present disclosure provides a method of expressing a therapeutic agent in a non-dividing cell type or cell population, comprising administering: i) a gRNA that comprises a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that comprises 15 consecutive nucleotides +/−10 nucleotides of the genomic coordinates listed for SEQ ID NOs: 2-33; ii) an RNA-guided DNA binding agent; and iii) a construct comprising a coding sequence for a heterologous polypeptide, thereby expressing the therapeutic agent in the non-dividing cell type or cell population.

In some embodiments, the gRNA comprises a guide sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.

In some embodiments, the method is performed in vivo. In some embodiments, the method is performed in vitro.

In some embodiments, the gRNA binds a region upstream of a protospacer adjacent motif (PAM). In some embodiments, the PAM is chosen from NGG, NNGRRT, NNGRR(N), NNAGAAW, NNNNG(A/C)TT, and NNNNRYAC.

In some embodiments, the gRNA is a dual gRNA (dgRNA). In some embodiments, the gRNA is a single gRNA (sgRNA). In some embodiments, the sgRNA and comprises one or more modified nucleosides. In some embodiments, the Cas nuclease is a class 2 Cas nuclease. In some embodiments, the Cas nuclease is selected from the group consisting of S. pyogenes nuclease, S. aureus nuclease, C. jejuni nuclease, S. thermophilus nuclease, N. meningitidis nuclease, and variants thereof. In some embodiments, the Cas nuclease is Cas9. In some embodiments, the Cas nuclease is a nickase.

In some embodiments, the construct is a bidirectional nucleic acid construct. In some embodiments, the construct comprises: i. a first segment comprising a coding sequence for a heterologous polypeptide; and ii. a second segment comprising a reverse complement of a coding sequence of the heterologous polypeptide. In some embodiments, the construct comprises a polyadenylation signal sequence. In some embodiments, the construct comprises a splice acceptor site. In some embodiments, the construct does not comprise a homology arm.

In some embodiments, the gRNA is administered in a vector and/or a lipid nanoparticle. In some embodiments, the RNA-guided DNA binding agent is administered in a vector and/or a lipid nanoparticle. In some embodiments, the construct comprising the heterologous gene is administered in a vector and/or a lipid nanoparticle. In some embodiments, the vector is a viral vector. In some embodiments, the viral vector is selected from the group consisting of adeno-associated viral (AAV) vector, adenovirus vector, retrovirus vector, and lentivirus vector. In some embodiments, the AAV vector is selected from the group consisting of AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof.

In some embodiments, the gRNA, the RNA-guided DNA binding agent, and the construct comprising a coding sequence for the heterologous polypeptide, individually or in any combination, are administered simultaneously. In some embodiments, the gRNA, the RNA-guided DNA binding agent, and the construct comprising a coding sequence for the heterologous polypeptide are administered sequentially, in any order and/or in any combination. In some embodiments, the RNA-guided DNA binding agent, or RNA-guided DNA binding agent and gRNA in combination, is administered prior to providing the construct. In some embodiments, the construct comprising a coding sequence for the heterologous polypeptide is administered prior to the gRNA and/or RNA-guided DNA binding agent.

In some embodiments, the heterologous polypeptide is a secreted polypeptide. In some embodiments, the heterologous polypeptide is an intracellular polypeptide.

In some embodiments, the cell is a liver cell. In some embodiments, the liver cell is a hepatocyte.

In some embodiments, expression of the heterologous polypeptide in the host cell is increased by at least about 2%, about 3%, about 4%, about 5%, about 6%, about 7%, about 8%, about 9%, about 10%, or more, relative to a level in the cell prior to administering the gRNA, RNA-guided DNA binding agent, and construct comprising a coding sequence for the heterologous polypeptide.

In some embodiments, the gRNA comprising SEQ ID NO: 301.

In some embodiments, the gRNA mediates target-specific cutting by an RNA-guided DNA binding agent, results in insertion of the coding sequence for the heterologous polypeptide within intron 1 of an albumin gene. In some embodiments, the cutting results in a rate of at least about 10% insertion of a heterologous nucleic acid in the cell population. In some embodiments, the cutting results in a rate of between about 30 and 35%, about 35 and 40%, about 40 and 45%, about 45 and 50%, about 50 and 55%, about 55 and 60%, about 60 and 65%, about 65 and 70%, about 70 and 75%, about 75 and 80%, about 80 and 85%, about 85 and 90%, about 90 and 95%, or about 95 and 99% insertion of the coding sequence for the heterologous polypeptide.

In some embodiments, the RNA-guided DNA-binding protein is an S. pyogenes Cas9 nuclease. In some embodiments, the nuclease is a cleavase or a nickase.

In some embodiments, the method further comprises administering an LNP comprising the gRNA. In some embodiments, the method further comprises administering an LNP comprising an mRNA that encodes the RNA-guided DNA-binding agent. In some embodiments, the LNP comprises the gRNA and the mRNA that encodes the RNA-guided DNA-binding agent. In some embodiments, the gRNA and the RNA-guided DNA-binding protein are administered as an RNP. In some embodiments, the construct is administered via a vector.

In one aspect, the present disclosure provides a host cell made by any one or more of the foregoing methods.

In one aspect, the present disclosure provides a cell comprising a bidirectional nucleic acid construct encoding a heterologous polypeptide integrated within intron 1 of an albumin locus of a host cell. In some embodiments, the host cell is a liver cell. In some embodiments, the liver cell is a hepatocyte.

### BRIEF DESCRIPTION OF THE DRAWINGS

FIG. 1 shows construct formats as represented in AAV genomes. SA=splice acceptor; pA=polyA signal sequence; HA=homology arm; LHA=left homology arm; RHA=right homology arm.

FIG. 2 shows vectors without homology arms are not effective in an immortalized liver cell line (Hepa1-6). An scAAV derived from plasmid P00204 comprising 200 bp homology arms resulted in expression of hFIX in the dividing cells. Use of the AAV vectors derived from P00123 (scAAV lacking homology arms) and P00147 (ssAAV bidirectional construct lacking homology arms) did not result in detectable expression of hFIX.

FIGS. 3A and 3B show results from in vivo testing of insertion templates with and without homology arms using vectors derived from P00123, P00147, or P00204. FIG. 3A shows liver editing levels as measured by indel formation of ˜60% were detected in each group of animals treated with LNPs comprising CRISPR/Cas9 components. FIG. 3B shows animals receiving the ssAAV vectors without homology arms (derived from P00147) in combination with LNP treatment resulted in the highest level of hFIX expression in serum.

FIGS. 4A and 4B show results from in vivo testing of ssAAV insertion templates with and without homology arms. FIG. 4A compares targeted insertion with vectors derived from plasmids P00350, P00356, P00362 (having asymmetrical homology arms as shown), and P00147 (bidirectional construct as shown in FIG. 4B). FIG. 4B compares insertion into a second site targeted with vectors derived from plasmids P00353, P00354 (having symmetrical homology arms as shown), and P00147.

FIGS. 5A-5D show results of targeted insertion of bidirectional constructs across 20 target sites in primary mouse hepatocytes. FIG. 5A shows the schematics of each of the vectors tested. FIG. 5B shows editing as measured by indel formation for each of the treatment groups across each combination tested. FIG. 5C and FIG. 5D show that significant levels of editing (as indel formation at a specific target site) did not necessarily result in more efficient insertion or expression of the transgenes. hSA=human F9 splice acceptor; mSA=mouse albumin splice acceptor; HiBit=tag for luciferase based detection; pA=polyA signal sequence; Nluc=nanoluciferase reporter; GFP=green fluorescent reporter.

FIG. 6 shows results from in vivo screening of targeted insertion with bidirectional constructs across 10 target sites using with ssAAV derived from P00147. As shown, significant levels of indel formation do not necessarily result in high levels of transgene expression.

FIGS. 7A-7D show results from in vivo screening of bidirectional constructs across 20 target sites using ssAAV derived from P00147. FIG. 7A shows varied levels of editing as measured by indel formation were detected for each of the treatment groups across each LNP/vector combination tested. FIG. 7B provides corresponding targeted insertion data. The results show poor correlation between indel formation and insertion or expression of the bidirectional constructs (FIG. 7B and FIG. 7D), and a positive correlation between in vitro and in vivo results (FIG. 7C).

FIGS. 8A and 8B show insertion of the bidirectional construct at the cellular level using in situ hybridization method using probes that can detect the junctions between the hFIX transgene and the mouse albumin exon 1 sequence (FIG. 8A). Circulating hFIX levels correlated with the number of cells that were positive for the hybrid transcript (FIG. 8B).

FIG. 9 shows the effect on targeted insertion of varying the timing between delivery of the ssAAV comprising the bidirectional hFIX construct and LNP.

FIG. 10 shows the effect on targeted insertion of repeat dosing (e.g., 1, 2, or 3 doses) of LNP following delivery of the bidirectional hFIX construct.

FIG. 11A shows the durability of hFIX expression in vivo. FIG. 11B demonstrates expression from intron 1 of albumin was sustained.

FIG. 12A and FIG. 12B show that varying the AAV or LNP dose can modulate the amount of expression of hFIX from intron 1 of the albumin gene in vivo.

FIGS. 13A-13C show results from screening bidirectional constructs across target sites in primary cynomolgus hepatocytes. FIG. 13A shows varied levels of editing as measured by indel formation detected for each of the samples. FIG. 13B and FIG. 13C show that significant levels of indel formation was not predictive for insertion or expression of the bidirectional constructs into intron 1 of the albumin gene.

FIGS. 14A-14D show results from screening bidirectional constructs across target sites in primary human hepatocytes. FIG. 14A shows editing as measured by indel formation detected for each of the samples. FIG. 14B, FIGS. 14C, and 14 D show that significant levels of indel formation was not predictive for insertion or expression of the bidirectional constructs into intron 1 of the albumin gene.

FIG. 15 shows the results of in vivo studies where non-human primates were dosed with LNPs along with a bi-directional hFIX insertion template (derived from P00147). Systemic hFIX levels were achieved only in animals treated with both LNPs and AAV, with no hFIX detectable using AAV or LNPs alone.

FIG. 16A and FIG. 16B show human Factor IX expression levels in the plasma samples at week 6 post-injection.

FIG. 17 shows week 7 serum levels and % positive cells across the multiple lobes for each animal.

### DETAILED DESCRIPTION

Reference will now be made in detail to certain embodiments of the invention, examples of which are illustrated in the accompanying drawings. While the present teachings are described in conjunction with various embodiments, it is not intended to limit the present teachings to those embodiments. On the contrary, the present teachings encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context dictates otherwise. Thus, for example, reference to “a conjugate” includes a plurality of conjugates and reference to “a cell” includes a plurality of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measureable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise. The term “about”, when used before a list, modifies each member of the list. The term “about” or “approximately” means an acceptable error for a particular value as determined by one of ordinary skill in the art, which depends in part on how the value is measured or determined.

The term “about”, when used before a list, modifies each member of the list. The term “about” or “approximately” means an acceptable error for a particular value as determined by one of ordinary skill in the art, which depends in part on how the value is measured or determined.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with optional substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleotides and one or more nucleotide analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to a guide that comprises a guide sequence, e.g. either a crRNA (also known as CRISPR RNA), or the combination of a crRNA and a trRNA (also known as tracrRNA). The crRNA and trRNA may be associated as a single RNA molecule (single guide RNA, sgRNA) or, for example, in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs, such as sgRNAs or dgRNAs, can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA-binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA-binding agents include both the positive and negative strands of genomic DNA (i.e., the sequence given and the sequence's reverse complement), as a nucleic acid substrate for an RNA-guided DNA-binding agent is a double stranded nucleic acid. Accordingly, where a guide sequence is said to be “complementary to a target sequence”, it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA-binding agent” means a polypeptide or complex of polypeptides having RNA and DNA binding activity, or a DNA-binding subunit of such a complex, wherein the DNA binding activity is sequence-specific and depends on the sequence of the RNA. The term RNA-guided DNA binding-agent also includes nucleic acids encoding such polypeptides. Exemplary RNA-guided DNA-binding agents include Cas cleavases/nickases. Exemplary RNA-guided DNA-binding agents may include inactivated forms thereof (“dCas DNA-binding agents”), e.g. if those agents are modified to permit DNA cleavage, e.g. via fusion with a FokI cleavase domain. “Cas nuclease”, as used herein, encompasses Cas cleavases and Cas nickases. Cas cleavases and Cas nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated”), if those agents are modified to permit DNA cleavage. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), also contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). As used herein, delivery of an RNA-guided DNA-binding agent (e.g. a Cas nuclease, a Cas9 nuclease, or an S. pyogenes Cas9 nuclease) includes delivery of the polypeptide or mRNA.

As used herein, “ribonucleoprotein” (RNP) or “RNP complex” refers to a guide RNA together with an RNA-guided DNA-binding agent, such as a Cas nuclease, e.g., a Cas cleavase, Cas nickase, Cas9 cleavase or Cas9 nickase. In some embodiments, the guide RNA guides the RNA-guided DNA-binding agent such as a Cas9 to a target sequence, and the guide RNA hybridizes with and the agent binds to the target sequence; and binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, a first sequence is considered to be “X % complementary to” a second sequence if X % of the bases of the first sequence base pair with the second sequence. For example, a first sequence 5′AAGA3′ is 100% complementary to a second sequence 3′TTCT5′, and the second sequence is 100% complementary to the first sequence. In some embodiments, a first sequence 5′AAGA3′ is 100% complementary to a second sequence 3′TTCTGTGA5′, whereas the second sequence is 50% complementary to the first sequence.

“mRNA” is used herein to refer to a polynucleotide that comprises an open reading frame that can be translated into a polypeptide (i.e., can serve as a substrate for translation by a ribosome and amino-acylated tRNAs). mRNA will predominantly comprise RNA or modified RNA and it can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. Bases of an mRNA can modified bases such as pseudouridine, N-1-methyl-psuedouridine, or other naturally occurring or non-naturally occurring bases.

Exemplary guide sequences useful in the compositions and methods described herein are shown in Table 1 and throughout the application.

As used herein, “indels” refer to insertion/deletion mutations consisting of a number of nucleotides that are either inserted or deleted at the site of double-stranded breaks (DSBs) in a target nucleic acid.

As used herein, “polypeptide” refers to a wild-type or variant protein (e.g., mutant, fragment, fusion, or combinations thereof). A variant polypeptide may possess at least or about 5%, 10%, 15%, 20%, 30%, 35%, 40%, 45%, 50%, 55%, 60%, 65%, 70%, 75%, 80%, 85%, 90%, 95%, or 100% functional activity of the wild-type polypeptide. In some embodiments, the variant is at least 70%, 75%, 80%, 85%, 90%, 92%, 93%, 94%, 95%, 96%, 97%, 98%, or 99% identical to the sequence of the wild-type polypeptide. In some embodiments, a variant polypeptide may be a hyperactive variant. In certain instances, the variant possesses between about 80% and about 120%, 140%, 160%, 180%, 200%, 300%, 400%, 500%, or more of a functional activity of the wild-type polypeptide.

As used herein, a “target sequence” refers to a sequence of nucleic acid in a target gene that has complementarity to the guide sequence of the gRNA. The interaction of the target sequence and the guide sequence directs an RNA-guided DNA-binding agent to bind, and potentially nick or cleave (depending on the activity of the agent), within the target sequence.

As used herein, a “heterologous gene” refers to a gene that has been introduced as an exogenous source to a site within a host cell genome (e.g., an albumin intron 1 site). That is, the introduced gene is heterologous with respect to its insertion site. A polypeptide expressed from such heterologous gene is referred to as a “heterologous polypeptide.” The heterologous gene can be naturally-occurring or engineered, and can be wild type or a variant. The heterologous gene may include nucleotide sequences other than the sequence that encodes the heterologous polypeptide (e.g., an internal ribosomal entry site). The heterologous gene can be a gene that occurs naturally in the host genome, as a wild type or a variant (e.g., mutant). For example, although the host cell contains the gene of interest (as a wild type or as a variant), the same gene or variant thereof can be introduced as an exogenous source for, e.g., expression at a locus that is highly expressed. The heterologous gene can also be a gene that is not naturally occurring in the host genome, or that expresses a heterologous polypeptide that does not naturally occur in the host genome. “Heterologous gene”, “exogenous gene”, and “transgene” are used interchangeably. In some embodiments, the heterologous gene or transgene includes an exogenous nucleic acid sequence, e.g. a nucleic acid sequence is not endogenous to the recipient cell. In some embodiments, the heterologous gene or transgene includes an exogenous nucleic acid sequence, e.g. a nucleic acid sequence that does not naturally occur in the recipient cell. For example, a heterologous gene may be heterologous with respect to its insertion site and with respect to its recipient cell.

A heterologous gene may be inserted into a safe harbor locus within the genome without significant deleterious effects on the host cell, e.g. hepatocyte, e.g., without causing apoptosis, necrosis, and/or senescence, or without causing more than 5%, 10%, 15%, 20%, 25%, 30%, or 40% apoptosis, necrosis, and/or senescence as compared to a control cell. See, e.g., Hsin et al., “Hepatocyte death in liver inflammation, fibrosis, and tumorigenesis,” 2017. In some embodiments, a safe harbor locus allows overexpression of an exogenous gene without significant deleterious effects on the host cell, e.g. hepatocyte, e.g., without causing apoptosis, necrosis, and/or senescence, or without causing more than 5%, 10%, 15%, 20%, 25%, 30%, or 40% apoptosis, necrosis, and/or senescence as compared to a control cell. In some embodiments, a desirable safe harbor locus may be one in which expression of the inserted gene sequence is not perturbed by read-through expression from neighboring genes. In some embodiments, a safe harbor locus allows expression of an exogenous gene without significant deleterious effects on the host cell or cell population, such as hepatocytes or liver cells, e.g. without causing apoptosis, necrosis, and/or senescence, or without causing more than 5%, 10%, 15%, 20%, 25%, 30%, or 40% apoptosis, necrosis, and/or senescence as compared to a control cell or cell population.

In some embodiments, the heterologous gene may be inserted into a safe harbor locus and use the safe harbor locus's endogenous signal sequence, e.g., the albumin signal sequence encoded by exon 1. For example, an coding sequence may be inserted into human albumin intron 1 such that it is downstream of and fuses to the signal sequence of human albumin exon 1.

In some embodiments, the gene may comprise its own signal sequence, may be inserted into the safe harbor locus, and may further use the safe habor locus's endogenous signal sequence. For example, an coding sequence comprising its native signal sequence may be inserted into human albumin intron 1 such that it is downstream of and fuses to the signal sequence of human albumin encoded by exon 1.

In some embodiments, the gene may comprise its own signal sequence and an internal ribosomal entry site (IRES), may be inserted into the safe harbor locus, and may further use the safe habor locus's endogenous signal sequence. For example, a coding sequence comprising its native signal sequence and an IRES sequence may be inserted into human albumin intron 1 such that it is downstream of and fuses to the signal sequence of human albumin encoded by exon 1.

In some embodiments, the gene may comprise its own signal sequence and IRES, may be inserted into the safe harbor locus, and does not use the safe habor locus's endogenous signal sequence. For example, a coding sequence comprising its native signal sequence and an IRES sequence may be inserted into human albumin intron 1 such that it does not fuse to the signal sequence of human albumin encoded by exon 1. In these embodiments, the protein is translated from the IRES site and is not chimeric (e.g., albumin signal peptide fused to heterologous protein), which may be advantageously non- or low-immunogenic. In some embodiments, the protein is not secreted and/or transported extracellularly.

In some embodiments, the gene may be inserted into the safe harbor locus and may comprise an IRES and does not use any signal sequence. For example, a coding sequence comprising an IRES sequence and no native signal sequence may be inserted into human albumin intron 1 such that it does not fuse to the signal sequence of human albumin encoded by exon 1. In some embodiments, the proteins is translated from the IRES site without any signal sequence. In some embodiments, the protein is not secreted and/or transported extracellularly.

As used herein, a “bidirectional nucleic acid construct” (interchangeably referred to herein as “bidirectional construct”) comprises at least two nucleic acid segments, wherein one segment (the first segment) comprises a coding sequence that encodes an agent of interest (the coding sequence may be referred to herein as “transgene” or a first transgene), while the other segment (the second segment) comprises a sequence wherein the complement of the sequence encodes an agent of interest, or a second transgene.

In one embodiment, a bidirectional construct comprise at least two nucleic acid segments in cis, wherein one segment (the first segment) comprises a coding sequence (sometimes interchangeably referred to herein as “transgene”), while the other segment (the second segment) comprises a sequence wherein the complement of the sequence encodes a transgene. The first transgene and the second transgene may be the same or different. The bidirectional constructs may comprise at least two nucleic acid segments in cis, wherein one segment (the first segment) comprises a coding sequence that encodes a heterologous gene in one orientation, while the other segment (the second segment) comprises a sequence wherein its complement encodes the heterologous gene in the other orientation. That is, the first segment is a complement of the second segment (not necessarily a perfect complement); the complement of the second segment is the reverse complement of the first segment (not necessarily a perfect reverse complement though both encode the same heterologous protein). A bidirectional construct may comprise a first coding sequence that encodes a heterologous gene linked to a splice acceptor and a second coding sequence wherein the complement encodes a heterologous gene in the other orientation, also linked to a splice acceptor.

The agent may be therapeutic agent, such as a polypeptide, functional RNA, mRNA, or the like. The transgene may code for an agent such as a polypeptide, functional RNA, or mRNA. In some embodiments, the bidirectional nucleic acid construct comprises at least two nucleic acid segments, wherein one segment (the first segment) comprises a coding sequence that encodes a polypeptide of interest, while the other segment (the second segment) comprises a sequence wherein the complement of the sequence encodes a polypeptide of interest, or a second transgene. That is, the at least two segments can encode identical or different polypeptides or identical or different agents. When the two segments encode an identical polypeptide, the coding sequence of the first segment need not be identical to the complement of the sequence of the second segment. In some embodiments, the sequence of the second segment is a reverse complement of the coding sequence of the first segment. A bidirectional construct can be single-stranded or double-stranded. The bidirectional construct disclosed herein encompasses a construct that is capable of expressing any polypeptide of interest. The bidirectional constructs are useful for genomic insertion of transgene sequences, in particular targeted insertion of the transgene.

In some embodiments, a bidirectional nucleic acid construct comprises a first segment that comprises a coding sequence that encodes a first polypeptide (a first transgene), and a second segment that comprises a sequence wherein the complement of the sequence encodes a second polypeptide (a second transgene). In some embodiments, the first and the second polypeptides are at least 50%, 55%, 60%, 65%, 70%, 75%, 80%, 85%, 90%, 91%, 92%, 93%, 94%, 95%, 96%, 97%, 98%, 99%, or 100% identical. In some embodiments, the first and the second polypeptides comprise an amino acid sequence that is at least 80%, 85%, 90%, 91%, 92%, 93%, 94%, 95%, 96%, 97%, 98%, 99%, or 100% identical, e.g. across 50, 100, 200, 500, 1000 or more amino acid residues.

As used herein, a “reverse complement” refers to a sequence that is a complement sequence of a reference sequence, wherein the complement sequence is written in the reverse orientation. For example, for a hypothetical sequence 5′ CTGGACCGA 3′ (SEQ ID NO: 500), the “perfect” complement sequence is 3′ GACCTGGCT 5′ (SEQ ID NO: 501), and the “perfect” reverse complement is written 5′ TCGGTCCAG 3′ (SEQ ID NO: 502). A reverse complement sequence need not be “perfect” and may still encode the same polypeptide or a similar polypeptide as the reference sequence. Due to codon usage redundancy, a reverse complement can diverge from a reference sequence that encodes the same polypeptide. As used herein, “reverse complement” also includes sequences that are, e.g., 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence.

### II. Compositions

A. Compositions Comprising Guide RNA (gRNAs)

Provided herein are compositions and methods useful for inserting and expressing a heterologous (exogenous) gene within a genomic locus, such as a safe harbor site, of a host cell. In particular, as exemplified herein, targeting and inserting an exogenous gene at the albumin locus (e.g., at intron 1) allows the use of albumin's endogenous promoter to drive robust expression of the exogenous gene. The present disclosure is based, in part, on the identification of guide RNAs that specifically target sites within intron 1 of the albumin gene, and which provide efficient insertion and expression of an exogenous gene. As shown in the Examples and further described herein, the ability of identified gRNAs to mediate high levels of editing as measured through indel forming activity, unexpectedly does not necessarily correlate with use of the same gRNAs to mediate efficient insertion of transgenes as measured through, e.g., expression of the transgene. That is, certain gRNAs that are able to achieve a significant level of indel formation are not necessarily able to mediate efficient insertion, and conversely, some gRNAs shown to achieve low levels of indel formation may mediate efficient insertion and expression of a transgene. Specifically, the data of the Examples indicate that gRNAs that effectively mediate indel formation (also called % editing) did not have indel editing activity that correlated with insertion editing activity.

In some embodiments, provided herein are compositions and methods useful for inserting and expressing an exogenous gene within intron 1 of the albumin gene in a host cell. In some embodiments, disclosed herein are compositions and methods useful for introducing or inserting a heterologous nucleic acid within an albumin locus of a host cell, e.g., using a guide RNA disclosed herein with an RNA-guided DNA binding agent, and a construct comprising a heterologous nucleic acid (“transgene”). In some embodiments, disclosed herein are compositions and methods useful for expressing a heterologous polypeptide at an albumin locus of a host cell, e.g., using a guide RNA disclosed herein with an RNA-guided DNA binding agent and a construct comprising a heterologous nucleic acid (“transgene”). In some embodiments, disclosed herein are compositions and methods useful for inducing a break (e.g., double-stranded break (DSB) or single-stranded break (nick)) within the albumin gene of a host cell, e.g., using a guide RNA disclosed herein with an RNA-guided DNA binding agent (e.g., a CRISPR/Cas system). The compositions and methods may be used in vitro or in vivo for, e.g., therapeutic purposes.

In some embodiments, the guide RNAs disclosed herein comprise a guide sequence that binds to, or is capable of binding, within an intron of an albumin locus. In some embodiments, the guide RNAs disclosed herein bind within a region of intron 1 of the human albumin gene (SEQ ID NO: 1). It will be appreciated that not every base of the guide sequence must bind within the recited regions. For example, in some embodiments, 15, 16, 17, 18, 19, 20, or more bases of the guide RNA sequence bind with the recited regions. For example, in some embodiments, 15, 16, 17, 18, 19, 20, or more contiguous bases of the guide RNA sequence bind with the recited regions.

In some embodiments, the guide RNAs disclosed herein mediate a target-specific cutting by an RNA-guided DNA binding agent (e.g., Cas nuclease) at a site within human albumin intron 1 (SEQ ID NO: 1). It will be appreciated that, in some embodiments, the guide RNAs comprise guide sequences that bind to, or are capable of binding to, a region in SEQ ID NO: 1.

In some embodiments, the guide RNAs disclosed herein comprise a guide sequence that is at least 95%, 90%, 85%, 80%, or 75% identical to a sequence selected from the group consisting of SEQ ID NOs:2-33. In some embodiments, the guide RNAs disclosed herein comprise a guide sequence that is at least 95%, 90%, 85%, 80%, or 75% identical to a sequence selected from the group consisting of SEQ ID NOs:164-196. In some embodiments, the guide RNAs disclosed herein comprise a guide sequence that is at least 95%, 90%, 85%, 80%, or 75% identical to a sequence selected from the group consisting of SEQ ID NOs:98-119. In some embodiments, the guide RNAs disclosed herein comprise a guide sequence that is at least 95%, 90%, 85%, 80%, or 75% identical to a sequence selected from Table 1. The gRNA may comprise one or more of the guide sequences shown in Table 1. The gRNA may comprise one or more of the sequences shown in Tables 1, 7 and 9. The gRNA may comprise one or more of the sequences shown in Tables 2, 8, and 10. The guide RNA may comprise one or more of SEQ ID NOs: 2-33. The gRNA may comprise one or more of SEQ ID NOs: 164-196. The gRNA may comprise one or more of SEQ ID NOs: 98-119.

In some embodiments, the guide RNAs disclosed herein comprise a guide sequence having at least 15, 16, 17, 18, 19, or 20 contiguous nucleotides of a sequence selected from the group consisting of SEQ ID NOs:2-33. In some embodiments, the guide RNAs disclosed herein comprise a guide sequence having at least 15, 16, 17, 18, 19, or 20 contiguous nucleotides of a sequence selected from the group consisting of SEQ ID NOs: 164-196. In some embodiments, the guide RNAs disclosed herein comprise a guide sequence having at least 15, 16, 17, 18, 19, or 20 contiguous nucleotides of a sequence selected from the group consisting of SEQ ID NOs: 98-119. In some embodiments, the guide RNAs disclosed herein comprise a guide sequence having at least 15, 16, 17, 18, 19, or 20 contiguous nucleotides of a sequence selected from the Table 1. In some embodiments, the guide RNA comprises a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.

In some embodiments, the albumin guide RNA (gRNA) comprises a guide sequence chosen from: a) a sequence that is at least 95%, 90%, 85%, 80%, or 75% identical to a sequence selected from the group consisting of SEQ ID NOs: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33. In some embodiments, the albumin guide RNA comprises a sequence selected from the group consisting of SEQ ID NO: 2, 8, 13, 19, 28, 29, 31, 32, 33. In some embodiments, the guide RNA comprises a sequence selected from the group consisting of SEQ ID NO: 4, 13, 17, 19, 27, 28, 30, and 31.

In some embodiments, the guide RNAs disclosed herein bind to a region upstream of a propospacer adjacent motif (PAM). As would be understood by those of skill in the art, the PAM sequence occurs on the strand opposite to the strand that contains the target sequence. That is, the PAM sequence is on the complement strand of the target strand (the strand that contains the target sequence to which the guide RNA binds). In some embodiments, the PAM is selected from the group consisting of NGG, NNGRRT, NNGRR(N), NNAGAAW, NNNNG(A/C)TT, and NNNNRYAC. In some embodiments, the PAM is NGG.

In some embodiments, the guide RNA sequences provided herein are complementary to a sequence adjacent to a PAM sequence.

In some embodiments, the guide RNA sequence comprises a sequence that is complementary to a sequence within a genomic region selected from Table 1 according to coordinates in human reference genome hg38. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides from within a genomic region selected from Table 1. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides spanning a genomic region selected from Table 1.

The guide RNAs disclosed herein mediate a target-specific cutting resulting in a double-stranded break (DSB). The guide RNAs disclosed herein mediate a target-specific cutting resulting in a single-stranded break (SSB or nick).

In some embodiments, the guide RNAs disclosed herein mediate target-specific cutting by an RNA-guided DNA binding agent (e.g., a Cas nuclease, as disclosed herein), resulting in insertion of a heterologous nucleic acid within intron 1 of an albumin gene. In some embodiments, the guide RNA and/or cutting at the cut site results in a rate of between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% insertion of a heterologous gene. In some embodiments, the guide RNA and/or cutting results in at least 20%, at least 30%, at least 40%, at least 50%, at least 60%, at least 70%, at least 80%, at least 90% insertion of a heterologous nucleic acid. Insertion rates can be measured in vitro or in vivo. For example, in some embodiments, rate of insertion can be determined by detecting and measuring the inserted nucleic acid within a population of cells, and calculating a percentage of the population that contains the inserted nucleic acid. Methods of measuring insertion rates are known and available in the art. In some embodiments, the guide RNA allows between 5 and 10%, 10 and 15%, 15 and 20%, 20 and 25%, 25 and 30%, 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, 95 and 99% or more increased expression of a heterologous gene. Increased expression of a heterologous gene can be measured in vitro or in vivo. For example, in some embodiments, increased expression can be determined by detecting and measuring the heterologous polypeptide level and comparing the level against the polypeptide level before, e.g., treating the cells or administration to a subject. In some embodiments, increased expression can be determined by detecting and measuring the heterologous polypeptide level and comparing the level against a known polypeptide level, e.g., a normal level of the polypeptide in a healthy subject.

Each of the guide sequences shown in Table 1 may further comprise additional nucleotides to form a crRNA, e.g., with the following exemplary nucleotide sequence following the guide sequence at its 3′ end: GUUUUAGAGCUAUGCUGUUUUG (SEQ ID NO: 300) in 5′ to 3′ orientation. Genomic coordinates are according to human reference genome hg38. In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUUGA AAAAGUGGCACCGAGUCGGUGCUUUU (SEQ ID NO: 301) or GUUUUAGAGCUAGAAAUAGCAAGUUAAAAUAAGGCUAGUCCGUUAUCAACUUGA AAAAGUGGCACCGAGUCGGUGC (SEQ ID NO: 302) in 5′ to 3′ orientation.

TABLE 1
Human guide RNA sequences and chromosomal coordinates

| | | | SEQ ID |
|---|---|---|---|
| Guide ID | Guide Sequence | Genomic Coordinates | NO: |
| G009844 | GAGCAACCUCACUCUUGUCU | chr4:73405113-73405133 | 2 |
| G009851 | AUGCAUUUGUUUCAAAAUAU | chr4:73405000-73405020 | 3 |
| G009852 | UGCAUUUGUUUCAAAAUAUU | chr4:73404999-73405019 | 4 |
| G009857 | AUUUAUGAGAUCAACAGCAC | chr4:73404761-73404781 | 5 |
| G009858 | GAUCAACAGCACAGGUUUUG | chr4:73404753-73404773 | 6 |
| G009859 | UUAAAUAAAGCAUAGUGCAA | chr4:73404727-73404747 | 7 |
| G009860 | UAAAGCAUAGUGCAAUGGAU | chr4:73404722-73404742 | 8 |
| G009861 | UAGUGCAAUGGAUAGGUCUU | chr4:73404715-73404735 | 9 |
| G009866 | UACUAAAACUUUAUUUUACU | chr4:73404452-73404472 | 10 |
| G009867 | AAAGUUGAACAAUAGAAAAA | chr4:73404418-73404438 | 11 |
| G009868 | AAUGCAUAAUCUAAGUCAAA | chr4:73405013-73405033 | 12 |
| G009874 | UAAUAAAAUUCAAACAUCCU | chr4:73404561-73404581 | 13 |
| G012747 | GCAUCUUUAAAGAAUUAUUU | chr4:73404478-73404498 | 14 |
| G012748 | UUUGGCAUUUAUUUCUAAAA | chr4:73404496-73404516 | 15 |
| G012749 | UGUAUUUGUGAAGUCUUACA | chr4:73404529-73404549 | 16 |
| G012750 | UCCUAGGUAAAAAAAAAAAA | chr4:73404577-73404597 | 17 |
| G012751 | UAAUUUUCUUUUGCGCACUA | chr4:73404620-73404640 | 18 |
| G012752 | UGACUGAAACUUCACAGAAU | chr4:73404664-73404684 | 19 |
| G012753 | GACUGAAACUUCACAGAAUA | chr4:73404665-73404685 | 20 |
| G012754 | UUCAUUUUAGUCUGUCUUCU | chr4:73404803-73404823 | 21 |
| G012755 | AUUAUCUAAGUUUGAAUAUA | chr4:73404859-73404879 | 22 |
| G012756 | AAUUUUUAAAAUAGUAUUCU | chr4:73404897-73404917 | 23 |
| G012757 | UGAAUUAUUCUUCUGUUUAA | chr4:73404924-73404944 | 24 |
| G012758 | AUCAUCCUGAGUUUUUCUGU | chr4:73404965-73404985 | 25 |
| G012759 | UUACUAAAACUUUAUUUUAC | chr4:73404453-73404473 | 26 |
| G012760 | ACCUUUUUUUUUUUUUACCU | chr4:73404581-73404601 | 27 |
| G012761 | AGUGCAAUGGAUAGGUCUUU | chr4:73404714-73404734 | 28 |
| G012762 | UGAUUCCUACAGAAAAACUC | chr4:73404973-73404993 | 29 |
| G012763 | UGGGCAAGGGAAGAAAAAAA | chr4:73405094-73405114 | 30 |
| G012764 | CCUCACUCUUGUCUGGGCAA | chr4:73405107-73405127 | 31 |
| G012765 | ACCUCACUCUUGUCUGGGCA | chr4:73405108-73405128 | 32 |
| G012766 | UGAGCAACCUCACUCUUGUC | chr4:73405114-73405134 | 33 |

The guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the sgRNA comprises one or more linkages between nucleotides that is not a phosphodiester linkage.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence shown in Table 1, and a second RNA molecule comprising a trRNA. The first and second RNA molecules may not be covalently linked, but may form a RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 1 covalently linked to a trRNA. The sgRNA may comprise 15, 16, 17, 18, 19, or 20 contiguous nucleotides of a guide sequence shown in Table 1. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, the target sequence or region within intron 1 of a human albumin locus (e.g., nucleotide sequences corresponding to a region within SEQ ID NO: 1) may be complementary to the guide sequence of the guide RNA. In some embodiments, the degree of complementarity or identity between a guide sequence of a guide RNA and its corresponding target sequence may be at least 75%, 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, 4, or 5 mismatches, where the total length of the guide sequence is about 20, or 20. In some embodiments, the target sequence and the guide sequence of the gRNA may contain 1-4 mismatches where the guide sequence is about 20, or 20 nucleotides.

As described and exemplified herein, the albumin guide RNAs can be used to insert and express a heterologous gene (e.g., a transgene) at intron 1 of an albumin gene. Thus, in some embodiments, the present disclosure includes compositions comprising one or more guide RNA (gRNA) comprising guide sequences that direct a RNA-guided DNA binding agent (e.g., Cas9) to a target DNA sequence in an albumin gene.

In some embodiments, a composition or formulation disclosed herein comprises an mRNA comprising an open reading frame (ORF) encoding an RNA-guided DNA binding agent, such as a Cas nuclease as described herein. As described below, the mRNA comprising a Cas nuclease may comprise a Cas9 nuclease, such as an S. pyogenes Cas9 nuclease having cleavase, nickase, and/or site-specific DNA binding activity. In some embodiments, the ORF encoding an RNA-guided DNA nuclease is a “modified RNA-guided DNA binding agent ORF” or simply a “modified ORF,” which is used as shorthand to indicate that the ORF is modified.

Cas9 ORFs, including modified Cas9 ORFs, are provided herein and are known in the art. As one example, the Cas9 ORF can be codon optimized, such that coding sequence includes one or more alternative codons for one or more amino acids. An “alternative codon” as used herein refers to variations in codon usage for a given amino acid, and may or may not be a preferred or optimized codon (codon optimized) for a given expression system. Preferred codon usage, or codons that are well-tolerated in a given system of expression, is known in the art. The Cas9 coding sequences, Cas9 mRNAs, and Cas9 protein sequences of WO2013/176772, WO2014/065596, WO2016/106121, and WO2019/067910 are hereby incorporated by reference. In particular, the ORFs and Cas9 amino acid sequences of the table at paragraph [0449] WO2019/067910, and the Cas9 mRNAs and ORFs of paragraphs [0214]-[0234] of WO2019/067910 are hereby incorporated by reference.

In some embodiments, an mRNA comprising an ORF encoding an RNA-guided DNA binding agent, such as a Cas nuclease, is provided, used, or administered.

B. Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs and/or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA. Certain gRNAs comprise at least one modified residue at or near the 5′ end and 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can be a 2′-H, which replaces the 2′ hydroxyl group with a hydrogen. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety.

In some embodiments, the sgRNA of the present disclosure comprises the modification patterns shown below in Table 2. “Full Sequence” in Table 2 refers to an sgRNA sequence for each of the guides listed in Table 1. “Full Sequence Modified” shows a modification pattern for each sgRNA.

TABLE 2
sgRNA and modification patterns to sgRNA of human albumin guide sequences

| | | SEQ | | SEQ |
|---|---|---|---|---|
| Guide | | ID | | ID |
| ID | Full Sequence | NO: | Full Sequence Modified | NO: |
| G009844 | GAGCAACCUCACUCUUGUCUGUU | 34 | mG*mA*mG*CAACCUCACUCU | 66 |
| | UUAGAGCUAGAAAUAGCAAGUUA | | UGUCUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009851 | AUGCAUUUGUUUCAAAAUAUGUU | 35 | mA*mU*mG*CAUUUGUUUCAA | 67 |
| | UUAGAGCUAGAAAUAGCAAGUUA | | AAUAUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009852 | UGCAUUUGUUUCAAAAUAUUGUU | 36 | mU*mG*mC*AUUUGUUUCAAA | 68 |
| | UUAGAGCUAGAAAUAGCAAGUUA | | AUAUUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009857 | AUUUAUGAGAUCAACAGCACGUU | 37 | mA*mU*mU*UAUGAGAUCAAC | 69 |
| | UUAGAGCUAGAAAUAGCAAGUUA | | AGCACGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009858 | GAUCAACAGCACAGGUUUUGGUU | 38 | mG*mA*mU*CAACAGCACAGG | 70 |
| | UUAGAGCUAGAAAUAGCAAGUUA | | UUUUGGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009859 | UUAAAUAAAGCAUAGUGCAAGUU | 39 | mU*mU*mA*AAUAAAGCAUAG | 71 |
| | UUAGAGCUAGAAAUAGCAAGUUA | | UGCAAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009860 | UAAAGCAUAGUGCAAUGGAUGUU | 40 | mU*mA*mA*AGCAUAGUGCAA | 72 |
| | UUAGAGCUAGAAAUAGCAAGUUA | | UGGAUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009861 | UAGUGCAAUGGAUAGGUCUUGUU | 41 | mU*mA*mG*UGCAAUGGAUAG | 73 |
| | UUAGAGCUAGAAAUAGCAAGUUA | | GUCUUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009866 | UACUAAAACUUUAUUUUACUGUU | 42 | mU*mA*mC*UAAAACUUUAUU | 74 |
| | UUAGAGCUAGAAAUAGCAAGUUA | | UUACUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009867 | AAAGUUGAACAAUAGAAAAAGUU | 43 | mA*mA*mA*GUUGAACAAUAG | 75 |
| | UUAGAGCUAGAAAUAGCAAGUUA | | AAAAAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009868 | AAUGCAUAAUCUAAGUCAAAGUU | 44 | mA*mA*mU*GCAUAAUCUAAG | 76 |
| | UUAGAGCUAGAAAUAGCAAGUUA | | UCAAAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009874 | UAAUAAAAUUCAAACAUCCUGUU | 45 | mU*mA*mA*UAAAAUUCAAAC | 77 |
| | UUAGAGCUAGAAAUAGCAAGUUA | | AUCCUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012747 | GCAUCUUUAAAGAAUUAUUUGUU | 46 | mG*mC*mA*UCUUUAAAGAAU | 78 |
| | UUAGAGCUAGAAAUAGCAAGUUA | | UAUUUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012748 | UUUGGCAUUUAUUUCUAAAAGUU | 47 | mU*mU*mU*GGCAUUUAUUUC | 79 |
| | UUAGAGCUAGAAAUAGCAAGUUA | | UAAAAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012749 | UGUAUUUGUGAAGUCUUACAGUU | 48 | mU*mG*mU*AUUUGUGAAGUC | 80 |
| | UUAGAGCUAGAAAUAGCAAGUUA | | UUACAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012750 | UCCUAGGUAAAAAAAAAAAAGUU | 49 | mU*mC*mC*UAGGUAAAAAAA | 81 |
| | UUAGAGCUAGAAAUAGCAAGUUA | | AAAAAGUUUUAGAmGmCmUm | |
| | AAAUAAGGCUAGUCCGUUAUCAA | | AmGmAmAmAmUmAmGmCAAG | |
| | | | UUAAAAUAAGGCUAGUCCGUU | |
| | CUUGAAAAAGUGGCACCGAGUCG | | AUCAmAmCmUmUmGmAmAmA | |
| | GUGCUUUU | | mAmAmGmUmGmGmCmAmCmC | |
| | | | mGmAmGmUmCmGmGmUmGmC | |
| | | | mU*mU*mU*mU | |
| G012751 | UAAUUUUCUUUUGCGCACUAGUU | 50 | mU*mA*mA*UUUUCUUUUGCG | 82 |
| | UUAGAGCUAGAAAUAGCAAGUUA | | CACUAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012752 | UGACUGAAACUUCACAGAAUGUU | 51 | mU*mG*mA*CUGAAACUUCAC | 83 |
| | UUAGAGCUAGAAAUAGCAAGUUA | | AGAAUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012753 | GACUGAAACUUCACAGAAUAGUU | 52 | mG*mA*mC*UGAAACUUCACA | 84 |
| | UUAGAGCUAGAAAUAGCAAGUUA | | GAAUAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012754 | UUCAUUUUAGUCUGUCUUCUGUU | 53 | mU*mU*mC*AUUUUAGUCUGU | 85 |
| | UUAGAGCUAGAAAUAGCAAGUUA | | CUUCUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012755 | AUUAUCUAAGUUUGAAUAUAGUU | 54 | mA*mU*mU*AUCUAAGUUUGA | 86 |
| | UUAGAGCUAGAAAUAGCAAGUUA | | AUAUAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012756 | AAUUUUUAAAAUAGUAUUCUGUU | 55 | mA*mA*mU*UUUUAAAAUAGU | 87 |
| | UUAGAGCUAGAAAUAGCAAGUUA | | AUUCUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012757 | UGAAUUAUUCUUCUGUUUAAGUU | 56 | mU*mG*mA*AUUAUUCUUCUG | 88 |
| | UUAGAGCUAGAAAUAGCAAGUUA | | UUUAAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012758 | AUCAUCCUGAGUUUUUCUGUGUU | 57 | mA*mU*mC*AUCCUGAGUUUU | 89 |
| | UUAGAGCUAGAAAUAGCAAGUUA | | UCUGUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012759 | UUACUAAAACUUUAUUUUACGUU | 58 | mU*mU*mA*CUAAAACUUUAU | 90 |
| | UUAGAGCUAGAAAUAGCAAGUUA | | UUUACGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012760 | ACCUUUUUUUUUUUUUACCUGUU | 59 | mA*mC*mC*UUUUUUUUUUUU | 91 |
| | UUAGAGCUAGAAAUAGCAAGUUA | | UACCUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012761 | AGUGCAAUGGAUAGGUCUUUGUU | 60 | mA*mG*mU*GCAAUGGAUAGG | 92 |
| | UUAGAGCUAGAAAUAGCAAGUUA | | UCUUUGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012762 | UGAUUCCUACAGAAAAACUCGUU | 61 | mU*mG*mA*UUCCUACAGAAA | 93 |
| | UUAGAGCUAGAAAUAGCAAGUUA | | AACUCGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012763 | UGGGCAAGGGAAGAAAAAAAGUU | 62 | mU*mG*mG*GCAAGGGAAGAA | 94 |
| | UUAGAGCUAGAAAUAGCAAGUUA | | AAAAAGUUUUAGAmGmCmUm | |
| | AAAUAAGGCUAGUCCGUUAUCAA | | AmGmAmAmAmUmAmGmCAAG | |
| | CUUGAAAAAGUGGCACCGAGUCG | | UUAAAAUAAGGCUAGUCCGUU | |
| | GUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G012764 | CCUCACUCUUGUCUGGGCAAGUU | 63 | mC*mC*mU*CACUCUUGUCUGG | 95 |
| | UUAGAGCUAGAAAUAGCAAGUUA | | GCAAGUUUUAGAmGmCmUmA | |
| | AAAUAAGGCUAGUCCGUUAUCAA | | mGmAmAmAmUmAmGmCAAGU | |
| | CUUGAAAAAGUGGCACCGAGUCG | | UAAAAUAAGGCUAGUCCGUUA | |
| | GUGCUUUU | | UCAmAmCmUmUmGmAmAmAm | |
| | | | AmAmGmUmGmGmCmAmCmCm | |
| | | | GmAmGmUmCmGmGmUmGmCm | |
| | | | U*mU*mU*mU | |
| G012765 | ACCUCACUCUUGUCUGGGCAGUU | 64 | mA*mC*mC*UCACUCUUGUCUG | 96 |
| | UUAGAGCUAGAAAUAGCAAGUUA | | GGCAGUUUUAGAmGmCmUmA | |
| | AAAUAAGGCUAGUCCGUUAUCAA | | mGmAmAmAmUmAmGmCAAGU | |
| | CUUGAAAAAGUGGCACCGAGUCG | | UAAAAUAAGGCUAGUCCGUUA | |
| | GUGCUUUU | | UCAmAmCmUmUmGmAmAmAm | |
| | | | AmAmGmUmGmGmCmAmCmCm | |
| | | | GmAmGmUmCmGmGmUmGmCm | |
| | | | U*mU*mU*mU | |
| G012766 | UGAGCAACCUCACUCUUGUCGUU | 65 | mU*mG*mA*GCAACCUCACUCU | 97 |
| | UUAGAGCUAGAAAUAGCAAGUUA | | UGUCGUUUUAGAmGmCmUmA | |
| | AAAUAAGGCUAGUCCGUUAUCAA | | mGmAmAmAmUmAmGmCAAGU | |
| | CUUGAAAAAGUGGCACCGAGUCG | | UAAAAUAAGGCUAGUCCGUUA | |
| | GUGCUUUU | | UCAmAmCmUmUmGmAmAmAm | |
| | | | AmAmGmUmGmGmCmAmCmCm | |
| | | | GmAmGmUmCmGmGmUmGmCm | |
| | | | U*mU*mU*mU | |

In some embodiments, the modified sgRNA comprises the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 350), where “N” may be any natural or non-natural nucleotide, and wherein the totality of N's comprise an albumin intron 1 guide sequence as described in Table 1. For example, encompassed herein is SEQ ID NO: 350, which omits the N's from SEQ ID NO: 350 but includes the modified conserved portion of a gRNA.

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

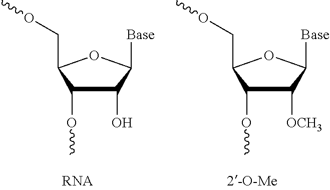

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

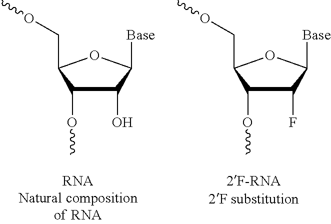

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S- into a nonbridging phosphate oxygen, generating a PS bond in lieu of a phosphodiester bond:

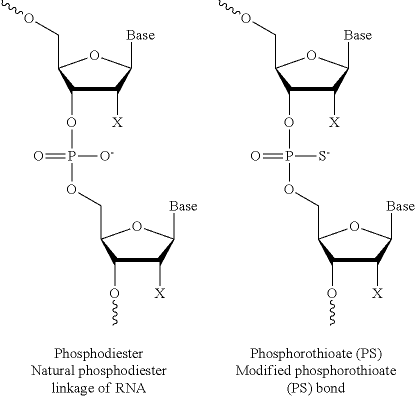

Abasic nucleotides refer to those which lack nitrogenous bases. The figure below depicts an oligonucleotide with an abasic (also known as apurinic) site that lacks a base:

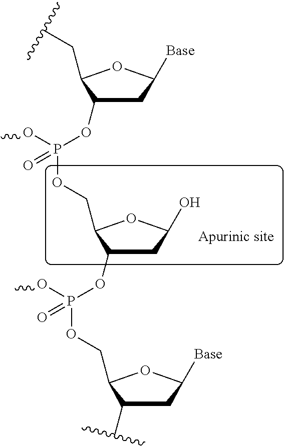

Inverted bases refer to those with linkages that are inverted from the normal 5′ to 3′ linkage (i.e., either a 5′ to 5′ linkage or a 3′ to 3′ linkage). For example:

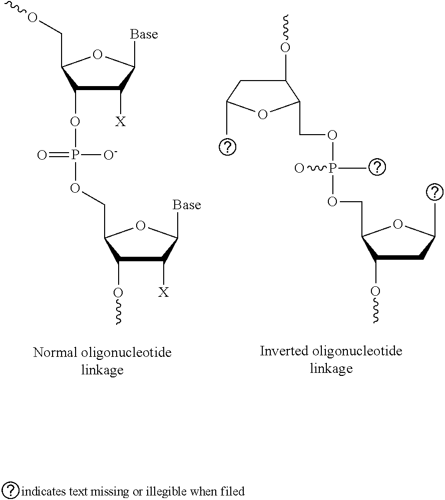

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in SEQ ID No: 350, where N is any natural or non-natural nucleotide, and where the totality of the N's comprise a guide sequence that directs a nuclease to a target sequence in human albumin intron 1, e.g., as shown in Table 1.

In some embodiments, the guide RNA comprises a sgRNA shown in any one of SEQ ID NOs: 34-97 or 120-163. In some embodiments, the guide RNA comprises a sgRNA shown in any one of SEQ ID NOs: 197-229. In some embodiments, the guide RNA comprises a sgRNA comprising any one of the guide sequences of SEQ ID NOs: 2-33 or 98-119 and the nucleotides of SEQ ID No: 301, wherein the nucleotides of SEQ ID NO: 301 are on the 3′ end of the guide sequence, and wherein the sgRNA may be modified as shown in Table 2 or SEQ ID NO: 350. In some embodiments, the guide RNA comprises a sgRNA comprising any one of the guide sequences of SEQ ID NOs: 2-33 or 197-229 and the nucleotides of SEQ ID NO: 301, wherein the nucleotides of SEQ ID NO: 301 are on the 3′ end of the guide sequence, and wherein the sgRNA may be modified as shown in Table 2 or SEQ ID NO: 350.

As noted above, in some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

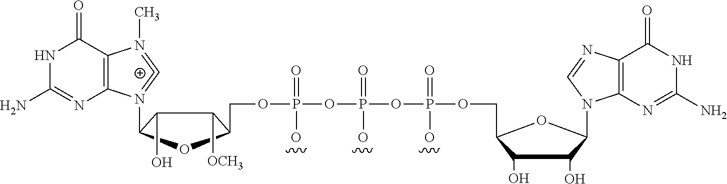

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

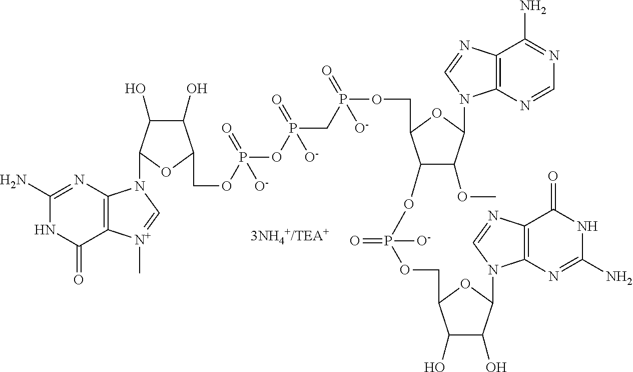 [SMILES: COC1C(OP(=O)([O-])OCC2OC(n3cnc4c(=O)[nH]c(N)nc43)C(O)C2O)C(COP(=O)([O-])CP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

C. RNA-Guided DNA Binding Agent

As described herein, the guide RNAs of the present disclosure are used in conjunction with an RNA-guided DNA binding agent for inserting and expressing a heterologous (exogenous) gene within a genomic locus, such as a safe harbor site, of a host cell. The RNA-guided DNA binding agent may be a protein or a nucleic acid encoding the protein such as an mRNA. In some embodiments, the methods of the present disclosure include the use of a composition that comprises a guide RNA comprising a guide sequence from Table 1 and an RNA-guided DNA binding agent, e.g., a nuclease, such as a Cas nuclease (e.g., Cas9), to form a ribonucleoprotein complex.

In some embodiments, the RNA-guided DNA-binding agent, such as a Cas9 nuclease, has cleavase activity, which can also be referred to as double-strand endonuclease activity. In some embodiments, the RNA-guided DNA-binding agent, such as a Cas9 nuclease, has nickase activity, which can also be referred to as single-strand endonuclease activity. In some embodiments, the RNA-guided DNA-binding agent comprises a Cas nuclease. Examples of Cas nucleases include those of the type II CRISPR systems of S. pyogenes, S. aureus, and other prokaryotes (see, e.g., the list in the next paragraph), and variant or mutant (e.g., engineered, non-naturally occurring, naturally occurring, or other variant) versions thereof. See, e.g., US2016/0312198 A1; US 2016/0312199 A1.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA. In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be linked at the C-terminus of the RNA-guided DNA-binding agent. One or more linkers are optionally included at the fusion site.

D. Donor Construct/Sequences

The compositions and methods described herein include the use of a nucleic acid construct that comprises a sequence encoding a heterologous gene to be inserted into a cut site created by a guide RNA of the present disclosure and an RNA-guided DNA binding agent. As used herein, such a construct is sometimes referred to as a “donor construct/template”. The constructs may encode any expressed nucleic acid (i.e., nucleic acid that can be expressed), for example, DNA, messenger RNA (mRNA), a functional RNA, small interfering RNA (siRNA), microRNA (miRNA), single stranded RNA (ssRNA), long non-coding RNAs, or antisense oligonucleotides.

The compositions and methods described herein include the use of a non-bidirectional or unidirectional construct, e.g., encoding a single transgene, encoding two transgenes in cis, etc. The unidirectional construct may comprise a coding sequence linked to a splice acceptor.

The compositions and methods described herein include the use of a bidirectional construct described herein comprising at least two nucleic acid segments in cis, wherein one segment (the first segment) comprises a coding sequence or transgene, while the other segment (the second segment) comprises a sequence wherein the complement of the sequence encodes a transgene. A bidirectional construct may comprise a first coding sequence that encodes a heterologous gene linked to a splice acceptor and a second coding sequence wherein the complement encodes a heterologous gene in the other orientation, also linked to a splice acceptor.

In some embodiments, the constructs disclosed herein comprise a splice acceptor site on either or both ends of the construct, e.g., 5′ of an open reading frame in the first and/or second segments, or 5′ of one or both transgene sequences. In some embodiments, the splice acceptor site comprises NAG. In further embodiments, the splice acceptor site consists of NAG. In some embodiments, the splice acceptor is an albumin splice acceptor, e.g., an albumin splice acceptor used in the splicing together of exons 1 and 2 of albumin. In some embodiments, the splice acceptor is derived from the human albumin gene. In some embodiments, the splice acceptor is derived from the mouse albumin gene. In some embodiments, the splice acceptor is a F9 (or “FIX”) splice acceptor, e.g., the F9 splice acceptor used in the splicing together of exons 1 and 2 of F9. In some embodiments, the splice acceptor is derived from the human F9 gene. In some embodiments, the splice acceptor is derived from the mouse F9 gene. Additional suitable splice acceptor sites useful in eukaryotes, including artificial splice acceptors are known and can be derived from the art. See, e.g., Shapiro, et al., 1987, Nucleic Acids Res., 15, 7155-7174, Burset, et al., 2001, Nucleic Acids Res., 29, 255-259.

In some embodiments, the polyadenylation tail sequence is encoded, e.g., as a “poly-A” stretch, at the 3′ end of the first and/or second segment. In some embodiments, a polyadenylation tail sequence is provided co-transcriptionally as a result of a polyadenylation signal sequence that is encoded at or near the 3′ end of the first and/or second segment. Methods of designing a suitable polyadenylation tail sequence and/or polyadenylation signal sequence are well known in the art. Suitable splice acceptor sequences are disclosed and exemplified herein, including mouse albumin and human FIX splice acceptor sites. In some embodiments, the polyadenylation signal sequence AAUAAA (SEQ ID NO: 800) is commonly used in mammalian systems, although variants such as UAUAAA (SEQ ID NO: 801) or AU/GUAAA (SEQ ID NO: 802) have been identified. See, e.g., NJ Proudfoot, Genes & Dev. 25(17):1770-82, 2011. In some embodiments, a polyA tail sequence is included. The length of the construct can vary, depending on the size of the gene to be inserted, and can be, for example, from 200 base pairs (bp) to about 5000 bp, such as about 200 bp to about 2000 bp, such as about 500 bp to about 1500 bp. In some embodiments, the length of the DNA donor template is about 200 bp, or is about 500 bp, or is about 800 bp, or is about 1000 base pairs, or is about 1500 base pairs. In other embodiments, the length of the donor template is at least 200 bp, or is at least 500 bp, or is at least 800 bp, or is at least 1000 bp, or is at least 1500 bp.

The construct can be DNA or RNA, single-stranded, double-stranded or partially single- and partially double-stranded and can be introduced into a host cell in linear or circular (e.g., minicircle) form. See, e.g., U.S. Patent Publication Nos. 2010/0047805, 2011/0281361, 2011/0207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters and genes encoding antibiotic resistance. A construct may omit viral elements. Moreover, donor constructs can be introduced as naked nucleic acid, as nucleic acid complexed with an agent such as a liposome or poloxamer, or can be delivered by viruses (e.g., adenovirus, AAV, herpesvirus, retrovirus, lentivirus).

In some embodiments, although not required for expression, the constructs disclosed herein may also include transcriptional or translational regulatory sequences, for example, promoters, enhancers, insulators, internal ribosome entry sites, sequences encoding peptides, and/or polyadenylation signals.

In some embodiments, the constructs comprising a coding sequence for a polypeptide of interest may include one or more of the following modifications: codon optimization (e.g., to human codons) and/or addition of one or more glycosylation sites. See, e.g., McIntosh et al. (2013) Blood (17):3335-44.

In some embodiments, the construct may be inserted so that its expression is driven by the endogenous promoter at the insertion site (e.g., the endogenous albumin promoter when the donor is integrated into the host cell's albumin locus). In such cases, the transgene may lack control elements (e.g., promoter and/or enhancer) that drive its expression (e.g., a promoterless construct). Nonetheless, it will be apparent that in other cases the construct may comprise a promoter and/or enhancer, for example a constitutive promoter or an inducible or tissue specific (e.g., liver- or platelet-specific) promoter that drives expression of the functional protein upon integration. The construct may comprise a sequence encoding a heterologous protein downstream of and operably linked to a signal sequence encoding a signal peptide, e.g., an albumin signal peptide, a signal peptide from a hepatocyte secreted protein. The construct may comprise a sequence encoding a heterologous protein downstream of and operably linked to a signal sequence encoding a signal peptide from the heterologous protein. In some embodiments, the nucleic acid construct works in homology-independent insertion of a nucleic acid that encodes a transgenic protein. In some embodiments, the nucleic acid construct works in non-dividing cells, e.g., cells in which NHEJ, not HR, is the primary mechanism by which double-stranded DNA breaks are repaired. The nucleic acid may be a homology-independent donor construct.

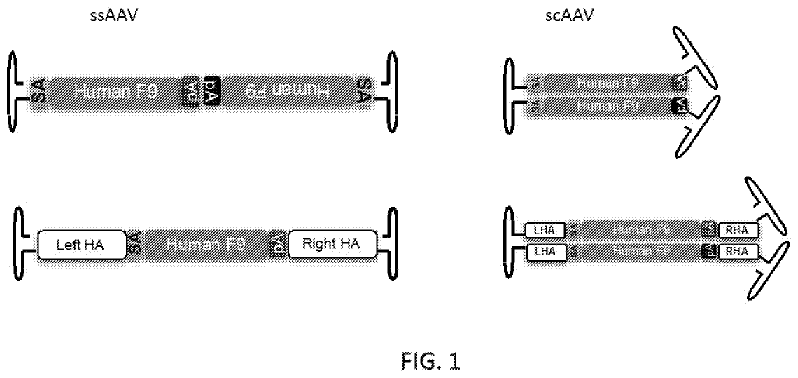

The construct may be a bidirectional nucleic acid constructs comprising at least two nucleic acid segments, wherein one segment (the first segment) comprises a coding sequence that encodes an agent of interest (the coding sequence may be referred to herein as “transgene” or a first transgene), while the other segment (the second segment) comprises a sequence wherein the complement of the sequence encodes an agent of interest, or a second transgene. In some embodiments, a coding sequence encodes a therapeutic agent, such as a polypeptide, functional RNA, or enhancer. The at least two segments can encode identical or different polypeptides or identical or different agents. In some embodiments, the bidirectional constructs disclosed herein comprise at least two nucleic acid segments, wherein one segment (the first segment) comprises a coding sequence that encodes a polypeptide of interest, while the other segment (the second segment) comprises a sequence wherein the complement of the sequence encodes a polypeptide of interest. When used in combination with a gene editing system as described herein, the bidirectionality of the nucleic acid constructs allows the construct to be inserted in either direction (is not limited to insertion in one direction) within a target insertion site, allowing the expression of the polypeptide of interest from either a) a coding sequence of one segment (e.g., the left segment encoding “Human F9” in the upper left ssAAV construct of FIG. 1), or 2) a complement of the other segment (e.g., the complement of the right segment encoding “Human F9” indicated upside down in in the upper left ssAAV construct FIG. 1), thereby enhancing insertion and expression efficiency, as exemplified herein. Targeted cleavage by a gene editing system can facilitate construct integration and/or transgene expression. Various known gene editing systems can be used in the practice of the present disclosure, including, e.g., site-specific DNA cleavage systems including a CRISPR/Cas system; zinc finger nuclease (ZFN) system; or transcription activator-like effector nuclease (TALEN) system.

In some embodiments, the bidirectional nucleic acid construct does not comprise a promoter that drives the expression of the agent or polypeptide. For example, the expression of the polypeptide is driven by a promoter of the host cell (e.g., the endogenous albumin promoter when the transgene is integrated into a host cell's albumin locus). In some embodiments, the bidirectional nucleic acid construct includes a first segment and a second segment, each having a splice acceptor upstream of a transgene. In certain embodiments, the splice acceptor is compatible with the splice donor sequence of the host cell's safe harbor site, e.g. the splice donor of intron 1 of a human albumin gene.

In some embodiments, the bidirectional nucleic acid construct comprises a first segment comprising a coding sequence for a polypeptide and a second segment comprising a reverse complement of a coding sequence of the polypeptide. The same is true for non-polypeptide agents. Thus, the coding sequence in the first segment is capable of expressing a polypeptide, while the complement of the reverse complement in the second segment is also capable of expressing the polypeptide. As used herein, “coding sequence” when referring to the second segment comprising a reverse complement sequence refers to the complementary (coding) strand of the second segment (i.e., the complement coding sequence of the reverse complement sequence in the second segment).

In some embodiments, the coding sequence that encodes Polypeptide A in the first segment is less than 100% complementary to the reverse complement of a coding sequence that also encodes Polypeptide A. That is, in some embodiments, the first segment comprises a coding sequence (1) for Polypeptide A, and the second segment is a reverse complement of a coding sequence (2) for Polypeptide A, wherein the coding sequence (1) is not identical to the coding sequence (2). For example, coding sequence (1) and/or coding sequence (2) that encodes for Polypeptide A can utilize different codons. In some embodiments, one or both sequences can be codon optimized, such that coding sequence (1) and the reverse complement of coding sequence (2) possess 100% or less than 100% complementarity. In some embodiments, the coding sequence of the second segment encodes the polypeptide using one or more alternative codons for one or more amino acids of the same polypeptide encoded by the coding sequence in the first segment. An “alternative codon” as used herein refers to variations in codon usage for a given amino acid, and may or may not be a preferred or optimized codon (codon optimized) for a given expression system. Preferred codon usages, or codons that are well-tolerated in a given system of expression, are known in the art.

In some embodiments, the second segment comprises a reverse complement sequence that adopts different codon usage from that of the coding sequence of the first segment in order to reduce hairpin formation. Such a reverse complement forms base pairs with fewer than all nucleotides of the coding sequence in the first segment, yet it optionally encodes the same polypeptide. In such cases, the coding sequence, e.g. for Polypeptide A, of the first segment many be homologous to, but not identical to, the coding sequence, e.g. for Polypeptide A of the second half of the bidirectional construct. In some embodiments, the second segment comprises a reverse complement sequence that is not substantially complementary (e.g., not more than 70% complementary) to the coding sequence in the first segment. In some embodiments, the second segment comprises a reverse complement sequence that is highly complementary (e.g., at least 90% complementary) to the coding sequence in the first segment. In some embodiments, the second segment comprises a reverse complement sequence having at least about 30%, about 35%, about 40%, about 45%, about 50%, about 55%, about 60%, about 65%, about 70%, about 75%, about 80%, about 85%, about 90%, about 95%, about 97%, or about 99% complementarity to the coding sequence in the first segment.

In some embodiments, the second segment comprises a reverse complement sequence having 100% complementarity to the coding sequence in the first segment. That is, the sequence in the second segment is a perfect reverse complement of the coding sequence in the first segment. By way of example, the first segment comprises a hypothetical sequence 5′ CTGGACCGA 3′ (SEQ ID NO: 500) and the second segment comprises the reverse complement of SEQ ID NO: 1—i.e., 5′ TCGGTCCAG 3′ (SEQ ID NO: 502).

In some embodiments, the bidirectional nucleic acid construct comprises a first segment comprising a coding sequence for a polypeptide or agent (e.g. a first polypeptide) and a second segment comprising a reverse complement of a coding sequence of a polypeptide or agent (e.g. a second polypeptide). In some embodiments, the first polypeptide and the second polypeptide are the same, as described above. In some embodiments, the first therapeutic agent and the second therapeutic agent are the same, as described above. In some embodiments, the first polypeptide and the second polypeptides are different. In some embodiments, the first therapeutic agent and the second therapeutic agent are different. For example, the first polypeptide is Polypeptide A and the second polypeptide is Polypeptide B. As a further example, the first polypeptide is Polypeptide A and the second polypeptide is a variant (e.g., a fragment (such as a functional fragment), mutant, fusion (including addition of as few as one amino acid at a polypeptide terminus), or combinations thereof) of Polypeptide A. A coding sequence that encodes a polypeptide may optionally comprise one or more additional sequences, such as sequences encoding amino- or carboxy-terminal amino acid sequences such as a signal sequence, label sequence (e.g. HiBit), or heterologous functional sequence (e.g. nuclear localization sequence (NLS) or self-cleaving peptide) linked to the polypeptide. A coding sequence that encodes a polypeptide may optionally comprise sequences encoding one or more amino-terminal signal peptide sequences. Each of these additional sequences can be the same or different in the first segment and second segment of the construct.

The bidirectional construct described herein can be used to express any polypeptide according to the methods disclosed herein. In some embodiments, the polypeptide is a secreted polypeptide. In some embodiments, the polypeptide is one in which its function is normally effected (e.g., functionally active) as a secreted polypeptide. A “secreted polypeptide” as used herein refers to a protein that is secreted by the cell and/or is functionally active as a soluble extracellular protein.

In some embodiments, the polypeptide is an intracellular polypeptide. In some embodiments, the polypeptide is one in which its function is normally effected (e.g., functionally active) inside a cell. An “intracellular polypeptide” as used herein refers to a protein that is not secreted by the cell, including soluble cytosolic polypeptides. In some embodiments, the polypeptide is a wild-type polypeptide.

In some embodiments, the polypeptide is a liver protein or variant thereof. As used herein, a “liver protein” is a protein that is, e.g., endogenously produced in the liver and/or functionally active in the liver. In some embodiments, the liver protein is a circulating protein produced by the liver or a variant thereof. In some embodiments, the liver protein is a protein that is functionally active in the liver or a variant thereof. In some embodiments, the liver protein exhibits an elevated expression in liver compared to one or more other tissue types. In some embodiments, the polypeptide is a non-liver protein.

In some embodiments, the bidirectional nucleic acid construct is linear. For example, the first and second segments are joined in a linear manner through a linker sequence. In some embodiments, the 5′ end of the second segment that comprises a reverse complement sequence is linked to the 3′ end of the first segment. In some embodiments, the 5′ end of the first segment is linked to the 3′ end of the second segment that comprises a reverse complement sequence. In some embodiments, the linker sequence is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 500, 1000, 1500, 2000 or more nucleotides in length. As would be appreciated by those of skill in the art, other structural elements in addition to, or instead of a linker sequence, can be inserted between the first and second segments.

The constructs disclosed herein can be modified to include any suitable structural feature as needed for any particular use and/or that confers one or more desired function. In some embodiments, the bidirectional nucleic acid construct disclosed herein does not comprise a homology arm. In some embodiments, the constructs, e.g. bidirectional nucleic acid constructs, are capable of insertion into a genomic locus by non-homologous end joining (NHEJ). In some embodiments, constructs disclosed herein are homology-independent donor constructs. In some embodiments, owing in part to the bidirectional function of the nucleic acid construct, the bidirectional construct can be inserted into a genomic locus in either direction (orientation) as described herein to allow for efficient insertion and/or expression of a polypeptide of interest.

In some embodiments, the composition described herein comprises one or more internal ribosome entry site (IRES). First identified as a feature of Picorna virus RNA, IRES plays an important role in initiating protein synthesis in absence of the 5′ cap structure. An IRES may act as the sole ribosome binding site, or may serve as one of multiple ribosome binding sites of polynucleotides. Constructs containing more than one functional ribosome binding site may encode several peptides or polypeptides that are translated independently by the ribosomes (“multicistronic nucleic acid molecules”). Alternatively, constructs may comprise an IRES in order to express a heterologous protein which is not fused to an endogenous polypeptide (i.e. an albumin signal peptide). Examples of IRES sequences that can be utilized include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In some embodiments, the nucleic acid construct comprises a sequence encoding a self cleaving peptide such as a 2A sequence or a 2A-like sequence. The self cleaving peptide may be a P2A peptide, a T2A peptide, or the like. In some embodiments, the self cleaving peptide is located upstream of the polypeptide of interest. In one embodiment, the sequence encoding the 2A peptide may be used to separate the coding region of two or more polypeptides of interest. In another embodiment, this sequence may be used to separate the coding sequence from the construct and the coding sequence from the endogenous locus (i.e. endogenous albumin signal sequence). As a non-limiting example, the sequence encoding the 2A peptide may be between region A and region B (A-2A-B). The presence of the 2A peptide would result in the cleavage of one long protein into protein A, protein B and the 2A peptide. Protein A and protein B may be the same or different polypeptides of interest.

In some embodiments, one or both of the first and second segment comprises a polyadenylation tail sequence and/or a polyadenylation signal sequence downstream of an open reading frame. In some embodiments, the polyadenylation tail sequence is encoded, e.g., as a “poly-A” stretch, at the 3′ end of the first and/or second segment.

### III. Delivery Methods

The guide RNAs disclosed herein can be delivered to a host cell or subject, in vivo or ex vivo, using various known and suitable methods available in the art. The guide RNAs can be delivered together (individually or combined) with a RNA-guided DNA-binding agent such as Cas or nucleic acid encoding a Cas9 (e.g., Cas9 or a nucleic acid encoding a Cas9) and a construct that comprises a sequence encoding a heterologous gene to be inserted into a cut site created by a guide RNA of the present disclosure, as described herein.

Conventional viral and non-viral based gene delivery methods can be used to introduce the guide RNA disclosed herein as well as the RNA-guided DNA binding agent and donor construct in cells (e.g., mammalian cells) and target tissues. As further provided herein, non-viral vector delivery systems nucleic acids such as non-viral vectors, plasmid vectors, and, e.g naked nucleic acid, and nucleic acid complexed with a delivery vehicle such as a liposome, lipid nanoparticle (LNP), or poloxamer. Viral vector delivery systems include DNA and RNA viruses.

Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by AmaxaBiosystems (Cologne, Germany), Maxcyte, Inc. (Rockville, Md.), BTX Molecular Delivery Systems (Holliston, Ma.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). The preparation of lipid:nucleic acid complexes, including targeted liposomes such as immunolipid complexes, is well known in the art, and as described herein.

Various delivery systems (e.g., vectors, liposomes, LNPs) containing the guide RNAs, RNA-guided DNA binding agent, and donor construct, singly or in combination, can also be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and well known to those of skill in the art.

In some embodiments, the guide RNA compositions described herein, alone or encoded on one or more vectors, are formulated in or administered via a lipid nanoparticle; see e.g., PCT/US2017/024973 the contents of which are hereby incorporated by reference in their entirety. Any lipid nanoparticle (LNP) formulation known to those of skill in the art to be capable of delivering nucleotides to subjects may be utilized with the guide RNAs described herein, as well as either mRNA encoding an RNA-guided DNA binding agent such as Cas or Cas9, or an RNA-guided DNA binding agent such as Cas or Cas9 protein itself.

In some embodiments, the guide RNAs disclosed herein can be delivered to a host cell (in vitro or in vivo) delivered via an LNP. In some embodiments, the gRNA/LNP is also associated with an RNA-guided DNA binding agent such as Cas9 or an mRNA encoding an RNA-guided DNA binding agent such as Cas9. In some embodiments, the gRNA/LNP is also associated with a donor construct as described herein.

In some embodiments, the present disclosure includes a method for delivering the gRNAs disclosed herein to a cell in vitro, wherein the gRNA is delivered via an LNP. In some embodiments, the gRNA is delivered by a non-LNP means, such as via an AAV system, and an RNA-guided DNA binding agent (e.g., Cas9) or an mRNA encoding a RNA-guided DNA binding agent (e.g., Cas9), and/or a donor construct is delivered by an LNP.

In some embodiments, the present disclosure provides a composition comprising any one of the gRNAs disclosed herein and an LNP. In some embodiments, the composition further comprises a Cas9 or an mRNA encoding Cas9, or another RNA-guided DNA binding agent described herein. In some embodiments, the composition further comprises a donor construct as described herein.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of PCT/US2018/053559 (filed Sep. 28, 2018), WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, any of the guide RNAs described herein, RNA-guided DNA binding agents, and/or donor constructs (e.g., bidirectional constructs) disclosed herein, alone or in combination, whether naked or as part of a vector, is formulated in or administered via a lipid nanoparticle; see e.g., WO/2017/173054 the contents of which are hereby incorporated by reference in their entirety.

Electroporation is also a well-known means for delivery of cargo, and any electroporation methodology may be used for delivery of any one of the gRNAs disclosed herein. In some embodiments, electroporation may be used to deliver any one of the gRNAs disclosed herein, optionally with an RNA-guided DNA binding agent such as Cas9 or an mRNA encoding an RNA-guided DNA binding agent such as Cas9 delivered by the same or different means. In some embodiments, electroporation may be used to deliver any one of the gRNAs disclosed herein and a donor construct as disclosed herein.

In certain embodiments, the present disclosure provides DNA or RNA vectors encoding any of the guide RNAs comprising any one or more of the guide sequences described herein. In certain embodiments, the invention comprises DNA or RNA vectors encoding any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, nucleic acids encoding an RNA-guided DNA binding agent, which can be a nuclease such as Cas9, and a donor construct comprising a heterologous gene. In some embodiments, the vector comprises one or more nucleotide sequence(s) encoding a crRNA, a trRNA, or a crRNA and trRNA, as disclosed herein.

In some embodiments, the vector comprises one or more nucleotide sequence(s) encoding a sgRNA and an mRNA encoding an RNA-guided DNA binding agent, which can be a Cas protein, such as Cas9 or Cpf1. In some embodiments, the vector comprises one or more nucleotide sequence(s) encoding a crRNA, a trRNA, and an mRNA encoding an RNA-guided DNA binding agent, which can be a Cas protein, such as, Cas9 or Cpf1. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector may be circular. In other embodiments, the vector may be linear. In some embodiments, the vector may be delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without a helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector.

In some embodiments, “AAV” refers all serotypes, subtypes, and naturally-occurring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV capside sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV).

In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding an RNA-guided DNA binding agent such as a Cas protein (e.g., Cas9), while a second AAV vector may contain one or more guide sequences.

In some embodiments, the vector may be capable of driving expression of one or more coding sequences in a cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding an RNA-guided DNA binding agent such as a Cas protein (e.g., Cas9) described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the vector may comprise any one or more of the constructs comprising a heterologous gene described herein. In some embodiments, the heterologous gene may be operably linked to at least one transcriptional or translational control sequence. In some embodiments, the heterologous gene may be operably linked to at least one promoter. In some embodiments, the heterologous gene is not linked to a promoter that drives the expression of the heterologous gene.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding the guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA may be located on the same vector comprising the nucleotide sequence encoding an RNA-guided DNA binding agent such as a Cas protein. In some embodiments, expression of the guide RNA and of the RNA-guided DNA binding agent such as a Cas protein may be driven by their own corresponding promoters. In some embodiments, expression of the guide RNA may be driven by the same promoter that drives expression of the RNA-guided DNA binding agent such as a Cas protein. In some embodiments, the guide RNA and the RNA-guided DNA binding agent such as a Cas protein transcript may be contained within a single transcript. For example, the guide RNA may be within an untranslated region (UTR) of the RNA-guided DNA binding agent such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors. In some embodiments, the vector system may further comprise a donor construct as described herein. In some embodiments, the vector system may further comprise nucleic acids that encode a nuclease. In some embodiments, the vector system may further comprise nucleic acids that encode guide RNAs and/or nucleic acid encoding an RNA-guided DNA-binding agent, which can be a Cas protein such as Cas9. In some embodiments, a nucleic acid encoding a guide RNA and/or a nucleic acid encoding an RNA-guided DNA-binding agent or nuclease are each or both on a separate vector from a vector that comprises the donor constructs disclosed herein. In any of the embodiments, the vector system may include other sequences that include, but are not limited to, promoters, enhancers, regulatory sequences, as described herein. In some embodiments, a promoter within the vector system does not drive the expression of a transgene of the donor construct (e.g., bidirectional construct). In some embodiments, the vector system comprises one or more nucleotide sequence(s) encoding a crRNA, a trRNA, or a crRNA and trRNA. In some embodiments, the vector system comprises one or more nucleotide sequence(s) encoding a sgRNA and an mRNA encoding an RNA-guided DNA binding agent, which can be a Cas nuclease (e.g., Cas9). In some embodiments, the vector system comprises one or more nucleotide sequence(s) encoding a crRNA, a trRNA, and an mRNA encoding an RNA-guided DNA binding agent, which can be a Cas nuclease, such as, Cas9. In some embodiments, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The vector system may comprise a nucleic acid comprising or consisting of the crRNA, trRNA, or crRNA and trRNA, wherein the vector system comprises or consists of nucleic acids that are not naturally found together with the crRNA, trRNA, or crRNA and trRNA.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

The vector or vector system may be delivered by liposome, a nanoparticle, an exosome, or a microvesicle. The vector may also be delivered by a lipid nanoparticle (LNP). One or more guide RNA, RNA-binding DNA binding agent (e.g. mRNA), or donor construct comprising a sequence encoding a heterologous protein, individually or in any combination, may be delivered by liposome, a nanoparticle, an exosome, or a microvesicle. One or more guide RNA, RNA-binding DNA binding agent (e.g. mRNA), or donor construct comprising a sequence encoding a heterologous protein, individually or in any combination, may be delivered by LNP. Any of the LNPs and LNP formulations described herein are suitable for delivery of the guides, Cas nuclease (or an mRNA encoding a Cas nuclease), combinations thereof, and/or a construct comprising a heterologous gene. In some embodiments, an LNP composition is encompassed comprising: an RNA component and a lipid component, wherein the lipid component comprises an amine lipid, such as biodegradable, ionizable lipid; and wherein the RNA component comprises a guid RNA and/or an mRNA encoding a Cas nuclease. In some instances, the lipid component comprises biodegradable, ionizable lipid, cholesterol, DSPC, and PEG-DMG.

It will be apparent that a guide RNA disclosed herein, an RNA-guided DNA binding agent (e.g. Cas nuclease or a nucleic acid encoding a Cas nuclease), and a donor construct can be delivered using the same or different systems. For example, the guide RNA, Cas nuclease, and construct can be carried by the same vector (e.g., AAV). Alternatively, the Cas nuclease (as a protein or mRNA) and/or gRNA can be carried by a plasmid or LNP, while the construct can be carried by a vector. Furthermore, the different delivery systems can be administered by the same or different routes.

In some embodiments, the method comprises administering a guide RNA and an RNA-guided DNA binding agent (such as an mRNA encoding a Cas9 nuclease) in an LNP. In further embodiments, the method comprises administering an AAV nucleic acid construct encoding a transgenic protein, such as an bidirectional construct. CRISPR/Cas9 LNP, comprising guide RNA and an mRNA encoding a Cas9, can be administered intravenously. AAV donor construct can be administered intravenously.

The different delivery systems can be delivered in vitro or in vivo simultaneously or in any sequential order. In some embodiments, the donor construct, guide RNA, and Cas nuclease can be delivered in vitro or in vivo simultaneously, e.g., in one vector, two vectors, individual vectors, one LNP, two LNPs, individual LNPs, or a combination thereof. In some embodiments, the donor construct can be delivered in vivo or in vitro, as a vector and/or associated with a LNP, prior to (e.g., about 1, 2, 3, 4, 5, 6, 7, 8, 9, 10, 11, 12, 13, 14, or more days) delivering the guide RNA and/or Cas nuclease, as a vector and/or associated with a LNP singly or together as a ribonucleoprotein (RNP). In some embodiments, the donor construct can be delivered in multiple administerations, e.g., every day, every two days, every three days, every four days, every week, every two weeks, every three weeks, or every four weeks. In some embodiments, the donor construct can be delivered at one-week intervals, e.g., at week 1, week 2, and week 3, etc. As a further example, the guide RNA and Cas nuclease, as a vector and/or associated with a LNP singly or together as a ribonucleoprotein (RNP), can be delivered in vivo or in vitro, prior to (e.g., about 1, 2, 3, 4, 5, 6, 7, 8, 9, 10, 11, 12, 13, 14, or more days) delivering the construct, as a vector and/or associated with a LNP. In some embodiments, the albumin guide RNA can be delivered in multiple administerations, e.g., every day, every two days, every three days, every four days, every week, every two weeks, every three weeks, or every four weeks. In some embodiments, the albumin guide RNA can be delivered at one-week intervals, e.g., at week 1, week 2, and week 3, etc. In some embodiments, the Cas nuclease can be delivered in multiple administerations, e.g., can be delivered every day, every two days, every three days, every four days, every week, every two weeks, every three weeks, or every four weeks. In some embodiments, the Cas nuclease can be delivered at one-week intervals, e.g., at week 1, week 2, and week 3, etc.

### IV. Methods of Use

The gRNAs and associated methods and compositions disclosed herein are useful for efficiently inserting a heterologous (exogenous) gene within intron 1 of a human albumin locus of a host cell. In some embodiments, the present disclosure provides a method of inserting a heterologous gene within intron 1 of a human albumin locus of a host cell, comprising administering to a host cell (in vivo or in vitro) a guide RNA as described herein (any one of SEQ ID NO: 2-33), an RNA-guided DNA binding agent (e.g., Cas nuclease as described herein), and a donor construct that comprises a sequence encoding a heterologous polypeptide of interest.

The gRNAs and associated methods and compositions disclosed herein are useful for expressing a heterologous (exogenous) gene within intron 1 of a human albumin locus of a host cell. In some embodiments, the present disclosure provides a method of expressing a heterologous gene within intron 1 of a human albumin locus of a host cell, comprising administering to a host cell (in vivo or in vitro) a guide RNA as described herein (any one of SEQ ID NO: 2-33), an RNA-guided DNA binding agent (e.g., Cas nuclease as described herein), and a donor construct that comprises a sequence encoding a heterologous polypeptide of interest.

The gRNAs and associated methods and compositions disclosed herein are useful for treating a liver-associated disorder in a subject, as described herein. In some embodiments, the present disclosure provides a method of treating a liver-associated disorder, comprising administering to a host cell (in vivo or in vitro) a guide RNA as described herein (any one of SEQ ID NO: 2-33), an RNA-guided DNA binding agent (e.g., Cas nuclease as described herein), and a donor construct that comprises a sequence encoding a polypeptide of interest.

The compositions and methods of the present disclosure are useful and applicable for a range of host cells. In some embodiments, the host cell is a liver cell, neuronal cell, or muscle cell. In some embodiments, the host cell is any suitable non-dividing cell. As used herein, a “non-dividing cell” refers to cells that are terminally differentiated and do not divide, as well as quiescent cells that do not divide but retains the ability to re-enter cell division and proliferation. Liver cells, for example, retain the ability to divide (e.g., when injured or resected), but do not typically divide. During mitotic cell division, homologous recombination is a mechanism by which the genome is protected and double-stranded breaks are repaired. In some embodiments, a “non-dividing” cell refers to a cell in which homologous recombination (HR) is not the primary mechanism by which double-stranded DNA breaks are repaired in the cell, e.g., as compared to a control dividing cell. In some embodiments, a “non-dividing” cell refers to a cell in which non-homologous end joining (NHEJ) is the primary mechanism by which double-stranded DNA breaks are repaired in the cell, e.g., as compared to a control dividing cell. Non-dividing cell types have been described in the literature, e.g. by active NHEJ double-stranded DNA break repair mechanisms. See, e.g. lyama, DNA Repair (Amst.) 2013, 12(8): 620-636. In some embodiments, the host cell includes, but is not limited to, a liver cell, a muscle cell, or a neuronal cell. In some embodiments, the host cell is a hepatocyte, such as a mouse, cyno, or human hepatocyte. In some embodiments, the host cell is a myocyte, such as a mouse, cyno, or human myocyte. In some embodiments, provided herein is a host cell, described above, that comprises the bidirectional construct disclosed herein. In some embodiments the host cell expresses the transgene polypeptide encoded by the bidirectional construct disclosed herein. In some embodiments, provided herein is a host cell made by a method disclosed herein. In certain embodiments, the host cell is made by administering or delivering to a host cell a bidirectional nucleic acid construct described herein, and a gene editing system such as a ZFN, TALEN, or CRISPR/Cas9 system.

In some embodiments, the method further comprises achieving a durable effect, e.g. at least 1 month, 2 months, 6 months, 1 year, or 2 year effect. In some embodiments, the method further comprises achieving the therapeutic effect in a durable and sustained manner, e.g. at least 1 month, 2 months, 6 months, 1 year, or 2 year effect. In some embodiments, the level of circulating Factor IX activity and/or level is stable for at least 1 month, 2 months, 6 months, 1 year, or more. In some embodiments a steady-state activity and/or level of FIX protein is achieved by at least 7 days, at least 14 days, or at least 28 days. In additional embodiments, the method comprises maintaining Factor IX activity and/or levels after a single dose for at least 1, 2, 4, or 6 months, or at least 1, 2, 3, 4, or 5 years.

In additional embodiments involving insertion into the albumin locus, the individual's circulating albumin levels are normal. The method may comprise maintaining the individual's circulating albumin levels within ±5%, ±10%, ±15%, ±20%, or ±50% of normal circulating albumin levels. In certain embodiments, the individual's albumin levels are unchanged as compared to the albumin levels of untreated individuals by at least week 4, week 8, week 12, or week 20. In certain embodiments, the individual's albumin levels transiently drop then return to normal levels. In particular, the methods may comprise detecting no significant alterations in levels of plasma albumin.

In some embodiments, the invention comprises a method or use of modifying (e.g., creating a double strand break in) an albumin gene, such as a human albumin gene, comprising, administering or delivering to a host cell or population of host cells any one or more of the gRNAs, donor construct (e.g., bidirectional construct comprising a sequence encoding Factor IX), and RNA-guided DNA binding agents (e.g., Cas nuclease) described herein. In some embodiments, the invention comprises a method or use of modifying (e.g., creating a double strand break in) an albumin intron 1 region, such as a human albumin intron 1, comprising, administering or delivering to a host cell or population of host cells any one or more of the gRNAs, donor construct (e.g., bidirectional construct comprising a sequence encoding Factor IX), and RNA-guided DNA binding agents (e.g., Cas nuclease) described herein. In some embodiments, the invention comprises a method or use of modifying (e.g., creating a double strand break in) a human genomic locus, such as a safe harbor site, such as liver tissue or hepatocyte host cell, comprising, administering or delivering to a host cell or population of host cells any one or more of the gRNAs, donor construct (e.g., bidirectional construct comprising a sequence encoding Factor IX), and RNA-guided DNA binding agents (e.g., Cas nuclease) described herein. Insertion within a genomic locus, such as a safe harbor site, such as an albumin locus safe harbor site (e.g., intron 1), allows overexpression of the Factor IX gene without significant deleterious effects on the host cell or cell population, such as hepatocytes or liver cells. In some embodiments, the invention comprises a method or use of modifying (e.g., creating a double strand break in) intron 1 of a human albumin locus comprising, administering or delivering to a host cell or population of host cells any one or more of the gRNAs, donor construct (e.g., bidirectional construct comprising a sequence encoding Factor IX), and RNA-guided DNA binding agents (e.g., Cas nuclease) described herein. In some embodiments, the guide RNA comprises a guide sequence that contains at least 15, 16, 17, 18, 19, or 20 contiguous nucleotides that bind within intron 1 of a human albumin locus (SEQ ID NO: 1). In some embodiments, the guide RNA comprises at least 15, 16, 17, 18, 19, or 20 contiguous nucleotides of a sequence selected from the group consisting of SEQ ID NOs: 2-33. In some embodiments, the guide RNA comprises a sequence that is at least 95%, 90%, 85%, 80%, or 75% identical to a sequence selected from the group consisting of SEQ ID NOs:2-33. In some embodiments, the guide RNAs disclosed herein comprise a guide sequence selected from the group consisting of SEQ ID NO: 2, 8, 13, 19, 28, 29, 31, 32, 33. In some embodiments, the guide RNAs disclosed herein comprise a guide sequence at least 17, 18, 19, or 20 contiguous nucleotides of a sequence selected from the group consisting of SEQ ID NOs: 2, 8, 13, 19, 28, 29, 31, 32, 33. In some embodiments, the guide RNAs disclosed herein comprise a guide sequence selected from the group consisting of SEQ ID NOs: 34, 40, 45, 51, 60, 61, 63, 64, 65, 66, 72, 77, 83, 92, 93, 95, 96, and 97. In some embodiments, the guide RNAs disclosed herein comprise a guide sequence that is at least 95%, 90%, 85%, 80%, or 75% identical to a sequence selected from the group consisting of SEQ ID NOs: 2-33. In some embodiments, the guide RNAs disclosed herein comprise a guide sequence that is at least 17, 18, 19, or 20 contiguous nucleotides of a sequence selected from the group consisting of SEQ ID NOs: 2-33. In some embodiments, the guide RNAs disclosed herein comprise a guide sequence that is selected from the group consisting of SEQ ID NOs: 34-97. In some embodiments, the method is performed in vitro. In some embodiments, the method is performed in vivo. In some embodiments, the donor construct is a bidirectional construct that comprises a sequence encoding Factor IX. In some embodiments, the host cell is a liver cell, such as. In additional embodiments, the liver cell is a hepatocyte.

In some embodiments, the invention comprises a method or use of introducing a Factor IX nucleic acid to a host cell or population of host cells comprising, administering or delivering any one or more of the gRNAs, donor construct (e.g., bidirectional construct comprising a sequence encoding Factor IX), and RNA-guided DNA binding agents (e.g., Cas nuclease) described herein. In some embodiments, the guide RNA comprises a guide sequence that contains at least 15, 16, 17, 18, 19, or 20 contiguous nucleotides that are capable of binding to a region within intron 1 of human albumin locus (SEQ ID NO: 1). In some embodiments, the guide RNA comprises at least 15, 16, 17, 18, 19, or 20 contiguous nucleotides of a sequence selected from the group consisting of SEQ ID NOs: 2-33. In some embodiments, the guide RNA comprises a sequence that is at least 95%, 90%, 85%, 80%, or 75% identical to a sequence selected from the group consisting of SEQ ID NOs: 2-33. In some embodiments, the guide RNAs disclosed herein comprise a guide sequence selected from the group consisting of SEQ ID NO: 2, 8, 13, 19, 28, 29, 31, 32, 33. In some embodiments, the guide RNAs disclosed herein comprise a guide sequence at least 17, 18, 19, or 20 contiguous nucleotides of a sequence selected from the group consisting of SEQ ID NOs: 2, 8, 13, 19, 28, 29, 31, 32, 33. In some embodiments, the guide RNAs disclosed herein comprise a guide sequence selected from the group consisting of SEQ ID NOs: 34, 40, 45, 51, 60, 61, 63, 64, 65, 66, 72, 77, 83, 92, 93, 95, 96, and 97. In some embodiments, the guide RNAs disclosed herein comprise a guide sequence that is at least 95%, 90%, 85%, 80%, or 75% identical to a sequence selected from the group consisting of SEQ ID NOs: 2-33. In some embodiments, the guide RNAs disclosed herein comprise a guide sequence that is at least 17, 18, 19, or 20 contiguous nucleotides of a sequence selected from the group consisting of SEQ ID NOs: 2-33. In some embodiments, the guide RNAs disclosed herein comprise a guide sequence that is selected from the group consisting of SEQ ID NOs: 34-97. In some embodiments, the method is in vitro. In some embodiments, the method is in vivo. In some embodiments, the donor construct is a bidirectional construct that comprises a sequence encoding Factor IX. In some embodiments, the host cell is a liver cell, or the population of host cells are liver cells, such as hepatocyte.

In some embodiments, the invention comprises a method or use of expressing Factor IX in a host cell or a population of host cells comprising, administering or delivering any one or more of the gRNAs, donor construct (e.g., bidirectional construct comprising a sequence encoding Factor IX), and RNA-guided DNA binding agents (e.g., Cas nuclease) described herein. In some embodiments, the guide RNA comprises a guide sequence that contains at least 15, 16, 17, 18, 19, or 20 contiguous nucleotides that are capable of binding to a region within intron 1 of human albumin locus (SEQ ID NO: 1). In some embodiments, the guide RNA comprises at least 15, 16, 17, 18, 19, or 20 contiguous nucleotides of a sequence selected from the group consisting of SEQ ID NOs: 2-33. In some embodiments, the guide RNA comprises a sequence that is at least 95%, 90%, 85%, 80%, or 75% identical to a sequence selected from the group consisting of SEQ ID NOs:2-33. In some embodiments, the guide RNAs disclosed herein comprise a guide sequence selected from the group consisting of SEQ ID NO: 2, 8, 13, 19, 28, 29, 31, 32, 33. In some embodiments, the guide RNAs disclosed herein comprise a guide sequence at least 17, 18, 19, or 20 contiguous nucleotides of a sequence selected from the group consisting of SEQ ID NOs: 2, 8, 13, 19, 28, 29, 31, 32, 33. In some embodiments, the guide RNAs disclosed herein comprise a guide sequence selected from the group consisting of SEQ ID NOs: 34, 40, 45, 51, 60, 61, 63, 64, 65, 66, 72, 77, 83, 92, 93, 95, 96, and 97. In some embodiments, the guide RNAs disclosed herein comprise a guide sequence that is at least 95%, 90%, 85%, 80%, or 75% identical to a sequence selected from the group consisting of SEQ ID NOs: 2-33. In some embodiments, the guide RNAs disclosed herein comprise a guide sequence that is at least 17, 18, 19, or 20 contiguous nucleotides of a sequence selected from the group consisting of SEQ ID NOs: 2-33. In some embodiments, the guide RNAs disclosed herein comprise a guide sequence that is selected from the group consisting of SEQ ID NOs: 34-97. In some embodiments, the method is in vitro. In some embodiments, the method is in vivo. In some embodiments, the donor construct is a bidirectional construct that comprises a sequence encoding Factor IX. In some embodiments, the host cell is a liver cell, or the population of host cells are liver cells, such as hepatocyte.

In some embodiments, the invention comprises a method or use of treating hemophilia (e.g., hemophilia A or hemophilia B) comprising, administering or delivering any one or more of the gRNAs, donor construct (e.g., bidirectional construct comprising a sequence encoding Factor IX), and RNA-guided DNA binding agents (e.g., Cas nuclease) described herein to a subject in need thereof. In some embodiments, the guide RNA comprises a guide sequence that contains at least 15, 16, 17, 18, 19, or 20 contiguous nucleotides that are capable of binding to a region within intron 1 of human albumin locus (SEQ ID NO: 1). In some embodiments, the guide RNA comprises at least 15, 16, 17, 18, 19, or 20 contiguous nucleotides of a sequence selected from the group consisting of SEQ ID NOs: 2-33. In some embodiments, the guide RNA comprises a sequence that is at least 95%, 90%, 85%, 80%, or 75% identical to a sequence selected from the group consisting of SEQ ID NOs: 2-33. In some embodiments, the guide RNAs disclosed herein comprise a guide sequence selected from the group consisting of SEQ ID NO: 2, 8, 13, 19, 28, 29, 31, 32, 33. In some embodiments, the guide RNAs disclosed herein comprise a guide sequence at least 17, 18, 19, or 20 contiguous nucleotides of a sequence selected from the group consisting of SEQ ID NOs: 2, 8, 13, 19, 28, 29, 31, 32, 33. In some embodiments, the guide RNAs disclosed herein comprise a guide sequence selected from the group consisting of SEQ ID NOs: 34, 40, 45, 51, 60, 61, 63, 64, 65, 66, 72, 77, 83, 92, 93, 95, 96, and 97. In some embodiments, the guide RNAs disclosed herein comprise a guide sequence that is at least 95%, 90%, 85%, 80%, or 75% identical to a sequence selected from the group consisting of SEQ ID NOs: 2-33. In some embodiments, the guide RNAs disclosed herein comprise a guide sequence that is at least 17, 18, 19, or 20 contiguous nucleotides of a sequence selected from the group consisting of SEQ ID NOs: 2-33. In some embodiments, the guide RNAs disclosed herein comprise a guide sequence that is selected from the group consisting of SEQ ID NOs: 34-97. In some embodiments, the donor construct is a bidirectional construct that comprises a sequence encoding a heterologous polypeptide. In some embodiments, the host cell is a liver cell, or the population of host cells are liver cells, such as hepatocytes.

As used herein, “hemophilia” refers to a disorder caused by a missing or defective Factor IX gene or polypeptide. Hemophilia also refers to a disorder caused by a missing or defective Factor VIII gene or polypeptide. The disorder includes conditions that are inherited and/or acquired (e.g., caused by a spontaneous mutation in the gene), and includes hemophilia A and hemophilia B. Hemophilia A is caused by Factor VIII deficiency. Hemophilia B is caused by Factor IX deficiency. In some embodiments, the defective Factor IX gene or polypeptide results in reduced Factor IX level in the plasma and/or a reduced coagulation activity of Factor IX. As used herein, hemophilia includes mild, moderate, and severe hemophilia. For example, individuals with less than about 1% active factor are classified as having severe hemophilia, those with about 1-5% active factor have moderate hemophilia, and those with mild hemophilia have between about 5-40% of normal levels of active clotting factor.

In some embodiments, the donor construct comprises a sequence encoding Factor IX, wherein the Factor IX sequence is wild type Factor IX. In some embodiments, the sequence encodes a variant of Factor IX. For example, the variant can possess increased coagulation activity than wild type Factor IX. For example, the variant Factor IX can comprise one or mutations, such as an amino acid substitution in position R338 (e.g., R338L) relative to wild-type Factor IX. In some embodiments, the sequence encodes a Factor IX variant that is 80%, 85%, 90%, 93%, 95%, 97%, 99% identical to wild-type Factor IX, having at least 80%, 85%, 90%, 92%, 94%, 96%, 98%, 99%, 100%, or more, activity as compared to wild type Factor IX. In some embodiments, the sequence encodes a fragment of Factor IX, wherein the fragment possesses at least 80%, 85%, 90%, 92%, 94%, 96%, 98%, 99%, 100%, or more, activity as compared to wild type Factor IX.

In some embodiments, the donor construct comprises a sequence encoding a Factor IX variant, wherein the Factor IX variant activates coagulation in the absence of its cofactor, Factor VIII (expression results in therapeutically relevant FVIII mimetic activity). Such Factor IX variants can further maintain the activity of wild type Factor IX. For example, such a Factor IX variant can comprise an amino acid substation at position L6, V181, K265, I383, E185, or a combination thereof relative to wild type Factor IX. For example, such a Factor IX variant can comprise an L6F mutation, a V181I mutation, a K265A mutation, an I383V mutation, an E185D mutation, or a combination thereof relative to wild type Factor IX.

The compositions and methods of the present disclosure are useful for efficient insertion of a heterologous gene of interest and safe expression of the heterologous polypeptide (e.g., a therapeutic polypeptide). In some embodiments, the polypeptide is a secreted polypeptide. In some embodiments, the polypeptide is one in which its function is normally effected (e.g., functionally active) as a secreted polypeptide. A “secreted polypeptide” as used herein refers to a protein that is secreted by the cell and/or is functionally active as a soluble extracellular protein.

In some embodiments, the polypeptide is an intracellular polypeptide. In some embodiments, the polypeptide is one in which its function is normally effected (e.g., functionally active) inside a cell. An “intracellular polypeptide” as used herein refers to a protein that is not secreted by the cell, including soluble cytosolic polypeptides. One or more IRES and/or self cleaving peptide sequences may flank an intracellular polypeptide, e.g. at or near an end of the polypeptide, such an amino terminal end of the polypeptide.

In some embodiments, the polypeptide is a wild-type polypeptide. In some embodiments, the polypeptide is variant (e.g., mutant) polypeptide (e.g., a hyperactive mutant of a wild-type polypeptide). In some embodiments, the polypeptide is a liver protein. In some embodiments, the polypeptide is a non-liver protein. In some embodiments, the polypeptide is Factor IX, or a variant thereof. In some embodiments, the liver polypeptide is, for example, a polypeptide to address a liver disorder such as, without limitation, tyrosinemia, Wilson's disease, Tay-Sachs disease, hyperbilirubinema (Crigler-Najjar), acute intermittent porphyria, citrullinemia type 1, progressive familiar intrahepatic cholestasis, or maple syrup urine disease.

In some embodiments, expression of the polypeptide by the host cell (whether in vitro or in vivo) is increased by at least 2%, 3%, 4%, 5%, 6%, 7%, 8%, 9%, 10%, 15%, 20%, 25%, 30%, 35%, 40%, 45%, 50%, 55%, 60%, 65%, 70%, or more relative to a level expressed by the host cell prior to providing the compositions disclosed herein. In additional embodiments, expression of the heterologous polypeptide may be increased to at least detectable levels or therapeutically effective levels.

In some embodiments, expression of the polypeptide by the host cell (whether in vitro or in vivo) is increased to at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, or more, of a known normal level (e.g., a level of a polypeptide in a healthy subject).

In some embodiments, expression of the polypeptide by the host cell (whether in vitro or in vivo) is increased to at least about 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 120 μg/ml, 140 μg/ml, 160 μg/ml, 180 μg/ml, 200 μg/ml, 225 μg/ml, 250 μg/ml, 275 μg/ml, 300 μg/ml, 325 μg/ml, 350 μg/ml, 400 μg/ml, 450 μg/ml, 500 μg/ml, 550 μg/ml, 600 μg/ml, 650 μg/ml, 700 μg/ml, 750 μg/ml, 800 μg/ml, 850 μg/ml, 900 μg/ml, 1000 μg/ml, 1100 μg/ml, 1200 μg/ml, 1300 μg/ml, 1400 μg/ml, 1500 μg/ml, 1600 μg/ml, 1700 μg/ml, 1800 μg/ml, 1900 μg/ml, 2000 μg/ml, or more, as determined, e.g., in the cell, plasma, and/or serum of a subject. Methods of detecting and measuring polypeptide levels in various samples are well known in the art.

In some embodiments, compositions and methods of the present disclosure are useful for treating a liver-associated disease. As used herein, a “liver-associated disease” refers to diseases that cause damage to the liver tissue directly, diseases that result from damage to the liver tissue, and/or disorders of non-liver organs or tissue that resulted from a defect in the liver. Examples of liver-associated disease include, without limitation, tyrosinemia, Wilson's disease, Tay-Sachs disease, hyperbilirubinema (Crigler-Najjar), acute intermittent porphyria, citrullinemia type 1, progressive familiar intrahepatic cholestasis, and maple syrup urine disease.

As described herein, any one or more of the guide RNA disclosed herein, RNA-guided DNA binding agent, and donor construct comprising a transgene, can be delivered using any suitable delivery system and method known in the art. The compositions can be delivered in vitro or in vivo simultaneously or in any sequential order. In some embodiments, the donor construct, guide RNA, and RNA-guided DNA binding agent can be delivered in vitro or in vivo simultaneously, e.g., in one vector, two vectors, individual vectors, one LNP, two LNPs, individual LNPs, or a combination thereof. In some embodiments, the donor construct can be delivered in vivo or in vitro, as a vector and/or associated with a LNP, prior to (e.g., about 1, 2, 3, 4, 5, 6, 7, 8, 9, 10, 11, 12, 13, 14, or more days) delivering the guide RNA and/or RNA-guided DNA binding agent, as a vector and/or associated with a LNP singly or together as a ribonucleoprotein (RNP). As a further example, the guide RNA and RNA-guided DNA binding agent, as a vector and/or associated with a LNP singly or together as a ribonucleoprotein (RNP), can be delivered in vivo or in vitro, prior to (e.g., about 1, 2, 3, 4, 5, 6, 7, 8, 9, 10, 11, 12, 13, 14, or more days) delivering the construct, as a vector and/or associated with a LNP. In some embodiments, the guide RNA and RNA-guided DNA binding agent are associated with an LNP and delivered to the host cell prior to delivering the donor construct.

In some embodiments, the donor construct comprises a sequence encoding Factor IX, or variants thereof. For example, the variant possesses increased activity than wild type polypeptide. In some embodiments, the sequence encodes a polypeptide variant that is 80%, 85%, 90%, 93%, 95%, 97%, 99% identical to a wild-type polypeptide sequence, having at least 80%, 85%, 90%, 92%, 94%, 96%, 98%, 99%, 100%, or more, activity as compared to wild type polypeptide. In some embodiments, the sequence encodes a fragment of a wild type polypeptide, wherein the fragment possesses at least 80%, 85%, 90%, 92%, 94%, 96%, 98%, 99%, 100%, or more, activity as compared to wild type polypeptide.

In some embodiments, a single administration of a donor construct comprising a heterologous gene, guide RNA, and RNA-guided DNA binding agent is sufficient to increase expression of a polypeptide of interest to a desirable level. In other embodiments, more than one administration of a composition comprising a donor construct comprising a heterologous gene, guide RNA, and RNA-guided DNA binding agent may be beneficial to maximize therapeutic effects.

In some embodiments, the guide RNAs, RNA-guided DNA binding agent, and donor construct are administered individually or in any combination intravenously. In some embodiments, the guide RNAs, RNA-guided DNA binding agent, and donor construct are administered individually or in any combination into the hepatic circulation.

In some embodiments, the host or subject is a mammal. In some embodiments, the host or subject is a human. In some embodiments, the host or subject is a rodent (e.g., mouse).

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### EXAMPLES

The following examples are provided to illustrate certain disclosed embodiments and are not to be construed as limiting the scope of this disclosure in any way.

### Example 1—Materials and Methods

### Cloning and Plasmid Preparation

A bidirectional insertion construct flanked by AAV2 ITRs was synthesized and cloned into pUC57-Kan by a commercial vendor. The resulting construct (P00147) was used as the parental cloning vector for other vectors. The other insertion constructs (without ITRs) were also commercially synthesized and cloned into pUC57. Purified plasmid was digested with BglII restriction enzyme (New England BioLabs, cat #R0144S), and the insertion constructs were cloned into the parental vector. Plasmid was propagated in Stbl3™ Chemically Competent E. coli (Thermo Fisher, Cat #C737303).

### AAV Production

Triple transfection in HEK293 cells was used to package genomes with constructs of interest for AAV8 and AAV-DJ production and resulting vectors were purified from both lysed cells and culture media through iodixanol gradient ultracentrifugation method (See, e.g., Lock et al., Hum Gene Ther. 2010 October; 21(10):1259-71). The plasmids used in the triple transfection that contained the genome with constructs of interest are referenced in the Examples by a “PXXXX” number, see also e.g., Table 9. Isolated AAV was dialyzed in storage buffer (PBS with 0.001% Pluronic F68). AAV titer was determined by qPCR using primers/probe located within the ITR region.

In Vitro Transcription (“IVT”) of Nuclease mRNA

Capped and polyadenylated Streptococcus pyogenes (“Spy”) Cas9 mRNA containing N1-methyl pseudo-U was generated by in vitro transcription using a linearized plasmid DNA template and T7 RNA polymerase. Generally, plasmid DNA containing a T7 promoter and a 100 nt poly (A/T) region was linearized by incubating at 37° C. with XbaI to complete digestion followed by heat inactivation of XbaI at 65° C. The linearized plasmid was purified from enzyme and buffer salts. The IVT reaction to generate Cas9 modified mRNA was incubated at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine Rnase inhibitor (NEB); 0.004 U/μL Inorganic E. coli pyrophosphatase (NEB); and 1× reaction buffer. TURBO Dnase (ThermoFisher) was added to a final concentration of 0.01 U/μL, and the reaction was incubated for an additional 30 minutes to remove the DNA template. The Cas9 mRNA was purified using a MegaClear Transcription Clean-up kit according to the manufacturer's protocol (ThermoFisher). Alternatively, the Cas9 mRNA was purified using LiCl precipitation, ammonium acetate precipitation, and sodium acetate precipitation or using a LiCl precipitation method followed by further purification by tangential flow filtration. The transcript concentration was determined by measuring the light absorbance at 260 nm (Nanodrop), and the transcript was analyzed by capillary electrophoresis by Bioanlayzer (Agilent).

The Cas9 mRNAs below comprise Cas9 ORF SEQ ID NO:703 or SEQ ID NO: 704 or a sequence of Table 24 of PCT/US2019/053423 (which is hereby incorporated by reference).

Lipid Formulations for Delivery of Cas9 mRNA and gRNA Cas9 mRNA and gRNA were delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG.

For experiments utilizing pre-mixed lipid formulations (referred to herein as “lipid packets”), the components were reconstituted in 100% ethanol at a molar ratio of ionizable lipid:cholesterol:DSPC:PEG2k-DMG of 50:38:9:3, prior to being mixed with RNA cargos (e.g., Cas9 mRNA and gRNA) at a lipid amine to RNA phosphate (N:P) molar ratio of about 6.0, as further described herein.

For experiments utilizing the components formulated as lipid nanoparticles (LNPs), the components were dissolved in 100% ethanol at various molar ratios. The RNA cargos (e.g., Cas9 mRNA and gRNA) were dissolved in 25 mM citrate, 100 mM NaCl, pH 5.0, resulting in a concentration of RNA cargo of approximately 0.45 mg/mL.

For the experiments described in Example 2, the LNPs were formed by microfluidic mixing of the lipid and RNA solutions using a Precision Nanosystems NanoAssemblr™ Benchtop Instrument, according to the manufacturer's protocol. A 2:1 ratio of aqueous to organic solvent was maintained during mixing using differential flow rates. After mixing, the LNPs were collected, diluted in water (approximately 1:1 v/v), held for 1 hour at room temperature, and further diluted with water (approximately 1:1 v/v) before final buffer exchange. The final buffer exchange into 50 mM Tris, 45 mM NaCl, 5% (w/v) sucrose, pH 7.5 (TSS) was completed with PD-10 desalting columns (GE). If required, formulations were concentrated by centrifugation with Amicon 100 kDa centrifugal filters (Millipore). The resulting mixture was then filtered using a 0.2 μm sterile filter. The final LNP was stored at −80° C. until further use. The LNPs were formulated at a molar ratio of ionizable lipid:cholesterol:DSPC:PEG2k-DMG of 45:44:9:2, with a lipid amine to RNA phosphate (N:P) molar ratio of about 4.5, and a ratio of gRNA to mRNA of 1:1 by weight.

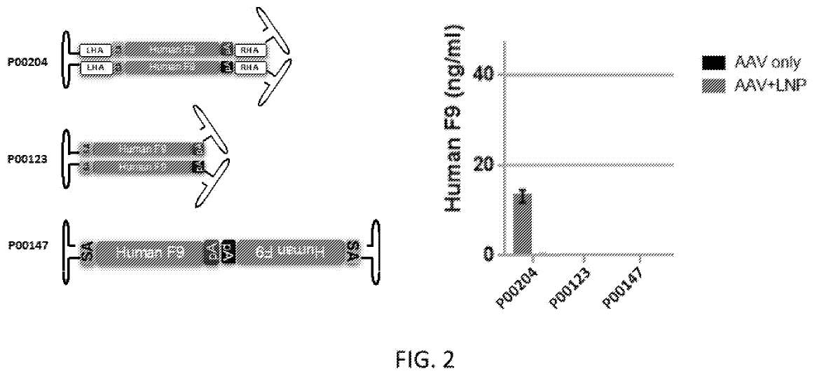

For the experiments described other examples, the LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, formulations were concentrated by centrifugation with Amicon 100 kDa centrifugal filters (Millipore). The resulting mixture was then filtered using a 0.2 μm sterile filter. The final LNP was stored at 4° C. or −80° C. until further use. The LNPs were formulated at a molar ratio of ionizable lipid:cholesterol:DSPC:PEG2k-DMG of 50:38:9:3, with a lipid amine to RNA phosphate (N:P) molar ratio of about 6.0, and a ratio of gRNA to mRNA of 1:1 by weight.

Cell Culture and In Vitro Delivery of Cas9 mRNA, gRNA, and Insertion Constructs

### Hepa1-6 Cells

Hepa 1-6 cells were plated at density of 10,000 cells/well in 96-well plates. 24 hours later, cells were treated with LNP and AAV. Before treatment the media was aspirated off from the wells. LNP was diluted to 4 ng/ul in DMEM+10% FBS media and further diluted to 2 ng/ul in 10% FBS (in DMEM) and incubated at 37° C. for 10 min (at a final concentration of 5% FBS). Target MOI of AAV was 1e6, diluted in DMEM+10% FBS media. 50 μl of the above diluted LNP at 2 ng/ul was added to the cells (delivering a total of 100 ng of RNA cargo) followed by 50 μl of AAV. The treatment of LNP and AAV were minutes apart. Total volume of media in cells was 100 μl. After 72 hours post-treatment and 30 days post-treatment, supernatant from these treated cells were collected for human FIX ELISA analysis as described below.

### Primary Hepatocytes

Primary mouse hepatocytes (PMH), primary cyno hepatocytes (PCH) and primary human hepatocytes (PHH) were thawed and resuspended in hepatocyte thawing medium with supplements (ThermoFisher) followed by centrifugation. The supernatant was discarded, and the pelleted cells resuspended in hepatocyte plating medium plus supplement pack (ThermoFisher). Cells were counted and plated on Bio-coat collagen I coated 96-well plates at a density of 33,000 cells/well for PHH and 50,000 cells/well for PCH and 15,000 cells/well for PMH. Plated cells were allowed to settle and adhere for 5 hours in a tissue culture incubator at 37° C. and 5% CO₂ atmosphere. After incubation cells were checked for monolayer formation and were washed thrice with hepatocyte maintenance prior and incubated at 37° C.

For experiments utilizing lipid packet delivery, Cas9 mRNA and gRNA were each separately diluted to 2 mg/ml in maintenance media and 2.9 μl of each were added to wells (in a 96-well Eppendorf plate) containing 12.5 μl of 50 mM sodium citrate, 200 mM sodium chloride at pH 5 and 6.9 μl of water. 12.5 μl of lipid packet formulation was then added, followed by 12.5 μl of water and 150 μl of TSS. Each well was diluted to 20 ng/μl (with respect to total RNA content) using hepatocyte maintenance media, and then diluted to 10 ng/μl (with respect to total RNA content) with 6% fresh mouse serum. Media was aspirated from the cells prior to transfection and 40 μl of the lipid packet/RNA mixtures were added to the cells, followed by addition of AAV (diluted in maintenance media) at an MOI of 1e5. Media was collected 72 hours post-treatment for analysis and cells were harvested for further analysis, as described herein

### Luciferase Assays

For experiments involving NanoLuc detection in cell media, one volume of Nano-Glo® Luciferase Assay Substrate was combined with 50 volumes of Nano-Glo® Luciferase Assay Buffer. The assay was run on a Promega Glomax runner at an integration time of 0.5 sec using 1:10 dilution of samples (50 μl of reagent+40 μl water+10 μl cell media).

For experiments involving detection of the HiBit tag in cell media, LgBiT Protein and Nano-GloR HiBiT Extracellular Substrate were diluted 1:100 and 1:50, respectively, in room temperature Nano-GloR HiBiT Extracellular Buffer. The assay was run on a Promega Glomax runner at an integration time of 1.0 sec using 1:10 dilution of samples (50 μl of reagent+40 μl water+10 μl cell media).

In Vivo Delivery of LNP and/or AAV

Mice were dosed with AAV, LNP, both AAV and LNP, or vehicle (PBS+0.001% Pluronic for AAV vehicle, TSS for LNP vehicle) via the lateral tail vein. AAV were administered in a volume of 0.1 mL per animal with amounts (vector genomes/mouse, “vg/ms”) as described herein. LNPs were diluted in TSS and administered at amounts as indicated herein, at about 5 μl/gram body weight. Typically, mice were injected first with AAV and then with LNP, if applicable. At various times points post-treatment, serum and/or liver tissue was collected for certain analyses as described further below.

Human Factor IX (hFIX) ELISA Analysis

For in vitro studies, total human Factor IX levels secreted in cell media were determined using a Human Factor IX ELISA Kit (Abcam, Cat #ab188393) according to manufacturer's protocol. Secreted hFIX levels were quantitated off a standard curve using 4 parameter logistic fit and expressed as ng/ml of media.

For in vivo studies, blood was collected and the serum or plasma was isolated as indicated. The total human Factor IX levels were determined using a Human Factor IX ELISA Kit (Abcam, Cat #ab188393) according to manufacturer's protocol. Serum or plasma hFIX levels were quantitated off a standard curve using 4 parameter logistic fit and expressed as μg/mL of serum.

### Next-Generation Sequencing (“NGS”) and Analysis for On-Target Cleavage Efficiency

Deep sequencing was utilized to identify the presence of insertions and deletions introduced by gene editing, e.g., within intron 1 of albumin. PCR primers were designed around the target site and the genomic area of interest was amplified. Primer sequence design was done as is standard in the field.

Additional PCR was performed according to the manufacturer's protocols (IIlumina) to add chemistry for sequencing. The amplicons were sequenced on an Illumina MiSeq instrument. The reads were aligned to the reference genome after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

### In Situ Hybridization Analysis

BaseScope (ACDbio, Newark, Calif.) is a specialized RNA in situ hybridization technology that can provide specific detection of exon junctions, e.g., in a hybrid mRNA transcript that contains an insertion transgene (hFIX) and coding sequence from the site of insertion (exon 1 of albumin). BaseScope was used to measure the percentage of liver cells expressing the hybrid mRNA.

To detect the hybrid mRNA, two probes against the hybrid mRNAs that may arise following insertion of a bidirectional construct were designed by ACDbio (Newark, Calif.). One of the probes was designed to detect a hybrid mRNA resulting from insertion of the construct in one orientation, while the other probe was designed to detect a hybrid mRNA resulting from insertion of the construct in the other orientation. Livers from different groups of mice were collected and fresh-frozen sectioned. The BaseScope assay, using a single probe or pooled probes was performed according to the manufacture's protocol. Slides were scanned and analyzed by the HALO software. The background (saline treated group) of this assay was 0.58%.

### Example 2—In Vitro Testing of Insertion Templates for Intron 1 of Albumin with and without Homology Arms

In this Example, Hepa1-6 cells were cultured and treated with AAV harboring insertion templates of various forms (e.g., having either a single-stranded genome (“ssAAV”) or a self-complementary genome (“scAAV”)), in the presence or absence of LNP delivering Cas9 mRNA and G000551 e.g., as described in Example 1 (n=3). The AAV and LNP were prepared as described in Example 1. Following treatment, the media was collected for human Factor IX levels as described in Example 1.

Hepa1-6 cells are an immortalized mouse liver cell line that continues to divide in culture. As shown in FIG. 2 (72 hour post-treatment time point), the vector (scAAV derived from plasmid P00204) comprising 200 bp homology arms resulted in detectable expression of hFIX, e.g., following insertion into intron 1 of albumin in the cycling cells. Use of the AAV vectors derived from P00123 (scAAV lacking homology arms) and P00147 (ssAAV bidirectional construct lacking homology arms) did not result in detectable expression of hFIX in this experiment. The cells were kept in culture and these results were confirmed when re-assayed at 30 days post-treatment (data not shown).

### Example 3—In Vivo Testing of Insertion Templates for Intron 1 of Albumin with and without Homology Arms

In this Example, mice were treated with AAV derived from the same plasmids (P00123, P00204, and P00147) as tested in vitro in Example 2. The dosing materials were prepared and dosed as described in Example 1. C57B1/6 mice were dosed (n=5 for each group) with 3e11 vector genomes each (vg/ms) followed by LNP comprising G000551 (“G551”) at a dose of 4 mg/kg (with respect to total RNA cargo content). Four weeks post dose, the animals were euthanized and liver tissue and sera were collected for editing and hFIX expression, respectively.

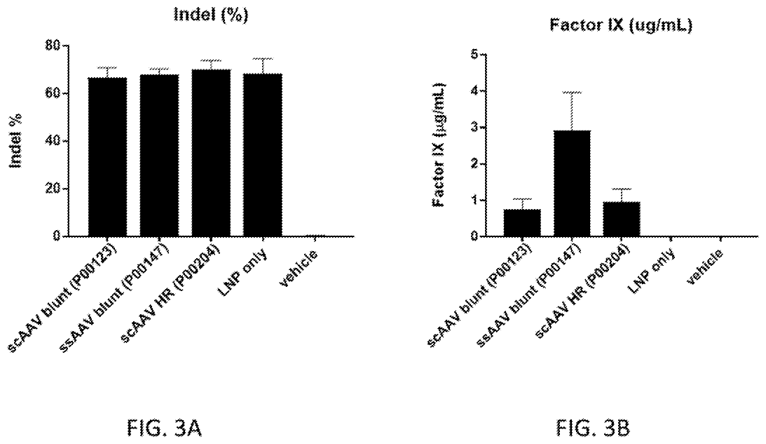

As shown in FIG. 3A and Table 12, liver editing levels of ˜60% were detected in each group of animals treated with LNP comprising gRNA targeting intron 1 of murine albumin.

However, despite robust and consistent levels of editing in each treatment group, animals receiving the ssAAV vector without homology arms (vector derived from P00147) in combination with LNP treatment resulted in the highest level of hFIX expression in serum (FIG. 3B and Table 13).

TABLE 12
Indel %

| Template | Average Indel (%) | St. Dev Indel (%) |
|---|---|---|
| scAAV Blunt (P00123) | 66.72 | 4.09 |
| ssAAV Blunt (P00147) | 68.10 | 2.27 |
| ssAAV HR (P00204) | 70.16 | 3.68 |
| LNP only | 68.24 | 6.47 |
| Vehicle | 0.28 | 0.08 |

TABLE 13
Factor IX Levels (ug/mL)

| | Average Factor IX | St. Dev Factor IX |
|---|---|---|
| Template | (ug/mL) | (ug/mL) |
| scAAV Blunt (P00123) | 0.75 | 0.28 |
| ssAAV Blunt (P00147) | 2.92 | 1.04 |
| ssAAV HR (P00204) | 0.96 | 0.35 |
| LNP only | 0 | 0 |
| Vehicle | 0 | 0 |

### Example 4—In Vivo Testing of ssAAV Insertion Templates for Intron 1 of Albumin with and without Homology Arms

The experiment described in this Example examined the effect of incorporating homology arms into ssAAV vectors in vivo.

The dosing materials used in this experiment were prepared and dosed as described in Example 1. C57B1/6 mice were dosed (n=5 for each group) with 3e11 vg/ms followed by LNP comprising G000666 (“G666”) or G000551 (“G551”) at a dose of 0.5 mg/kg (with respect to total RNA cargo content). Four weeks post dose, the animals sera was collected for hFIX expression.

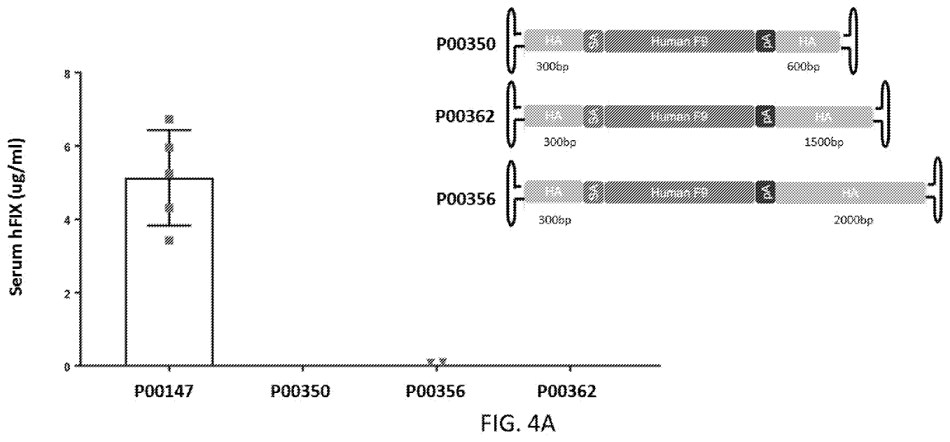

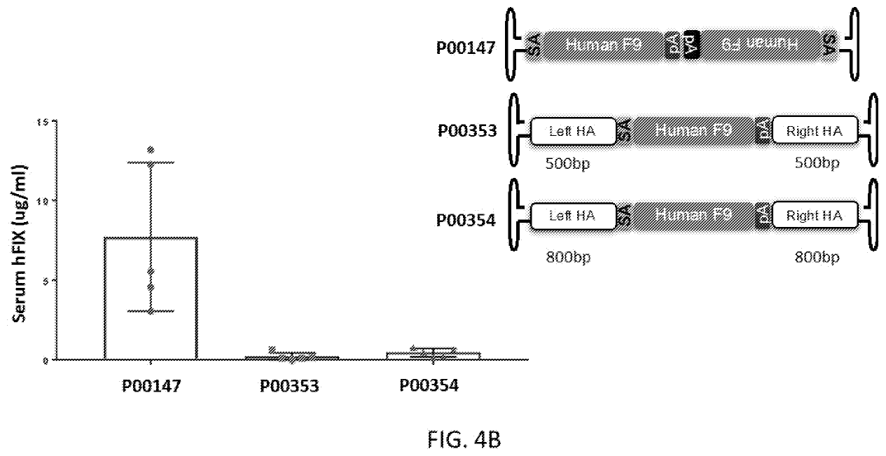

As shown in FIG. 4A and Table 14, use of the ssAAV vectors with asymmetrical homology arms (300/600 bp arms, 300/2000 bp arms, and 300/1500 bp arms for vectors derived from plasmids P00350, P00356, and P00362, respectively) for insertion into the albumin intron 1 site targeted by G551 resulted in levels of circulating hFIX that were below the lower limit of detection for the assay. However, use of the ssAAV vector (derived from P00147) without homology arms and having two hFIX open reading frames (ORF) in a bidirectional orientation resulted in detectable levels of circulating hFIX in each animal.

Similarly, use of the ssAAV vectors with symmetrical homology arm from plasmids P00353 and P00354, respectively) for insertion into the albumin intron 1 site targeted by G666 resulted in lower but detectable levels, as compared to use of the bidirectional vector without homology arms (derived from P00147) (see FIG. 4B and Table 15).

TABLE 14
hFIX Serum Levels

| | Average Serum FIX | St. Dev Serum FIX |
|---|---|---|
| AAV | (ug/mL) | (ug/mL) |
| P00147 | 5.13 | 1.31 |
| P00350 | −0.22 | 0.08 |
| P00356 | −0.23 | 0.04 |
| P00362 | −0.09 | 0.16 |

TABLE 15
hFIX Serum Levels

| | Average Serum FIX | St. Dev Serum FIX |
|---|---|---|
| AAV | (ug/mL) | (ug/mL) |
| P00147 | 7.72 | 4.67 |
| P00353 | 0.20 | 0.23 |
| P00354 | 0.46 | 0.26 |

### Example 5—In Vitro Screening of Bidirectional Constructs Across 20 Target Sites in Intron 1 of Albumin in Primary Mouse Hepatocytes

Having demonstrated that bidirectional constructs lacking homology arms outperformed vectors with other configurations for insertion into intron 1 of albumin, the experiment described in this Example examined the effects of ald the splice acceptors. These varied bidirectional constructs were tested across a panel of target sites utilizing 20 different gRNAs targeting intron 1 of murine albumin in primary mouse hepatocytes (PMH).

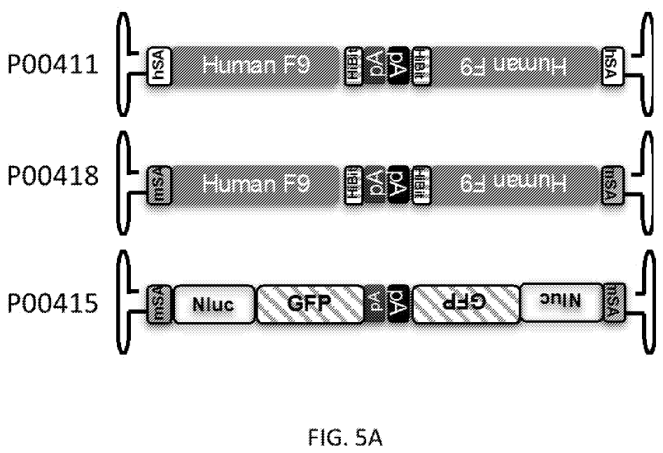

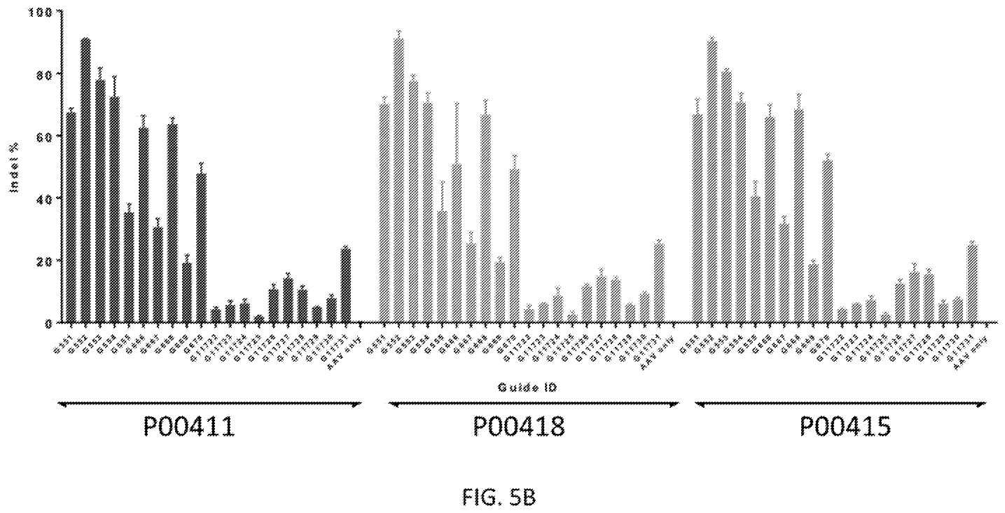

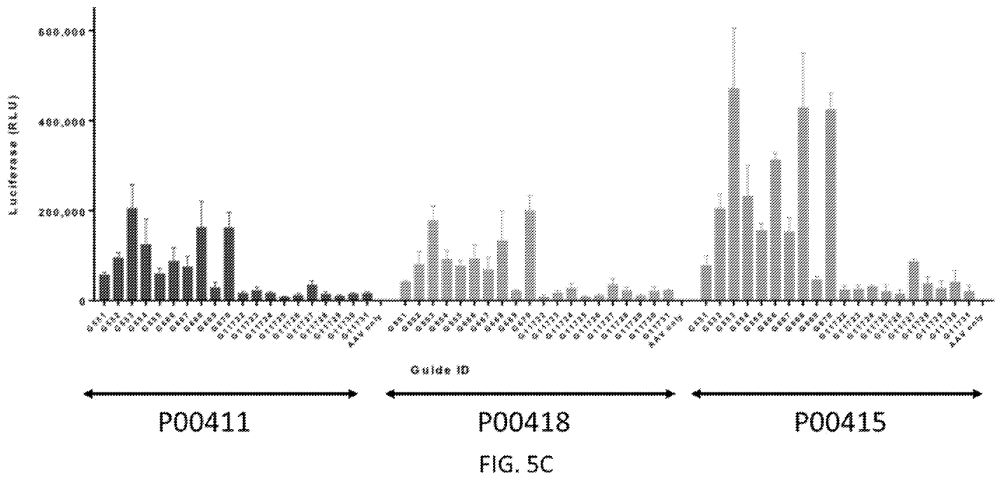

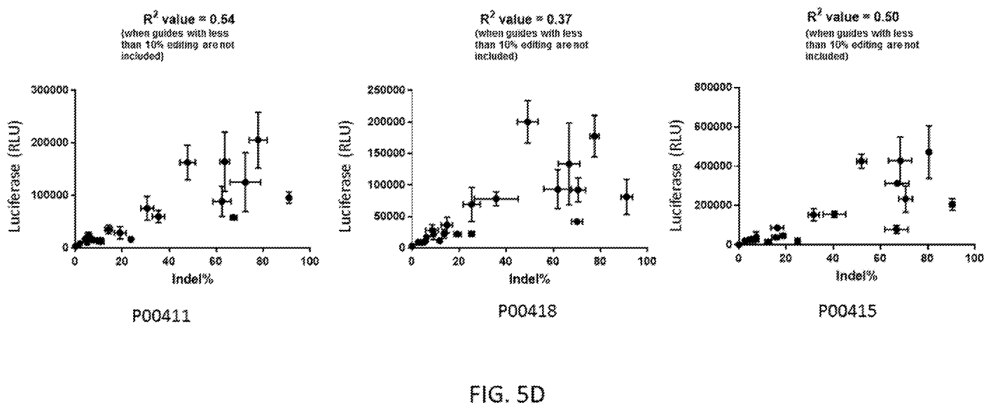

The ssAAV and lipid packet delivery materials tested in this Example were prepared and delivered to PMH as described in Example 1, with the AAV at an MOI of 1e5. Following treatment, isolated genomic DNA and cell media was collected for editing and transgene expression analysis, respectively. Each of the vectors comprised a reporter that can be measured through luciferase-based fluorescence detection as described in Example 1, plotted in FIG. 5C as relative luciferase units (“RLU”). For example, the AAV vectors comprising the hFIX ORFs contained a HiBit peptide fused at their 3′ ends, and the AAV vector comprising only reporter genes comprised a NanoLuc ORF (in addition to GFP). Schematics of each of the vectors tested are provided in FIG. 5A. The gRNAs tested are shown in FIG. 5B and FIG. 5C, using a shortened number for those listed in Table 5 (e.g., where the leading zeros are omitted, for example where “G551” corresponds to “G000551” in Table 5).

As shown in FIG. 5B and Table 16, consistent but varied levels of editing were detected for each of the treatment groups across each combination tested. Transgene expression using various combinations of template and guide RNA is shown in FIG. 5C. As shown in FIG. 5D, a significant level of indel formation did not necessarily result in more efficient expression of the transgenes. Not all guides that generated in significant indels resulted in high levels of proteins with the same insertion template as measured by the relative luciferase activities. Using P00411- and P00418-derived templates, the R² values were 0.54 and 0.37 between indel and luciferase activity, respectively, when guides with less than 10% editing are not included (FIG. 5D). Interestingly, despite differing ORFs and splice acceptors, the relative levels of expression as measured in RLUs was consistent between the three vectors tested, demonstrating the robustness, reproducibility and modularity of the bidirectional construct system, e.g., for use in inserting transgenes of interest into intron 1 of albumin (see FIG. 5C and Table 17). The mouse albumin splice acceptor and human FIX splice acceptor each resulted in effective transgene expression.

TABLE 16
% Indel

| | P00411 | | P00418 | | P00415 | |
|---|---|---|---|---|---|---|
| | Average | St. Dev | Average | St. Dev | Average | St. Dev |
| | Indel | Indel | Indel | Indel | Indel | Indel |
| Guide ID | (%) | (%) | (%) | (%) | (%) | (%) |
| G000551 | 67.4 | 1.42 | 70.67 | 2.29 | 66.73 | 4.90 |
| G000552 | 90.93 | 0.15 | 91.10 | 2.43 | 90.37 | 1.01 |
| G000553 | 77.80 | 3.83 | 77.47 | 1.87 | 80.50 | 0.85 |
| G000554 | 72.37 | 6.49 | 70.53 | 3.16 | 70.60 | 2.91 |
| G000555 | 35.37 | 2.63 | 35.77 | 9.34 | 40.47 | 4.75 |
| G000666 | 62.47 | 3.87 | 50.90 | 19.41 | 65.90 | 3.99 |
| G000667 | 30.57 | 2.73 | 25.30 | 3.67 | 31.67 | 2.29 |
| G000668 | 63.60 | 2.02 | 66.65 | 4.60 | 68.30 | 4.90 |
| G000669 | 19.10 | 2.51 | 19.33 | 1.53 | 18.70 | 1.25 |
| G000670 | 47.80 | 3.27 | 49.10 | 4.42 | 51.97 | 2.06 |
| G011722 | 4.20 | 0.72 | 4.27 | 1.20 | 4.20 | 0.26 |
| G011723 | 5.63 | 1.27 | 6.07 | 0.15 | 5.93 | 0.15 |
| G011724 | 6.10 | 1.28 | 8.50 | 2.69 | 7.13 | 1.27 |
| G011725 | 1.93 | 0.29 | 2.60 | 0.79 | 2.53 | 0.65 |
| G011726 | 10.73 | 1.46 | 11.70 | 0.50 | 12.43 | 1.33 |
| G011727 | 14.20 | 1.56 | 14.80 | 2.36 | 16.20 | 2.69 |
| G011728 | 10.55 | 1.20 | 13.65 | 0.92 | 15.50 | 1.56 |
| G011729 | 5.00 | 0.10 | 5.63 | 0.25 | 6.00 | 1.01 |
| G011730 | 7.83 | 0.97 | 9.13 | 0.59 | 7.33 | 0.59 |
| G011731 | 23.70 | 0.66 | 25.27 | 1.21 | 24.87 | 1.01 |
| AAV Only | 0.15 | 0.07 | 0.05 | 0.07 | 0.10 | 0.00 |

TABLE 17
Luciferase Expression

| | P00411 | | P00418 | | P00415 | |
|---|---|---|---|---|---|---|
| | Average | St. Dev | Average | St. Dev | Average | St. Dev |
| | Luciferase | Luciferase | Luciferase | Luciferase | Luciferase | Luciferase |
| Guide ID | (RLU) | (RLU) | (RLU) | (RLU) | (RLU) | (RLU) |
| G000551 | 58000.00 | 4331.28 | 41800.00 | 2165.64 | 78633.33 | 20274.70 |
| G000552 | 95700.00 | 10573.08 | 80866.67 | 27911.35 | 205333.33 | 30664.86 |
| G000553 | 205333.33 | 52993.71 | 177333.33 | 32929.22 | 471666.67 | 134001.00 |
| G000554 | 125333.33 | 55949.38 | 91933.33 | 19194.10 | 232666.67 | 67002.49 |
| G000555 | 59933.33 | 11566.04 | 77733.33 | 11061.80 | 155666.67 | 15947.83 |
| G000666 | 88500.00 | 28735.87 | 93266.67 | 30861.19 | 313000.00 | 15394.80 |
| G000667 | 75333.33 | 22653.11 | 68966.67 | 27222.11 | 153000.00 | 30805.84 |
| G000668 | 164000.00 | 56320.51 | 133400.00 | 65111.29 | 429000.00 | 120751.80 |
| G000669 | 28933.33 | 11636.29 | 22033.33 | 2413.16 | 46466.67 | 6543.19 |
| G000670 | 162666.67 | 32959.57 | 200000.00 | 33867.39 | 424666.67 | 36473.73 |
| G011722 | 16766.67 | 3384.28 | 8583.33 | 4103.10 | 24000.00 | 8915.16 |
| G011723 | 22733.33 | 7252.82 | 17133.33 | 4905.44 | 26100.00 | 8109.87 |
| G011724 | 17300.00 | 2400.00 | 28033.33 | 9091.94 | 30933.33 | 3365.02 |
| G011725 | 8253.33 | 1163.20 | 8890.00 | 1429.27 | 20366.67 | 13955.05 |
| G011726 | 12223.33 | 3742.54 | 11610.00 | 2490.44 | 14950.00 | 8176.03 |
| G011727 | 35600.00 | 8128.35 | 36300.00 | 12301.22 | 86700.00 | 5023.94 |
| G011728 | 14900.00 | 5011.99 | 22466.67 | 7130.45 | 38166.67 | 13829.08 |
| G011729 | 10460.00 | 2543.95 | 11223.33 | 2220.28 | 26966.67 | 16085.50 |
| G011730 | 14833.33 | 2307.24 | 21700.00 | 8681.59 | 41233.33 | 25687.03 |
| G011731 | 16433.33 | 3274.65 | 22566.67 | 2205.30 | 20756.67 | 13096.20 |
| AAV Only | 217.00 | 15.56 | 215.00 | 15.56 | 207.00 | 1.41 |

### Example 6—In Vivo Screening of Bidirectional Constructs Across Albumin Intron 1 Target Sites

The ssAAV and LNPs tested in this Example were prepared and delivered to C57B1/6 mice as described in Example 1 to assess the performance of the bidirectional constructs across target sites in vivo. Four weeks post dose, the animals were euthanized and liver tissue and sera were collected for editing and hFIX expression, respectively.

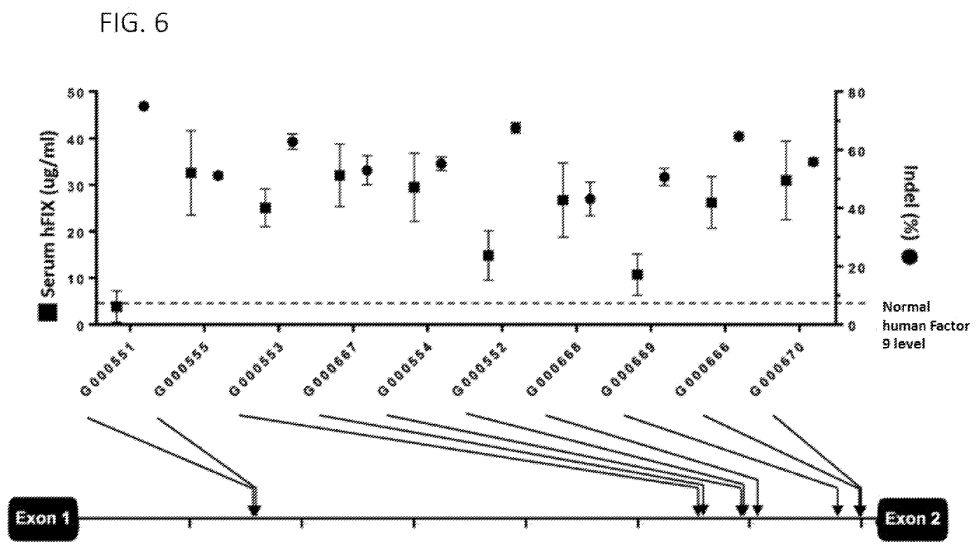

In an initial experiment, 10 different LNP formulations containing 10 different gRNA targeting intron 1 of albumin were delivered to mice along with ssAAV derived from P00147. The AAV and LNP were delivered at 3e11 vg/ms and 4 mg/kg (with respect to total RNA cargo content), respectively (n=5 for each group). The gRNAs tested in this experiment are shown in FIG. 6. As shown in FIG. 6 and Table 18 as observed in vitro, a significant level of indel formation was not predictive for insertion or expression of the transgenes.

TABLE 18
hFIX Serum Levels and % Indel

| | Average | St. Dev | Average | St. Dev |
|---|---|---|---|---|
| | Indel | Indel | Luciferase | Luciferase |
| Guide | (%) | (%) | (RLU) | (RLU) |
| G000551 | 75.02 | 1.27 | 3.82 | 3.38 |
| G000555 | 51.18 | 1.19 | 32.56 | 9.05 |
| G000553 | 62.78 | 2.64 | 25.07 | 4.04 |
| G000667 | 52.96 | 4.96 | 32.03 | 6.74 |
| G000554 | 55.24 | 2.28 | 29.48 | 7.34 |
| G000552 | 67.56 | 1.73 | 14.79 | 5.34 |
| G000668 | 43.14 | 5.78 | 26.72 | 7.97 |
| G000669 | 50.68 | 2.97 | 10.70 | 4.43 |
| G000666 | 64.62 | 1.34 | 26.19 | 5.56 |
| G000670 | 55.90 | 1.30 | 30.96 | 8.44 |

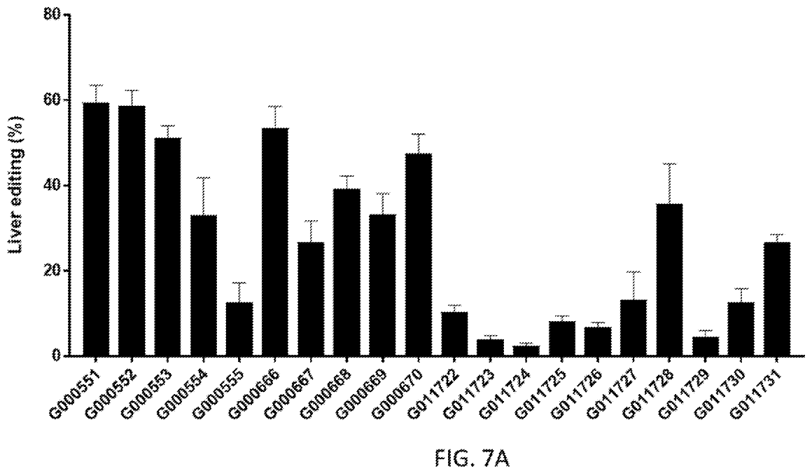

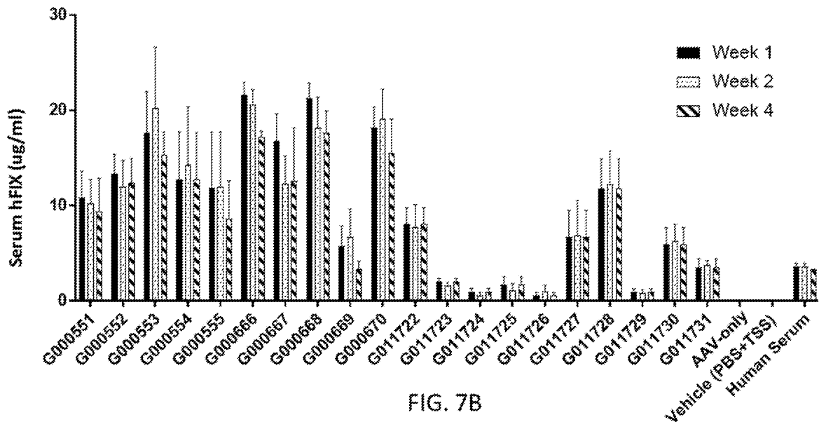

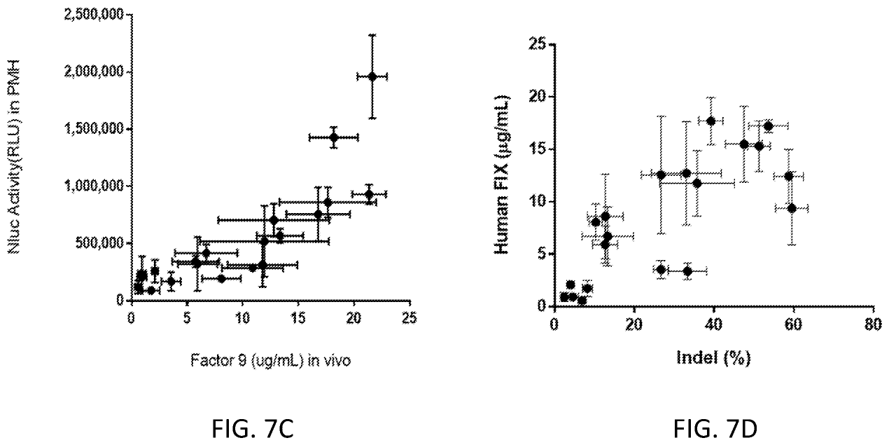

In a separate experiment, a panel of 20 gRNAs targeting the 20 different target sites tested in vitro in Example 5 were tested in vivo. To this end, LNP formulations containing the 20 gRNAs targeting intron 1 of albumin were delivered to mice along with ssAAV derived from P00147. The AAV and LNP were delivered at 3e11 vg/ms and 1 mg/kg (with respect to total RNA cargo content), respectively. The gRNAs tested in this experiment are shown in FIG. 7A and FIG. 7B.

TABLE 19
Editing in the Liver

| | Average Liver Editing | St. Dev Liver Editing |
|---|---|---|
| Guide | (%) | (%) |
| G000551 | 59.48 | 4.02 |
| G000555 | 58.72 | 3.65 |
| G000553 | 51.26 | 2.81 |
| G000554 | 33.04 | 8.76 |
| G000555 | 12.72 | 4.46 |
| G000666 | 53.60 | 4.92 |
| G000667 | 26.74 | 4.98 |
| G000668 | 39.22 | 3.04 |
| G000669 | 33.34 | 4.77 |
| G000670 | 47.50 | 5.58 |
| G011722 | 10.34 | 1.68 |
| G011723 | 4.02 | 0.84 |
| G011724 | 2.46 | 0.64 |
| G011725 | 8.26 | 1.24 |
| G011726 | 6.90 | 1.01 |
| G011727 | 13.33 | 6.43 |
| G011728 | 35.78 | 9.34 |
| G011729 | 4.62 | 1.46 |
| G011730 | 12.68 | 3.14 |
| G011731 | 26.70 | 1.86 |

TABLE 20
Serum hFIX Levels

| | Week 1 | | Week 2 | | Week 4 | |
|---|---|---|---|---|---|---|
| | Average | St. Dev | Average | St. Dev | Average | St. Dev |
| | FIX | FIX | FIX | FIX | FIX | FIX |
| Guide | (ug/mL) | (ug/mL) | (ug/mL) | (ug/mL) | (ug/mL) | (ug/mL) |
| G000551 | 10.88 | 2.74 | 10.25 | 2.51 | 9.39 | 3.48 |
| G000555 | 13.34 | 2.09 | 12.00 | 2.75 | 12.43 | 2.57 |
| G000553 | 17.64 | 4.34 | 20.27 | 6.35 | 15.31 | 2.43 |
| G000554 | 12.79 | 4.99 | 14.29 | 6.09 | 12.74 | 4.93 |
| G000555 | 11.94 | 5.79 | 11.99 | 5.76 | 8.61 | 4.02 |
| G000666 | 21.63 | 1.32 | 20.65 | 1.55 | 17.23 | 0.62 |
| G000667 | 16.77 | 2.86 | 12.35 | 2.85 | 12.57 | 5.60 |
| G000668 | 21.35 | 1.51 | 18.20 | 3.18 | 17.72 | 2.25 |
| G000669 | 5.76 | 2.10 | 6.72 | 2.93 | 3.39 | 0.78 |
| G000670 | 18.18 | 2.17 | 19.16 | 3.05 | 15.49 | 3.61 |
| G011722 | 8.07 | 1.74 | 7.74 | 2.41 | 8.07 | 1.74 |
| G011723 | 2.11 | 0.28 | 1.65 | 0.28 | 2.11 | 0.28 |
| G011724 | 0.92 | 0.43 | 0.60 | 0.30 | 0.92 | 0.43 |
| G011725 | 1.75 | 0.77 | 1.14 | 0.67 | 1.75 | 0.77 |
| G011726 | 0.59 | 0.30 | 1.01 | 0.64 | 0.59 | 0.30 |
| G011727 | 6.71 | 2.80 | 6.90 | 3.68 | 6.71 | 2.80 |
| G011728 | 11.77 | 3.12 | 12.29 | 3.43 | 11.77 | 3.12 |
| G011729 | 0.94 | 0.35 | 0.89 | 0.29 | 0.94 | 0.35 |
| G011730 | 5.93 | 1.77 | 6.33 | 1.73 | 5.93 | 1.77 |
| G011731 | 3.56 | 0.87 | 3.78 | 0.50 | 3.56 | 0.87 |
| AAV Only | 0.00 | 0.00 | 0.00 | 0.00 | 0.00 | 0.00 |
| Vehicle | 0.00 | 0.00 | 0.00 | 0.00 | 0.00 | 0.00 |
| Human Serum | 3.63 | 0.32 | 3.61 | 0.35 | 3.28 | 0.03 |

As shown, in FIG. 7A and Table 19, varied levels of editing were detected for each of the treatment groups across each LNP/vector combination tested. However, as shown in FIG. 7B and consistent with the in vitro data described in Example 5, higher levels of editing did not necessarily result in higher levels of expression of the transgenes in vivo, indicating a lack of correlation between editing and insertion/expression of the bidirectional constructs. Indeed, very little correlation exists between the amount of editing achieved and the amount of hFIX expression as viewed in the plot provided in FIG. 7D and Table 20. In particular, an R² value of only 0.34 is calculated between the editing and expression data sets for this experiment, when those gRNAs achieving less than 10% editing are removed from the analysis. Interestingly, as shown in FIG. 7C, a correlation plot is provided comparing the levels of expression as measured in RLU from the in vitro experiment of Example 5 to the transgene expression levels in vivo detected in this experiment, with an R² value of 0.70, demonstrating a positive correlation between the primary cell screening and the in vivo treatments.

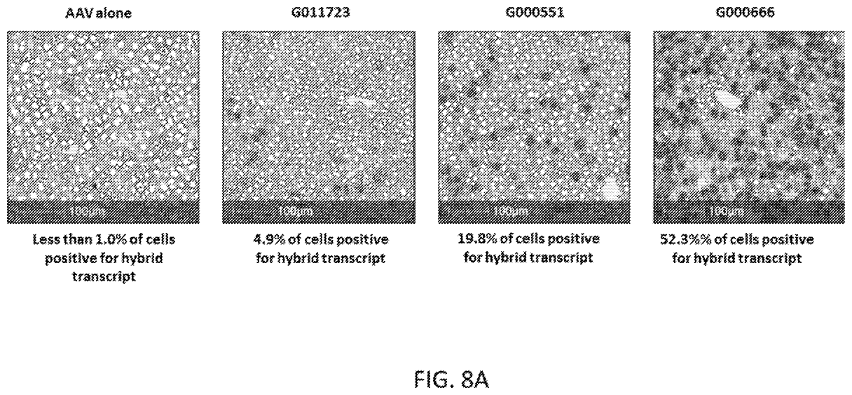

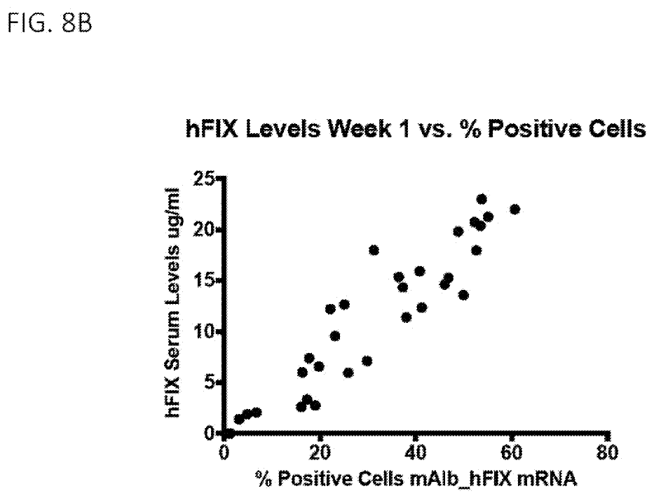

To assess insertion of the bidirectional construct at the cellular level, liver tissues from treated animals were assayed using an in situ hybridization method (BaseScope), e.g., as described in Example 1. This assay utilized probes that can detect the junctions between the hFIX transgene and the mouse albumin exon 1 sequence, as a hybrid transcript. As shown in FIG. 8A, cells positive for the hybrid transcript were detected in animals that received both AAV and LNP. Specifically, when AAV alone is administered, less than 1.0% of cells were positive for the hybrid transcript. With administration of LNPs comprising G011723, G000551, or G000666, 4.9%, 19.8%, or 52.3% of cells were positive for the hybrid transcript. Additionally, as shown in FIG. 8B, circulating hFIX levels correlated with the number of cells that were positive for the hybrid transcript. Lastly, the assay utilized pooled probes that can detect insertion of the bidirectional hFIX construct in either orientation. However, when a single probe was used that only detects a single orientation, the amount of cells that were positive for the hybrid transcript was about half that detected using the pooled probes (in one example, 4.46% vs 9.68%), suggesting that the bidirectional construct indeed is capable of inserting into intron 1 of albumin in either orientation giving rise to expressed hybrid transcripts that correlate with the amount of transgene expression at the protein level. These data show that the circulating protein levels achieved are dependent on the guide used for insertion.

### Example 7—Timing of AAV and LNP Delivery In Vivo

In this Example, the timing between delivery of ssAAV comprising the bidirectional hFIX construct and LNP for targeted insertion into intron 1 of albumin was examined.

The ssAAV and LNPs tested in this Example were prepared and delivered to mice as described in Example 1. The LNP formulation contained G000551 and the bidirectional template was delivered as ssAAV derived from P00147. The AAV and LNP were delivered at 3e11 vg/ms and 4 mg/kg (with respect to total RNA cargo content), respectively (n=5 for each group). A “Template only” cohort received AAV only, and a “PBS” cohort received no AAV or LNP. One cohort received AAV and LNP sequentially (minutes apart) at day 0 (“Template+LNP day 0”); another cohort received AAV at day 0 and LNP at day 1 (“Template+LNP day 1”); and a final cohort received AAV at day 0 and LNP at day 7 (“Template+LNP day 7”). At 1 week, 2 weeks and 6 weeks, plasma was collected for hFIX expression analysis.

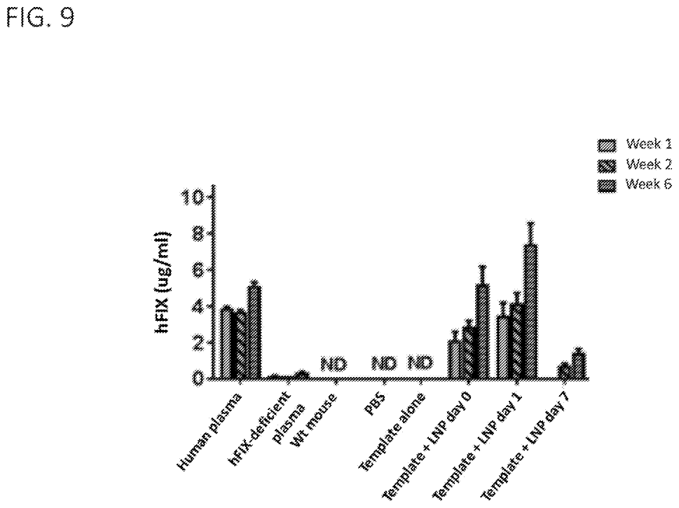

As shown in FIG. 9, hFIX was detected in each cohort at each time assayed, except for the 1 week timepoint for the cohort that received the LNP dose the same day at week 1 post AAV delivery.

### Example 8—Multiple Dosing of LNP Following Delivery of AAV

In this Example, the effects of repeat dosing of LNP following administration of ssAAV for targeted insertion into intron 1 of albumin was examined.

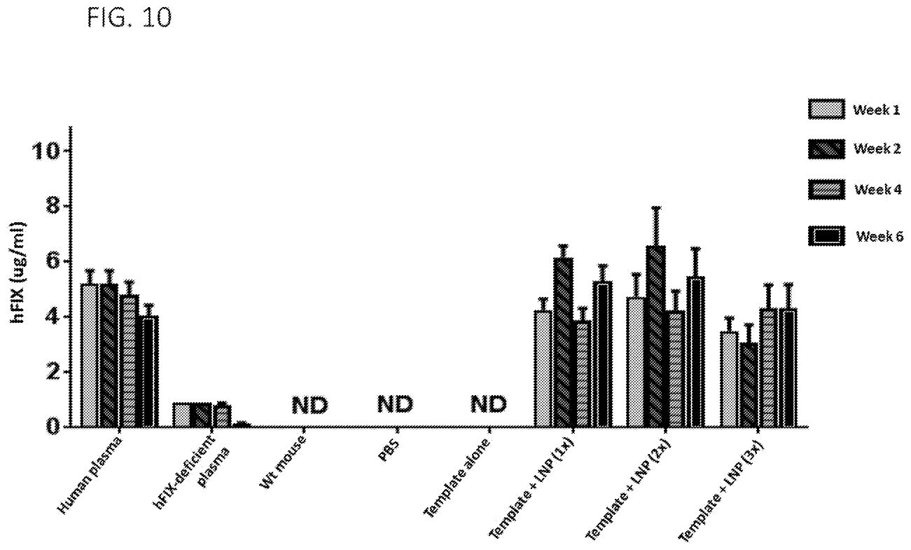

The ssAAV and LNPs tested in this Example were prepared and delivered to C57B1/6 mice as described in Example 1. The LNP formulation contained G000551 and the ssAAV was derived from P00147. The AAV and LNP were delivered at 3e11 vg/ms and 0.5 mg/kg (with respect to total RNA cargo content), respectively (n=5 for each group). A “Template only” cohort received AAV only, and a “PBS” cohort received no AAV or LNP. One cohort received AAV and LNP sequentially (minutes apart) at day 0 with no further treatments (“Template+LNP(1x)” in FIG. 10); another cohort received AAV and LNP sequentially (minutes apart) at day 0 and a second dose at day 7 (“Template+LNP(2x)” in FIG. 10); and a final cohort received AAV and LNP sequentially (minutes apart) at day 0, a second dose of LNP at day 7 and a third dose of LNP at day 14 (“Template+LNP(3x)” in FIG. 10). At 1, 2, 4 and 6 weeks post-administration of AAV, plasma was collected for hFIX expression analysis.

As shown in FIG. 10, hFIX was detected in each cohort at each time assayed, and multiple subsequent doses of LNP did not significantly increase the amount of hFIX expression.

### Example 9—Durability of hFIX Expression In Vivo

The durability of hFIX expression following targeted insertion into intron 1 of albumin over time in treated animals was assessed in this Example. To this end, hFIX was measured in the serum of treated animals as part of a one-year durability study.

The ssAAV and LNPs tested in this Example were prepared and delivered to C57B1/6 mice as described in Example 1. The LNP formulation contained G000551 and the ssAAV was derived from P00147. The AAV was delivered at 3e11 vg/ms and the LNP was delivered at either 0.25 or 1.0 mg/kg (with respect to total RNA cargo content) (n=5 for each group).

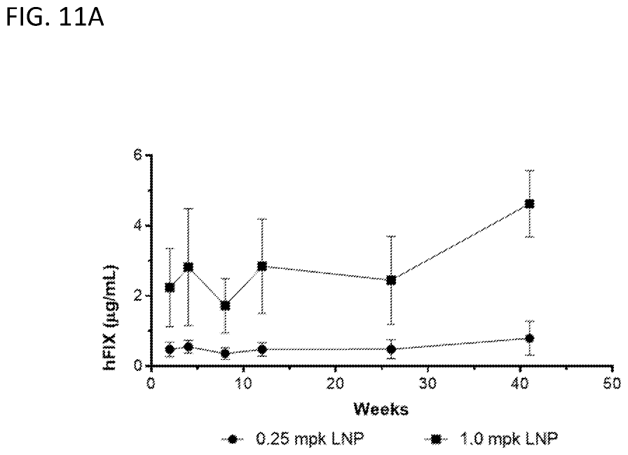

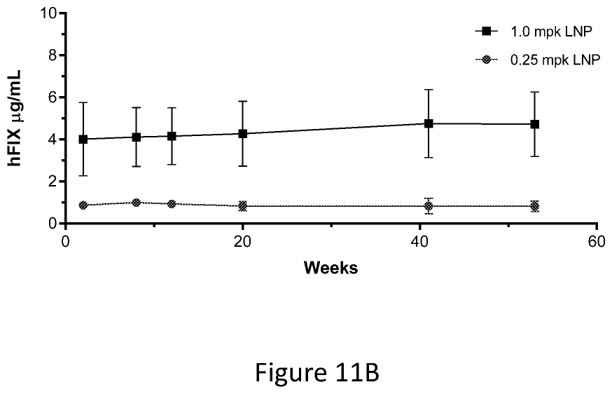

As shown in FIG. 11A and FIG. 11B and Tables 21-22, hFIX expression from intron 1 of albumin was sustained at each time point assessed for both groups out to 12 weeks. A drop in the levels observed at 8 weeks is believed to be due to the variability of the ELISA assay. Serum albumin levels were measured by ELISA at week 2 and week 41, showing that circulating albumin levels are maintained across the study.

TABLE 21
FIX Levels

| | Dose | | | |
|---|---|---|---|---|
| | 0.25 mpk LNP | | 1 mpk LNP | |
| | Average hFIX | StDev hFIX | Average hFIX | StDev hFIX |
| Week | (ug/mL) | (ug/mL) | (ug/mL) | (ug/mL) |
| 2 | 0.48 | 0.21 | 2.24 | 1.12 |
| 4 | 0.55 | 0.18 | 2.82 | 1.67 |
| 8 | 0.40 | 0.17 | 1.72 | 0.77 |
| 12 | 0.48 | 0.20 | 2.85 | 1.34 |
| 20 | 0.48 | 0.27 | 2.45 | 1.26 |
| 41 | 0.79 | 0.49 | 4.63 | 0.95 |

TABLE 22
hFIX Levels

| | Dose | | | |
|---|---|---|---|---|
| | 0.25 mpk LNP | | 1 mpk LNP | |
| | Average hFIX | StDev hFIX | Average hFIX | StDev hFIX |
| Week | (ug/mL) | (ug/mL) | (ug/mL) | (ug/mL) |
| 2 | 0.87 | 0.15 | 4.02 | 1.75 |
| 8 | 0.99 | 0.15 | 4.11 | 1.41 |
| 12 | 0.93 | 0.14 | 4.15 | 1.35 |
| 20 | 0.83 | 0.22 | 4.27 | 1.54 |
| 41 | 0.83 | 0.37 | 4.76 | 1.62 |
| 52 | 0.82 | 0.25 | 4.72 | 1.54 |

### Example 10—Effects of Varied Doses of AAV and LNP to Modulate hFIX Expression In Vivo

In this Example, the effects of varying the dose of both AAV and LNP to modulate expression of hFIX following targeted insertion into intron 1 of albumin was assessed in C57B1/6 mice.

The ssAAV and LNPs tested in this Example were prepared and delivered to mice as described in Example 1. The LNP formulation contained G000553 and the ssAAV was derived from P00147. The AAV was delivered at 1e11, 3e11, 1e12 or 3e12 vg/ms and the LNP was delivered at 0.1, 0.3, or 1.0 mg/kg (with respect to total RNA cargo content) (n=5 for each group). Two weeks post-dose, the animals were euthanized and sera were collected for hFIX expression analysis.

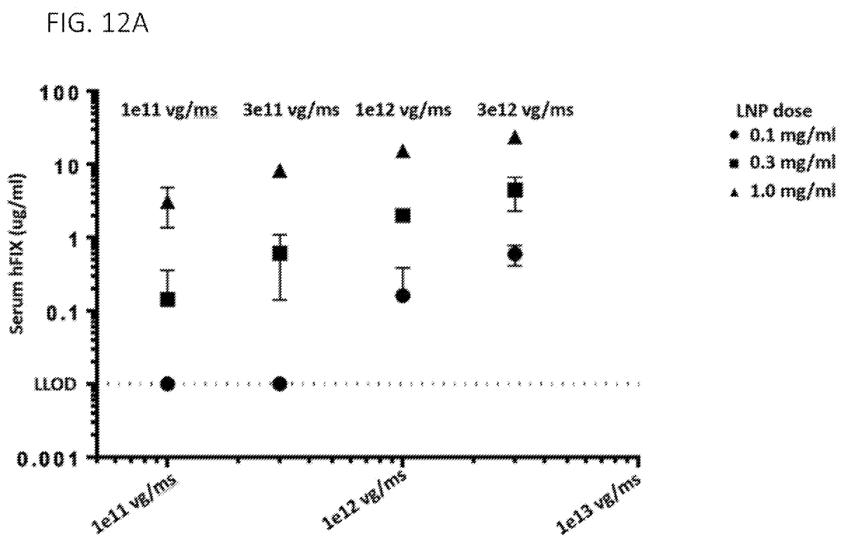

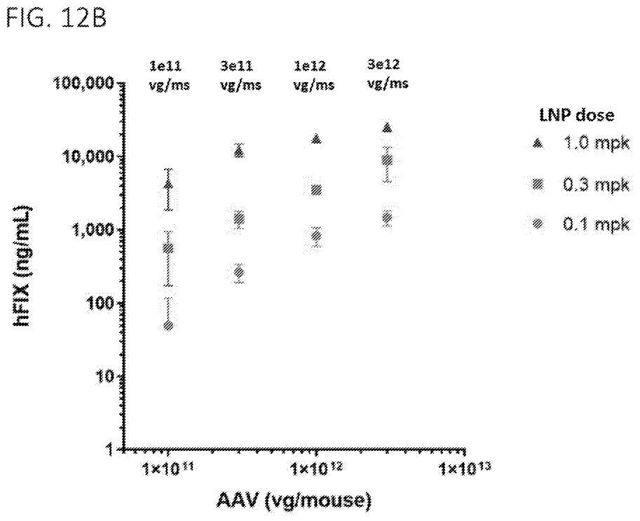

As shown in FIG. 12A (1 week) and FIG. 12B (2 weeks) and Table 23, varying the dose of either AAV or LNP can modulate the amount of expression of hFIX from intron 1 of albumin in vivo.

TABLE 23
Serum hFIX

| | RNP Dose | AAV Dose | Mean FIX | | |
|---|---|---|---|---|---|
| Timepoint | (mg/kg) | (MOI) | (ng/ml) | SD | N |
| Week 1 | 0.1 | 1E+11 | 0.08 | 0.02 | 2 |
| | | 3E+11 | 0.11 | 0.04 | 5 |
| | | 1E+12 | 0.41 | 0.15 | 5 |
| | | 3E+12 | 0.61 | 0.17 | 5 |
| | 0.3 | 1E+11 | 0.36 | 0.14 | 5 |
| | | 3E+11 | 0.67 | 0.26 | 5 |
| | | 1E+12 | 1.76 | 0.14 | 5 |
| | | 3E+12 | 4.70 | 2.40 | 5 |
| | 1.0 | 1E+11 | 3.71 | 0.31 | 4 |
| | | 3E+11 | 8.00 | 0.51 | 5 |
| | | 1E+12 | 14.17 | 1.38 | 5 |
| | | 3E+12 | 20.70 | 2.79 | 5 |
| | Human serum 1:1000 | | 6.62 | — | 1 |
| Week 2 | 0.1 | 1E+11 | 0.12 | 0.01 | 2 |
| | | 3E+11 | 0.26 | 0.07 | 5 |
| | | 1E+12 | 0.83 | 0.24 | 5 |
| | | 3E+12 | 1.48 | 0.35 | 5 |
| | 0.3 | 1E+11 | 0.70 | 0.26 | 4 |
| | | 3E+11 | 1.42 | 0.37 | 5 |
| | | 1E+12 | 3.53 | 0.49 | 5 |
| | | 3E+12 | 8.94 | 4.39 | 5 |
| | 1.0 | 1E+11 | 5.40 | 0.47 | 4 |
| | | 3E+11 | 12.31 | 2.45 | 5 |
| | | 1E+12 | 17.89 | 1.95 | 5 |
| | | 3E+12 | 25.52 | 3.62 | 5 |

| | | | |
|---|---|---|---|
| Human serum 1:1000 | 4.47 | — | 1 |

### Example 11—In Vitro Screening of Bidirectional Constructs Across Target Sites in Primary Cynomolgus and Primary Human Hepatocytes

In this Example, ssAAV vectors comprising a bidirectional construct were tested across a panel of target sites utilizing gRNAs targeting intron 1 of cynomolgus (“cyno”) and human albumin in primary cyno (PCH) and primary human hepatocytes (PHH), respectively.

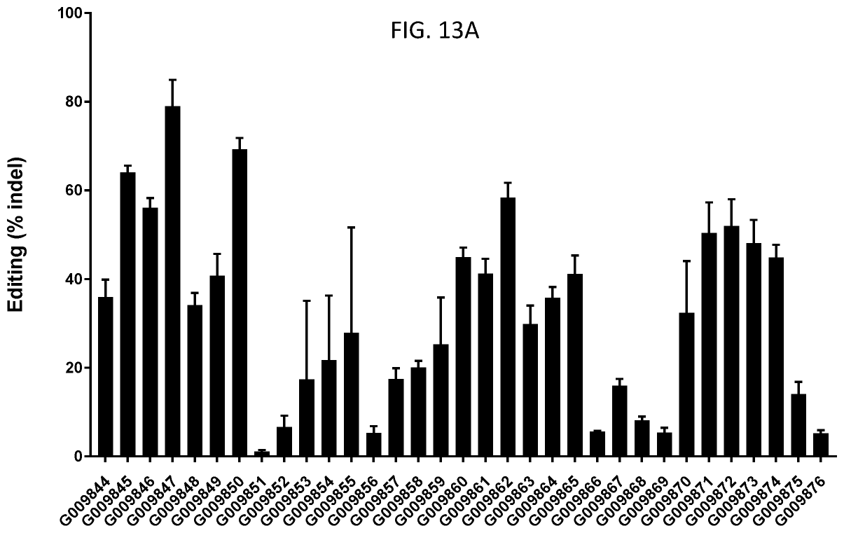

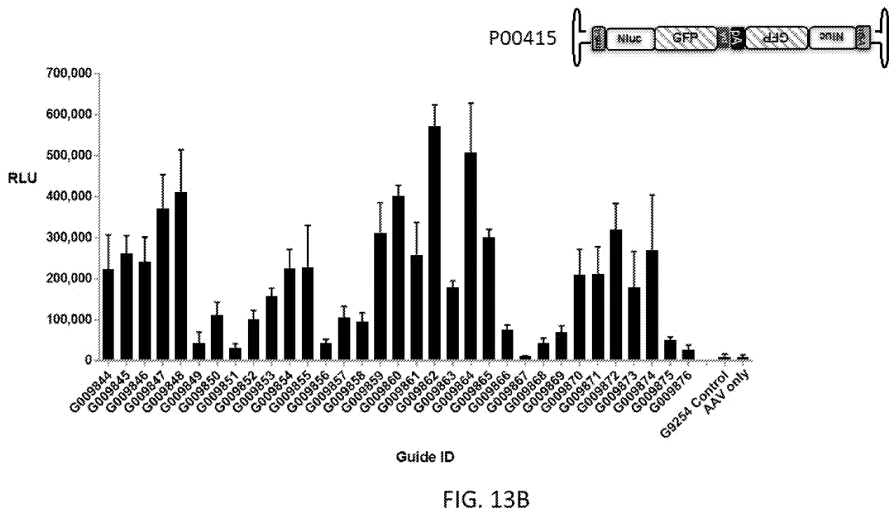

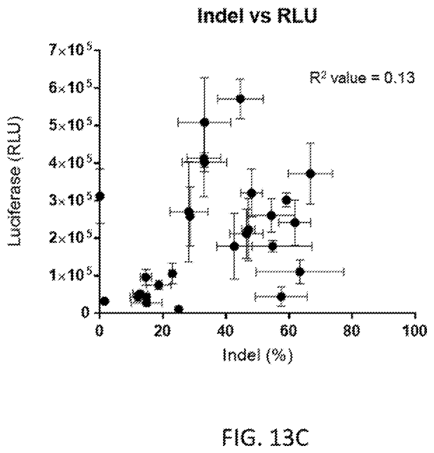

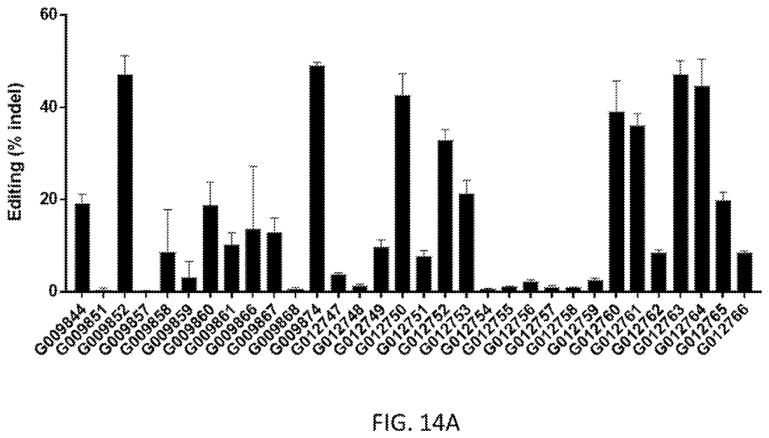

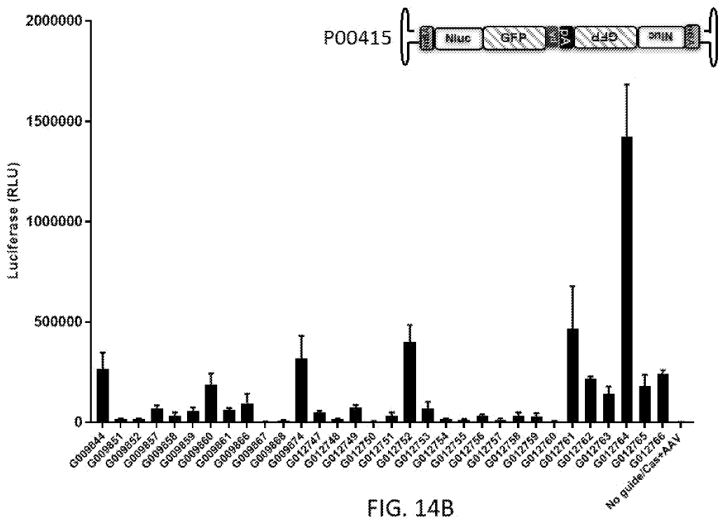

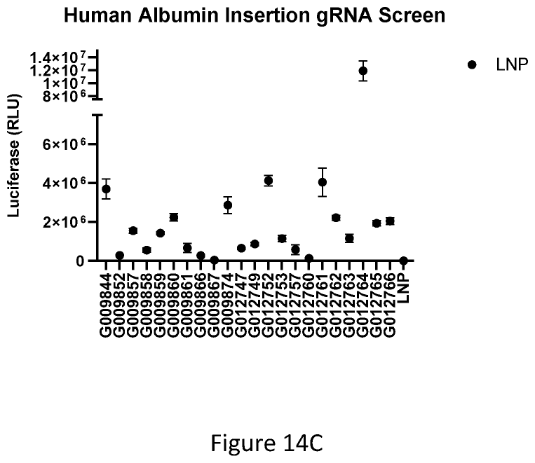

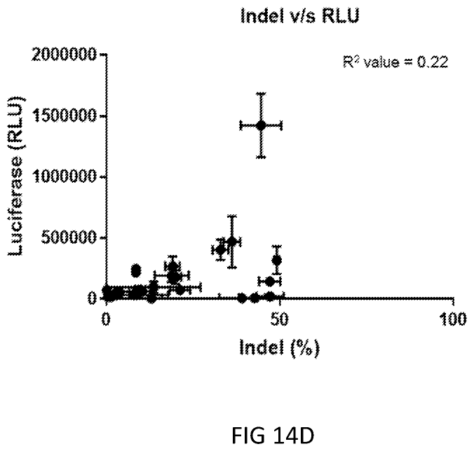

The ssAAV and lipid packet delivery materials tested in this Example were prepared and delivered to PCH and PHH as described in Example 1. Following treatment, isolated genomic DNA and cell media was collected for editing and transgene expression analysis, respectively. Each of the vectors comprised a reporter that can be measured through luciferase-based fluorescence detection as described in Example 1 (derived from plasmid P00415), plotted in FIG. 13B and FIG. 14B as relative luciferase units (“RLU”). The RLU data shown in FIG. 13B and FIG. 14B graphically, are reproduced numerically in Table 3 and Table 4 below. For example, the AAV vectors contained the NanoLuc ORF (in addition to GFP). Schematics of the vectors tested are provided in FIG. 13B and FIG. 14B. The gRNAs tested are shown in each of the Figures using a shortened number for those listed in Table 1 and Table 3.

As shown in FIG. 13A for PCH and FIG. 14A for PHH, varied levels of editing were detected for each of the combinations tested (editing data for some combinations tested in the PCH experiment are not reported in FIG. 13A and Table 3 due to failure of certain primer pairs used for the amplicon based sequencing). The editing data shown in FIG. 13A and FIG. 14A graphically, are reproduced numerically in Table 3 and Table 4 below. However, as shown in FIG. 13B, FIG. 13C and FIG. 14B and FIG. 14C, a significant level of indel formation was not predictive for insertion or expression of the transgenes, indicating little correlation between editing and insertion/expression of the bidirectional constructs in PCH and PHH, respectively. As one measure, the R² value calculated in FIG. 13C is 0.13, and the R² value of FIG. 14D is 0.22.

TABLE 3
Albumin intron 1 editing and transgene expression data
for sgRNAs delivered to primary cynomolgus hepatocytes

| | Avg % | Std Dev % | Avg | Std Dev |
|---|---|---|---|---|
| GUIDE ID | Edit | Edit | RLU | RLU |
| G009867 | 25.05 | 0.21 | 10650.67 | 1455.97 |
| G009866 | 18.7 | 3.96 | 75556.67 | 12182.98 |
| G009876 | 14.85 | 4.88 | 27463.33 | 10833.53 |
| G009875 | 12.85 | 2.33 | 51660.00 | 6362.36 |
| G009874 | 28.25 | 6.01 | 270433.30 | 133734.10 |
| G009873 | 42.65 | 5.59 | 178600.00 | 87607.25 |
| G009865 | 59.15 | 0.21 | 301666.70 | 18610.03 |
| G009872 | 48.15 | 3.46 | 320233.30 | 63517.43 |
| G009871 | 46.5 | 5.23 | 211966.70 | 65852.44 |
| G009864 | 33.2 | 8.34 | 210033.30 | 61201.33 |
| G009863 | 54.8 | 12.45 | 69853.33 | 15216.92 |
| G009862 | 44.6 | 7.21 | 508666.70 | 119876.30 |
| G009861 | 28.65 | 0.21 | 178666.70 | 15821.93 |
| G009860 | 33.2 | 7.07 | 571333.30 | 52728.87 |
| G009859 | 0.05 | 0.07 | 258333.30 | 79052.73 |
| G009858 | 14.65 | 1.77 | 402333.30 | 25579.94 |
| G009857 | 23 | 0.99 | 312333.30 | 73036.52 |
| G009856 | 14.8 | 0.99 | 95900.00 | 21128.42 |
| G009851 | 1.5 | 0.42 | 105766.70 | 27048.91 |
| G009868 | 12.15 | 2.47 | 43033.33 | 9141.85 |
| G009850 | 63.45 | 13.93 | 228200.00 | 101542.10 |
| G009849 | 57.55 | 8.27 | 225400.00 | 46001.30 |
| G009848 | 33 | 5.37 | 156333.30 | 20647.84 |
| G009847 | 66.75 | 7 | 100866.70 | 22159.72 |
| G009846 | 61.85 | 5.02 | 31766.67 | 10107.59 |
| G009845 | 54.4 | 7.5 | 43020.00 | 11582.23 |
| G009844 | 47.15 | 2.05 | 110466.70 | 32031.44 |

TABLE 4
Albumin intron 1 editing and transgene expression data
for sgRNAs delivered to primary human hepatocytes

| | Avg % | Std Dev % | Avg | Std Dev |
|---|---|---|---|---|
| GUIDE ID | Edit | Edit | RLU | RLU |
| G009844 | 19.07 | 2.07 | 268333.30 | 80432.17 |
| G009851 | 0.43 | 0.35 | 18033.33 | 2145.54 |
| G009852 | 47.20 | 3.96 | 18400.00 | 2251.67 |
| G009857 | 0.10 | 0.14 | 71100.00 | 14609.24 |
| G009858 | 8.63 | 9.16 | 32000.00 | 18366.55 |
| G009859 | 3.07 | 3.50 | 59500.00 | 16014.99 |
| G009860 | 18.80 | 4.90 | 190333.30 | 54307.76 |
| G009861 | 10.27 | 2.51 | 62233.33 | 9865.26 |
| G009866 | 13.60 | 13.55 | 96200.00 | 46573.81 |
| G009867 | 12.97 | 3.04 | 3916.67 | 1682.03 |
| G009868 | 0.63 | 0.32 | 10176.67 | 2037.80 |
| G009874 | 49.13 | 0.60 | 318000.00 | 114118.40 |
| G012747 | 3.83 | 0.23 | 51000.00 | 6161.17 |
| G012748 | 1.30 | 0.35 | 17433.33 | 2709.86 |
| G012749 | 9.77 | 1.50 | 75066.67 | 11809.04 |
| G012750 | 42.73 | 4.58 | 5346.67 | 2977.35 |
| G012751 | 7.77 | 1.16 | 32066.67 | 18537.62 |
| G012752 | 32.93 | 2.27 | 402000.00 | 83144.45 |
| G012753 | 21.20 | 2.95 | 71800.00 | 32055.73 |
| G012754 | 0.60 | 0.10 | 16933.33 | 4254.80 |
| G012755 | 1.10 | 0.10 | 13833.33 | 3685.56 |
| G012756 | 2.17 | 0.40 | 35600.00 | 6055.58 |
| G012757 | 1.07 | 0.25 | 13993.33 | 6745.08 |
| G012758 | 0.90 | 0.10 | 34900.00 | 15308.82 |
| G012759 | 2.60 | 0.35 | 30566.67 | 15287.36 |
| G012760 | 39.10 | 6.58 | 6596.67 | 2133.13 |
| G012761 | 36.17 | 2.43 | 467666.70 | 210965.20 |
| G012762 | 8.50 | 0.57 | 217000.00 | 13000.00 |
| G012763 | 47.07 | 3.07 | 142333.30 | 37581.02 |
| G012764 | 44.57 | 5.83 | 1423333.00 | 261023.60 |
| G012765 | 19.90 | 1.68 | 179666.70 | 57011.69 |
| G012766 | 8.50 | 0.28 | 243333.30 | 17473.79 |

Additionally, ssAAV vectors comprising a bidirectional construct were tested across a panel of target sites utilizing single guide RNAs targeting intron 1 of human albumin in primary human hepatocytes (PHH).

The ssAAV and LNP materials were prepared and delivered to PHH as described in Example 1. Following treatment, isolated genomic DNA and cell media was collected for editing and transgene expression analysis, respectively. Each of the vectors comprised a reporter that can be measured through luciferase-based fluorescence detection as described in Example 1 (derived from plasmid P00415), plotted in FIG. 14D and shown in Table 24 as relative luciferase units (“RLU”). For example, the AAV vectors contained the NanoLuc ORF (in addition to GFP). Schematics of the vectors tested are provided in FIG. 13B and FIG. 14B. The gRNAs tested are shown in FIG. 14D using a shortened number for those listed in Table 1. and Table 7

TABLE 24
Albumin intron 1 transgene expression data for sgRNAs
delivered to primary cynomolgus hepatocytes

| | Average Luciferase | St. Dev Luciferase |
|---|---|---|
| Guide | (RLU) | (RLU) |
| G009844 | 3,700,000 | 509,117 |
| G009852 | 281,000 | 69,296 |
| G009857 | 1,550,000 | 127,279 |
| G009858 | 551,000 | 108,894 |
| G009859 | 1,425,000 | 77,782 |
| G009860 | 2,240,000 | 183,848 |
| G009861 | 663,500 | 238,295 |
| G009866 | 274,000 | 11,314 |
| G009867 | 44,700 | 566 |
| G009874 | 2,865,000 | 431,335 |
| G012747 | 651,000 | 59,397 |
| G012749 | 867,000 | 93,338 |
| G012752 | 4,130,000 | 268,701 |
| G012753 | 1,145,000 | 162,635 |
| G012757 | 579,000 | 257,387 |
| G012760 | 129,000 | 36,770 |
| G012761 | 4,045,000 | 728,320 |
| G012762 | 2,220,000 | 127,279 |
| G012763 | 1,155,000 | 205,061 |
| G012764 | 11,900,000 | 1,555,635 |
| G012765 | 1,935,000 | 134,350 |
| G012766 | 2,050,000 | 169,706 |
| LNP | 8,430 | 212 |

### Example 12—In Vivo Testing of the Human Factor 9 Gene Insertion in Non-Human Primates

In this example, an 8 week study was performed to evaluate the human Factor 9 gene insertion and hFIX protein expression in cynomolgus monkeys through administration of adeno-associated virus (AAV) and/or lipid nanoparticles (LNP) with various guides. This study was conducted with LNP formulations and AAV formulations prepared as described above. Each LNP formulation contained Cas9 mRNA and guide RNA (gRNA) with an mRNA:gRNA ratio of 2:1 by weight. The ssAAV was derived from P00147.

Male cynomolgus monkeys were treated in cohorts of n=3. Animals were dosed with AAV by slow bolus injection or infusion in the doses described in Table 5. Following AAV treatment, animals received buffer or LNP as described in Table 5 by slow bolus or infusion.

Two weeks post-dose, liver specimens were collected through single ultrasound-guided percutaneous biopsy. Each biopsy specimen was flash frozen in liquid nitrogen and stored at −86 to −60° C. Editing analysis of the liver specimens was performed by NGS Sequencing as previously described.

For Factor IX ELISA analysis, blood samples were collected from the animals on days 7, 14, 28, and 56 post-dose. Blood samples were collected and processed to plasma following blood draw and stored at −86 to −60° C. until analysis.

The total human Factor IX levels were determined from plasma samples by ELISA. Briefly, Reacti-Bind 96-well microplate (VWR Cat #PI15041) were coated with capture antibody (mouse mAB to human Factor IX antibody (HTI, Cat #AHIX-5041)) at a concentration of 1 μg/ml then blocked using 1×PBS with 5% Bovine Serum Albumin. Test samples or standards of purified human Factor IX protein (ERL, Cat #HFIX 1009, Lot #HFIX4840) diluted in Cynomolgus monkey plasma were next incubated in individual wells. The detection antibody (Sheep anti-human Factor 9 polyclonal antibody, Abcam, Cat #ab128048) was adsorbed at a concentration of 100 ng/ml. The secondary antibody (Donkey anti-Sheep IgG pAbs with HRP, Abcam, Cat #ab97125) was used at 100 ng/mL. TMB Substrate Reagent set (BD OptEIA Cat #555214) was used to develop the plate. Optical density was assessed spectrophotometrically at 450 nm on a microplate reader (Molecular Devices i3 system) and analyzed using SoftMax pro 6.4.

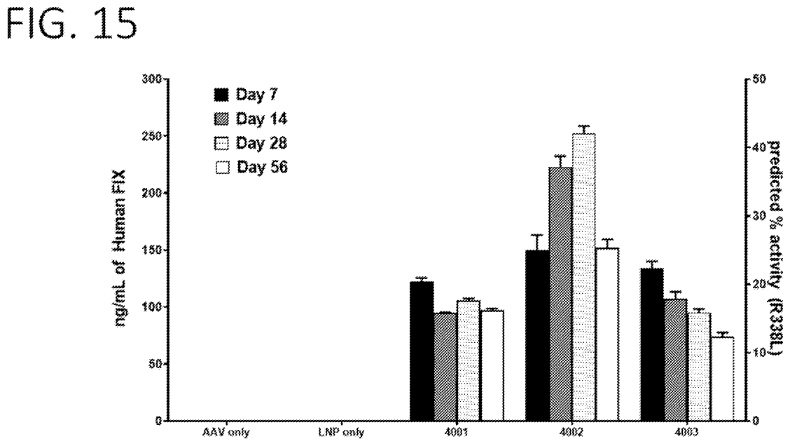

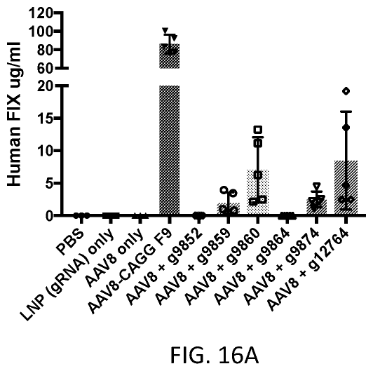

Indel formation was detected, confirming that editing occurred. The NGS data showed effective indel formation. Expression of hFIX from the albumin locus in NHPs was measured by ELISA and is depicted in Table 6 and FIG. 15. Plasma levels of hFIX reached levels previously described as therapeutically effective (George, et al., NEJM 377(23), 2215-27, 2017).

As measured, circulating hFIX protein levels were sustained through the eight week study (see FIG. 15, showing day 7, 14, 28, and 56 average levels of ˜135, ˜140, ˜150, and ˜110 ng/mL, respectively), achieving protein levels ranging from ˜75 ng/mL to ˜250 ng/mL. Plasma hFIX levels were calculated using a specific activity of ˜8 fold higher for the R338L hyperfunctional hFIX variant (Simioni et al., NEJM 361(17), 1671-75, 2009) (which reports a protein-specific activity of hFIX-R338L of 390±28 U per milligram, and a protein-specific activity for wild-type factor IX of 45±2.4 U per milligram). Calculating the functionally normalized Factor IX activity for the hyperfunctional Factor IX variant tested in this example, the experiment achieved stable levels of human Factor IX protein in the NHPs over the 8 week study that correspond to about 20-40% of wild type Factor IX activity (range spans 12-67% of wild type Factor IX activity).

TABLE 5
Editing in liver

| | | | F9-AAV | | LNP |
|---|---|---|---|---|---|
| | | F9-AAV | Volume | LNP | Volume |
| Animal ID | Guide ID | (vg/kg) | (mL/kg) | (mg/kg) | (mL/kg) |
| 4001 | G009860 | 3E+13 | 1 | 3 | 2 |
| 4002 | G009860 | 3E+13 | 1 | 3 | 2 |
| 4003 | G009860 | 3E+13 | 1 | 3 | 2 |
| 5001 | TSS | 3E+13 | 1 | 0 | 0 |
| 5002 | TSS | 3E+13 | 1 | 0 | 0 |
| 5003 | TSS | 3E+13 | 1 | 0 | 0 |
| 6001 | G009862 | 0 | 0 | 3 | 2 |
| 6002 | G009862 | 0 | 0 | 3 | 2 |
| 6003 | G009862 | 0 | 0 | 3 | 2 |

TABLE 6
hFIX expression

| | Day 7 | Day 14 | Day 28 | Day 56 |
|---|---|---|---|---|
| | Factor IX | Factor IX | Factor IX | Factor IX |
| Animal ID | (ng/mL) | (ng/mL) | (ng/mL) | (ng/mL) |
| 4001 | 122.84/+−2.85 | 94.93/+−0.56 | 105.65/+−1.94 | 97.31/+−1.49 |
| 4002 | 149.77/+−13.5 | 222.92/+−9.61 | 252.49/+−6.46 | 152.05/+−7.46 |
| 4003 | 134.06/+−6.17 | 107.04/+−6.46 | 95.30/+−3.18 | 74.23/+−3.53 |
| 5001 | ND | ND | ND | ND |
| 5002 | ND | ND | ND | ND |
| 5003 | ND | ND | ND | ND |
| 6001 | ND | ND | ND | ND |
| 6002 | ND | ND | ND | ND |
| 6003 | ND | ND | ND | ND |

### Example 13 In Vivo Testing of Factor 9 Insertion in Non-Human Primates

In this example, a study was performed to evaluate the Factor 9 gene insertion and hFIX protein expression in cynomolgus monkeys following administration of ssAAV derived from P00147 and/or CRISPR/Cas9 lipid nanoparticles (LNP) with various guides including G009860 and various LNP components.

Indel formation was measured by NGS, confirming that editing occurred. Total human Factor IX levels were determined from plasma samples by ELISA using a mouse mAB to human Factor IX antibody (HTI, Cat #AHIX-5041), sheep anti-human Factor 9 polyclonal antibody (Abcam, Cat #ab128048), and donkey anti-Sheep IgG pAbs with HRP (Abcam, Cat #ab97125), as described in Example 12. Human FIX protein levels >3 fold higher than those achieved in the experiment of Example 12 were obtained from the bidirectional template using alternative CRISPR/Cas9 LNP. In the study, ELISA assay results indicate that circulating hFIX protein levels at or above the normal range of human FIX levels (3-5 ug/mL; Amiral et al., Clin. Chem., 30(9), 1512-16, 1984) were achieved using G009860 in the NHPs by at least the day 14 and 28 timepoints. Initial data indicate circulating human FIX protein levels of ˜3-4 μg/mL at day 14 after a single dose, with levels sustained through the first 28 days (˜3-5 μg/mL) of the study. The human FIX levels were measured at the conclusion of the study by the same method and data are presented in the Table 25.

TABLE 25
Serum human Factor IX protein levels -ELISA Method of Example 13

| | Day 7 | | Day 14 | | Day 28 | | Day 42 | | Day 56 | |
|---|---|---|---|---|---|---|---|---|---|---|
| | FIX | STD | FIX | STD | FIX | STD | FIX | STD | FIX | STD |
| | ng/mL | DEV | ng/mL | DEV | ng/mL | DEV | ng/mL | DEV | ng/mL | DEV |
| 3001 | 2532.8 | 145.6 | 2562.6 | 99.0 | 3011.7 | 62.7 | 2936.7 | 72.4 | 2748.5 | 86.0 |
| 3002 | 2211.4 | 95.8 | 2958.5 | 119.2 | 3350.2 | 98.4 | 3049.7 | 112.7 | 3036.7 | 90.6 |
| 3003 | 3195.1 | 475.6 | 4433.9 | 238.7 | 3367.2 | 157.7 | 3746.1 | 95.6 | 3925.0 | 157.4 |

Circulating albumin levels were measured by ELISA, indicating that baseline albumin levels are maintained at 28 days. Tested albumin levels in untreated animals varied ±˜15% in the study. In treated animals, circulating albumin levels changed minimally and did not drop out of the normal range, and the levels recovered to baseline within one month.

Circulating human FIX protein levels were also determined by a sandwich immunoassay with a greater dynamic range. Briefly, an MSD GOLD 96-well Streptavidin SECTOR Plate (Meso Scale Diagnostics, Cat. L15SA-1) was blocked with 1% ECL Blocking Agent (Sigma, GERPN2125). After tapping out the blocking solution, biotinylated capture antibody (Sino Biological, 11503-R044) was immobilized on the plate. Recombinant human FIX protein (Enzyme Research Laboratories, HFIX 1009) was used to prepare a calibration standard in 0.5% ECL Blocking Agent. Following a wash, calibration standards and plasma samples were added to the plate and incubated. Following a wash, a detection antibody (Haematologic Technologies, AHIX-5041) conjugated with a sulfo-tag label was added to the wells and incubated. After washing away any unbound detection antibody, Read Buffer T was applied to the wells. Without any additional incubation, the plate was imaged with an MSD Quick Plex SQ120 instrument and data was analyzed with Discovery Workbench 4.0 software package (Meso Scale Discovery). Concentrations are expressed as mean calculated concentrations in ug/m. For the samples, N=3 unless indicated with an asterisk, in which case N=2. Expression of hFIX from the albumin locus in the treated study group as measured by the MSD ELISA is depicted in Table 26.

TABLE 26
Serum human Factor IX protein levels

| | Mean Calc. Conc. (ug/mL) | | | | | |
|---|---|---|---|---|---|---|
| | 3001 | | 3002 | | 3003 | |
| Time | | Inter- | | Inter- | | Inter- |
| Point | Conc. | Assay CV | Conc. | Assay CV | Conc. | Assay CV |
| Day 7 | 7.85 | 20% | 5.63 | 14% | 11.20 | 26% |
| Day 14 | 8.65 | 15% | 11.06 | 18% | 14.70 | 28% |
| Day 28 | 9.14 | 7% | 14.12 | 7% | 10.85 | 25% |
| Day 42 | 9.03 | 10% | 33.12* | 0% | 13.22 | 13% |
| Day 56 | 10.24 | 13% | 16.72 | 12% | 33.84* | 4% |

### Example 14 Off-Target Analysis of Albumin Human Guides

A biochemical method (See, e.g., Cameron et al., Nature Methods. 6, 600-606; 2017) was used to determine potential off-target genomic sites cleaved by Cas9 targeting albumin. In this experiment, 13 sgRNA targeting human albumin and two control guides with known off-target profiles were screened using isolated HEK293 genomic DNA. The number of potential off-target sites detected using a guide concentration of 16 nM in the biochemical assay were shown in Table 27. The assay identified potential off-target sites for the sgRNAs tested.

TABLE 27
Off-Target Analysis

| | | Guide Sequence | Off-Target |
|---|---|---|---|
| gRNA ID | Target | (SEQ ID NO:) | Site Count |
| G012753 | Albumin | GACUGAAACUUCACAGAAUA | 62 |
| | | (SEQ ID NO: 20) | |
| G012761 | Albumin | AGUGCAAUGGAUAGGUCUUU | 75 |
| | | (SEQ ID NO: 28) | |
| G012752 | Albumin | UGACUGAAACUUCACAGAAU | 223 |
| | | (SEQ ID NO: 19) | |
| G012764 | Albumin | CCUCACUCUUGUCUGGGCAA | 3985 |
| | | (SEQ ID NO: 31) | |
| G012763 | Albumin | UGGGCAAGGGAAGAAAAAAA | 5443 |
| | | (SEQ ID NO: 30) | |
| G009857 | Albumin | AUUUAUGAGAUCAACAGCAC | 131 |
| | | (SEQ ID NO: 5) | |
| G009859 | Albumin | UUAAAUAAAGCAUAGUGCAA | 91 |
| | | (SEQ ID NO: 7) | |
| G009860 | Albumin | UAAAGCAUAGUGCAAUGGAU | 133 |
| | | (SEQ ID NO: 8) | |
| G012762 | Albumin | UGAUUCCUACAGAAAAACUC | 68 |
| | | (SEQ ID NO: 29) | |
| G009844 | Albumin | GAGCAACCUCACUCUUGUCU | 107 |
| | | (SEQ ID NO: 2) | |
| G012765 | Albumin | ACCUCACUCUUGUCUGGGCA | 41 |
| | | (SEQ ID NO: 32) | |
| G012766 | Albumin | UGAGCAACCUCACUCUUGUC | 78 |
| | | (SEQ ID NO: 33) | |
| G009874 | Albumin | UAAUAAAAUUCAAACAUCCU | 53 |
| | | (SEQ ID NO: 13) | |
| G000644 | EMX1 | GAGUCCGAGCAGAAGAAGAA | 304 |
| | | (SEQ ID NO 1129) | |
| G000645 | VEGFA | GACCCCCUCCACCCCGCCUC | 1641 |
| | | (SEQ ID NO 1130) | |

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by this method are validated using targeted sequencing of the identified potential off-target sites.

### Example 15 Construction of Constructs for the Expression of Secretory or Non Secretory Proteins

Constructs, such as bidirectional constructs, can be designed such that they express secretory or non secretory proteins. For the production of a secretory protein, a construct may comprise a signal sequence which aids in translocating the polypeptide to the ER lumen. Alternatively, a construct may utilize the endogenous signal sequence of the host cell (e.g., the endogenous albumin signal sequence when the transgene is integrated into a host cell's albumin locus).

In contrast, constructs for the expression of non secretory proteins may be designed such that they do not comprise a signal sequence and such that they do not utilize the endogenous signal sequence of the host cell. Some methods by which this may be achieved include the incorporation of an Internal ribosome entry site (IRES) sequence in the construct. IRES sequences, such as EMCV IRES, allow for the initiation of translation from any position within an mRNA immediately downstream from where the IRES is located. This would allow for the expression of a protein which lacks the endogenous signal sequence of the host cell from an insertion site that contains a signal sequence upstream (e.g. the signal sequence found in Exon 1 of albumin locus would not be included in the expressed protein). In the absence of a signal sequence, the protein would not be secreted. Examples of IRES sequences that can be used in a construct, include those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SW) or cricket paralysis viruses (CrPV).

An alternative approach for expressing non secretory proteins is to include one or more self-cleaving peptides upstream of the polypeptide of interest in the construct. A self cleaving peptide, such as 2A or 2A-like sequences, serve as ribosome skipping signals to produce multiple individual proteins from a single mRNA transcript. As shown in Plasmid ID P00415 from Table 11, a self cleaving peptide (e.g. P2A) can be used to generate a bicistronic vector which expresses two transgenes (e.g., nanoluciferase and GFP). Alternatively, a self cleaving peptide can be used to express a protein which lacks the endogenous signal sequence of the host cell (e.g. the 2A sequence located upstream of the protein of interest would result in cleavage between the endogenous albumin signal sequence and the protein of interest). Representative 2A peptides which could be utilized are shown in Table 28. Additionally, (GSG) residues may be added to the 5′ end of the peptide to improve cleavage efficiency as shown in Table 12.

TABLE 28
Self cleaving peptides for use in constructs

| Peptide | Amino Acid Sequence |
|---|---|
| T2A (SEQ ID NO: 1131) | EGRGSLLTCGDVEENPGP |
| P2A (SEQ ID NO: 1132) | ATNFSLLKQAGDVEENPGP |
| E2A (SEQ ID NO: 1133) | QCTNYALLKLAGDVESNPGP |
| F2A (SEQ ID NO: 1134) | VKQTLNFDLLKLAGDVESNPGP |
| T2A with GSG residues | GSGEGRGSLLTCGDVEENPGP |
| (SEQ ID NO: 1135) | |
| P2A with GSG residues | GSGATNFSLLKQAGDVEENPGP |
| (SEQ ID NO: 1136) | |
| E2A with GSG residues | GSGQCTNYALLKLAGDVESNPGP |
| (SEQ ID NO: 1137) | |
| F2A with GSG residues | GSGVKQTLNFDLLKLAGDVESNPGP |
| (SEQ ID NO: 1138) | |

### Example 16. Use of Humanized Albumin Mice to Screen Guide RNAs for Human F9 Insertion In Vivo

We aimed to identify effective guide RNAs for hF9 insertion into the human albumin locus. To this end, we utilized mice in which the mouse albumin locus was replaced with the corresponding human albumin genomic sequence, including the first intron (ALB^{hu/hu} mice). This allowed us to test the insertion efficiency of guide RNAs targeting the first intron of human albumin in the context of an adult liver in vivo. Two separate mouse experiments were set up using the ALB^{hu/hu} mice to screen a total of 11 guide RNAs, each targeting the first intron of the human albumin locus. All mice were weighed and injected via tail vein at day 0 of the experiment. Blood was collected at weeks 1, 3, 4, and 6 via tail bleed, and plasma was separated. Mice were terminated at week 7. Blood was collected via the vena cava, and plasma was separated. Livers and spleens were dissected as well.

In the first experiment, 6 LNPs comprising Cas9 mRNA and the following guides were prepared as in Example 1 and tested: G009852, G009859, G009860, G009864, G009874, and G012764. LNPs were diluted to 0.3 mg/kg (using an average weight of 30 grams) and co-injected with AAV8 packaged with the bi-directional hF9 insertion template at a dose of 3E11 viral genomes per mouse. Five ALB^{hu/hu} male mice between 12 and 14 weeks old were injected per group. Five mice from same cohort were injected with AAV8 packaged with a CAGG promoter operably linked to hF9, which leads to episomal expression of hF9 (at 3E11 viral genomes per mouse). There were three negative control groups with three mice per group that were injected with buffer alone, AAV8 packaged with the bi-directional hF9 insertion template alone, or LNP-G009874 alone.

In the experiment, the following LNPs comprising Cas9 mRNA and the following guides were prepared as in Example 1 and tested: G009860, G012764, G009844, G009857, G012752, G012753, and G012761. All were diluted to 0.3 mg/kg (using an average weight of 40 grams) and co-injected with AAV8 packaged with the bi-directional hF9 insertion template at a dose of 3E11 viral genomes per mouse. Five ALB^{hu/hu} male mice 30 weeks old were injected per group. Five mice from same cohort were injected with AAV8 packaged with a CAGG promoter operably linked to hF9, which leads to episomal expression of hF9 (at 3E11 viral genomes per mouse). There were three negative control groups with three mice per group that were injected with buffer alone, AAV8 packaged with the bi-directional hF9 insertion template alone, or LNP-G009874 alone.

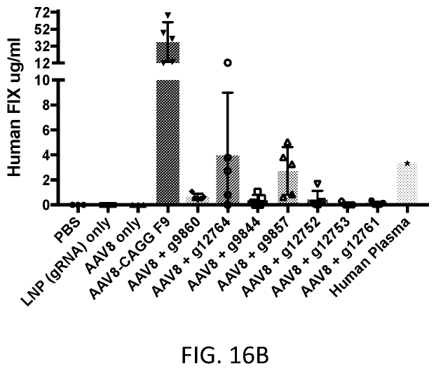

For analysis, an ELISA was performed to measure levels of hFIX circulating in the mice at each timepoint. Human Factor IX ELISA Kits (ab188393) were used for this purpose, and all plates were run with human pooled normal plasma from George King Bio-Medical as a positive assay control. Human Factor IX expression levels in the plasma samples in each group at week 6 post-injection are shown in FIG. 16A and FIG. 16B. Consistent with the in vitro insertion data, low to no Factor IX serum levels were detected when guide RNA G009852 was used. Consistent with the lack of an adjacent PAM sequence in human albumin, Factor IX serum levels were not detectable when guide RNA G009864 was used. Factor IX expression in the serum was observed for the groups using guide RNAs G009859, G009860, G009874, and G0012764.

Spleens and a portion of the left lateral lobe of all livers were submitted for next-generation sequencing (NGS) analysis. NGS was used to assess the percentage of liver cells with insertions/deletions (indels) at the humanized albumin locus at week 7 post-injection with AAV-hF9 donor and LNP-CRISPR/Cas9. Consistent with the lack of an adjacent PAM sequence in human albumin, no editing was detectable in the liver when guide RNA G009864 was used. Editing in the liver was observed for the groups using guide RNAs G009859, G009860, G009874, and G012764 (data not shown).

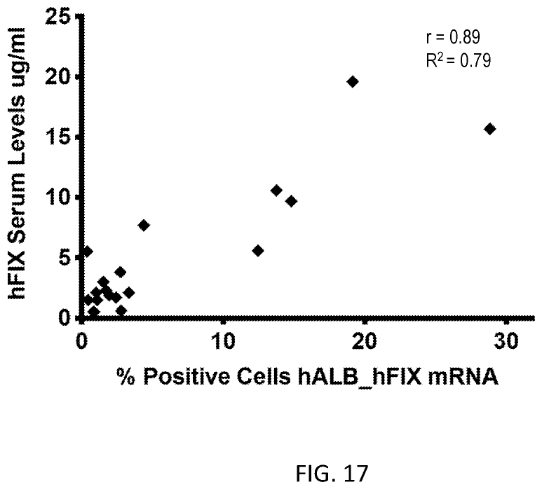

The remaining liver was fixed for 24 hours in 10% neutral buffered formalin and then transferred to 70% ethanol. Four to five samples from separate lobes were cut and shipped to HistoWisz and were processed and embedded in paraffin blocks. Five-micron sections were then cut from each paraffin block, and BASESCOPE™ was performed on the Ventana Ultra Discovery (Roche) using the universal BASESCOPE™ procedure and reagents by Advanced Cell Diagnostics and a custom designed probe that targets the unique mRNA junction formed between the human albumin signal sequence from the first intron of the ALB^{hu/hu} albumin locus and the hF9 transgene when successful integration and transcription is achieved. HALO imaging software (Indica Labs) was then used to quantify the percentage of positive cells in each sample. The average of percentage positive cells across the multiple lobes for each animal was then correlated to the hFIX levels in the serum at week 7. The results are shown in FIG. 17 and Table 29. The week 7 serum levels and the % positive cells for the hALB-hFIX mRNA strongly correlated (r=0.89; R²=0.79).

TABLE 29
Week 7 hFIX and BASESCOPE ™ Data.

| | | hFIX | % mRNA | STD | Total |
|---|---|---|---|---|---|
| | | ug/mL | Probe (4-5 | % mRNA | Cells |
| Mouse | Guide | (Week 7) | Sections) | Probe | Counted |
| 1 | Buffer | ND | 0.09 | 0.03 | 152833 |
| 4 | AAV Only | ND | 0.53 | 0.67 | 351084 |
| 7 | LNP Only | ND | 0.48 | 0.33 | 75160 |
| 10 | CAG F9 | 211.8 | 0.20 | 0.22 | 190277 |
| 15 | G009852 | ND | 0.30 | 0.09 | 144518 |
| 20 | G009859 | 0.5 | 0.82 | 0.45 | 143817 |
| 21 | G009859 | 0.5 | 0.88 | 0.43 | 160172 |
| 22 | G009859 | 2.3 | 1.71 | 1.54 | 26015 |
| 23 | G009859 | 3.8 | 2.74 | 0.59 | 183085 |
| 24 | G009859 | 0.6 | 2.78 | 1.96 | 152424 |
| 25 | G009860 | 5.6 | 12.46 | 5.80 | 78935 |
| 26 | G009860 | 10.6 | 13.76 | 5.32 | 112252 |
| 27 | G009860 | 9.7 | 14.80 | 5.45 | 201592 |
| 28 | G009860 | 2.1 | 3.32 | 0.76 | 84710 |
| 29 | G009860 | 3.0 | 1.52 | 0.35 | 203277 |
| 30 | G009864 | ND | 1.94 | 1.78 | 145807 |
| 35 | G009874 | 1.7 | 2.42 | 1.14 | 126665 |
| 36 | G009874 | 1.5 | 1.08 | 0.53 | 195861 |
| 37 | G009874 | 2.1 | 1.02 | 1.29 | 181679 |
| 38 | G009874 | 5.5 | 0.40 | 0.43 | 175359 |
| 39 | G009874 | 1.5 | 0.44 | 0.18 | 205417 |
| 40 | G012764 | 15.7 | 28.85 | 7.11 | 167824 |
| 41 | G012764 | 19.6 | 19.17 | 8.23 | 70081 |
| 42 | G012764 | 1.9 | 1.95 | 1.79 | 154742 |
| 43 | G012764 | 7.7 | 4.38 | 0.68 | 114060 |
| 44 | G012764 | 3.0 | 1.64 | 1.04 | 238623 |
| 43 | DapB (−) | — | 0.12 | 0.07 | 144730 |
| 1 | Buffer | ND | 0.09 | 0.03 | 152833 |
| 4 | AAV Only | ND | 0.53 | 0.67 | 351084 |
| 7 | LNP Only | ND | 0.48 | 0.33 | 75160 |
| 10 | CAG F9 | 211.8 | 0.20 | 0.22 | 190277 |
| 15 | G009852 | ND | 0.30 | 0.09 | 144518 |
| 20 | G009859 | 0.5 | 0.82 | 0.45 | 143817 |
| 21 | G009859 | 0.5 | 0.88 | 0.43 | 160172 |
| 22 | G009859 | 2.3 | 1.71 | 1.54 | 26015 |
| 23 | G009859 | 3.8 | 2.74 | 0.59 | 183085 |
| 24 | G009859 | 0.6 | 2.78 | 1.96 | 152424 |
| 25 | G009860 | 5.6 | 12.46 | 5.80 | 78935 |
| 26 | G009860 | 10.6 | 13.76 | 5.32 | 112252 |
| 27 | G009860 | 9.7 | 14.80 | 5.45 | 201592 |
| 28 | G009860 | 2.1 | 3.32 | 0.76 | 84710 |
| 29 | G009860 | 3.0 | 1.52 | 0.35 | 203277 |
| 30 | G009864 | ND | 1.94 | 1.78 | 145807 |
| 35 | G009874 | 1.7 | 2.42 | 1.14 | 126665 |
| 36 | G009874 | 1.5 | 1.08 | 0.53 | 195861 |
| 37 | G009874 | 2.1 | 1.02 | 1.29 | 181679 |
| 38 | G009874 | 5.5 | 0.40 | 0.43 | 175359 |
| 39 | G009874 | 1.5 | 0.44 | 0.18 | 205417 |
| 40 | G012764 | 15.7 | 28.85 | 7.11 | 167824 |
| 41 | G012764 | 19.6 | 19.17 | 8.23 | 70081 |
| 42 | G012764 | 1.9 | 1.95 | 1.79 | 154742 |
| 43 | G012764 | 7.7 | 4.38 | 0.68 | 114060 |
| 44 | G012764 | 3.0 | 1.64 | 1.04 | 238623 |
| 43 | DapB (−) | — | 0.12 | 0.07 | 144730 |

Human albumin intron 1:
(SEQ ID NO: 1)
GTAAGAAATCCATTTTTCTATTGTTCAACTTTTATTCTATTTTCCCAGTA
AAATAAAGTTTTAGTAAACTCTGCATCTTTAAAGAATTATTTTGGCATTT
ATTTCTAAAATGGCATAGTATTTTGTATTTGTGAAGTCTTACAAGGTTAT
CTTATTAATAAAATTCAAACATCCTAGGTAAAAAAAAAAAAAGGTCAGAA
TTGTTTAGTGACTGTAATTTTCTTTTGCGCACTAAGGAAAGTGCAAAGTA
ACTTAGAGTGACTGAAACTTCACAGAATAGGGTTGAAGATTGAATTCATA
ACTATCCCAAAGACCTATCCATTGCACTATGCTTTATTTAAAAACCACAA
AACCTGTGCTGTTGATCTCATAAATAGAACTTGTATTTATATTTATTTTC
ATTTTAGTCTGTCTTCTTGGTTGCTGTTGATAGACACTAAAAGAGTATTA
GATATTATCTAAGTTTGAATATAAGGCTATAAATATTTAATAATTTTTAA
AATAGTATTCTTGGTAATTGAATTATTCTTCTGTTTAAAGGCAGAAGAAA
TAATTGAACATCATCCTGAGTTTTTCTGTAGGAATCAGAGCCCAATATTT
TGAAACAAATGCATAATCTAAGTCAAATGGAAAGAAATATAAAAAGTAAC
ATTATTACTTCTTGTTTTCTTCAGTATTTAACAATCCTTTTTTTTCTTCC
CTTGCCCAG

TABLE 7
Mouse albumin guide RNA

| | | | SEQ |
|---|---|---|---|
| | | | ID |
| Guide ID | Guide Sequence | Genomic Coordinates | NO: |
| G000551 | AUUUGCAUCUGAGAACCCUU | chr5: 90461148-90461168 | 98 |
| G000552 | AUCGGGAACUGGCAUCUUCA | chr5: 90461590-90461610 | 99 |
| G000553 | GUUACAGGAAAAUCUGAAGG | chr5: 90461569-90461589 | 100 |
| G000554 | GAUCGGGAACUGGCAUCUUC | chr5: 90461589-90461609 | 101 |
| G000555 | UGCAUCUGAGAACCCUUAGG | chr5: 90461151-90461171 | 102 |
| G000666 | CACUCUUGUCUGUGGAAACA | chr5: 90461709-90461729 | 103 |
| G000667 | AUCGUUACAGGAAAAUCUGA | chr5: 90461572-90461592 | 104 |
| G000668 | GCAUCUUCAGGGAGUAGCUU | chr5: 90461601-90461621 | 105 |
| G000669 | CAAUCUUUAAAUAUGUUGUG | chr5: 90461674-90461694 | 106 |
| G000670 | UCACUCUUGUCUGUGGAAAC | chr5: 90461710-90461730 | 107 |
| G011722 | UGCUUGUAUUUUUCUAGUAA | chr5: 90461039-90461059 | 108 |
| G011723 | GUAAAUAUCUACUAAGACAA | chr5: 90461425-90461445 | 109 |
| G011724 | UUUUUCUAGUAAUGGAAGCC | chr5: 90461047-90461067 | 110 |
| G011725 | UUAUAUUAUUGAUAUAUUUU | chr5: 90461174-90461194 | 111 |
| G011726 | GCACAGAUAUAAACACUUAA | chr5: 90461480-90461500 | 112 |
| G011727 | CACAGAUAUAAACACUUAAC | chr5: 90461481-90461501 | 113 |
| G011728 | GGUUUUAAAAAUAAUAAUGU | chr5: 90461502-90461522 | 114 |
| G011729 | UCAGAUUUUCCUGUAACGAU | chr5: 90461572-90461592 | 115 |
| G011730 | CAGAUUUUCCUGUAACGAUC | chr5: 90461573-90461593 | 116 |
| G011731 | CAAUGGUAAAUAAGAAAUAA | chr5: 90461408-90461428 | 117 |
| G013018 | GGAAAAUCUGAAGGUGGCAA | chr5: 90461563-90461583 | 118 |
| G013019 | GGCGAUCUCACUCUUGUCUG | chr5: 90461717-90461737 | 119 |

TABLE 8
Mouse albumin sgRNAs and modification pattern

| | | SEQ | | SEQ |
|---|---|---|---|---|
| | | ID | | ID |
| Guide ID | Full Sequence | NO: | Full Sequence Modified | NO: |
| G000551 | AUUUGCAUCUGAGAACCCUUGU | 120 | mA*mU*mU*UGCAUCUGAGAACC | 142 |
| | UUUAGAGCUAGAAAUAGCAAGU | | CUUGUUUUAGAmGmCmUmAmGm | |
| | UAAAAUAAGGCUAGUCCGUUAU | | AmAmAmUmAmGmCAAGUUAAAA | |
| | CAACUUGAAAAAGUGGCACCGA | | UAAGGCUAGUCCGUUAUCAmAmC | |
| | GUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000552 | AUCGGGAACUGGCAUCUUCA | 121 | mA*mU*mC*GGGAACUGGCAUCU | 143 |
| | GUUUUAGAGCUAGAAAUAGC | | UCAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000553 | GUUACAGGAAAAUCUGAAGG | 122 | mG*mU*mU*ACAGGAAAAUCUGA | 144 |
| | GUUUUAGAGCUAGAAAUAGC | | AGGGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000554 | GAUCGGGAACUGGCAUCUUC | 123 | mG*mA*mU*CGGGAACUGGCAUC | 145 |
| | GUUUUAGAGCUAGAAAUAGC | | UUCGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000555 | UGCAUCUGAGAACCCUUAGG | 124 | mU*mG*mC*AUCUGAGAACCCUU | 146 |
| | GUUUUAGAGCUAGAAAUAGC | | AGGGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000666 | CACUCUUGUCUGUGGAAACA | 125 | mC*mA*mC*UCUUGUCUGUGGAA | 147 |
| | GUUUUAGAGCUAGAAAUAGC | | ACAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000667 | AUCGUUACAGGAAAAUCUGA | 126 | mA*mU*mC*GUUACAGGAAAAUC | 148 |
| | GUUUUAGAGCUAGAAAUAGC | | UGAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000668 | GCAUCUUCAGGGAGUAGCUU | 127 | mG*mC*mA*UCUUCAGGGAGUAG | 149 |
| | GUUUUAGAGCUAGAAAUAGC | | CUUGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000669 | CAAUCUUUAAAUAUGUUGUG | 128 | mC*mA*mA*UCUUUAAAUAUGUU | 150 |
| | GUUUUAGAGCUAGAAAUAGC | | GUGGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G000670 | UCACUCUUGUCUGUGGAAAC | 129 | mU*mC*mA*CUCUUGUCUGUGGA | 151 |
| | GUUUUAGAGCUAGAAAUAGC | | AACGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011722 | UGCUUGUAUUUUUCUAGUAA | 130 | mU*mG*mC*UUGUAUUUUUCUAG | 152 |
| | GUUUUAGAGCUAGAAAUAGC | | UAAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011723 | GUAAAUAUCUACUAAGACAA | 131 | mG*mU*mA*AAUAUCUACUAAGA | 153 |
| | GUUUUAGAGCUAGAAAUAGC | | CAAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011724 | UUUUUCUAGUAAUGGAAGCC | 132 | mU*mU*mU*UUCUAGUAAUGGAA | 154 |
| | GUUUUAGAGCUAGAAAUAGC | | GCCGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011725 | UUAUAUUAUUGAUAUAUUUU | 133 | mU*mU*mA*UAUUAUUGAUAUAU | 155 |
| | GUUUUAGAGCUAGAAAUAGC | | UUUGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011726 | GCACAGAUAUAAACACUUAA | 134 | mG*mC*mA*CAGAUAUAAACACU | 156 |
| | GUUUUAGAGCUAGAAAUAGC | | UAAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011727 | CACAGAUAUAAACACUUAAC | 135 | mC*mA*mC*AGAUAUAAACACUU | 157 |
| | GUUUUAGAGCUAGAAAUAGC | | AACGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011728 | GGUUUUAAAAAUAAUAAUGU | 136 | mG*mG*mU*UUUAAAAAUAAUAA | 158 |
| | GUUUUAGAGCUAGAAAUAGC | | UGUGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011729 | UCAGAUUUUCCUGUAACGAU | 137 | mU*mC*mA*GAUUUUCCUGUAAC | 159 |
| | GUUUUAGAGCUAGAAAUAGC | | GAUGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011730 | CAGAUUUUCCUGUAACGAUC | 138 | mC*mA*mG*AUUUUCCUGUAACG | 160 |
| | GUUUUAGAGCUAGAAAUAGC | | AUCGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G011731 | CAAUGGUAAAUAAGAAAUAA | 139 | mC*mA*mA*UGGUAAAUAAGAAA | 161 |
| | GUUUUAGAGCUAGAAAUAGC | | UAAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G013018 | GGAAAAUCUGAAGGUGGCAA | 140 | mG*mG*mA*AAAUCUGAAGGUGG | 162 |
| | GUUUUAGAGCUAGAAAUAGC | | CAAGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |
| G013019 | GGCGAUCUCACUCUUGUCUG | 141 | mG*mG*mC*GAUCUCACUCUUGU | 163 |
| | GUUUUAGAGCUAGAAAUAGC | | CUGGUUUUAGAmGmCmUmAmGm | |
| | AAGUUAAAAUAAGGCUAGUC | | AmAmAmUmAmGmCAAGUUAAAA | |
| | CGUUAUCAACUUGAAAAAGU | | UAAGGCUAGUCCGUUAUCAmAmC | |
| | GGCACCGAGUCGGUGCUUUU | | mUmUmGmAmAmAmAmAmGmUm | |
| | | | GmGmCmAmCmCmGmAmGmUmCm | |
| | | | GmGmUmGmCmU*mU*mU*mU | |

TABLE 9
Cyno albumin guide RNA

| Guide ID | Guide Sequence | Genomic Coordinates | SEQ ID NO: |
|---|---|---|---|
| G009844 | GAGCAACCUCACUCUUGUCU | chr5: 61198711-61198731 | 164 |
| G009845 | AGCAACCUCACUCUUGUCUG | chr5: 61198712-61198732 | 165 |
| G009846 | ACCUCACUCUUGUCUGGGGA | chr5: 61198716-61198736 | 166 |
| G009847 | CCUCACUCUUGUCUGGGGAA | chr5: 61198717-61198737 | 167 |
| G009848 | CUCACUCUUGUCUGGGGAAG | chr5: 61198718-61198738 | 168 |
| G009849 | GGGGAAGGGGAGAAAAAAAA | chr5: 61198731-61198751 | 169 |
| G009850 | GGGAAGGGGAGAAAAAAAAA | chr5: 61198732-61198752 | 170 |
| G009851 | AUGCAUUUGUUUCAAAAUAU | chr5: 61198825-61198845 | 171 |
| G009852 | UGCAUUUGUUUCAAAAUAUU | chr5: 61198826-61198846 | 172 |
| G009853 | UGAUUCCUACAGAAAAAGUC | chr5: 61198852-61198872 | 173 |
| G009854 | UACAGAAAAAGUCAGGAUAA | chr5: 61198859-61198879 | 174 |
| G009855 | UUUCUUCUGCCUUUAAACAG | chr5: 61198889-61198909 | 175 |
| G009856 | UUAUAGUUUUAUAUUCAAAC | chr5: 61198957-61198977 | 176 |
| G009857 | AUUUAUGAGAUCAACAGCAC | chr5: 61199062-61199082 | 177 |
| G009858 | GAUCAACAGCACAGGUUUUG | chr5: 61199070-61199090 | 178 |
| G009859 | UUAAAUAAAGCAUAGUGCAA | chr5: 61199096-61199116 | 179 |
| G009860 | UAAAGCAUAGUGCAAUGGAU | chr5: 61199101-61199121 | 180 |
| G009861 | UAGUGCAAUGGAUAGGUCUU | chr5: 61199108-61199128 | 181 |
| G009862 | AGUGCAAUGGAUAGGUCUUA | chr5: 61199109-61199129 | 182 |
| G009863 | UUACUUUGCACUUUCCUUAG | chr5: 61199186-61199206 | 183 |
| G009864 | UACUUUGCACUUUCCUUAGU | chr5: 61199187-61199207 | 184 |
| G009865 | UCUGACCUUUUAUUUUACCU | chr5: 61199238-61199258 | 185 |
| G009866 | UACUAAAACUUUAUUUUACU | chr5: 61199367-61199387 | 186 |
| G009867 | AAAGUUGAACAAUAGAAAAA | chr5: 61199401-61199421 | 187 |
| G009868 | AAUGCAUAAUCUAAGUCAAA | chr5: 61198812-61198832 | 188 |
| G009869 | AUUAUCCUGACUUUUUCUGU | chr5: 61198860-61198880 | 189 |
| G009870 | UGAAUUAUUCCUCUGUUUAA | chr5: 61198901-61198921 | 190 |
| G009871 | UAAUUUUCUUUUGCCCACUA | chr5: 61199203-61199223 | 191 |
| G009872 | AAAAGGUCAGAAUUGUUUAG | chr5: 61199229-61199249 | 192 |
| G009873 | AACAUCCUAGGUAAAAUAAA | chr5: 61199246-61199266 | 193 |
| G009874 | UAAUAAAAUUCAAACAUCCU | chr5: 61199258-61199278 | 194 |
| G009875 | UUGUCAUGUAUUUCUAAAAU | chr5: 61199322-61199342 | 195 |
| G009876 | UUUGUCAUGUAUUUCUAAAA | chr5: 61199323-61199343 | 196 |

TABLE 10
Cyno sgRNA and modification patterns

| | | SEQ | | SEQ |
|---|---|---|---|---|
| | | ID | | ID |
| Guide ID | Full Sequence | NO: | Full Sequence Modified | NO: |
| G009844 | GAGCAACCUCACUCUUGUCU | 197 | mG*mA*mG*CAACCUCACUCUU | 230 |
| | GUUUUAGAGCUAGAAAUAGC | | GUCUGUUUUAGAmGmCmUmA | |
| | AAGUUAAAAUAAGGCUAGUC | | mGmAmAmAmUmAmGmCAAGU | |
| | CGUUAUCAACUUGAAAAAGU | | UAAAAUAAGGCUAGUCCGUUA | |
| | GGCACCGAGUCGGUGCUUUU | | UCAmAmCmUmUmGmAmAmAm | |
| | | | AmAmGmUmGmGmCmAmCmCm | |
| | | | GmAmGmUmCmGmGmUmGmCm | |
| | | | U*mU*mU*mU | |
| G009845 | AGCAACCUCACUCUUGUCUG | 198 | mA*mG*mC*AACCUCACUCUUG | 231 |
| | GUUUUAGAGCUAGAAAUAGC | | UCUGGUUUUAGAmGmCmUmA | |
| | AAGUUAAAAUAAGGCUAGUC | | mGmAmAmAmUmAmGmCAAGU | |
| | CGUUAUCAACUUGAAAAAGU | | UAAAAUAAGGCUAGUCCGUUA | |
| | GGCACCGAGUCGGUGCUUUU | | UCAmAmCmUmUmGmAmAmAm | |
| | | | AmAmGmUmGmGmCmAmCmCm | |
| | | | GmAmGmUmCmGmGmUmGmCm | |
| | | | U*mU*mU*mU | |
| G009846 | ACCUCACUCUUGUCUGGGGA | 199 | mA*mC*mC*UCACUCUUGUCUG | 232 |
| | GUUUUAGAGCUAGAAAUAGC | | GGGAGUUUUAGAmGmCmUmA | |
| | AAGUUAAAAUAAGGCUAGUC | | mGmAmAmAmUmAmGmCAA | |
| | CGUUAUCAACUUGAAAAAGU | | GUUAAAAUAAGGCUAGUCCGU | |
| | GGCACCGAGUCGGUGCUUUU | | UAUCAmAmCmUmUmGmAmAm | |
| | | | AmAmAmGmUmGmGmCmAmCm | |
| | | | CmGmAmGmUmCmGmGmUmGm | |
| | | | CmU*mU*mU*mU | |
| G009847 | CCUCACUCUUGUCUGGGGAA | 200 | mC*mC*mU*CACUCUUGUCUGG | 233 |
| | GUUUUAGAGCUAGAAAUAGC | | GGAAGUUUUAGAmGmCmUmA | |
| | AAGUUAAAAUAAGGCUAGUC | | mGmAmAmAmUmAmGmCAAGU | |
| | CGUUAUCAACUUGAAAAAGU | | UAAAAUAAGGCUAGUCCGUUA | |
| | GGCACCGAGUCGGUGCUUUU | | UCAmAmCmUmUmGmAmAmAm | |
| | | | AmAmGmUmGmGmCmAmCmCm | |
| | | | GmAmGmUmCmGmGmUmGmCm | |
| | | | U*mU*mU*mU | |
| G009848 | CUCACUCUUGUCUGGGGAAG | 201 | mC*mU*mC*ACUCUUGUCUGG | 234 |
| | GUUUUAGAGCUAGAAAUAGC | | GGAAGGUUUUAGAmGmCmUm | |
| | AAGUUAAAAUAAGGCUAGUC | | AmGmAmAmAmUmAmGmCAA | |
| | CGUUAUCAACUUGAAAAAGU | | GUUAAAAUAAGGCUAGUCCGU | |
| | GGCACCGAGUCGGUGCUUUU | | UAUCAmAmCmUmUmGmAmAm | |
| | | | AmAmAmGmUmGmGmCmAmCm | |
| | | | CmGmAmGmUmCmGmGmUmGm | |
| | | | CmU*mU*mU*mU | |
| G009849 | GGGGAAGGGGAGAAAAAAAA | 202 | mG*mG*mG*GAAGGGGAGAAA | 235 |
| | GUUUUAGAGCUAGAAAUAGC | | AAAAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009850 | GGGAAGGGGAGAAAAAAAAA | 203 | mG*mG*mG*AAGGGGAGAAAA | 236 |
| | GUUUUAGAGCUAGAAAUAGC | | AAAAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009851 | AUGCAUUUGUUUCAAAAUAU | 204 | mA*mU*mG*CAUUUGUUUCAA | 237 |
| | GUUUUAGAGCUAGAAAUAGC | | AAUAUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009852 | UGCAUUUGUUUCAAAAUAUU | 205 | mU*mG*mC*AUUUGUUUCAAA | 238 |
| | GUUUUAGAGCUAGAAAUAGC | | AUAUUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009853 | UGAUUCCUACAGAAAAAGUC | 206 | mU*mG*mA*UUCCUACAGAAA | 239 |
| | GUUUUAGAGCUAGAAAUAGC | | AAGUCGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009854 | UACAGAAAAAGUCAGGAUAA | 207 | mU*mA*mC*AGAAAAAGUCAG | 240 |
| | GUUUUAGAGCUAGAAAUAGC | | GAUAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009855 | UUUCUUCUGCCUUUAAACAG | 208 | mU*mU*mU*CUUCUGCCUUUA | 241 |
| | GUUUUAGAGCUAGAAAUAGC | | AACAGGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009856 | UUAUAGUUUUAUAUUCAAAC | 209 | mU*mU*mA*UAGUUUUAUAUU | 242 |
| | GUUUUAGAGCUAGAAAUAGC | | CAAACGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009857 | AUUUAUGAGAUCAACAGCAC | 210 | mA*mU*mU*UAUGAGAUCAAC | 243 |
| | GUUUUAGAGCUAGAAAUAGC | | AGCACGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009858 | GAUCAACAGCACAGGUUUUG | 211 | mG*mA*mU*CAACAGCACAGG | 244 |
| | GUUUUAGAGCUAGAAAUAGC | | UUUUGGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009859 | UUAAAUAAAGCAUAGUGCAA | 212 | mU*mU*mA*AAUAAAGCAUAG | 245 |
| | GUUUUAGAGCUAGAAAUAGC | | UGCAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009860 | UAAAGCAUAGUGCAAUGGAU | 213 | mU*mA*mA*AGCAUAGUGCAA | 246 |
| | GUUUUAGAGCUAGAAAUAGC | | UGGAUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009861 | UAGUGCAAUGGAUAGGUCUU | 214 | mU*mA*mG*UGCAAUGGAUAG | 247 |
| | GUUUUAGAGCUAGAAAUAGC | | GUCUUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009862 | AGUGCAAUGGAUAGGUCUUA | 215 | mA*mG*mU*GCAAUGGAUAGG | 248 |
| | GUUUUAGAGCUAGAAAUAGC | | UCUUAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009863 | UUACUUUGCACUUUCCUUAG | 216 | mU*mU*mA*CUUUGCACUUUC | 249 |
| | GUUUUAGAGCUAGAAAUAGC | | CUUAGGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009864 | UACUUUGCACUUUCCUUAGU | 217 | mU*mA*mC*UUUGCACUUUCC | 250 |
| | GUUUUAGAGCUAGAAAUAGC | | UUAGUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009865 | UCUGACCUUUUAUUUUACCU | 218 | mU*mC*mU*GACCUUUUAUUU | 251 |
| | GUUUUAGAGCUAGAAAUAGC | | UACCUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009866 | UACUAAAACUUUAUUUUACU | 219 | mU*mA*mC*UAAAACUUUAUU | 252 |
| | GUUUUAGAGCUAGAAAUAGC | | UUACUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009867 | AAAGUUGAACAAUAGAAAAA | 220 | mA*mA*mA*GUUGAACAAUAG | 253 |
| | GUUUUAGAGCUAGAAAUAGC | | AAAAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009868 | AAUGCAUAAUCUAAGUCAAA | 221 | mA*mA*mU*GCAUAAUCUAAG | 254 |
| | GUUUUAGAGCUAGAAAUAGC | | UCAAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009869 | AUUAUCCUGACUUUUUCUGU | 222 | mA*mU*mU*AUCCUGACUUUU | 255 |
| | GUUUUAGAGCUAGAAAUAGC | | UCUGUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009870 | UGAAUUAUUCCUCUGUUUAA | 223 | mU*mG*mA*AUUAUUCCUCUG | 256 |
| | GUUUUAGAGCUAGAAAUAGC | | UUUAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009871 | UAAUUUUCUUUUGCCCACUA | 224 | mU*mA*mA*UUUUCUUUUGCC | 257 |
| | GUUUUAGAGCUAGAAAUAGC | | CACUAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009872 | AAAAGGUCAGAAUUGUUUAG | 225 | mA*mA*mA*AGGUCAGAAUUG | 258 |
| | GUUUUAGAGCUAGAAAUAGC | | UUUAGGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009873 | AACAUCCUAGGUAAAAUAAA | 226 | mA*mA*mC*AUCCUAGGUAAA | 259 |
| | GUUUUAGAGCUAGAAAUAGC | | AUAAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009874 | UAAUAAAAUUCAAACAUCCU | 227 | mU*mA*mA*UAAAAUUCAAAC | 260 |
| | GUUUUAGAGCUAGAAAUAGC | | AUCCUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009875 | UUGUCAUGUAUUUCUAAAAU | 228 | mU*mU*mG*UCAUGUAUUUCU | 261 |
| | GUUUUAGAGCUAGAAAUAGC | | AAAAUGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |
| G009876 | UUUGUCAUGUAUUUCUAAAA | 229 | mU*mU*mU*GUCAUGUAUUUC | 262 |
| | GUUUUAGAGCUAGAAAUAGC | | UAAAAGUUUUAGAmGmCmUm | |
| | AAGUUAAAAUAAGGCUAGUC | | AmGmAmAmAmUmAmGmCAAG | |
| | CGUUAUCAACUUGAAAAAGU | | UUAAAAUAAGGCUAGUCCGUU | |
| | GGCACCGAGUCGGUGCUUUU | | AUCAmAmCmUmUmGmAmAmA | |
| | | | mAmAmGmUmGmGmCmAmCmC | |
| | | | mGmAmGmUmCmGmGmUmGmC | |
| | | | mU*mU*mU*mU | |

TABLE 11
Vector Components and Sequences

| | | Splice | | | | | Splice | |
|---|---|---|---|---|---|---|---|---|
| Plasmid | | Acceptor (1^{st} | Transgene (1^{st} | Poly-A (1^{st} | Poly-A (2^{nd} | Transgene (2^{nd} | Acceptor (2^{nd} | |
| ID | 5′ ITR | orientation) | orientation) | orientation) | orientation) | orientation) | orientation) | 3′ ITR |
| P00147 | (SEQ ID | Mouse | Human | SEQ ID | SEQ ID | Human | Mouse | (SEQ ID |
| | NO: 263) | Albumin | Factor IX | NO: 266 | NO: 267 | Factor IX | Albumin | NO: 270) |
| | | Splice | (R338L) | | | (R338L) | Splice | |
| | | Acceptor | (SEQ ID | | | (SEQ ID | Acceptor | |
| | | (SEQ ID | NO: 265) | | | NO: 268) | (SEQ ID | |
| | | NO: 264) | | | | | NO: 269) | |
| P00411 | (SEQ ID | Human | Human | SEQ ID | SEQ ID | Human | Human | (SEQ ID |
| | NO: 263) | Factor IX | Factor IX | NO: 266 | NO: 267 | Factor IX | Factor IX | NO: 270) |
| | | Splice | (R338L)- | | | (R338L)- | Splice | |
| | | Acceptor | HiBit | | | HiBit | Acceptor | |
| | | (SEQ ID | (SEQ ID | | | (SEQ ID | (SEQ ID | |
| | | NO: 271) | NO: 272) | | | NO: 273) | NO: 274) | |
| P00415 | (SEQ ID | Mouse | Nluc- | SEQ ID | SEQ ID | Nluc- | Mouse | (SEQ ID |
| | NO: 263) | Albumin | P2A-GFP | NO: 266 | NO: 267 | P2A-GFP | Albumin | NO: 270) |
| | | Splice | (SEQ ID | | | (SEQ ID | Splice | |
| | | Acceptor | NO: 275) | | | NO: 276) | Acceptor | |
| | | (SEQ ID | | | | | (SEQ ID | |
| | | NO: 264) | | | | | NO: 269) | |
| P00418 | (SEQ ID | Mouse | Human | SEQ ID | SEQ ID | Human | Mouse | (SEQ ID |
| | NO: 263) | Albumin | Factor IX | NO: 266 | NO: 267 | Factor IX | Albumin | NO: 270) |
| | | Splice | (R338L)- | | | (R338L)- | Splice | |
| | | Acceptor | HiBit | | | HiBit | Acceptor | |
| | | (SEQ ID | (SEQ ID | | | (SEQ ID | (SEQ ID | |
| | | NO: 264) | NO: 272) | | | NO: 273) | NO: 269) | |

5′ ITR Sequence (SEQ ID NO: 263):
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCT
Mouse Albumin Splice Acceptor (1^{st} orientation) (SEQ ID NO: 264):
TAGGTCAGTGAAGAGAAGAACAAAAAGCAGCATATTACAGTTAGTTGTCTTCATCA
ATCTTTAAATATGTTGTGTGGTTTTTCTCTCCCTGTTTCCACAG
Human Factor IX (R338L), 1^{st} Orientation (SEQ ID NO: 265):
TTTCTTGATCATGAAAACGCCAACAAAATTCTGAATCGGCCAAAGAGGTATAATTCA
GGTAAATTGGAAGAGTTTGTTCAAGGGAACCTTGAGAGAGAATGTATGGAAGAAAA
GTGTAGTTTTGAAGAAGCACGAGAAGTTTTTGAAAACACTGAAAGAACAACTGAAT
TTTGGAAGCAGTATGTTGATGGAGATCAGTGTGAGTCCAATCCATGTTTAAATGGCG
GCAGTTGCAAGGATGACATTAATTCCTATGAATGTTGGTGTCCCTTTGGATTTGAAG
GAAAGAACTGTGAATTAGATGTAACATGTAACATTAAGAATGGCAGATGCGAGCAG
TTTTGTAAAAATAGTGCTGATAACAAGGTGGTTTGCTCCTGTACTGAGGGATATCGA
CTTGCAGAAAACCAGAAGTCCTGTGAACCAGCAGTGCCATTTCCATGTGGAAGAGTT
TCTGTTTCACAAACTTCTAAGCTCACCCGTGCTGAGACTGTTTTTCCTGATGTGGACT
ATGTAAATTCTACTGAAGCTGAAACCATTTTGGATAACATCACTCAAAGCACCCAAT
CATTTAATGACTTCACTCGGGTTGTTGGTGGAGAAGATGCCAAACCAGGTCAATTCC
CTTGGCAGGTTGTTTTGAATGGTAAAGTTGATGCATTCTGTGGAGGCTCTATCGTTA
ATGAAAAATGGATTGTAACTGCTGCCCACTGTGTTGAAACTGGTGTTAAAATTACAG
TTGTCGCAGGTGAACATAATATTGAGGAGACAGAACATACAGAGCAAAAGCGAAAT
GTGATTCGAATTATTCCTCACCACAACTACAATGCAGCTATTAATAAGTACAACCAT
GACATTGCCCTTCTGGAACTGGACGAACCCTTAGTGCTAAACAGCTACGTTACACCT
ATTTGCATTGCTGACAAGGAATACACGAACATCTTCCTCAAATTTGGATCTGGCTAT
GTAAGTGGCTGGGGAAGAGTCTTCCACAAAGGGAGATCAGCTTTAGTTCTTCAGTAC
CTTAGAGTTCCACTTGTTGACCGAGCCACATGTCTTCTATCTACAAAGTTCACCATCT
ATAACAACATGTTCTGTGCTGGCTTCCATGAAGGAGGTAGAGATTCATGTCAAGGAG
ATAGTGGGGGACCCCATGTTACTGAAGTGGAAGGGACCAGTTTCTTAACTGGAATTA
TTAGCTGGGGTGAAGAGTGTGCAATGAAAGGCAAATATGGAATATATACCAAGGTA
TCCCGGTATGTCAACTGGATTAAGGAAAAAACAAAGCTCACTTAA
Poly-A (1^{st} orientation) (SEQ ID NO: 266):
CCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAG
CAAGGGGGAGGATTGGGAAGACAATAGCAGGCATGCTGGGGATGCGGTGGGCTCTA
TGGCTTCTGAGGCGGAAAGAACCAGCTGGGGCTCTAGGGGGTATCCCC
Poly-A (2^{nd} orientation) (SEQ ID NO: 267):
AAAAAACCTCCCACACCTCCCCCTGAACCTGAAACATAAAATGAATGCAATTGTTGT
TGTTAACTTGTTTATTGCAGCTTATAATGGTTACAAATAAAGCAATAGCATCACAAA
TTTCACAAATAAAGCATTTTTTTCACTGCATTCTAGTTGTGGTTTGTCCAAACTCATC
AATGTATCTTATCATGTCTG
Human Factor IX (R338L), 2^{nd} Orientation (SEQ ID NO: 268):
TTAGGTGAGCTTAGTCTTTTCTTTTATCCAATTCACGTAGCGAGAGACCTTCGTATAG
ATGCCATATTTCCCCTTCATCGCACATTCCTCCCCCCAACTTATTATCCCGGTCAAGA
AACTTGTTCCTTCGACTTCAGTGACGTGTGGTCCACCTGAATCACCTTGGCATGAGTC
GCGACCGCCCTCGTGAAACCCAGCACAAAACATGTTATTGTAAATCGTAAATTTCGT
GGACAGAAGACAGGTCGCTCTATCGACCAACGGGACGCGCAAATATTGCAGAACGA
GGGCTGATCGACCTTTGTGGAAGACCCGCCCCCACCCACTCACATATCCGCTCCCAA
ATTTCAAGAAGATATTTGTATATTCTTTATCGGCTATACAAATCGGGGTAACATAGG
AGTTAAGTACGAGTGGCTCGTCCAGCTCCAGGAGGGCTATATCATGGTTGTACTTGT
TTATAGCGGCATTATAATTGTGATGGGGTATGATCCTGATAACATTCCTTTTCTGTTC
AGTATGCTCAGTTTCTTCAATGTTGTGTTCGCCAGCCACGACCGTAATCTTAACCCCC
GTCTCGACACAGTGTGCGGCCGTTACAATCCACTTTTCATTGACTATGGAGCCCCCA
CAAAACGCGTCGACTTTTCCGTTGAGCACCACCTGCCATGGAAATTGGCCAGGTTTA
GCGTCCTCGCCCCCGACAACCCTAGTAAAGTCATTAAATGACTGTGTGGATTGTGTT
ATATTATCAAGAATCGTTTCGGCTTCAGTAGAGTTAACGTAGTCCACATCGGGAAAA
ACTGTCTCGGCCCTTGTCAACTTTGATGTCTGGGACACACTTACCCGACCGCACGGG
AAGGGCACCGCCGGTTCACAGCTCTTTTGATTCTCAGCGAGCCGGTAGCCCTCAGTG
CAACTACACACAACTTTGTTGTCGGCGGAATTTTTACAGAATTGCTCGCATCGTCCA
TTTTTAATGTTGCAGGTGACGTCCAACTCGCAGTTTTTTCCTTCAAAACCAAAAGGG
CACCAACACTCGTAGGAATTTATATCGTCTTTACAACTCCCCCCATTCAGACATGGA
TTAGATTCGCATTGGTCCCCATCGACATATTGCTTCCAGAACTCAGTGGTCCGTTCTG
TATTCTCAAACACCTCGCGCGCTTCTTCAAAACTGCATTTTTCCTCCATACACTCTCG
CTCCAAGTTCCCTTGCACGAATTCTTCAAGCTTTCCTGAGTTATACCTTTTAGGCCGG
TTAAGTATCTTATTCGCGTTTTCGTGGTCCAGAAA
Mouse Albumin Splice Acceptor (2^{nd} orientation) (SEQ ID NO: 269):
CTGTGGAAACAGGGAGAGAAAAACCACACAACATATTTAAAGATTGATGAAGACAA
CTAACTGTAATATGCTGCTTTTTGTTCTTCTCTTCACTGACCTA
3′ ITR Sequence (SEQ ID NO: 270):
AGGAACCCCTAGTGATGGAGTTGGCCACTCCCTCTCTGCGCGCTCGCTCGCTCACTG
AGGCCGCCCGGGCAAAGCCCGGGCGTCGGGCGACCTTTGGTCGCCCGGCCTCAGTG
AGCGAGCGAGCGCGCAGAGAGGGAGTGGCCAA
Human Factor IX Splice Acceptor (1^{st} Orientation) (SEQ ID NO: 271):
GATTATTTGGATTAAAAACAAAGACTTTCTTAAGAGATGTAAAATTTTCATGATGTT
TTCTTTTTTGCTAAAACTAAAGAATTATTCTTTTACATTTCAG
Human Factor IX (R338L)-HiBit (1^{st} Orientation) (SEQ ID NO: 272):
TTTCTTGATCATGAAAACGCCAACAAAATTCTGAATCGGCCAAAGAGGTATAATTCA
GGTAAATTGGAAGAGTTTGTTCAAGGGAACCTTGAGAGAGAATGTATGGAAGAAAA
GTGTAGTTTTGAAGAAGCACGAGAAGTTTTTGAAAACACTGAAAGAACAACTGAAT
TTTGGAAGCAGTATGTTGATGGAGATCAGTGTGAGTCCAATCCATGTTTAAATGGCG
GCAGTTGCAAGGATGACATTAATTCCTATGAATGTTGGTGTCCCTTTGGATTTGAAG
GAAAGAACTGTGAATTAGATGTAACATGTAACATTAAGAATGGCAGATGCGAGCAG
TTTTGTAAAAATAGTGCTGATAACAAGGTGGTTTGCTCCTGTACTGAGGGATATCGA
CTTGCAGAAAACCAGAAGTCCTGTGAACCAGCAGTGCCATTTCCATGTGGAAGAGTT
TCTGTTTCACAAACTTCTAAGCTCACCCGTGCTGAGACTGTTTTTCCTGATGTGGACT
ATGTAAATTCTACTGAAGCTGAAACCATTTTGGATAACATCACTCAAAGCACCCAAT
CATTTAATGACTTCACTCGGGTTGTTGGTGGAGAAGATGCCAAACCAGGTCAATTCC
CTTGGCAGGTTGTTTTGAATGGTAAAGTTGATGCATTCTGTGGAGGCTCTATCGTTA
ATGAAAAATGGATTGTAACTGCTGCCCACTGTGTTGAAACTGGTGTTAAAATTACAG
TTGTCGCAGGTGAACATAATATTGAGGAGACAGAACATACAGAGCAAAAGCGAAAT
GTGATTCGAATTATTCCTCACCACAACTACAATGCAGCTATTAATAAGTACAACCAT
GACATTGCCCTTCTGGAACTGGACGAACCCTTAGTGCTAAACAGCTACGTTACACCT
ATTTGCATTGCTGACAAGGAATACACGAACATCTTCCTCAAATTTGGATCTGGCTAT
GTAAGTGGCTGGGGAAGAGTCTTCCACAAAGGGAGATCAGCTTTAGTTCTTCAGTAC
CTTAGAGTTCCACTTGTTGACCGAGCCACATGTCTTCTATCTACAAAGTTCACCATCT
ATAACAACATGTTCTGTGCTGGCTTCCATGAAGGAGGTAGAGATTCATGTCAAGGAG
ATAGTGGGGGACCCCATGTTACTGAAGTGGAAGGGACCAGTTTCTTAACTGGAATTA
TTAGCTGGGGTGAAGAGTGTGCAATGAAAGGCAAATATGGAATATATACCAAGGTC
TCCCGGTATGTCAACTGGATTAAGGAAAAAACAAAGCTCACTGTCAGCGGATGGAG
ACTGTTCAAGAAGATCAGCTAA
Human Factor IX (R338L)-HiBit (2^{nd} Orientation) (SEQ ID NO: 273):
TTAGGAAATCTTCTTAAACAGCCGCCAGCCGCTCACGGTGAGCTTAGTCTTTTCTTTT
ATCCAATTCACGTAGCGAGAGACCTTCGTATAGATGCCATATTTCCCCTTCATCGCA
CATTCCTCCCCCCAACTTATTATCCCGGTCAAGAAACTTGTTCCTTCGACTTCAGTGA
CGTGTGGTCCACCTGAATCACCTTGGCATGAGTCGCGACCGCCCTCGTGAAACCCAG
CACAAAACATGTTATTGTAAATCGTAAATTTCGTGGACAGAAGACAGGTCGCTCTAT
CGACCAACGGGACGCGCAAATATTGCAGAACGAGGGCTGATCGACCTTTGTGGAAG
ACCCGCCCCCACCCACTCACATATCCGCTCCCAAATTTCAAGAAGATATTTGTATAT
TCTTTATCGGCTATACAAATCGGGGTAACATAGGAGTTAAGTACGAGTGGCTCGTCC
AGCTCCAGGAGGGCTATATCATGGTTGTACTTGTTTATAGCGGCATTATAATTGTGA
TGGGGTATGATCCTGATAACATTCCTTTTCTGTTCAGTATGCTCAGTTTCTTCAATGT
TGTGTTCGCCAGCCACGACCGTAATCTTAACCCCCGTCTCGACACAGTGTGCGGCCG
TTACAATCCACTTTTCATTGACTATGGAGCCCCCACAAAACGCGTCGACTTTTCCGTT
GAGCACCACCTGCCATGGAAATTGGCCAGGTTTAGCGTCCTCGCCCCCGACAACCCT
AGTAAAGTCATTAAATGACTGTGTGGATTGTGTTATATTATCAAGAATCGTTTCGGC
TTCAGTAGAGTTAACGTAGTCCACATCGGGAAAAACTGTCTCGGCCCTTGTCAACTT
TGATGTCTGGGACACACTTACCCGACCGCACGGGAAGGGCACCGCCGGTTCACAGC
TCTTTTGATTCTCAGCGAGCCGGTAGCCCTCAGTGCAACTACACACAACTTTGTTGTC
GGCGGAATTTTTACAGAATTGCTCGCATCGTCCATTTTTAATGTTGCAGGTGACGTCC
AACTCGCAGTTTTTTCCTTCAAAACCAAAAGGGCACCAACACTCGTAGGAATTTATA
TCGTCTTTACAACTCCCCCCATTCAGACATGGATTAGATTCGCATTGGTCCCCATCGA
CATATTGCTTCCAGAACTCAGTGGTCCGTTCTGTATTCTCAAACACCTCGCGCGCTTC
TTCAAAACTGCATTTTTCCTCCATACACTCTCGCTCCAAGTTCCCTTGCACGAATTCT
TCAAGCTTTCCTGAGTTATACCTTTTAGGCCGGTTAAGTATCTTATTCGCGTTTTCGT
GGTCCAGAAA
Human Factor IX Splice Acceptor (2^{nd} Orientation) (SEQ ID NO: 274):
CTGAAATGTAAAAGAATAATTCTTTAGTTTTAGCAAAAAAGAAAACATCATGAAAA
TTTTACATCTCTTAAGAAAGTCTTTGTTTTTAATCCAAATAATC
Nluc-P2A-GFP (1^{st} Orientation) (SEQ ID NO: 275):
TTTCTTGATCATGAAAACGCCAACAAAATTCTGAATCGGCCAAAGAGGTATAATTCA
GGTAAATTGGAAGAGTTTGTTCAAGGGAACCTTGAGAGAGAATGTATGGAAGAAAA
GTGTAGTTTTGAAGAAGCAGTATTCACTTTGGAGGACTTTGTCGGTGACTGGAGGCA
AACCGCTGGTTATAATCTCGACCAAGTACTGGAACAGGGCGGGGTAAGTTCCCTCTT
TCAGAATTTGGGTGTAAGCGTCACACCAATCCAGCGGATTGTGTTGTCTGGAGAGAA
CGGACTCAAAATTGACATCCATGTTATCATTCCATATGAAGGTCTCAGTGGAGACCA
AATGGGGCAGATCGAGAAGATTTTCAAGGTAGTTTACCCAGTCGACGATCACCACTT
CAAAGTCATTCTCCACTATGGCACACTTGTTATCGACGGAGTAACTCCTAATATGAT
TGATTACTTTGGTCGCCCGTATGAGGGCATCGCAGTGTTTGATGGCAAAAAGATCAC
CGTAACAGGAACGTTGTGGAATGGGAACAAGATAATCGACGAGAGATTGATAAATC
CAGACGGGTCACTCCTGTTCAGGGTTACAATTAACGGCGTCACAGGATGGAGACTCT
GTGAACGAATACTGGCCACAAATTTTTCACTCCTGAAGCAGGCCGGAGACGTGGAG
GAAAACCCAGGGCCCGTGAGCAAGGGCGAGGAGCTGTTCACCGGGGTGGTGCCCAT
CCTGGTCGAGCTGGACGGCGACGTAAACGGCCACAAGTTCAGCGTGTCCGGCGAGG
GCGAGGGCGATGCCACCTACGGCAAGCTGACCCTGAAGTTCATCTGCACCACCGGC
AAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCC
GAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGG
GCATCGACTTCAAGGAGGACGGCAACATCCTGGGGCACAAGCTGGAGTACAACTAC
AACAGCCACAACGTCTATATCATGGCCGACAAGCAGAAGAACGGCATCAAGGTGAA
CTTCAAGATCCGCCACAACATCGAGGACGGCAGCGTGCAGCTCGCCGACCACTACC
AGCAGAACACCCCCATCGGCGACGGCCCCGTGCTGCTGCCCGACAACCACTACCTG
AGCACCCAGTCCGCCCTGAGCAAAGACCCCAACGAGAAGCGCGATCACATGGTCCT
GCTGGAGTTCGTGACCGCCGCCGGGATCACTCTCGGCATGGACGAGCTGTACAAGG
GAGGAGGAAGCCCGAAGAAGAAGAGAAAGGTCTAA
Nluc-P2A-GFP (2^{nd} Orientation) (SEQ ID NO: 276):
TTACACCTTCCTCTTCTTCTTGGGGCTGCCGCCGCCCTTGTACAGCTCGTCCATGCCC
AGGGTGATGCCGGCGGCGGTCACGAACTCCAGCAGCACCATGTGGTCCCTCTTCTCG
TTGGGGTCCTTGCTCAGGGCGCTCTGGGTGCTCAGGTAGTGGTTGTCGGGCAGCAGC
ACGGGGCCGTCGCCGATGGGGGTGTTCTGCTGGTAGTGGTCGGCCAGCTGCACGCTG
CCGTCCTCGATGTTGTGCCTGATCTTGAAGTTCACCTTGATGCCGTTCTTCTGCTTGT
CGGCCATGATGTACACGTTGTGGCTGTTGTAGTTGTACTCCAGCTTGTGGCCCAGGA
TGTTGCCGTCCTCCTTGAAGTCGATGCCCTTCAGCTCGATCCTGTTCACCAGGGTGTC
GCCCTCGAACTTCACCTCGGCCCTGGTCTTGTAGTTGCCGTCGTCCTTGAAGAAGAT
GGTCCTCTCCTGCACGTAGCCCTCGGGCATGGCGCTCTTGAAGAAGTCGTGCTGCTT
CATGTGGTCGGGGTACCTGCTGAAGCACTGCACGCCGTAGGTCAGGGTGGTCACCA
GGGTGGGCCAGGGCACGGGCAGCTTGCCGGTGGTGCAGATGAACTTCAGGGTCAGC
TTGCCGTAGGTGGCGTCGCCCTCGCCCTCGCCGCTCACGCTGAACTTGTGGCCGTTC
ACGTCGCCGTCCAGCTCCACCAGGATGGGCACCACGCCGGTGAACAGCTCCTCGCC
CTTGCTCACGGGGCCGGGGTTCTCCTCCACGTCGCCGGCCTGCTTCAGCAGGCTGAA
GTTGGTGGCCAGGATCCTCTCGCACAGCCTCCAGCCGGTCACGCCGTTGATGGTCAC
CCTGAACAGCAGGCTGCCGTCGGGGTTGATCAGCCTCTCGTCGATGATCTTGTTGCC
GTTCCACAGGGTGCCGGTCACGGTGATCTTCTTGCCGTCGAACACGGCGATGCCCTC
GTAGGGCCTGCCGAAGTAGTCGATCATGTTGGGGGTCACGCCGTCGATCACCAGGG
TGCCGTAGTGCAGGATCACCTTGAAGTGGTGGTCGTCCACGGGGTACACCACCTTGA
AAATCTTCTCGATCTGGCCCATCTGGTCGCCGCTCAGGCCCTCGTAGGGGATGATCA
CGTGGATGTCGATCTTCAGGCCGTTCTCGCCGCTCAGCACGATCCTCTGGATGGGGG
TCACGCTCACGCCCAGGTTCTGGAACAGGCTGCTCACGCCGCCCTGCTCCAGCACCT
GGTCCAGGTTGTAGCCGGCGGTCTGCCTCCAGTCGCCCACGAAGTCCTCCAGGGTGA
ACACGGCCTCCTCGAAGCTGCACTTCTCCTCCATGCACTCCCTCTCCAGGTTGCCCTG
CACGAACTCCTCCAGCTTGCCGCTGTTGTACCTCTTGGGCCTGTTCAGGATCTTGTTG
GCGTTCTCGTGGTCCAGGAA
P00147 full sequence (from ITR to ITR): (SEQ ID NO: 277)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTCTTAGGTCAGTGAAGAGA
AGAACAAAAAGCAGCATATTACAGTTAGTTGTCTTCATCAATCTTTAAATATGTTGT
GTGGTTTTTCTCTCCCTGTTTCCACAGTTTTTCTTGATCATGAAAACGCCAACAAAAT
TCTGAATCGGCCAAAGAGGTATAATTCAGGTAAATTGGAAGAGTTTGTTCAAGGGA
ACCTTGAGAGAGAATGTATGGAAGAAAAGTGTAGTTTTGAAGAAGCACGAGAAGTT
TTTGAAAACACTGAAAGAACAACTGAATTTTGGAAGCAGTATGTTGATGGAGATCA
GTGTGAGTCCAATCCATGTTTAAATGGCGGCAGTTGCAAGGATGACATTAATTCCTA
TGAATGTTGGTGTCCCTTTGGATTTGAAGGAAAGAACTGTGAATTAGATGTAACATG
TAACATTAAGAATGGCAGATGCGAGCAGTTTTGTAAAAATAGTGCTGATAACAAGG
TGGTTTGCTCCTGTACTGAGGGATATCGACTTGCAGAAAACCAGAAGTCCTGTGAAC
CAGCAGTGCCATTTCCATGTGGAAGAGTTTCTGTTTCACAAACTTCTAAGCTCACCC
GTGCTGAGACTGTTTTTCCTGATGTGGACTATGTAAATTCTACTGAAGCTGAAACCA
TTTTGGATAACATCACTCAAAGCACCCAATCATTTAATGACTTCACTCGGGTTGTTG
GTGGAGAAGATGCCAAACCAGGTCAATTCCCTTGGCAGGTTGTTTTGAATGGTAAAG
TTGATGCATTCTGTGGAGGCTCTATCGTTAATGAAAAATGGATTGTAACTGCTGCCC
ACTGTGTTGAAACTGGTGTTAAAATTACAGTTGTCGCAGGTGAACATAATATTGAGG
AGACAGAACATACAGAGCAAAAGCGAAATGTGATTCGAATTATTCCTCACCACAAC
TACAATGCAGCTATTAATAAGTACAACCATGACATTGCCCTTCTGGAACTGGACGAA
CCCTTAGTGCTAAACAGCTACGTTACACCTATTTGCATTGCTGACAAGGAATACACG
AACATCTTCCTCAAATTTGGATCTGGCTATGTAAGTGGCTGGGGAAGAGTCTTCCAC
AAAGGGAGATCAGCTTTAGTTCTTCAGTACCTTAGAGTTCCACTTGTTGACCGAGCC
ACATGTCTTCTATCTACAAAGTTCACCATCTATAACAACATGTTCTGTGCTGGCTTCC
ATGAAGGAGGTAGAGATTCATGTCAAGGAGATAGTGGGGGACCCCATGTTACTGAA
GTGGAAGGGACCAGTTTCTTAACTGGAATTATTAGCTGGGGTGAAGAGTGTGCAAT
GAAAGGCAAATATGGAATATATACCAAGGTATCCCGGTATGTCAACTGGATTAAGG
AAAAAACAAAGCTCACTTAACCTCGACTGTGCCTTCTAGTTGCCAGCCATCTGTTGT
TTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATG
CTGGGGATGCGGTGGGCTCTATGGCTTCTGAGGCGGAAAGAACCAGCTGGGGCTCT
AGGGGGTATCCCCAAAAAACCTCCCACACCTCCCCCTGAACCTGAAACATAAAATG
AATGCAATTGTTGTTGTTAACTTGTTTATTGCAGCTTATAATGGTTACAAATAAAGCA
ATAGCATCACAAATTTCACAAATAAAGCATTTTTTTCACTGCATTCTAGTTGTGGTTT
GTCCAAACTCATCAATGTATCTTATCATGTCTGTTAGGTGAGCTTAGTCTTTTCTTTT
ATCCAATTCACGTAGCGAGAGACCTTCGTATAGATGCCATATTTCCCCTTCATCGCA
CATTCCTCCCCCCAACTTATTATCCCGGTCAAGAAACTTGTTCCTTCGACTTCAGTGA
CGTGTGGTCCACCTGAATCACCTTGGCATGAGTCGCGACCGCCCTCGTGAAACCCAG
CACAAAACATGTTATTGTAAATCGTAAATTTCGTGGACAGAAGACAGGTCGCTCTAT
CGACCAACGGGACGCGCAAATATTGCAGAACGAGGGCTGATCGACCTTTGTGGAAG
ACCCGCCCCCACCCACTCACATATCCGCTCCCAAATTTCAAGAAGATATTTGTATAT
TCTTTATCGGCTATACAAATCGGGGTAACATAGGAGTTAAGTACGAGTGGCTCGTCC
AGCTCCAGGAGGGCTATATCATGGTTGTACTTGTTTATAGCGGCATTATAATTGTGA
TGGGGTATGATCCTGATAACATTCCTTTTCTGTTCAGTATGCTCAGTTTCTTCAATGT
TGTGTTCGCCAGCCACGACCGTAATCTTAACCCCCGTCTCGACACAGTGTGCGGCCG
TTACAATCCACTTTTCATTGACTATGGAGCCCCCACAAAACGCGTCGACTTTTCCGTT
GAGCACCACCTGCCATGGAAATTGGCCAGGTTTAGCGTCCTCGCCCCCGACAACCCT
AGTAAAGTCATTAAATGACTGTGTGGATTGTGTTATATTATCAAGAATCGTTTCGGC
TTCAGTAGAGTTAACGTAGTCCACATCGGGAAAAACTGTCTCGGCCCTTGTCAACTT
TGATGTCTGGGACACACTTACCCGACCGCACGGGAAGGGCACCGCCGGTTCACAGC
TCTTTTGATTCTCAGCGAGCCGGTAGCCCTCAGTGCAACTACACACAACTTTGTTGTC
GGCGGAATTTTTACAGAATTGCTCGCATCGTCCATTTTTAATGTTGCAGGTGACGTCC
AACTCGCAGTTTTTTCCTTCAAAACCAAAAGGGCACCAACACTCGTAGGAATTTATA
TCGTCTTTACAACTCCCCCCATTCAGACATGGATTAGATTCGCATTGGTCCCCATCGA
CATATTGCTTCCAGAACTCAGTGGTCCGTTCTGTATTCTCAAACACCTCGCGCGCTTC
TTCAAAACTGCATTTTTCCTCCATACACTCTCGCTCCAAGTTCCCTTGCACGAATTCT
TCAAGCTTTCCTGAGTTATACCTTTTAGGCCGGTTAAGTATCTTATTCGCGTTTTCGT
GGTCCAGAAAAACTGTGGAAACAGGGAGAGAAAAACCACACAACATATTTAAAGA
TTGATGAAGACAACTAACTGTAATATGCTGCTTTTTGTTCTTCTCTTCACTGACCTAA
GAGATCTAGGAACCCCTAGTGATGGAGTTGGCCACTCCCTCTCTGCGCGCTCGCTCG
CTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAA
P00411 full sequence (from ITR to ITR): (SEQ ID NO: 278)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTCTGATTATTTGGATTAAAA
ACAAAGACTTTCTTAAGAGATGTAAAATTTTCATGATGTTTTCTTTTTTGCTAAAACT
AAAGAATTATTCTTTTACATTTCAGTTTTTCTTGATCATGAAAACGCCAACAAAATTC
TGAATCGGCCAAAGAGGTATAATTCAGGTAAATTGGAAGAGTTTGTTCAAGGGAAC
CTTGAGAGAGAATGTATGGAAGAAAAGTGTAGTTTTGAAGAAGCACGAGAAGTTTT
TGAAAACACTGAAAGAACAACTGAATTTTGGAAGCAGTATGTTGATGGAGATCAGT
GTGAGTCCAATCCATGTTTAAATGGCGGCAGTTGCAAGGATGACATTAATTCCTATG
AATGTTGGTGTCCCTTTGGATTTGAAGGAAAGAACTGTGAATTAGATGTAACATGTA
ACATTAAGAATGGCAGATGCGAGCAGTTTTGTAAAAATAGTGCTGATAACAAGGTG
GTTTGCTCCTGTACTGAGGGATATCGACTTGCAGAAAACCAGAAGTCCTGTGAACCA
GCAGTGCCATTTCCATGTGGAAGAGTTTCTGTTTCACAAACTTCTAAGCTCACCCGT
GCTGAGACTGTTTTTCCTGATGTGGACTATGTAAATTCTACTGAAGCTGAAACCATTT
TGGATAACATCACTCAAAGCACCCAATCATTTAATGACTTCACTCGGGTTGTTGGTG
GAGAAGATGCCAAACCAGGTCAATTCCCTTGGCAGGTTGTTTTGAATGGTAAAGTTG
ATGCATTCTGTGGAGGCTCTATCGTTAATGAAAAATGGATTGTAACTGCTGCCCACT
GTGTTGAAACTGGTGTTAAAATTACAGTTGTCGCAGGTGAACATAATATTGAGGAGA
CAGAACATACAGAGCAAAAGCGAAATGTGATTCGAATTATTCCTCACCACAACTAC
AATGCAGCTATTAATAAGTACAACCATGACATTGCCCTTCTGGAACTGGACGAACCC
TTAGTGCTAAACAGCTACGTTACACCTATTTGCATTGCTGACAAGGAATACACGAAC
ATCTTCCTCAAATTTGGATCTGGCTATGTAAGTGGCTGGGGAAGAGTCTTCCACAAA
GGGAGATCAGCTTTAGTTCTTCAGTACCTTAGAGTTCCACTTGTTGACCGAGCCACA
TGTCTTCTATCTACAAAGTTCACCATCTATAACAACATGTTCTGTGCTGGCTTCCATG
AAGGAGGTAGAGATTCATGTCAAGGAGATAGTGGGGGACCCCATGTTACTGAAGTG
GAAGGGACCAGTTTCTTAACTGGAATTATTAGCTGGGGTGAAGAGTGTGCAATGAA
AGGCAAATATGGAATATATACCAAGGTCTCCCGGTATGTCAACTGGATTAAGGAAA
AAACAAAGCTCACTGTCAGCGGATGGAGACTGTTCAAGAAGATCAGCTAACCTCGA
CTGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGCT
TCTGAGGCGGAAAGAACCAGCTGGGGCTCTAGGGGGTATCCCCAAAAAACCTCCCA
CACCTCCCCCTGAACCTGAAACATAAAATGAATGCAATTGTTGTTGTTAACTTGTTT
ATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTTATC
ATGTCTGTTAGGAAATCTTCTTAAACAGCCGCCAGCCGCTCACGGTGAGCTTAGTCT
TTTCTTTTATCCAATTCACGTAGCGAGAGACCTTCGTATAGATGCCATATTTCCCCTT
CATCGCACATTCCTCCCCCCAACTTATTATCCCGGTCAAGAAACTTGTTCCTTCGACT
TCAGTGACGTGTGGTCCACCTGAATCACCTTGGCATGAGTCGCGACCGCCCTCGTGA
AACCCAGCACAAAACATGTTATTGTAAATCGTAAATTTCGTGGACAGAAGACAGGT
CGCTCTATCGACCAACGGGACGCGCAAATATTGCAGAACGAGGGCTGATCGACCTT
TGTGGAAGACCCGCCCCCACCCACTCACATATCCGCTCCCAAATTTCAAGAAGATAT
TTGTATATTCTTTATCGGCTATACAAATCGGGGTAACATAGGAGTTAAGTACGAGTG
GCTCGTCCAGCTCCAGGAGGGCTATATCATGGTTGTACTTGTTTATAGCGGCATTAT
AATTGTGATGGGGTATGATCCTGATAACATTCCTTTTCTGTTCAGTATGCTCAGTTTC
TTCAATGTTGTGTTCGCCAGCCACGACCGTAATCTTAACCCCCGTCTCGACACAGTG
TGCGGCCGTTACAATCCACTTTTCATTGACTATGGAGCCCCCACAAAACGCGTCGAC
TTTTCCGTTGAGCACCACCTGCCATGGAAATTGGCCAGGTTTAGCGTCCTCGCCCCC
GACAACCCTAGTAAAGTCATTAAATGACTGTGTGGATTGTGTTATATTATCAAGAAT
CGTTTCGGCTTCAGTAGAGTTAACGTAGTCCACATCGGGAAAAACTGTCTCGGCCCT
TGTCAACTTTGATGTCTGGGACACACTTACCCGACCGCACGGGAAGGGCACCGCCG
GTTCACAGCTCTTTTGATTCTCAGCGAGCCGGTAGCCCTCAGTGCAACTACACACAA
CTTTGTTGTCGGCGGAATTTTTACAGAATTGCTCGCATCGTCCATTTTTAATGTTGCA
GGTGACGTCCAACTCGCAGTTTTTTCCTTCAAAACCAAAAGGGCACCAACACTCGTA
GGAATTTATATCGTCTTTACAACTCCCCCCATTCAGACATGGATTAGATTCGCATTGG
TCCCCATCGACATATTGCTTCCAGAACTCAGTGGTCCGTTCTGTATTCTCAAACACCT
CGCGCGCTTCTTCAAAACTGCATTTTTCCTCCATACACTCTCGCTCCAAGTTCCCTTG
CACGAATTCTTCAAGCTTTCCTGAGTTATACCTTTTAGGCCGGTTAAGTATCTTATTC
GCGTTTTCGTGGTCCAGAAAAACTGAAATGTAAAAGAATAATTCTTTAGTTTTAGCA
AAAAAGAAAACATCATGAAAATTTTACATCTCTTAAGAAAGTCTTTGTTTTTAATCC
AAATAATCAGAGATCTAGGAACCCCTAGTGATGGAGTTGGCCACTCCCTCTCTGCGC
GCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAA
P00415 full sequence (from ITR to ITR): (SEQ ID NO: 279)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTCTTAGGTCAGTGAAGAGA
AGAACAAAAAGCAGCATATTACAGTTAGTTGTCTTCATCAATCTTTAAATATGTTGT
GTGGTTTTTCTCTCCCTGTTTCCACAGTTTTTCTTGATCATGAAAACGCCAACAAAAT
TCTGAATCGGCCAAAGAGGTATAATTCAGGTAAATTGGAAGAGTTTGTTCAAGGGA
ACCTTGAGAGAGAATGTATGGAAGAAAAGTGTAGTTTTGAAGAAGCAGTATTCACT
TTGGAGGACTTTGTCGGTGACTGGAGGCAAACCGCTGGTTATAATCTCGACCAAGTA
CTGGAACAGGGCGGGGTAAGTTCCCTCTTTCAGAATTTGGGTGTAAGCGTCACACCA
ATCCAGCGGATTGTGTTGTCTGGAGAGAACGGACTCAAAATTGACATCCATGTTATC
ATTCCATATGAAGGTCTCAGTGGAGACCAAATGGGGCAGATCGAGAAGATTTTCAA
GGTAGTTTACCCAGTCGACGATCACCACTTCAAAGTCATTCTCCACTATGGCACACT
TGTTATCGACGGAGTAACTCCTAATATGATTGATTACTTTGGTCGCCCGTATGAGGG
CATCGCAGTGTTTGATGGCAAAAAGATCACCGTAACAGGAACGTTGTGGAATGGGA
ACAAGATAATCGACGAGAGATTGATAAATCCAGACGGGTCACTCCTGTTCAGGGTT
ACAATTAACGGCGTCACAGGATGGAGACTCTGTGAACGAATACTGGCCACAAATTT
TTCACTCCTGAAGCAGGCCGGAGACGTGGAGGAAAACCCAGGGCCCGTGAGCAAGG
GCGAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCT
CGTGACCACCCTGACCTACGGCGTGCAGTGCTTCAGCCGCTACCCCGACCACATGAA
GCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAA
CATCCTGGGGCACAAGCTGGAGTACAACTACAACAGCCACAACGTCTATATCATGG
CCGACAAGCAGAAGAACGGCATCAAGGTGAACTTCAAGATCCGCCACAACATCGAG
GACGGCAGCGTGCAGCTCGCCGACCACTACCAGCAGAACACCCCCATCGGCGACGG
CCCCGTGCTGCTGCCCGACAACCACTACCTGAGCACCCAGTCCGCCCTGAGCAAAG
ACCCCAACGAGAAGCGCGATCACATGGTCCTGCTGGAGTTCGTGACCGCCGCCGGG
ATCACTCTCGGCATGGACGAGCTGTACAAGGGAGGAGGAAGCCCGAAGAAGAAGA
GAAAGGTCTAACCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGATGC
GGTGGGCTCTATGGCTTCTGAGGCGGAAAGAACCAGCTGGGGCTCTAGGGGGTATC
CCCAAAAAACCTCCCACACCTCCCCCTGAACCTGAAACATAAAATGAATGCAATTGT
TGTTGTTAACTTGTTTATTGCAGCTTATAATGGTTACAAATAAAGCAATAGCATCAC
AAATTTCACAAATAAAGCATTTTTTTCACTGCATTCTAGTTGTGGTTTGTCCAAACTC
ATCAATGTATCTTATCATGTCTGTTACACCTTCCTCTTCTTCTTGGGGCTGCCGCCGC
CCTTGTACAGCTCGTCCATGCCCAGGGTGATGCCGGCGGCGGTCACGAACTCCAGCA
GCACCATGTGGTCCCTCTTCTCGTTGGGGTCCTTGCTCAGGGCGCTCTGGGTGCTCAG
GTAGTGGTTGTCGGGCAGCAGCACGGGGCCGTCGCCGATGGGGGTGTTCTGCTGGT
AGTGGTCGGCCAGCTGCACGCTGCCGTCCTCGATGTTGTGCCTGATCTTGAAGTTCA
CCTTGATGCCGTTCTTCTGCTTGTCGGCCATGATGTACACGTTGTGGCTGTTGTAGTT
GTACTCCAGCTTGTGGCCCAGGATGTTGCCGTCCTCCTTGAAGTCGATGCCCTTCAG
CTCGATCCTGTTCACCAGGGTGTCGCCCTCGAACTTCACCTCGGCCCTGGTCTTGTAG
TTGCCGTCGTCCTTGAAGAAGATGGTCCTCTCCTGCACGTAGCCCTCGGGCATGGCG
CTCTTGAAGAAGTCGTGCTGCTTCATGTGGTCGGGGTACCTGCTGAAGCACTGCACG
CCGTAGGTCAGGGTGGTCACCAGGGTGGGCCAGGGCACGGGCAGCTTGCCGGTGGT
GCAGATGAACTTCAGGGTCAGCTTGCCGTAGGTGGCGTCGCCCTCGCCCTCGCCGCT
CACGCTGAACTTGTGGCCGTTCACGTCGCCGTCCAGCTCCACCAGGATGGGCACCAC
GCCGGTGAACAGCTCCTCGCCCTTGCTCACGGGGCCGGGGTTCTCCTCCACGTCGCC
GGCCTGCTTCAGCAGGCTGAAGTTGGTGGCCAGGATCCTCTCGCACAGCCTCCAGCC
GGTCACGCCGTTGATGGTCACCCTGAACAGCAGGCTGCCGTCGGGGTTGATCAGCCT
CTCGTCGATGATCTTGTTGCCGTTCCACAGGGTGCCGGTCACGGTGATCTTCTTGCCG
TCGAACACGGCGATGCCCTCGTAGGGCCTGCCGAAGTAGTCGATCATGTTGGGGGTC
ACGCCGTCGATCACCAGGGTGCCGTAGTGCAGGATCACCTTGAAGTGGTGGTCGTCC
ACGGGGTACACCACCTTGAAAATCTTCTCGATCTGGCCCATCTGGTCGCCGCTCAGG
CCCTCGTAGGGGATGATCACGTGGATGTCGATCTTCAGGCCGTTCTCGCCGCTCAGC
ACGATCCTCTGGATGGGGGTCACGCTCACGCCCAGGTTCTGGAACAGGCTGCTCACG
CCGCCCTGCTCCAGCACCTGGTCCAGGTTGTAGCCGGCGGTCTGCCTCCAGTCGCCC
ACGAAGTCCTCCAGGGTGAACACGGCCTCCTCGAAGCTGCACTTCTCCTCCATGCAC
TCCCTCTCCAGGTTGCCCTGCACGAACTCCTCCAGCTTGCCGCTGTTGTACCTCTTGG
GCCTGTTCAGGATCTTGTTGGCGTTCTCGTGGTCCAGGAA
P00418 full sequence (from ITR to ITR): (SEQ ID NO: 280)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTCTTAGGTCAGTGAAGAGA
AGAACAAAAAGCAGCATATTACAGTTAGTTGTCTTCATCAATCTTTAAATATGTTGT
GTGGTTTTTCTCTCCCTGTTTCCACAGTTTTTCTTGATCATGAAAACGCCAACAAAAT
TCTGAATCGGCCAAAGAGGTATAATTCAGGTAAATTGGAAGAGTTTGTTCAAGGGA
ACCTTGAGAGAGAATGTATGGAAGAAAAGTGTAGTTTTGAAGAAGCACGAGAAGTT
TTTGAAAACACTGAAAGAACAACTGAATTTTGGAAGCAGTATGTTGATGGAGATCA
GTGTGAGTCCAATCCATGTTTAAATGGCGGCAGTTGCAAGGATGACATTAATTCCTA
TGAATGTTGGTGTCCCTTTGGATTTGAAGGAAAGAACTGTGAATTAGATGTAACATG
TAACATTAAGAATGGCAGATGCGAGCAGTTTTGTAAAAATAGTGCTGATAACAAGG
TGGTTTGCTCCTGTACTGAGGGATATCGACTTGCAGAAAACCAGAAGTCCTGTGAAC
CAGCAGTGCCATTTCCATGTGGAAGAGTTTCTGTTTCACAAACTTCTAAGCTCACCC
GTGCTGAGACTGTTTTTCCTGATGTGGACTATGTAAATTCTACTGAAGCTGAAACCA
TTTTGGATAACATCACTCAAAGCACCCAATCATTTAATGACTTCACTCGGGTTGTTG
GTGGAGAAGATGCCAAACCAGGTCAATTCCCTTGGCAGGTTGTTTTGAATGGTAAAG
TTGATGCATTCTGTGGAGGCTCTATCGTTAATGAAAAATGGATTGTAACTGCTGCCC
ACTGTGTTGAAACTGGTGTTAAAATTACAGTTGTCGCAGGTGAACATAATATTGAGG
AGACAGAACATACAGAGCAAAAGCGAAATGTGATTCGAATTATTCCTCACCACAAC
TACAATGCAGCTATTAATAAGTACAACCATGACATTGCCCTTCTGGAACTGGACGAA
CCCTTAGTGCTAAACAGCTACGTTACACCTATTTGCATTGCTGACAAGGAATACACG
AACATCTTCCTCAAATTTGGATCTGGCTATGTAAGTGGCTGGGGAAGAGTCTTCCAC
AAAGGGAGATCAGCTTTAGTTCTTCAGTACCTTAGAGTTCCACTTGTTGACCGAGCC
ACATGTCTTCTATCTACAAAGTTCACCATCTATAACAACATGTTCTGTGCTGGCTTCC
ATGAAGGAGGTAGAGATTCATGTCAAGGAGATAGTGGGGGACCCCATGTTACTGAA
GTGGAAGGGACCAGTTTCTTAACTGGAATTATTAGCTGGGGTGAAGAGTGTGCAAT
GAAAGGCAAATATGGAATATATACCAAGGTCTCCCGGTATGTCAACTGGATTAAGG
AAAAAACAAAGCTCACTGTCAGCGGATGGAGACTGTTCAAGAAGATCAGCTAACCT
CGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATG
GCTTCTGAGGCGGAAAGAACCAGCTGGGGCTCTAGGGGGTATCCCCAAAAAACCTC
CCACACCTCCCCCTGAACCTGAAACATAAAATGAATGCAATTGTTGTTGTTAACTTG
TTTATTGCAGCTTATAATGGTTACAAATAAAGCAATAGCATCACAAATTTCACAAAT
AAAGCATTTTTTTCACTGCATTCTAGTTGTGGTTTGTCCAAACTCATCAATGTATCTT
ATCATGTCTGTTAGGAAATCTTCTTAAACAGCCGCCAGCCGCTCACGGTGAGCTTAG
TCTTTTCTTTTATCCAATTCACGTAGCGAGAGACCTTCGTATAGATGCCATATTTCCC
CTTCATCGCACATTCCTCCCCCCAACTTATTATCCCGGTCAAGAAACTTGTTCCTTCG
ACTTCAGTGACGTGTGGTCCACCTGAATCACCTTGGCATGAGTCGCGACCGCCCTCG
TGAAACCCAGCACAAAACATGTTATTGTAAATCGTAAATTTCGTGGACAGAAGACA
GGTCGCTCTATCGACCAACGGGACGCGCAAATATTGCAGAACGAGGGCTGATCGAC
CTTTGTGGAAGACCCGCCCCCACCCACTCACATATCCGCTCCCAAATTTCAAGAAGA
TATTTGTATATTCTTTATCGGCTATACAAATCGGGGTAACATAGGAGTTAAGTACGA
GTGGCTCGTCCAGCTCCAGGAGGGCTATATCATGGTTGTACTTGTTTATAGCGGCAT
TATAATTGTGATGGGGTATGATCCTGATAACATTCCTTTTCTGTTCAGTATGCTCAGT
TTCTTCAATGTTGTGTTCGCCAGCCACGACCGTAATCTTAACCCCCGTCTCGACACA
GTGTGCGGCCGTTACAATCCACTTTTCATTGACTATGGAGCCCCCACAAAACGCGTC
GACTTTTCCGTTGAGCACCACCTGCCATGGAAATTGGCCAGGTTTAGCGTCCTCGCC
CCCGACAACCCTAGTAAAGTCATTAAATGACTGTGTGGATTGTGTTATATTATCAAG
AATCGTTTCGGCTTCAGTAGAGTTAACGTAGTCCACATCGGGAAAAACTGTCTCGGC
CCTTGTCAACTTTGATGTCTGGGACACACTTACCCGACCGCACGGGAAGGGCACCGC
CGGTTCACAGCTCTTTTGATTCTCAGCGAGCCGGTAGCCCTCAGTGCAACTACACAC
AACTTTGTTGTCGGCGGAATTTTTACAGAATTGCTCGCATCGTCCATTTTTAATGTTG
CAGGTGACGTCCAACTCGCAGTTTTTTCCTTCAAAACCAAAAGGGCACCAACACTCG
TAGGAATTTATATCGTCTTTACAACTCCCCCCATTCAGACATGGATTAGATTCGCATT
GGTCCCCATCGACATATTGCTTCCAGAACTCAGTGGTCCGTTCTGTATTCTCAAACAC
CTCGCGCGCTTCTTCAAAACTGCATTTTTCCTCCATACACTCTCGCTCCAAGTTCCCT
TGCACGAATTCTTCAAGCTTTCCTGAGTTATACCTTTTAGGCCGGTTAAGTATCTTAT
TCGCGTTTTCGTGGTCCAGAAAAACTGTGGAAACAGGGAGAGAAAAACCACACAAC
ATATTTAAAGATTGATGAAGACAACTAACTGTAATATGCTGCTTTTTGTTCTTCTCTT
CACTGACCTAAGAGATCTAGGAACCCCTAGTGATGGAGTTGGCCACTCCCTCTCTGC
GCGCTCGCTCGCTCACTGAGGCCGCCCGGGCAAAGCCCGGGCGTCGGGCGACCTTT
GGTCGCCCGGCCTCAGTGAGCGAGCGAGCGCGCAGAGAGGGAGTGGCCAA
P00123 full sequence (from ITR to ITR): (SEQ ID NO: 281)
GGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGAGAGG
GAGTGGCCAACTCCATCACTAGGGGTTCCTGGAGGGGTGGAGTCGTGATAGGTCAG
TGAAGAGAAGAACAAAAAGCAGCATATTACAGTTAGTTGTCTTCATCAATCTTTAAA
TATGTTGTGTGGTTTTTCTCTCCCTGTTTCCACAGTTTTTCTTGATCATGAAAACGCCA
ACAAAATTCTGAATCGGCCAAAGAGGTATAATTCAGGTAAATTGGAAGAGTTTGTTC
AAGGGAACCTTGAGAGAGAATGTATGGAAGAAAAGTGTAGTTTTGAAGAAGCACGA
GAAGTTTTTGAAAACACTGAAAGAACAACTGAATTTTGGAAGCAGTATGTTGATGG
AGATCAGTGTGAGTCCAATCCATGTTTAAATGGCGGCAGTTGCAAGGATGACATTAA
TTCCTATGAATGTTGGTGTCCCTTTGGATTTGAAGGAAAGAACTGTGAATTAGATGT
AACATGTAACATTAAGAATGGCAGATGCGAGCAGTTTTGTAAAAATAGTGCTGATA
ACAAGGTGGTTTGCTCCTGTACTGAGGGATATCGACTTGCAGAAAACCAGAAGTCCT
GTGAACCAGCAGTGCCATTTCCATGTGGAAGAGTTTCTGTTTCACAAACTTCTAAGC
TCACCCGTGCTGAGACTGTTTTTCCTGATGTGGACTATGTAAATTCTACTGAAGCTGA
AACCATTTTGGATAACATCACTCAAAGCACCCAATCATTTAATGACTTCACTCGGGT
TGTTGGTGGAGAAGATGCCAAACCAGGTCAATTCCCTTGGCAGGTTGTTTTGAATGG
TAAAGTTGATGCATTCTGTGGAGGCTCTATCGTTAATGAAAAATGGATTGTAACTGC
TGCCCACTGTGTTGAAACTGGTGTTAAAATTACAGTTGTCGCAGGTGAACATAATAT
TGAGGAGACAGAACATACAGAGCAAAAGCGAAATGTGATTCGAATTATTCCTCACC
ACAACTACAATGCAGCTATTAATAAGTACAACCATGACATTGCCCTTCTGGAACTGG
ACGAACCCTTAGTGCTAAACAGCTACGTTACACCTATTTGCATTGCTGACAAGGAAT
ACACGAACATCTTCCTCAAATTTGGATCTGGCTATGTAAGTGGCTGGGGAAGAGTCT
TCCACAAAGGGAGATCAGCTTTAGTTCTTCAGTACCTTAGAGTTCCACTTGTTGACC
GAGCCACATGTCTTCTATCTACAAAGTTCACCATCTATAACAACATGTTCTGTGCTG
GCTTCCATGAAGGAGGTAGAGATTCATGTCAAGGAGATAGTGGGGGACCCCATGTT
ACTGAAGTGGAAGGGACCAGTTTCTTAACTGGAATTATTAGCTGGGGTGAAGAGTG
TGCAATGAAAGGCAAATATGGAATATATACCAAGGTATCCCGGTATGTCAACTGGA
TTAAGGAAAAAACAAAGCTCACTTAACCTCGACTGTGCCTTCTAGTTGCCAGCCATC
TGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATAGCAG
GCATGCTGGGGATGCGGTGGGCTCTATGGCTTCTGAGGCGGAAAGAACCAGCTGGG
GCTCTAGGGGGTATCCCCACTAGTCCACTCCCTCTCTGCGCGCTCGCTCGCTCACTG
AGGCCGGGCGACCAAAGGTCGCCCGACGCCCGGGCTTTGCCCGGGCGGCCTCAGTG
AGCGAGCGAGCGCGCAGAGAGGGA
P00204 full sequence (from ITR to ITR): (SEQ ID NO: 282)
GGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGAGAGG
GAGTGGCCAACTCCATCACTAGGGGTTCCTGGAGGGGTGGAGTCGTGACCTAGGTC
GTCTCCGGCTCTGCTTTTTCCAGGGGTGTGTTTCGCCGAGAAGCACGTAAGAGTTTT
ATGTTTTTTCATCTCTGCTTGTATTTTTCTAGTAATGGAAGCCTGGTATTTTAAAATA
GTTAAATTTTCCTTTAGTGCTGATTTCTAGATTATTATTACTGTTGTTGTTGTTATTAT
TGTCATTATTTGCATCTGAGAACTAGGTCAGTGAAGAGAAGAACAAAAAGCAGCAT
ATTACAGTTAGTTGTCTTCATCAATCTTTAAATATGTTGTGTGGTTTTTCTCTCCCTGT
TTCCACAGTTTTTCTTGATCATGAAAACGCCAACAAAATTCTGAATCGGCCAAAGAG
GTATAATTCAGGTAAATTGGAAGAGTTTGTTCAAGGGAACCTTGAGAGAGAATGTA
TGGAAGAAAAGTGTAGTTTTGAAGAAGCACGAGAAGTTTTTGAAAACACTGAAAGA
ACAACTGAATTTTGGAAGCAGTATGTTGATGGAGATCAGTGTGAGTCCAATCCATGT
TTAAATGGCGGCAGTTGCAAGGATGACATTAATTCCTATGAATGTTGGTGTCCCTTT
GGATTTGAAGGAAAGAACTGTGAATTAGATGTAACATGTAACATTAAGAATGGCAG
ATGCGAGCAGTTTTGTAAAAATAGTGCTGATAACAAGGTGGTTTGCTCCTGTACTGA
GGGATATCGACTTGCAGAAAACCAGAAGTCCTGTGAACCAGCAGTGCCATTTCCAT
GTGGAAGAGTTTCTGTTTCACAAACTTCTAAGCTCACCCGTGCTGAGACTGTTTTTCC
TGATGTGGACTATGTAAATTCTACTGAAGCTGAAACCATTTTGGATAACATCACTCA
AAGCACCCAATCATTTAATGACTTCACTCGGGTTGTTGGTGGAGAAGATGCCAAACC
AGGTCAATTCCCTTGGCAGGTTGTTTTGAATGGTAAAGTTGATGCATTCTGTGGAGG
CTCTATCGTTAATGAAAAATGGATTGTAACTGCTGCCCACTGTGTTGAAACTGGTGT
TAAAATTACAGTTGTCGCAGGTGAACATAATATTGAGGAGACAGAACATACAGAGC
AAAAGCGAAATGTGATTCGAATTATTCCTCACCACAACTACAATGCAGCTATTAATA
AGTACAACCATGACATTGCCCTTCTGGAACTGGACGAACCCTTAGTGCTAAACAGCT
ACGTTACACCTATTTGCATTGCTGACAAGGAATACACGAACATCTTCCTCAAATTTG
GATCTGGCTATGTAAGTGGCTGGGGAAGAGTCTTCCACAAAGGGAGATCAGCTTTA
GTTCTTCAGTACCTTAGAGTTCCACTTGTTGACCGAGCCACATGTCTTCTATCTACAA
AGTTCACCATCTATAACAACATGTTCTGTGCTGGCTTCCATGAAGGAGGTAGAGATT
CATGTCAAGGAGATAGTGGGGGACCCCATGTTACTGAAGTGGAAGGGACCAGTTTC
TTAACTGGAATTATTAGCTGGGGTGAAGAGTGTGCAATGAAAGGCAAATATGGAAT
ATATACCAAGGTATCCCGGTATGTCAACTGGATTAAGGAAAAAACAAAGCTCACTT
AACCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCT
CTATGGCTTCTGAGGCGGAAAGAACCAGCTGGGGCTCTAGGGGGTATCCCCCTTAG
GTGGTTATATTATTGATATATTTTTGGTATCTTTGATGACAATAATGGGGGATTTTGA
AAGCTTAGCTTTAAATTTCTTTTAATTAAAAAAAAATGCTAGGCAGAATGACTCAAA
TTACGTTGGATACAGTTGAATTTATTACGGTCTCATAGGGCCTGCCTGCTCGACCAT
GCTATACTAAAAATTAAAAGTGTACTAGTCCACTCCCTCTCTGCGCGCTCGCTCGCT
CACTGAGGCCGGGCGACCAAAGGTCGCCCGACGCCCGGGCTTTGCCCGGGCGGCCT
CAGTGAGCGAGCGAGCGCGCAGAGAGGGA
P00353 full sequence (from ITR to ITR): (SEQ ID NO: 283)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTGATTTTGAAAGCTTAGCTT
TAAATTTCTTTTAATTAAAAAAAAATGCTAGGCAGAATGACTCAAATTACGTTGGAT
ACAGTTGAATTTATTACGGTCTCATAGGGCCTGCCTGCTCGACCATGCTATACTAAA
AATTAAAAGTGTGTGTTACTAATTTTATAAATGGAGTTTCCATTTATATTTACCTTTA
TTTCTTATTTACCATTGTCTTAGTAGATATTTACAAACATGACAGAAACACTAAATCT
TGAGTTTGAATGCACAGATATAAACACTTAACGGGTTTTAAAAATAATAATGTTGGT
GAAAAAATATAACTTTGAGTGTAGCAGAGAGGAACCATTGCCACCTTCAGATTTTCC
TGTAACGATCGGGAACTGGCATCTTCAGGGAGTAGCTTAGGTCAGTGAAGAGAAGA
ACAAAAAGCAGCATATTACAGTTAGTTGTCTTCATCAATCTTTAAATATGTTGTGTG
GTTTTTCTCTCCCTGTTTCCACAGTTTTTCTTGATCATGAAAACGCCAACAAAATTCT
GAATCGGCCAAAGAGGTTTCTTGATCATGAAAACGCCAACAAAATTCTGAATCGGC
CAAAGAGGTATAATTCAGGTAAATTGGAAGAGTTTGTTCAAGGGAACCTTGAGAGA
GAATGTATGGAAGAAAAGTGTAGTTTTGAAGAAGCACGAGAAGTTTTTGAAAACAC
TGAAAGAACAACTGAATTTTGGAAGCAGTATGTTGATGGAGATCAGTGTGAGTCCA
ATCCATGTTTAAATGGCGGCAGTTGCAAGGATGACATTAATTCCTATGAATGTTGGT
GTCCCTTTGGATTTGAAGGAAAGAACTGTGAATTAGATGTAACATGTAACATTAAGA
ATGGCAGATGCGAGCAGTTTTGTAAAAATAGTGCTGATAACAAGGTGGTTTGCTCCT
GTACTGAGGGATATCGACTTGCAGAAAACCAGAAGTCCTGTGAACCAGCAGTGCCA
TTTCCATGTGGAAGAGTTTCTGTTTCACAAACTTCTAAGCTCACCCGTGCTGAGACTG
TTTTTCCTGATGTGGACTATGTAAATTCTACTGAAGCTGAAACCATTTTGGATAACAT
CACTCAAAGCACCCAATCATTTAATGACTTCACTCGGGTTGTTGGTGGAGAAGATGC
CAAACCAGGTCAATTCCCTTGGCAGGTTGTTTTGAATGGTAAAGTTGATGCATTCTG
TGGAGGCTCTATCGTTAATGAAAAATGGATTGTAACTGCTGCCCACTGTGTTGAAAC
TGGTGTTAAAATTACAGTTGTCGCAGGTGAACATAATATTGAGGAGACAGAACATA
CAGAGCAAAAGCGAAATGTGATTCGAATTATTCCTCACCACAACTACAATGCAGCT
ATTAATAAGTACAACCATGACATTGCCCTTCTGGAACTGGACGAACCCTTAGTGCTA
AACAGCTACGTTACACCTATTTGCATTGCTGACAAGGAATACACGAACATCTTCCTC
AAATTTGGATCTGGCTATGTAAGTGGCTGGGGAAGAGTCTTCCACAAAGGGAGATC
AGCTTTAGTTCTTCAGTACCTTAGAGTTCCACTTGTTGACCGAGCCACATGTCTTCTA
TCTACAAAGTTCACCATCTATAACAACATGTTCTGTGCTGGCTTCCATGAAGGAGGT
AGAGATTCATGTCAAGGAGATAGTGGGGGACCCCATGTTACTGAAGTGGAAGGGAC
CAGTTTCTTAACTGGAATTATTAGCTGGGGTGAAGAGTGTGCAATGAAAGGCAAAT
ATGGAATATATACCAAGGTATCCCGGTATGTCAACTGGATTAAGGAAAAAACAAAG
CTCACTTAACCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGGATGCG
GTGGGCTCTATGGCTTCTGAGGCGGAAAGAACCAGCTGGGGCTCTAGGGGGTATCC
CCGTGAGATCGCCCATCGGTATAATGATTTGGGAGAACAACATTTCAAAGGCCTGTA
AGTTATAATGCTGAAAGCCCACTTAATATTTCTGGTAGTATTAGTTAAAGTTTTAAA
ACACCTTTTTCCACCTTGAGTGTGAGAATTGTAGAGCAGTGCTGTCCAGTAGAAATG
TGTGCATTGACAGAAAGACTGTGGATCTGTGCTGAGCAATGTGGCAGCCAGAGATC
ACAAGGCTATCAAGCACTTTGCACATGGCAAGTGTAACTGAGAAGCACACATTCAA
ATAATAGTTAATTTTAATTGAATGTATCTAGCCATGTGTGGCTAGTAGCTCCTTTCCT
GGAGAGAGAATCTGGAGCCCACATCTAACTTGTTAAGTCTGGAATCTTATTTTTTAT
TTCTGGAAAGGTCTATGAACTATAGTTTTGGGGGCAGCTCACTTACTAACTTTTAAT
GCAATAAGAATCTCATGGTATCTTGAGAACATTATTTTGTCTCTTTGTAGATCTAGGA
ACCCCTAGTGATGGAGTTGGCCACTCCCTCTCTGCGCGCTCGCTCGCTCACTGAGGC
CGCCCGGGCAAAGCCCGGGCGTCGGGCGACCTTTGGTCGCCCGGCCTCAGTGAGCG
AGCGAGCGCGCAGAGAGGGAGTGGCCAA
P00354 full sequence (from ITR to ITR): (SEQ ID NO: 284)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTTAGCCTCTGGCAAAATGAA
GTGGGTAACCTTTCTCCTCCTCCTCTTCGTCTCCGGCTCTGCTTTTTCCAGGGGTGTGT
TTCGCCGAGAAGCACGTAAGAGTTTTATGTTTTTTCATCTCTGCTTGTATTTTTCTAG
TAATGGAAGCCTGGTATTTTAAAATAGTTAAATTTTCCTTTAGTGCTGATTTCTAGAT
TATTATTACTGTTGTTGTTGTTATTATTGTCATTATTTGCATCTGAGAACCCTTAGGTG
GTTATATTATTGATATATTTTTGGTATCTTTGATGACAATAATGGGGGATTTTGAAAG
CTTAGCTTTAAATTTCTTTTAATTAAAAAAAAATGCTAGGCAGAATGACTCAAATTA
CGTTGGATACAGTTGAATTTATTACGGTCTCATAGGGCCTGCCTGCTCGACCATGCT
ATACTAAAAATTAAAAGTGTGTGTTACTAATTTTATAAATGGAGTTTCCATTTATATT
TACCTTTATTTCTTATTTACCATTGTCTTAGTAGATATTTACAAACATGACAGAAACA
CTAAATCTTGAGTTTGAATGCACAGATATAAACACTTAACGGGTTTTAAAAATAATA
ATGTTGGTGAAAAAATATAACTTTGAGTGTAGCAGAGAGGAACCATTGCCACCTTCA
GATTTTCCTGTAACGATCGGGAACTGGCATCTTCAGGGAGTAGCTTAGGTCAGTGAA
GAGAAGAACAAAAAGCAGCATATTACAGTTAGTTGTCTTCATCAATCTTTAAATATG
TTGTGTGGTTTTTCTCTCCCTGTTTCCACAGTTTTTCTTGATCATGAAAACGCCAACA
AAATTCTGAATCGGCCAAAGAGGTATAATTCAGGTAAATTGGAAGAGTTTGTTCAA
GGGAACCTTGAGAGAGAATGTATGGAAGAAAAGTGTAGTTTTGAAGAAGCACGAGA
AGTTTTTGAAAACACTGAAAGAACAACTGAATTTTGGAAGCAGTATGTTGATGGAG
ATCAGTGTGAGTCCAATCCATGTTTAAATGGCGGCAGTTGCAAGGATGACATTAATT
CCTATGAATGTTGGTGTCCCTTTGGATTTGAAGGAAAGAACTGTGAATTAGATGTAA
CATGTAACATTAAGAATGGCAGATGCGAGCAGTTTTGTAAAAATAGTGCTGATAAC
AAGGTGGTTTGCTCCTGTACTGAGGGATATCGACTTGCAGAAAACCAGAAGTCCTGT
GAACCAGCAGTGCCATTTCCATGTGGAAGAGTTTCTGTTTCACAAACTTCTAAGCTC
ACCCGTGCTGAGACTGTTTTTCCTGATGTGGACTATGTAAATTCTACTGAAGCTGAA
ACCATTTTGGATAACATCACTCAAAGCACCCAATCATTTAATGACTTCACTCGGGTT
GTTGGTGGAGAAGATGCCAAACCAGGTCAATTCCCTTGGCAGGTTGTTTTGAATGGT
AAAGTTGATGCATTCTGTGGAGGCTCTATCGTTAATGAAAAATGGATTGTAACTGCT
GCCCACTGTGTTGAAACTGGTGTTAAAATTACAGTTGTCGCAGGTGAACATAATATT
GAGGAGACAGAACATACAGAGCAAAAGCGAAATGTGATTCGAATTATTCCTCACCA
CAACTACAATGCAGCTATTAATAAGTACAACCATGACATTGCCCTTCTGGAACTGGA
CGAACCCTTAGTGCTAAACAGCTACGTTACACCTATTTGCATTGCTGACAAGGAATA
CACGAACATCTTCCTCAAATTTGGATCTGGCTATGTAAGTGGCTGGGGAAGAGTCTT
CCACAAAGGGAGATCAGCTTTAGTTCTTCAGTACCTTAGAGTTCCACTTGTTGACCG
AGCCACATGTCTTCTATCTACAAAGTTCACCATCTATAACAACATGTTCTGTGCTGGC
TTCCATGAAGGAGGTAGAGATTCATGTCAAGGAGATAGTGGGGGACCCCATGTTAC
TGAAGTGGAAGGGACCAGTTTCTTAACTGGAATTATTAGCTGGGGTGAAGAGTGTG
CAATGAAAGGCAAATATGGAATATATACCAAGGTATCCCGGTATGTCAACTGGATT
AAGGAAAAAACAAAGCTCACTTAACCTCGACTGTGCCTTCTAGTTGCCAGCCATCTG
TTGTTTGCCCCTCCCCCGTGCCTTCCTTGACCCTGGAAGGTGCCACTCCCACTGTCCT
TTCCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAGCAGG
CATGCTGGGGATGCGGTGGGCTCTATGGCTTCTGAGGCGGAAAGAACCAGCTGGGG
CTCTAGGGGGTATCCCCGTGAGATCGCCCATCGGTATAATGATTTGGGAGAACAACA
TTTCAAAGGCCTGTAAGTTATAATGCTGAAAGCCCACTTAATATTTCTGGTAGTATT
AGTTAAAGTTTTAAAACACCTTTTTCCACCTTGAGTGTGAGAATTGTAGAGCAGTGC
TGTCCAGTAGAAATGTGTGCATTGACAGAAAGACTGTGGATCTGTGCTGAGCAATGT
GGCAGCCAGAGATCACAAGGCTATCAAGCACTTTGCACATGGCAAGTGTAACTGAG
AAGCACACATTCAAATAATAGTTAATTTTAATTGAATGTATCTAGCCATGTGTGGCT
AGTAGCTCCTTTCCTGGAGAGAGAATCTGGAGCCCACATCTAACTTGTTAAGTCTGG
AATCTTATTTTTTATTTCTGGAAAGGTCTATGAACTATAGTTTTGGGGGCAGCTCACT
TACTAACTTTTAATGCAATAAGAATCTCATGGTATCTTGAGAACATTATTTTGTCTCT
TTGTAGTACTGAAACCTTATACATGTGAAGTAAGGGGTCTATACTTAAGTCACATCT
CCAACCTTAGTAATGTTTTAATGTAGTAAAAAAATGAGTAATTAATTTATTTTTAGA
AGGTCAATAGTATCATGTATTCCAAATAACAGAGGTATATGGTTAGAAAAGAAACA
ATTCAAAGGACTTATATAATATCTAGCCTTGACAATGAATAAATTTAGAGAGTAGTT
TGCCTGTTTGCCTCATGTTCATAAATCTATTGACACATATGTGCATCTGCACTTCAGC
ATGGTAGAAGTCCATATTCAGATCTAGGAACCCCTAGTGATGGAGTTGGCCACTCCC
TCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCA
A
P00350: The 300/600 bp HA F9 construct (for G551) (SEQ ID NO: 285)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTAAGTATATTAGAGCGAGTC
TTTCTGCACACAGATCACCTTTCCTATCAACCCCACTAGCCTCTGGCAAAATGAAGT
GGGTAACCTTTCTCCTCCTCCTCTTCGTCTCCGGCTCTGCTTTTTCCAGGGGTGTGTTT
CGCCGAGAAGCACGTAAGAGTTTTATGTTTTTTCATCTCTGCTTGTATTTTTCTAGTA
ATGGAAGCCTGGTATTTTAAAATAGTTAAATTTTCCTTTAGTGCTGATTTCTAGATTA
TTATTACTGTTGTTGTTGTTATTATTGTCATTATTTGCATCTGAGAACCTTTTTCTTGA
TCATGAAAACGCCAACAAAATTCTGAATCGGCCAAAGAGGTATAATTCAGGTAAAT
TGGAAGAGTTTGTTCAAGGGAACCTTGAGAGAGAATGTATGGAAGAAAAGTGTAGT
TTTGAAGAAGCACGAGAAGTTTTTGAAAACACTGAAAGAACAACTGAATTTTGGAA
GCAGTATGTTGATGGAGATCAGTGTGAGTCCAATCCATGTTTAAATGGCGGCAGTTG
CAAGGATGACATTAATTCCTATGAATGTTGGTGTCCCTTTGGATTTGAAGGAAAGAA
CTGTGAATTAGATGTAACATGTAACATTAAGAATGGCAGATGCGAGCAGTTTTGTAA
AAATAGTGCTGATAACAAGGTGGTTTGCTCCTGTACTGAGGGATATCGACTTGCAGA
AAACCAGAAGTCCTGTGAACCAGCAGTGCCATTTCCATGTGGAAGAGTTTCTGTTTC
ACAAACTTCTAAGCTCACCCGTGCTGAGACTGTTTTTCCTGATGTGGACTATGTAAA
TTCTACTGAAGCTGAAACCATTTTGGATAACATCACTCAAAGCACCCAATCATTTAA
TGACTTCACTCGGGTTGTTGGTGGAGAAGATGCCAAACCAGGTCAATTCCCTTGGCA
GGTTGTTTTGAATGGTAAAGTTGATGCATTCTGTGGAGGCTCTATCGTTAATGAAAA
ATGGATTGTAACTGCTGCCCACTGTGTTGAAACTGGTGTTAAAATTACAGTTGTCGC
AGGTGAACATAATATTGAGGAGACAGAACATACAGAGCAAAAGCGAAATGTGATTC
GAATTATTCCTCACCACAACTACAATGCAGCTATTAATAAGTACAACCATGACATTG
CCCTTCTGGAACTGGACGAACCCTTAGTGCTAAACAGCTACGTTACACCTATTTGCA
TTGCTGACAAGGAATACACGAACATCTTCCTCAAATTTGGATCTGGCTATGTAAGTG
GCTGGGGAAGAGTCTTCCACAAAGGGAGATCAGCTTTAGTTCTTCAGTACCTTAGAG
TTCCACTTGTTGACCGAGCCACATGTCTTCTATCTACAAAGTTCACCATCTATAACAA
CATGTTCTGTGCTGGCTTCCATGAAGGAGGTAGAGATTCATGTCAAGGAGATAGTGG
GGGACCCCATGTTACTGAAGTGGAAGGGACCAGTTTCTTAACTGGAATTATTAGCTG
GGGTGAAGAGTGTGCAATGAAAGGCAAATATGGAATATATACCAAGGTATCCCGGT
ATGTCAACTGGATTAAGGAAAAAACAAAGCTCACTTAACCTCGACTGTGCCTTCTAG
TTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAA
GAACCAGCTGGGGCTCTAGGGGGTATCCCCCTTAGGTGGTTATATTATTGATATATT
TTTGGTATCTTTGATGACAATAATGGGGGATTTTGAAAGCTTAGCTTTAAATTTCTTT
TAATTAAAAAAAAATGCTAGGCAGAATGACTCAAATTACGTTGGATACAGTTGAAT
TTATTACGGTCTCATAGGGCCTGCCTGCTCGACCATGCTATACTAAAAATTAAAAGT
GTGTGTTACTAATTTTATAAATGGAGTTTCCATTTATATTTACCTTTATTTCTTATTTA
CCATTGTCTTAGTAGATATTTACAAACATGACAGAAACACTAAAGATCTAGGAACCC
CTAGTGATGGAGTTGGCCACTCCCTCTCTGCGCGCTCGCTCGCTCACTGAGGCCGCC
CGGGCAAAGCCCGGGCGTCGGGCGACCTTTGGTCGCCCGGCCTCAGTGAGCGAGCG
AGCGCGCAGAGAGGGAGTGGCCAA
P00356: The 300/2000 bp HA F9 construct (for G551) (SEQ ID NO: 286)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTAAGTATATTAGAGCGAGTC
TTTCTGCACACAGATCACCTTTCCTATCAACCCCACTAGCCTCTGGCAAAATGAAGT
GGGTAACCTTTCTCCTCCTCCTCTTCGTCTCCGGCTCTGCTTTTTCCAGGGGTGTGTTT
CGCCGAGAAGCACGTAAGAGTTTTATGTTTTTTCATCTCTGCTTGTATTTTTCTAGTA
ATGGAAGCCTGGTATTTTAAAATAGTTAAATTTTCCTTTAGTGCTGATTTCTAGATTA
TTATTACTGTTGTTGTTGTTATTATTGTCATTATTTGCATCTGAGAACCTTTTTCTTGA
TCATGAAAACGCCAACAAAATTCTGAATCGGCCAAAGAGGTATAATTCAGGTAAAT
TGGAAGAGTTTGTTCAAGGGAACCTTGAGAGAGAATGTATGGAAGAAAAGTGTAGT
TTTGAAGAAGCACGAGAAGTTTTTGAAAACACTGAAAGAACAACTGAATTTTGGAA
GCAGTATGTTGATGGAGATCAGTGTGAGTCCAATCCATGTTTAAATGGCGGCAGTTG
CAAGGATGACATTAATTCCTATGAATGTTGGTGTCCCTTTGGATTTGAAGGAAAGAA
CTGTGAATTAGATGTAACATGTAACATTAAGAATGGCAGATGCGAGCAGTTTTGTAA
AAATAGTGCTGATAACAAGGTGGTTTGCTCCTGTACTGAGGGATATCGACTTGCAGA
AAACCAGAAGTCCTGTGAACCAGCAGTGCCATTTCCATGTGGAAGAGTTTCTGTTTC
ACAAACTTCTAAGCTCACCCGTGCTGAGACTGTTTTTCCTGATGTGGACTATGTAAA
TTCTACTGAAGCTGAAACCATTTTGGATAACATCACTCAAAGCACCCAATCATTTAA
TGACTTCACTCGGGTTGTTGGTGGAGAAGATGCCAAACCAGGTCAATTCCCTTGGCA
GGTTGTTTTGAATGGTAAAGTTGATGCATTCTGTGGAGGCTCTATCGTTAATGAAAA
ATGGATTGTAACTGCTGCCCACTGTGTTGAAACTGGTGTTAAAATTACAGTTGTCGC
AGGTGAACATAATATTGAGGAGACAGAACATACAGAGCAAAAGCGAAATGTGATTC
GAATTATTCCTCACCACAACTACAATGCAGCTATTAATAAGTACAACCATGACATTG
CCCTTCTGGAACTGGACGAACCCTTAGTGCTAAACAGCTACGTTACACCTATTTGCA
TTGCTGACAAGGAATACACGAACATCTTCCTCAAATTTGGATCTGGCTATGTAAGTG
GCTGGGGAAGAGTCTTCCACAAAGGGAGATCAGCTTTAGTTCTTCAGTACCTTAGAG
TTCCACTTGTTGACCGAGCCACATGTCTTCTATCTACAAAGTTCACCATCTATAACAA
CATGTTCTGTGCTGGCTTCCATGAAGGAGGTAGAGATTCATGTCAAGGAGATAGTGG
GGGACCCCATGTTACTGAAGTGGAAGGGACCAGTTTCTTAACTGGAATTATTAGCTG
GGGTGAAGAGTGTGCAATGAAAGGCAAATATGGAATATATACCAAGGTATCCCGGT
ATGTCAACTGGATTAAGGAAAAAACAAAGCTCACTTAACCTCGACTGTGCCTTCTAG
TTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAA
GAACCAGCTGGGGCTCTAGGGGGTATCCCCCTTAGGTGGTTATATTATTGATATATT
TTTGGTATCTTTGATGACAATAATGGGGGATTTTGAAAGCTTAGCTTTAAATTTCTTT
TAATTAAAAAAAAATGCTAGGCAGAATGACTCAAATTACGTTGGATACAGTTGAAT
TTATTACGGTCTCATAGGGCCTGCCTGCTCGACCATGCTATACTAAAAATTAAAAGT
GTGTGTTACTAATTTTATAAATGGAGTTTCCATTTATATTTACCTTTATTTCTTATTTA
CCATTGTCTTAGTAGATATTTACAAACATGACAGAAACACTAAATCTTGAGTTTGAA
TGCACAGATATAAACACTTAACGGGTTTTAAAAATAATAATGTTGGTGAAAAAATAT
AACTTTGAGTGTAGCAGAGAGGAACCATTGCCACCTTCAGATTTTCCTGTAACGATC
GGGAACTGGCATCTTCAGGGAGTAGCTTAGGTCAGTGAAGAGAAGAACAAAAAGCA
GCATATTACAGTTAGTTGTCTTCATCAATCTTTAAATATGTTGTGTGGTTTTTCTCTCC
CTGTTTCCACAGACAAGAGTGAGATCGCCCATCGGTATAATGATTTGGGAGAACAA
CATTTCAAAGGCCTGTAAGTTATAATGCTGAAAGCCCACTTAATATTTCTGGTAGTA
TTAGTTAAAGTTTTAAAACACCTTTTTCCACCTTGAGTGTGAGAATTGTAGAGCAGT
GCTGTCCAGTAGAAATGTGTGCATTGACAGAAAGACTGTGGATCTGTGCTGAGCAAT
GTGGCAGCCAGAGATCACAAGGCTATCAAGCACTTTGCACATGGCAAGTGTAACTG
AGAAGCACACATTCAAATAATAGTTAATTTTAATTGAATGTATCTAGCCATGTGTGG
CTAGTAGCTCCTTTCCTGGAGAGAGAATCTGGAGCCCACATCTAACTTGTTAAGTCT
GGAATCTTATTTTTTATTTCTGGAAAGGTCTATGAACTATAGTTTTGGGGGCAGCTCA
CTTACTAACTTTTAATGCAATAAGATCCATGGTATCTTGAGAACATTATTTTGTCTCT
TTGTAGTACTGAAACCTTATACATGTGAAGTAAGGGGTCTATACTTAAGTCACATCT
CCAACCTTAGTAATGTTTTAATGTAGTAAAAAAATGAGTAATTAATTTATTTTTAGA
AGGTCAATAGTATCATGTATTCCAAATAACAGAGGTATATGGTTAGAAAAGAAACA
ATTCAAAGGACTTATATAATATCTAGCCTTGACAATGAATAAATTTAGAGAGTAGTT
TGCCTGTTTGCCTCATGTTCATAAATCTATTGACACATATGTGCATCTGCACTTCAGC
ATGGTAGAAGTCCATATTCCTTTGCTTGGAAAGGCAGGTGTTCCCATTACGCCTCAG
AGAATAGCTGACGGGAAGAGGCTTTCTAGATAGTTGTATGAAAGATATACAAAATC
TCGCAGGTATACACAGGCATGATTTGCTGGTTGGGAGAGCCACTTGCCTCATACTGA
GGTTTTTGTGTCTGCTTTTCAGAGTCCTGATTGCCTTTTCCCAGTATCTCCAGAAATG
CTCATACGATGAGCATGCCAAATTAGTGCAGGAAGTAACAGACTTTGCAAAGACGT
GTGTTGCCGATGAGTCTGCCGCCAACTGTGACAAATCCCTTGTGAGTACCTTCTGAT
TTTGTGGATCTACTTTCCTGCTTTCTGGAACTCTGTTTCAAAGCCAATCATGACTCCA
TCACTTAAGGCCCCGGGAACACTGTGGCAGAGGGCAGCAGAGAGATTGATAAAGCC
AGGGTGATGGGAATTTTCTGTGGGACTCCATTTCATAGTAATTGCAGAAGCTACAAT
ACACTCAAAAAGTCTCACCACATGACTGCCCAAATGGGAGCTTGACAGTGACAGTG
ACAGTAGATATGCCAAAGTGGATGAGGGAAAGACCACAAGAGCTAAACCCTGTAAA
AAGAACTGTAGGCAACTAAGGAATGCAGAGAGAAAGATCTAGGAACCCCTAGTGAT
GGAGTTGGCCACTCCCTCTCTGCGCGCTCGCTCGCTCACTGAGGCCGCCCGGGCAAA
GCCCGGGCGTCGGGCGACCTTTGGTCGCCCGGCCTCAGTGAGCGAGCGAGCGCGCA
GAGAGGGAGTGGCCAA
P00362: The 300/1500 bp HA F9 construct (for G551) (SEQ ID NO: 287)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTAAGTATATTAGAGCGAGTC
TTTCTGCACACAGATCACCTTTCCTATCAACCCCACTAGCCTCTGGCAAAATGAAGT
GGGTAACCTTTCTCCTCCTCCTCTTCGTCTCCGGCTCTGCTTTTTCCAGGGGTGTGTTT
CGCCGAGAAGCACGTAAGAGTTTTATGTTTTTTCATCTCTGCTTGTATTTTTCTAGTA
ATGGAAGCCTGGTATTTTAAAATAGTTAAATTTTCCTTTAGTGCTGATTTCTAGATTA
TTATTACTGTTGTTGTTGTTATTATTGTCATTATTTGCATCTGAGAACCTTTTTCTTGA
TCATGAAAACGCCAACAAAATTCTGAATCGGCCAAAGAGGTATAATTCAGGTAAAT
TGGAAGAGTTTGTTCAAGGGAACCTTGAGAGAGAATGTATGGAAGAAAAGTGTAGT
TTTGAAGAAGCACGAGAAGTTTTTGAAAACACTGAAAGAACAACTGAATTTTGGAA
GCAGTATGTTGATGGAGATCAGTGTGAGTCCAATCCATGTTTAAATGGCGGCAGTTG
CAAGGATGACATTAATTCCTATGAATGTTGGTGTCCCTTTGGATTTGAAGGAAAGAA
CTGTGAATTAGATGTAACATGTAACATTAAGAATGGCAGATGCGAGCAGTTTTGTAA
AAATAGTGCTGATAACAAGGTGGTTTGCTCCTGTACTGAGGGATATCGACTTGCAGA
AAACCAGAAGTCCTGTGAACCAGCAGTGCCATTTCCATGTGGAAGAGTTTCTGTTTC
ACAAACTTCTAAGCTCACCCGTGCTGAGACTGTTTTTCCTGATGTGGACTATGTAAA
TTCTACTGAAGCTGAAACCATTTTGGATAACATCACTCAAAGCACCCAATCATTTAA
TGACTTCACTCGGGTTGTTGGTGGAGAAGATGCCAAACCAGGTCAATTCCCTTGGCA
GGTTGTTTTGAATGGTAAAGTTGATGCATTCTGTGGAGGCTCTATCGTTAATGAAAA
ATGGATTGTAACTGCTGCCCACTGTGTTGAAACTGGTGTTAAAATTACAGTTGTCGC
AGGTGAACATAATATTGAGGAGACAGAACATACAGAGCAAAAGCGAAATGTGATTC
GAATTATTCCTCACCACAACTACAATGCAGCTATTAATAAGTACAACCATGACATTG
CCCTTCTGGAACTGGACGAACCCTTAGTGCTAAACAGCTACGTTACACCTATTTGCA
TTGCTGACAAGGAATACACGAACATCTTCCTCAAATTTGGATCTGGCTATGTAAGTG
GCTGGGGAAGAGTCTTCCACAAAGGGAGATCAGCTTTAGTTCTTCAGTACCTTAGAG
TTCCACTTGTTGACCGAGCCACATGTCTTCTATCTACAAAGTTCACCATCTATAACAA
CATGTTCTGTGCTGGCTTCCATGAAGGAGGTAGAGATTCATGTCAAGGAGATAGTGG
GGGACCCCATGTTACTGAAGTGGAAGGGACCAGTTTCTTAACTGGAATTATTAGCTG
GGGTGAAGAGTGTGCAATGAAAGGCAAATATGGAATATATACCAAGGTATCCCGGT
ATGTCAACTGGATTAAGGAAAAAACAAAGCTCACTTAACCTCGACTGTGCCTTCTAG
TTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAA
GAACCAGCTGGGGCTCTAGGGGGTATCCCCCTTAGGTGGTTATATTATTGATATATT
TTTGGTATCTTTGATGACAATAATGGGGGATTTTGAAAGCTTAGCTTTAAATTTCTTT
TAATTAAAAAAAAATGCTAGGCAGAATGACTCAAATTACGTTGGATACAGTTGAAT
TTATTACGGTCTCATAGGGCCTGCCTGCTCGACCATGCTATACTAAAAATTAAAAGT
GTGTGTTACTAATTTTATAAATGGAGTTTCCATTTATATTTACCTTTATTTCTTATTTA
CCATTGTCTTAGTAGATATTTACAAACATGACAGAAACACTAAATCTTGAGTTTGAA
TGCACAGATATAAACACTTAACGGGTTTTAAAAATAATAATGTTGGTGAAAAAATAT
AACTTTGAGTGTAGCAGAGAGGAACCATTGCCACCTTCAGATTTTCCTGTAACGATC
GGGAACTGGCATCTTCAGGGAGTAGCTTAGGTCAGTGAAGAGAAGAACAAAAAGCA
GCATATTACAGTTAGTTGTCTTCATCAATCTTTAAATATGTTGTGTGGTTTTTCTCTCC
CTGTTTCCACAGACAAGAGTGAGATCGCCCATCGGTATAATGATTTGGGAGAACAA
CATTTCAAAGGCCTGTAAGTTATAATGCTGAAAGCCCACTTAATATTTCTGGTAGTA
TTAGTTAAAGTTTTAAAACACCTTTTTCCACCTTGAGTGTGAGAATTGTAGAGCAGT
GCTGTCCAGTAGAAATGTGTGCATTGACAGAAAGACTGTGGATCTGTGCTGAGCAAT
GTGGCAGCCAGAGATCACAAGGCTATCAAGCACTTTGCACATGGCAAGTGTAACTG
AGAAGCACACATTCAAATAATAGTTAATTTTAATTGAATGTATCTAGCCATGTGTGG
CTAGTAGCTCCTTTCCTGGAGAGAGAATCTGGAGCCCACATCTAACTTGTTAAGTCT
GGAATCTTATTTTTTATTTCTGGAAAGGTCTATGAACTATAGTTTTGGGGGCAGCTCA
CTTACTAACTTTTAATGCAATAAGATCCATGGTATCTTGAGAACATTATTTTGTCTCT
TTGTAGTACTGAAACCTTATACATGTGAAGTAAGGGGTCTATACTTAAGTCACATCT
CCAACCTTAGTAATGTTTTAATGTAGTAAAAAAATGAGTAATTAATTTATTTTTAGA
AGGTCAATAGTATCATGTATTCCAAATAACAGAGGTATATGGTTAGAAAAGAAACA
ATTCAAAGGACTTATATAATATCTAGCCTTGACAATGAATAAATTTAGAGAGTAGTT
TGCCTGTTTGCCTCATGTTCATAAATCTATTGACACATATGTGCATCTGCACTTCAGC
ATGGTAGAAGTCCATATTCCTTTGCTTGGAAAGGCAGGTGTTCCCATTACGCCTCAG
AGAATAGCTGACGGGAAGAGGCTTTCTAGATAGTTGTATGAAAGATATACAAAATC
TCGCAGGTATACACAGGCATGATTTGCTGGTTGGGAGAGCCACTTAGATCTAGGAAC
CCCTAGTGATGGAGTTGGCCACTCCCTCTCTGCGCGCTCGCTCGCTCACTGAGGCCG
CCCGGGCAAAGCCCGGGCGTCGGGCGACCTTTGGTCGCCCGGCCTCAGTGAGCGAG
CGAGCGCGCAGAGAGGGAGTGGCCAA
Cas9 ORF (SEQ ID NO: 703)
ATGGATAAGAAGTACTCAATCGGGCTGGATATCGGAACTAATTCCGTGGGTTGGGC
AGTGATCACGGATGAATACAAAGTGCCGTCCAAGAAGTTCAAGGTCCTGGGGAACA
CCGATAGACACAGCATCAAGAAAAATCTCATCGGAGCCCTGCTGTTTGACTCCGGC
GAAACCGCAGAAGCGACCCGGCTCAAACGTACCGCGAGGCGACGCTACACCCGGCG
GAAGAATCGCATCTGCTATCTGCAAGAGATCTTTTCGAACGAAATGGCAAAGGTCG
ACGACAGCTTCTTCCACCGCCTGGAAGAATCTTTCCTGGTGGAGGAGGACAAGAAG
CATGAACGGCATCCTATCTTTGGAAACATCGTCGACGAAGTGGCGTACCACGAAAA
GTACCCGACCATCTACCATCTGCGGAAGAAGTTGGTTGACTCAACTGACAAGGCCG
ACCTCAGATTGATCTACTTGGCCCTCGCCCATATGATCAAATTCCGCGGACACTTCC
TGATCGAAGGCGATCTGAACCCTGATAACTCCGACGTGGATAAGCTTTTCATTCAAC
TGGTGCAGACCTACAACCAACTGTTCGAAGAAAACCCAATCAATGCTAGCGGCGTC
GATGCCAAGGCCATCCTGTCCGCCCGGCTGTCGAAGTCGCGGCGCCTCGAAAACCT
GATCGCACAGCTGCCGGGAGAGAAAAAGAACGGACTTTTCGGCAACTTGATCGCTC
TCTCACTGGGACTCACTCCCAATTTCAAGTCCAATTTTGACCTGGCCGAGGACGCGA
AGCTGCAACTCTCAAAGGACACCTACGACGACGACTTGGACAATTTGCTGGCACAA
ATTGGCGATCAGTACGCGGATCTGTTCCTTGCCGCTAAGAACCTTTCGGACGCAATC
TTGCTGTCCGATATCCTGCGCGTGAACACCGAAATAACCAAAGCGCCGCTTAGCGCC
TCGATGATTAAGCGGTACGACGAGCATCACCAGGATCTCACGCTGCTCAAAGCGCT
CGTGAGACAGCAACTGCCTGAAAAGTACAAGGAGATCTTCTTCGACCAGTCCAAGA
ATGGGTACGCAGGGTACATCGATGGAGGCGCTAGCCAGGAAGAGTTCTATAAGTTC
ATCAAGCCAATCCTGGAAAAGATGGACGGAACCGAAGAACTGCTGGTCAAGCTGAA
CAGGGAGGATCTGCTCCGGAAACAGAGAACCTTTGACAACGGATCCATTCCCCACC
AGATCCATCTGGGTGAGCTGCACGCCATCTTGCGGCGCCAGGAGGACTTTTACCCAT
TCCTCAAGGACAACCGGGAAAAGATCGAGAAAATTCTGACGTTCCGCATCCCGTATT
ACGTGGGCCCACTGGCGCGCGGCAATTCGCGCTTCGCGTGGATGACTAGAAAATCA
GAGGAAACCATCACTCCTTGGAATTTCGAGGAAGTTGTGGATAAGGGAGCTTCGGC
ACAAAGCTTCATCGAACGAATGACCAACTTCGACAAGAATCTCCCAAACGAGAAGG
TGCTTCCTAAGCACAGCCTCCTTTACGAATACTTCACTGTCTACAACGAACTGACTA
AAGTGAAATACGTTACTGAAGGAATGAGGAAGCCGGCCTTTCTGTCCGGAGAACAG
AAGAAAGCAATTGTCGATCTGCTGTTCAAGACCAACCGCAAGGTGACCGTCAAGCA
GCTTAAAGAGGACTACTTCAAGAAGATCGAGTGTTTCGACTCAGTGGAAATCAGCG
GGGTGGAGGACAGATTCAACGCTTCGCTGGGAACCTATCATGATCTCCTGAAGATCA
TCAAGGACAAGGACTTCCTTGACAACGAGGAGAACGAGGACATCCTGGAAGATATC
GTCCTGACCTTGACCCTTTTCGAGGATCGCGAGATGATCGAGGAGAGGCTTAAGACC
TACGCTCATCTCTTCGACGATAAGGTCATGAAACAACTCAAGCGCCGCCGGTACACT
GGTTGGGGCCGCCTCTCCCGCAAGCTGATCAACGGTATTCGCGATAAACAGAGCGG
TAAAACTATCCTGGATTTCCTCAAATCGGATGGCTTCGCTAATCGTAACTTCATGCA
ATTGATCCACGACGACAGCCTGACCTTTAAGGAGGACATCCAAAAAGCACAAGTGT
CCGGACAGGGAGACTCACTCCATGAACACATCGCGAATCTGGCCGGTTCGCCGGCG
ATTAAGAAGGGAATTCTGCAAACTGTGAAGGTGGTCGACGAGCTGGTGAAGGTCAT
GGGACGGCACAAACCGGAGAATATCGTGATTGAAATGGCCCGAGAAAACCAGACTA
CCCAGAAGGGCCAGAAAAACTCCCGCGAAAGGATGAAGCGGATCGAAGAAGGAAT
CAAGGAGCTGGGCAGCCAGATCCTGAAAGAGCACCCGGTGGAAAACACGCAGCTG
CAGAACGAGAAGCTCTACCTGTACTATTTGCAAAATGGACGGGACATGTACGTGGA
CCAAGAGCTGGACATCAATCGGTTGTCTGATTACGACGTGGACCACATCGTTCCACA
GTCCTTTCTGAAGGATGACTCGATCGATAACAAGGTGTTGACTCGCAGCGACAAGA
ACAGAGGGAAGTCAGATAATGTGCCATCGGAGGAGGTCGTGAAGAAGATGAAGAA
TTACTGGCGGCAGCTCCTGAATGCGAAGCTGATTACCCAGAGAAAGTTTGACAATCT
CACTAAAGCCGAGCGCGGCGGACTCTCAGAGCTGGATAAGGCTGGATTCATCAAAC
GGCAGCTGGTCGAGACTCGGCAGATTACCAAGCACGTGGCGCAGATCTTGGACTCC
CGCATGAACACTAAATACGACGAGAACGATAAGCTCATCCGGGAAGTGAAGGTGAT
TACCCTGAAAAGCAAACTTGTGTCGGACTTTCGGAAGGACTTTCAGTTTTACAAAGT
GAGAGAAATCAACAACTACCATCACGCGCATGACGCATACCTCAACGCTGTGGTCG
GTACCGCCCTGATCAAAAAGTACCCTAAACTTGAATCGGAGTTTGTGTACGGAGACT
ACAAGGTCTACGACGTGAGGAAGATGATAGCCAAGTCCGAACAGGAAATCGGGAA
AGCAACTGCGAAATACTTCTTTTACTCAAACATCATGAACTTTTTCAAGACTGAAAT
TACGCTGGCCAATGGAGAAATCAGGAAGAGGCCACTGATCGAAACTAACGGAGAA
ACGGGCGAAATCGTGTGGGACAAGGGCAGGGACTTCGCAACTGTTCGCAAAGTGCT
CTCTATGCCGCAAGTCAATATTGTGAAGAAAACCGAAGTGCAAACCGGCGGATTTTC
AAAGGAATCGATCCTCCCAAAGAGAAATAGCGACAAGCTCATTGCACGCAAGAAAG
ACTGGGACCCGAAGAAGTACGGAGGATTCGATTCGCCGACTGTCGCATACTCCGTC
CTCGTGGTGGCCAAGGTGGAGAAGGGAAAGAGCAAAAAGCTCAAATCCGTCAAAG
AGCTGCTGGGGATTACCATCATGGAACGATCCTCGTTCGAGAAGAACCCGATTGATT
TCCTCGAGGCGAAGGGTTACAAGGAGGTGAAGAAGGATCTGATCATCAAACTCCCC
AAGTACTCACTGTTCGAACTGGAAAATGGTCGGAAGCGCATGCTGGCTTCGGCCGG
AGAACTCCAAAAAGGAAATGAGCTGGCCTTGCCTAGCAAGTACGTCAACTTCCTCTA
TCTTGCTTCGCACTACGAAAAACTCAAAGGGTCACCGGAAGATAACGAACAGAAGC
AGCTTTTCGTGGAGCAGCACAAGCATTATCTGGATGAAATCATCGAACAAATCTCCG
AGTTTTCAAAGCGCGTGATCCTCGCCGACGCCAACCTCGACAAAGTCCTGTCGGCCT
ACAATAAGCATAGAGATAAGCCGATCAGAGAACAGGCCGAGAACATTATCCACTTG
TTCACCCTGACTAACCTGGGAGCCCCAGCCGCCTTCAAGTACTTCGATACTACTATC
GATCGCAAAAGATACACGTCCACCAAGGAAGTTCTGGACGCGACCCTGATCCACCA
AAGCATCACTGGACTCTACGAAACTAGGATCGATCTGTCGCAGCTGGGTGGCGAT
U-dep Cas9 ORF (SEQ ID NO: 704)
ATGGACAAGAAGTACAGCATCGGACTGGACATCGGAACAAACAGCGTCGGATGGGC
AGTCATCACAGACGAATACAAGGTCCCGAGCAAGAAGTTCAAGGTCCTGGGAAACA
CAGACAGACACAGCATCAAGAAGAACCTGATCGGAGCACTGCTGTTCGACAGCGGA
GAAACAGCAGAAGCAACAAGACTGAAGAGAACAGCAAGAAGAAGATACACAAGAA
GAAAGAACAGAATCTGCTACCTGCAGGAAATCTTCAGCAACGAAATGGCAAAGGTC
GACGACAGCTTCTTCCACAGACTGGAAGAAAGCTTCCTGGTCGAAGAAGACAAGAA
GCACGAAAGACACCCGATCTTCGGAAACATCGTCGACGAAGTCGCATACCACGAAA
AGTACCCGACAATCTACCACCTGAGAAAGAAGCTGGTCGACAGCACAGACAAGGCA
GACCTGAGACTGATCTACCTGGCACTGGCACACATGATCAAGTTCAGAGGACACTTC
CTGATCGAAGGAGACCTGAACCCGGACAACAGCGACGTCGACAAGCTGTTCATCCA
GCTGGTCCAGACATACAACCAGCTGTTCGAAGAAAACCCGATCAACGCAAGCGGAG
TCGACGCAAAGGCAATCCTGAGCGCAAGACTGAGCAAGAGCAGAAGACTGGAAAA
CCTGATCGCACAGCTGCCGGGAGAAAAGAAGAACGGACTGTTCGGAAACCTGATCG
CACTGAGCCTGGGACTGACACCGAACTTCAAGAGCAACTTCGACCTGGCAGAAGAC
GCAAAGCTGCAGCTGAGCAAGGACACATACGACGACGACCTGGACAACCTGCTGGC
ACAGATCGGAGACCAGTACGCAGACCTGTTCCTGGCAGCAAAGAACCTGAGCGACG
CAATCCTGCTGAGCGACATCCTGAGAGTCAACACAGAAATCACAAAGGCACCGCTG
AGCGCAAGCATGATCAAGAGATACGACGAACACCACCAGGACCTGACACTGCTGAA
GGCACTGGTCAGACAGCAGCTGCCGGAAAAGTACAAGGAAATCTTCTTCGACCAGA
GCAAGAACGGATACGCAGGATACATCGACGGAGGAGCAAGCCAGGAAGAATTCTA
CAAGTTCATCAAGCCGATCCTGGAAAAGATGGACGGAACAGAAGAACTGCTGGTCA
AGCTGAACAGAGAAGACCTGCTGAGAAAGCAGAGAACATTCGACAACGGAAGCAT
CCCGCACCAGATCCACCTGGGAGAACTGCACGCAATCCTGAGAAGACAGGAAGACT
TCTACCCGTTCCTGAAGGACAACAGAGAAAAGATCGAAAAGATCCTGACATTCAGA
ATCCCGTACTACGTCGGACCGCTGGCAAGAGGAAACAGCAGATTCGCATGGATGAC
AAGAAAGAGCGAAGAAACAATCACACCGTGGAACTTCGAAGAAGTCGTCGACAAG
GGAGCAAGCGCACAGAGCTTCATCGAAAGAATGACAAACTTCGACAAGAACCTGCC
GAACGAAAAGGTCCTGCCGAAGCACAGCCTGCTGTACGAATACTTCACAGTCTACA
ACGAACTGACAAAGGTCAAGTACGTCACAGAAGGAATGAGAAAGCCGGCATTCCTG
AGCGGAGAACAGAAGAAGGCAATCGTCGACCTGCTGTTCAAGACAAACAGAAAGG
TCACAGTCAAGCAGCTGAAGGAAGACTACTTCAAGAAGATCGAATGCTTCGACAGC
GTCGAAATCAGCGGAGTCGAAGACAGATTCAACGCAAGCCTGGGAACATACCACGA
CCTGCTGAAGATCATCAAGGACAAGGACTTCCTGGACAACGAAGAAAACGAAGACA
TCCTGGAAGACATCGTCCTGACACTGACACTGTTCGAAGACAGAGAAATGATCGAA
GAAAGACTGAAGACATACGCACACCTGTTCGACGACAAGGTCATGAAGCAGCTGAA
GAGAAGAAGATACACAGGATGGGGAAGACTGAGCAGAAAGCTGATCAACGGAATC
AGAGACAAGCAGAGCGGAAAGACAATCCTGGACTTCCTGAAGAGCGACGGATTCGC
AAACAGAAACTTCATGCAGCTGATCCACGACGACAGCCTGACATTCAAGGAAGACA
TCCAGAAGGCACAGGTCAGCGGACAGGGAGACAGCCTGCACGAACACATCGCAAA
CCTGGCAGGAAGCCCGGCAATCAAGAAGGGAATCCTGCAGACAGTCAAGGTCGTCG
ACGAACTGGTCAAGGTCATGGGAAGACACAAGCCGGAAAACATCGTCATCGAAATG
GCAAGAGAAAACCAGACAACACAGAAGGGACAGAAGAACAGCAGAGAAAGAATG
AAGAGAATCGAAGAAGGAATCAAGGAACTGGGAAGCCAGATCCTGAAGGAACACC
CGGTCGAAAACACACAGCTGCAGAACGAAAAGCTGTACCTGTACTACCTGCAGAAC
GGAAGAGACATGTACGTCGACCAGGAACTGGACATCAACAGACTGAGCGACTACGA
CGTCGACCACATCGTCCCGCAGAGCTTCCTGAAGGACGACAGCATCGACAACAAGG
TCCTGACAAGAAGCGACAAGAACAGAGGAAAGAGCGACAACGTCCCGAGCGAAGA
AGTCGTCAAGAAGATGAAGAACTACTGGAGACAGCTGCTGAACGCAAAGCTGATCA
CACAGAGAAAGTTCGACAACCTGACAAAGGCAGAGAGAGGAGGACTGAGCGAACT
GGACAAGGCAGGATTCATCAAGAGACAGCTGGTCGAAACAAGACAGATCACAAAG
CACGTCGCACAGATCCTGGACAGCAGAATGAACACAAAGTACGACGAAAACGACA
AGCTGATCAGAGAAGTCAAGGTCATCACACTGAAGAGCAAGCTGGTCAGCGACTTC
AGAAAGGACTTCCAGTTCTACAAGGTCAGAGAAATCAACAACTACCACCACGCACA
CGACGCATACCTGAACGCAGTCGTCGGAACAGCACTGATCAAGAAGTACCCGAAGC
TGGAAAGCGAATTCGTCTACGGAGACTACAAGGTCTACGACGTCAGAAAGATGATC
GCAAAGAGCGAACAGGAAATCGGAAAGGCAACAGCAAAGTACTTCTTCTACAGCAA
CATCATGAACTTCTTCAAGACAGAAATCACACTGGCAAACGGAGAAATCAGAAAGA
GACCGCTGATCGAAACAAACGGAGAAACAGGAGAAATCGTCTGGGACAAGGGAAG
AGACTTCGCAACAGTCAGAAAGGTCCTGAGCATGCCGCAGGTCAACATCGTCAAGA
AGACAGAAGTCCAGACAGGAGGATTCAGCAAGGAAAGCATCCTGCCGAAGAGAAA
CAGCGACAAGCTGATCGCAAGAAAGAAGGACTGGGACCCGAAGAAGTACGGAGGA
TTCGACAGCCCGACAGTCGCATACAGCGTCCTGGTCGTCGCAAAGGTCGAAAAGGG
AAAGAGCAAGAAGCTGAAGAGCGTCAAGGAACTGCTGGGAATCACAATCATGGAA
AGAAGCAGCTTCGAAAAGAACCCGATCGACTTCCTGGAAGCAAAGGGATACAAGGA
AGTCAAGAAGGACCTGATCATCAAGCTGCCGAAGTACAGCCTGTTCGAACTGGAAA
ACGGAAGAAAGAGAATGCTGGCAAGCGCAGGAGAACTGCAGAAGGGAAACGAACT
GGCACTGCCGAGCAAGTACGTCAACTTCCTGTACCTGGCAAGCCACTACGAAAAGC
TGAAGGGAAGCCCGGAAGACAACGAACAGAAGCAGCTGTTCGTCGAACAGCACAA
GCACTACCTGGACGAAATCATCGAACAGATCAGCGAATTCAGCAAGAGAGTCATCC
TGGCAGACGCAAACCTGGACAAGGTCCTGAGCGCATACAACAAGCACAGAGACAA
GCCGATCAGAGAACAGGCAGAAAACATCATCCACCTGTTCACACTGACAAACCTGG
GAGCACCGGCAGCATTCAAGTACTTCGACACAACAATCGACAGAAAGAGATACACA
AGCACAAAGGAAGTCCTGGACGCAACACTGATCCACCAGAGCATCACAGGACTGTA
CGAAACAAGAATCGACCTGAGCCAGCTGGGAGGAGACGGAGGAGGAAGCCCGAAG
AAGAAGAGAAAGGTCTAG
mRNA comprising U dep Cas9 (SEQ ID NO: 705)
GGGUCCCGCAGUCGGCGUCCAGCGGCUCUGCUUGUUCGUGUGUGUGUCGUUGCAG
GCCUUAUUCGGAUCCGCCACCAUGGACAAGAAGUACAGCAUCGGACUGGACAUCG
GAACAAACAGCGUCGGAUGGGCAGUCAUCACAGACGAAUACAAGGUCCCGAGCAA
GAAGUUCAAGGUCCUGGGAAACACAGACAGACACAGCAUCAAGAAGAACCUGAU
CGGAGCACUGCUGUUCGACAGCGGAGAAACAGCAGAAGCAACAAGACUGAAGAG
AACAGCAAGAAGAAGAUACACAAGAAGAAAGAACAGAAUCUGCUACCUGCAGGA
AAUCUUCAGCAACGAAAUGGCAAAGGUCGACGACAGCUUCUUCCACAGACUGGAA
GAAAGCUUCCUGGUCGAAGAAGACAAGAAGCACGAAAGACACCCGAUCUUCGGA
AACAUCGUCGACGAAGUCGCAUACCACGAAAAGUACCCGACAAUCUACCACCUGA
GAAAGAAGCUGGUCGACAGCACAGACAAGGCAGACCUGAGACUGAUCUACCUGGC
ACUGGCACACAUGAUCAAGUUCAGAGGACACUUCCUGAUCGAAGGAGACCUGAAC
CCGGACAACAGCGACGUCGACAAGCUGUUCAUCCAGCUGGUCCAGACAUACAACC
AGCUGUUCGAAGAAAACCCGAUCAACGCAAGCGGAGUCGACGCAAAGGCAAUCCU
GAGCGCAAGACUGAGCAAGAGCAGAAGACUGGAAAACCUGAUCGCACAGCUGCCG
GGAGAAAAGAAGAACGGACUGUUCGGAAACCUGAUCGCACUGAGCCUGGGACUG
ACACCGAACUUCAAGAGCAACUUCGACCUGGCAGAAGACGCAAAGCUGCAGCUGA
GCAAGGACACAUACGACGACGACCUGGACAACCUGCUGGCACAGAUCGGAGACCA
GUACGCAGACCUGUUCCUGGCAGCAAAGAACCUGAGCGACGCAAUCCUGCUGAGC
GACAUCCUGAGAGUCAACACAGAAAUCACAAAGGCACCGCUGAGCGCAAGCAUGA
UCAAGAGAUACGACGAACACCACCAGGACCUGACACUGCUGAAGGCACUGGUCAG
ACAGCAGCUGCCGGAAAAGUACAAGGAAAUCUUCUUCGACCAGAGCAAGAACGG
AUACGCAGGAUACAUCGACGGAGGAGCAAGCCAGGAAGAAUUCUACAAGUUCAU
CAAGCCGAUCCUGGAAAAGAUGGACGGAACAGAAGAACUGCUGGUCAAGCUGAA
CAGAGAAGACCUGCUGAGAAAGCAGAGAACAUUCGACAACGGAAGCAUCCCGCAC
CAGAUCCACCUGGGAGAACUGCACGCAAUCCUGAGAAGACAGGAAGACUUCUACC
CGUUCCUGAAGGACAACAGAGAAAAGAUCGAAAAGAUCCUGACAUUCAGAAUCC
CGUACUACGUCGGACCGCUGGCAAGAGGAAACAGCAGAUUCGCAUGGAUGACAA
GAAAGAGCGAAGAAACAAUCACACCGUGGAACUUCGAAGAAGUCGUCGACAAGG
GAGCAAGCGCACAGAGCUUCAUCGAAAGAAUGACAAACUUCGACAAGAACCUGCC
GAACGAAAAGGUCCUGCCGAAGCACAGCCUGCUGUACGAAUACUUCACAGUCUAC
AACGAACUGACAAAGGUCAAGUACGUCACAGAAGGAAUGAGAAAGCCGGCAUUC
CUGAGCGGAGAACAGAAGAAGGCAAUCGUCGACCUGCUGUUCAAGACAAACAGA
AAGGUCACAGUCAAGCAGCUGAAGGAAGACUACUUCAAGAAGAUCGAAUGCUUC
GACAGCGUCGAAAUCAGCGGAGUCGAAGACAGAUUCAACGCAAGCCUGGGAACA
UACCACGACCUGCUGAAGAUCAUCAAGGACAAGGACUUCCUGGACAACGAAGAAA
ACGAAGACAUCCUGGAAGACAUCGUCCUGACACUGACACUGUUCGAAGACAGAGA
AAUGAUCGAAGAAAGACUGAAGACAUACGCACACCUGUUCGACGACAAGGUCAU
GAAGCAGCUGAAGAGAAGAAGAUACACAGGAUGGGGAAGACUGAGCAGAAAGCU
GAUCAACGGAAUCAGAGACAAGCAGAGCGGAAAGACAAUCCUGGACUUCCUGAA
GAGCGACGGAUUCGCAAACAGAAACUUCAUGCAGCUGAUCCACGACGACAGCCUG
ACAUUCAAGGAAGACAUCCAGAAGGCACAGGUCAGCGGACAGGGAGACAGCCUGC
ACGAACACAUCGCAAACCUGGCAGGAAGCCCGGCAAUCAAGAAGGGAAUCCUGCA
GACAGUCAAGGUCGUCGACGAACUGGUCAAGGUCAUGGGAAGACACAAGCCGGA
AAACAUCGUCAUCGAAAUGGCAAGAGAAAACCAGACAACACAGAAGGGACAGAA
GAACAGCAGAGAAAGAAUGAAGAGAAUCGAAGAAGGAAUCAAGGAACUGGGAAG
CCAGAUCCUGAAGGAACACCCGGUCGAAAACACACAGCUGCAGAACGAAAAGCUG
UACCUGUACUACCUGCAGAACGGAAGAGACAUGUACGUCGACCAGGAACUGGACA
UCAACAGACUGAGCGACUACGACGUCGACCACAUCGUCCCGCAGAGCUUCCUGAA
GGACGACAGCAUCGACAACAAGGUCCUGACAAGAAGCGACAAGAACAGAGGAAA
GAGCGACAACGUCCCGAGCGAAGAAGUCGUCAAGAAGAUGAAGAACUACUGGAG
ACAGCUGCUGAACGCAAAGCUGAUCACACAGAGAAAGUUCGACAACCUGACAAAG
GCAGAGAGAGGAGGACUGAGCGAACUGGACAAGGCAGGAUUCAUCAAGAGACAG
CUGGUCGAAACAAGACAGAUCACAAAGCACGUCGCACAGAUCCUGGACAGCAGAA
UGAACACAAAGUACGACGAAAACGACAAGCUGAUCAGAGAAGUCAAGGUCAUCA
CACUGAAGAGCAAGCUGGUCAGCGACUUCAGAAAGGACUUCCAGUUCUACAAGG
UCAGAGAAAUCAACAACUACCACCACGCACACGACGCAUACCUGAACGCAGUCGU
CGGAACAGCACUGAUCAAGAAGUACCCGAAGCUGGAAAGCGAAUUCGUCUACGG
AGACUACAAGGUCUACGACGUCAGAAAGAUGAUCGCAAAGAGCGAACAGGAAAU
CGGAAAGGCAACAGCAAAGUACUUCUUCUACAGCAACAUCAUGAACUUCUUCAAG
ACAGAAAUCACACUGGCAAACGGAGAAAUCAGAAAGAGACCGCUGAUCGAAACA
AACGGAGAAACAGGAGAAAUCGUCUGGGACAAGGGAAGAGACUUCGCAACAGUC
AGAAAGGUCCUGAGCAUGCCGCAGGUCAACAUCGUCAAGAAGACAGAAGUCCAG
ACAGGAGGAUUCAGCAAGGAAAGCAUCCUGCCGAAGAGAAACAGCGACAAGCUG
AUCGCAAGAAAGAAGGACUGGGACCCGAAGAAGUACGGAGGAUUCGACAGCCCG
ACAGUCGCAUACAGCGUCCUGGUCGUCGCAAAGGUCGAAAAGGGAAAGAGCAAG
AAGCUGAAGAGCGUCAAGGAACUGCUGGGAAUCACAAUCAUGGAAAGAAGCAGC
UUCGAAAAGAACCCGAUCGACUUCCUGGAAGCAAAGGGAUACAAGGAAGUCAAG
AAGGACCUGAUCAUCAAGCUGCCGAAGUACAGCCUGUUCGAACUGGAAAACGGA
AGAAAGAGAAUGCUGGCAAGCGCAGGAGAACUGCAGAAGGGAAACGAACUGGCA
CUGCCGAGCAAGUACGUCAACUUCCUGUACCUGGCAAGCCACUACGAAAAGCUGA
AGGGAAGCCCGGAAGACAACGAACAGAAGCAGCUGUUCGUCGAACAGCACAAGCA
CUACCUGGACGAAAUCAUCGAACAGAUCAGCGAAUUCAGCAAGAGAGUCAUCCUG
GCAGACGCAAACCUGGACAAGGUCCUGAGCGCAUACAACAAGCACAGAGACAAGC
CGAUCAGAGAACAGGCAGAAAACAUCAUCCACCUGUUCACACUGACAAACCUGGG
AGCACCGGCAGCAUUCAAGUACUUCGACACAACAAUCGACAGAAAGAGAUACACA
AGCACAAAGGAAGUCCUGGACGCAACACUGAUCCACCAGAGCAUCACAGGACUGU
ACGAAACAAGAAUCGACCUGAGCCAGCUGGGAGGAGACGGAGGAGGAAGCCCGA
AGAAGAAGAGAAAGGUCUAGCUAGCCAUCACAUUUAAAAGCAUCUCAGCCUACC
AUGAGAAUAAGAGAAAGAAAAUGAAGAUCAAUAGCUUAUUCAUCUCUUUUUCUU
UUUCGUUGGUGUAAAGCCAACACCCUGUCUAAAAAACAUAAAUUUCUUUAAUCA
UUUUGCCUCUUUUCUCUGUGCUUCAAUUAAUAAAAAAUGGAAAGAACCUCGAGA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAA

1. A method of inserting a nucleic acid encoding a heterologous polypeptide into an albumin locus of a liver cell or liver cell population in a subject, the method comprising administering to the subject:
i) a single guide (sgRNA) that comprises the sequence of SEQ ID NO: 40, wherein the sgRNA comprises one or more modified nucleosides;
ii) a Cas9 nuclease; and
iii) a construct comprising the nucleic acid encoding the heterologous polypeptide,
thereby inserting the nucleic acid encoding the heterologous polypeptide into the albumin locus of the liver cell or liver cell population in the subject.

2. (canceled)

3. The method of claim 1, wherein the sgRNA that comprises the sequence of SEQ ID NO: 72.

4-11. (canceled)

12. The method of claim 1, wherein the Cas9 nuclease is administered as a Cas9 nuclease enzyme or a nucleic acid encoding a Cas9 nuclease.

13. The method of claim 12, wherein the Cas9 nuclease is administered as a nucleic acid encoding the the Cas9 nuclease.

14. The method of claim 13, wherein the nucleic acid encoding the Cas9 nuclease is an mRNA.

15. The method of claim 14, wherein the mRNA is a modified mRNA.

16-19. (canceled)

20. The method of claim 1, wherein the Cas9 nuclease is an S. pyogenes Cas9 nuclease.

21-27. (canceled)

28. The method of claim 1, wherein the construct comprises a polyadenylation signal sequence.

29. The method of claim 1, wherein the construct comprises a splice acceptor site.

30. The method of claim 1, wherein the construct does not comprise a homology arm.

31. The method of claim 1, wherein the sgRNA is administered in a lipid nanoparticle.

32. The method of claim 1, wherein the Cas9 nuclease is administered in a lipid nanoparticle.

33. The method of claim 1, wherein the construct comprising the nucleic acid encoding the heterologous polypeptide is administered in a viral vector.

34. (canceled)

35. The method of claim 33, wherein the viral vector is an adeno-associated viral (AAV) vector.

36. The method of claim 35, wherein the AAV vector is selected from AAV2, AAV3, AAV3B, AAV5, AAV8, AAV9, AAV-DJ, AAV2/8, AAVrh10, and AAVLK03.

37. The method of claim 1, wherein the sgRNA, the Cas9 nuclease, and the construct comprising for the nucleic acid encoding the heterologous polypeptide are administered simultaneously.

38-47. (canceled)

48. The method of claim 1, wherein the sgRNA mediates target-specific cutting by the Cas9 nuclease, resulting in insertion of the nucleic acid encoding the heterologous polypeptide within intron 1 of an albumin gene.

49. The method of claim 48, wherein the target-specific cutting results in a rate of at least 10% insertion of the nucleic acid encoding the heterologous polypeptide in the cell population.

50-52. (canceled)

53. The method of claim 1, wherein the sgRNA and the Cas9 nuclease are administered in a lipid nanoparticle.

54-55. (canceled)

56. The method of claim 1, wherein the sgRNA and the Cas9 nuclease are administered as a ribonucleoprotein (RNP).

57-117. (canceled)

118. The method of claim 1, wherein the liver cell or liver cell population is a human liver cell or a human liver cell population.

119. A method of inserting a nucleic acid encoding a heterologous polypeptide into intron 1 of an albumin locus of a liver cell or liver cell population in a subject, the method comprising administering to the subject:
i) a single guide RNA (sgRNA) that comprises the sequence of SEQ ID NO: 72;
ii) a Cas9 nuclease; and
iii) an adeno-associated viral (AAV) vector comprising a construct comprising, in 5′ to 3′ order, a splice acceptor site, the nucleic acid encoding the heterologous polypeptide, and a polyadenylation sequence, wherein the construct does not comprise a homology arm;
wherein the sgRNA mediates target-specific cutting by the Cas9 nuclease, resulting in insertion of the nucleic acid encoding the heterologous polypeptide within intron 1 of an albumin locus of the liver cell or liver cell population in the subject.

120. The method of 119, wherein the Cas9 nuclease is administered as a Cas9 nuclease enzyme or a nucleic acid encoding a Cas9 nuclease.

121. The method of claim 120, wherein the nucleic acid encoding the Cas9 nuclease is an mRNA.

122. The method of claim 119, wherein the AAV vector is selected from AAV2, AAV3, AAV3B, AAV5, AAV8, AAV9, AAV-DJ, AAV2/8, AAVrh10, and AAVLK03.

123. The method of claim 119, wherein the sgRNA and the Cas9 nuclease are administered in a lipid nanoparticle.

124. The method of claim 119, wherein the liver cell or liver cell population is a human liver cell or a human liver cell population.

125. A single guide RNA comprising the nucleotide sequence of SEQ ID NO: 40.

126. A composition comprising an sgRNA comprising the nucleotide sequence of SEQ ID NO: 40 and a Cas9 nuclease.


SEQUENCE LISTING (1138 sequences)

SEQ ID NO: 1 - DNA, 709 bp, Homo sapiens
gtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccag

SEQ ID NO: 2 - RNA, 20 nt, Artificial Sequence
  source: /note=“Description of Artificial Sequence: Synthetic oligonucleotide”
gagcaaccucacucuugucu

SEQ ID NO: 3 - RNA, 20 nt, Artificial Sequence
  source: /note=“Description of Artificial Sequence: Synthetic oligonucleotide”
augcauuuguuucaaaauau

SEQ ID NO: 4 - RNA, 20 nt, Artificial Sequence
  source: /note=“Description of Artificial Sequence: Synthetic oligonucleotide”
ugcauuuguuucaaaauauu

SEQ ID NO: 5 - RNA, 20 nt, Artificial Sequence
  source: /note=“Description of Artificial Sequence: Synthetic oligonucleotide”
auuuaugagaucaacagcac

SEQ ID NO: 6 - RNA, 20 nt, Artificial Sequence
  source: /note=“Description of Artificial Sequence: Synthetic oligonucleotide”
gaucaacagcacagguuuug

SEQ ID NO: 7 - RNA, 20 nt, Artificial Sequence
  source: /note=“Description of Artificial Sequence: Synthetic oligonucleotide”
uuaaauaaagcauagugcaa

SEQ ID NO: 8 - RNA, 20 nt, Artificial Sequence
  source: /note=“Description of Artificial Sequence: Synthetic oligonucleotide”
uaaagcauagugcaauggau

SEQ ID NO: 9 - RNA, 20 nt, Artificial Sequence
  source: /note=“Description of Artificial Sequence: Synthetic oligonucleotide”
uagugcaauggauaggucuu

SEQ ID NO: 10 - RNA, 20 nt, Artificial Sequence
  source: /note=“Description of Artificial Sequence: Synthetic oligonucleotide”
uacuaaaacuuuauuuuacu

SEQ ID NO: 11 - RNA, 20 nt, Artificial Sequence
  source: /note=“Description of Artificial Sequence: Synthetic oligonucleotide”
aaaguugaacaauagaaaaa

SEQ ID NO: 12 - RNA, 20 nt, Artificial Sequence
  source: /note=“Description of Artificial Sequence: Synthetic oligonucleotide”
aaugcauaaucuaagucaaa

SEQ ID NO: 13 - RNA, 20 nt, Artificial Sequence
  source: /note=“Description of Artificial Sequence: Synthetic oligonucleotide”
uaauaaaauucaaacauccu

SEQ ID NO: 14 - RNA, 20 nt, Artificial Sequence
  source: /note=“Description of Artificial Sequence: Synthetic oligonucleotide”
gcaucuuuaaagaauuauuu

SEQ ID NO: 15 - RNA, 20 nt, Artificial Sequence
  source: /note=“Description of Artificial Sequence: Synthetic oligonucleotide”
uuuggcauuuauuucuaaaa

SEQ ID NO: 16 - RNA, 20 nt, Artificial Sequence
  source: /note=“Description of Artificial Sequence: Synthetic oligonucleotide”
uguauuugugaagucuuaca

SEQ ID NO: 17 - RNA, 20 nt, Artificial Sequence
  source: /note=“Description of Artificial Sequence: Synthetic oligonucleotide”
uccuagguaaaaaaaaaaaa

SEQ ID NO: 18 - RNA, 20 nt, Artificial Sequence
  source: /note=“Description of Artificial Sequence: Synthetic oligonucleotide”
uaauuuucuuuugcgcacua

SEQ ID NO: 19 - RNA, 20 nt, Artificial Sequence
  source: /note=“Description of Artificial Sequence: Synthetic oligonucleotide”
ugacugaaacuucacagaau

SEQ ID NO: 20 - RNA, 20 nt, Artificial Sequence
  source: /note=“Description of Artificial Sequence: Synthetic oligonucleotide”
gacugaaacuucacagaaua

SEQ ID NO: 21 - RNA, 20 nt, Artificial Sequence
  source: /note=“Description of Artificial Sequence: Synthetic oligonucleotide”
uucauuuuagucugucuucu

SEQ ID NO: 22 - RNA, 20 nt, Artificial Sequence
  source: /note=“Description of Artificial Sequence: Synthetic oligonucleotide”
auuaucuaaguuugaauaua

SEQ ID NO: 23 - RNA, 20 nt, Artificial Sequence
  source: /note=“Description of Artificial Sequence: Synthetic oligonucleotide”
aauuuuuaaaauaguauucu

SEQ ID NO: 24 - RNA, 20 nt, Artificial Sequence
  source: /note=“Description of Artificial Sequence: Synthetic oligonucleotide”
ugaauuauucuucuguuuaa

SEQ ID NO: 25 - RNA, 20 nt, Artificial Sequence
  source: /note=“Description of Artificial Sequence: Synthetic oligonucleotide”
aucauccugaguuuuucugu

SEQ ID NO: 26 - RNA, 20 nt, Artificial Sequence
  source: /note=“Description of Artificial Sequence: Synthetic oligonucleotide”
uuacuaaaacuuuauuuuac

SEQ ID NO: 27 - RNA, 20 nt, Artificial Sequence
  source: /note=“Description of Artificial Sequence: Synthetic oligonucleotide”
accuuuuuuuuuuuuuaccu

SEQ ID NO: 28 - RNA, 20 nt, Artificial Sequence
  source: /note=“Description of Artificial Sequence: Synthetic oligonucleotide”
agugcaauggauaggucuuu

SEQ ID NO: 29 - RNA, 20 nt, Artificial Sequence
  source: /note=“Description of Artificial Sequence: Synthetic oligonucleotide”
ugauuccuacagaaaaacuc

SEQ ID NO: 30 - RNA, 20 nt, Artificial Sequence
  source: /note=“Description of Artificial Sequence: Synthetic oligonucleotide”
ugggcaagggaagaaaaaaa

SEQ ID NO: 31 - RNA, 20 nt, Artificial Sequence
  source: /note=“Description of Artificial Sequence: Synthetic oligonucleotide”
ccucacucuugucugggcaa

SEQ ID NO: 32 - RNA, 20 nt, Artificial Sequence
  source: /note=“Description of Artificial Sequence: Synthetic oligonucleotide”
accucacucuugucugggca

SEQ ID NO: 33 - RNA, 20 nt, Artificial Sequence
  source: /note=“Description of Artificial Sequence: Synthetic oligonucleotide”
ugagcaaccucacucuuguc

SEQ ID NO: 34 - RNA, 100 nt, Artificial Sequence
  source: /note=“Description of Artificial Sequence: Synthetic polynucleotide”
gagcaaccucacucuugucuguuuuagagcuagaaauagcaaguuaaaauaaggcuaguccguuaucaacuugaaaaaguggcaccgagucggugcuuuu

SEQ ID NO: 35 - RNA, 100 nt, Artificial Sequence
  source: /note=“Description of Artificial Sequence: Synthetic polynucleotide”
augcauuuguuucaaaauauguuuuagagcuagaaauagcaaguuaaaauaaggcuaguccguuaucaacuugaaaaaguggcaccgagucggugcuuuu

SEQ ID NO: 36 - RNA, 100 nt, Artificial Sequence
  source: /note=“Description of Artificial Sequence: Synthetic polynucleotide”
ugcauuuguuucaaaauauuguuuuagagcuagaaauagcaaguuaaaauaaggcuaguccguuaucaacuugaaaaaguggcaccgagucggugcuuuu

SEQ ID NO: 37 - RNA, 100 nt, Artificial Sequence
  source: /note=“Description of Artificial Sequence: Synthetic polynucleotide”
auuuaugagaucaacagcacguuuuagagcuagaaauagcaaguuaaaauaaggcuaguccguuaucaacuugaaaaaguggcaccgagucggugcuuuu

SEQ ID NO: 38 - RNA, 100 nt, Artificial Sequence
  source: /note=“Description of Artificial Sequence: Synthetic polynucleotide”
gaucaacagcacagguuuugguuuuagagcuagaaauagcaaguuaaaauaaggcuaguccguuaucaacuugaaaaaguggcaccgagucggugcuuuu

SEQ ID NO: 39 - RNA, 100 nt, Artificial Sequence
  source: /note=“Description of Artificial Sequence: Synthetic polynucleotide”
uuaaauaaagcauagugcaaguuuuagagcuagaaauagcaaguuaaaauaaggcuaguccguuaucaacuugaaaaaguggcaccgagucggugcuuuu

SEQ ID NO: 40 - RNA, 100 nt, Artificial Sequence
  source: /note=“Description of Artificial Sequence: Synthetic polynucleotide”
uaaagcauagugcaauggauguuuuagagcuagaaauagcaaguuaaaauaaggcuaguccguuaucaacuugaaaaaguggcaccgagucggugcuuuu

SEQ ID NO: 41 - RNA, 100 nt, Artificial Sequence
  source: /note=“Description of Artificial Sequence: Synthetic polynucleotide”
uagugcaauggauaggucuuguuuuagagcuagaaauagcaaguuaaaauaaggcuaguccguuaucaacuugaaaaaguggcaccgagucggugcuuuu

SEQ ID NO: 42 - RNA, 100 nt, Artificial Sequence
  source: /note=“Description of Artificial Sequence: Synthetic polynucleotide”
uacuaaaacuuuauuuuacuguuuuagagcuagaaauagcaaguuaaaauaaggcuaguccguuaucaacuugaaaaaguggcaccgagucggugcuuuu

SEQ ID NO: 43 - RNA, 100 nt, Artificial Sequence
  source: /note=“Description of Artificial Sequence: Synthetic polynucleotide”
aaaguugaacaauagaaaaaguuuuagagcuagaaauagcaaguuaaaauaaggcuaguccguuaucaacuugaaaaaguggcaccgagucggugcuuuu

SEQ ID NO: 44 - RNA, 100 nt, Artificial Sequence
  source: /note=“Description of Artificial Sequence: Synthetic polynucleotide”
aaugcauaaucuaagucaaaguuuuagagcuagaaauagcaaguuaaaauaaggcuaguccguuaucaacuugaaaaaguggcaccgagucggugcuuuu

SEQ ID NO: 45 - RNA, 100 nt, Artificial Sequence
  source: /note=“Description of Artificial Sequence: Synthetic polynucleotide”
uaauaaaauucaaacauccuguuuuagagcuagaaauagcaaguuaaaauaaggcuaguccguuaucaacuugaaaaaguggcaccgagucggugcuuuu

SEQ ID NO: 46 - RNA, 100 nt, Artificial Sequence
  source: /note=“Description of Artificial Sequence: Synthetic polynucleotide”
gcaucuuuaaagaauuauuuguuuuagagcuagaaauagcaaguuaaaauaaggcuaguccguuaucaacuugaaaaaguggcaccgagucggugcuuuu

SEQ ID NO: 47 - RNA, 100 nt, Artificial Sequence
  source: /note=“Description of Artificial Sequence: Synthetic polynucleotide”
uuuggcauuuauuucuaaaaguuuuagagcuagaaauagcaaguuaaaauaaggcuaguccguuaucaacuugaaaaaguggcaccgagucggugcuuuu

SEQ ID NO: 48 - RNA, 100 nt, Artificial Sequence
  source: /note=“Description of Artificial Sequence: Synthetic polynucleotide”
uguauuugugaagucuuacaguuuuagagcuagaaauagcaaguuaaaauaaggcuaguccguuaucaacuugaaaaaguggcaccgagucggugcuuuu

SEQ ID NO: 49 - RNA, 100 nt, Artificial Sequence
  source: /note=“Description of Artificial Sequence: Synthetic polynucleotide”
uccuagguaaaaaaaaaaaaguuuuagagcuagaaauagcaaguuaaaauaaggcuaguccguuaucaacuugaaaaaguggcaccgagucggugcuuuu

SEQ ID NO: 50 - RNA, 100 nt, Artificial Sequence
  source: /note=“Description of Artificial Sequence: Synthetic polynucleotide”
uaauuuucuuuugcgcacuaguuuuagagcuagaaauagcaaguuaaaauaaggcuaguccguuaucaacuugaaaaaguggcaccgagucggugcuuuu

SEQ ID NO: 51 - RNA, 100 nt, Artificial Sequence
  source: /note=“Description of Artificial Sequence: Synthetic polynucleotide”
ugacugaaacuucacagaauguuuuagagcuagaaauagcaaguuaaaauaaggcuaguccguuaucaacuugaaaaaguggcaccgagucggugcuuuu

SEQ ID NO: 52 - RNA, 100 nt, Artificial Sequence
  source: /note=“Description of Artificial Sequence: Synthetic polynucleotide”
gacugaaacuucacagaauaguuuuagagcuagaaauagcaaguuaaaauaaggcuaguccguuaucaacuugaaaaaguggcaccgagucggugcuuuu

SEQ ID NO: 53 - RNA, 100 nt, Artificial Sequence
  source: /note=“Description of Artificial Sequence: Synthetic polynucleotide”
uucauuuuagucugucuucuguuuuagagcuagaaauagcaaguuaaaauaaggcuaguccguuaucaacuugaaaaaguggcaccgagucggugcuuuu

SEQ ID NO: 54 - RNA, 100 nt, Artificial Sequence
  source: /note=“Description of Artificial Sequence: Synthetic polynucleotide”
auuaucuaaguuugaauauaguuuuagagcuagaaauagcaaguuaaaauaaggcuaguccguuaucaacuugaaaaaguggcaccgagucggugcuuuu

SEQ ID NO: 55 - RNA, 100 nt, Artificial Sequence
  source: /note=“Description of Artificial Sequence: Synthetic polynucleotide”
aauuuuuaaaauaguauucuguuuuagagcuagaaauagcaaguuaaaauaaggcuaguccguuaucaacuugaaaaaguggcaccgagucggugcuuuu

SEQ ID NO: 56 - RNA, 100 nt, Artificial Sequence
  source: /note=“Description of Artificial Sequence: Synthetic polynucleotide”
ugaauuauucuucuguuuaaguuuuagagcuagaaauagcaaguuaaaauaaggcuaguccguuaucaacuugaaaaaguggcaccgagucggugcuuuu

SEQ ID NO: 57 - RNA, 100 nt, Artificial Sequence
  source: /note=“Description of Artificial Sequence: Synthetic polynucleotide”
aucauccugaguuuuucuguguuuuagagcuagaaauagcaaguuaaaauaaggcuaguccguuaucaacuugaaaaaguggcaccgagucggugcuuuu

SEQ ID NO: 58 - RNA, 100 nt, Artificial Sequence
  source: /note=“Description of Artificial Sequence: Synthetic polynucleotide”
uuacuaaaacuuuauuuuacguuuuagagcuagaaauagcaaguuaaaauaaggcuaguccguuaucaacuugaaaaaguggcaccgagucggugcuuuu

SEQ ID NO: 59 - RNA, 100 nt, Artificial Sequence
  source: /note=“Description of Artificial Sequence: Synthetic polynucleotide”
accuuuuuuuuuuuuuaccuguuuuagagcuagaaauagcaaguuaaaauaaggcuaguccguuaucaacuugaaaaaguggcaccgagucggugcuuuu

SEQ ID NO: 60 - RNA, 100 nt, Artificial Sequence
  source: /note=“Description of Artificial Sequence: Synthetic polynucleotide”
agugcaauggauaggucuuuguuuuagagcuagaaauagcaaguuaaaauaaggcuaguccguuaucaacuugaaaaaguggcaccgagucggugcuuuu

SEQ ID NO: 61 - RNA, 100 nt, Artificial Sequence
  source: /note=“Description of Artificial Sequence: Synthetic polynucleotide”
ugauuccuacagaaaaacucguuuuagagcuagaaauagcaaguuaaaauaaggcuaguccguuaucaacuugaaaaaguggcaccgagucggugcuuuu

SEQ ID NO: 62 - RNA, 100 nt, Artificial Sequence
  source: /note=“Description of Artificial Sequence: Synthetic polynucleotide”
ugggcaagggaagaaaaaaaguuuuagagcuagaaauagcaaguuaaaauaaggcuaguccguuaucaacuugaaaaaguggcaccgagucggugcuuuu

SEQ ID NO: 63 - RNA, 100 nt, Artificial Sequence
  source: /note=“Description of Artificial Sequence: Synthetic polynucleotide”
ccucacucuugucugggcaaguuuuagagcuagaaauagcaaguuaaaauaaggcuaguccguuaucaacuugaaaaaguggcaccgagucggugcuuuu

SEQ ID NO: 64 - RNA, 100 nt, Artificial Sequence
  source: /note=“Description of Artificial Sequence: Synthetic polynucleotide”
accucacucuugucugggcaguuuuagagcuagaaauagcaaguuaaaauaaggcuaguccguuaucaacuugaaaaaguggcaccgagucggugcuuuu

SEQ ID NO: 65 - RNA, 100 nt, Artificial Sequence
  source: /note=“Description of Artificial Sequence: Synthetic polynucleotide”
ugagcaaccucacucuugucguuuuagagcuagaaauagcaaguuaaaauaaggcuaguccguuaucaacuugaaaaaguggcaccgagucggugcuuuu

SEQ ID NO: 66 - RNA, 100 nt, Artificial Sequence; identical to SEQ ID NO: 34
  source: /note=“Description of Artificial Sequence: Synthetic polynucleotide”

SEQ ID NO: 67 - RNA, 100 nt, Artificial Sequence; identical to SEQ ID NO: 35
  source: /note=“Description of Artificial Sequence: Synthetic polynucleotide”

SEQ ID NO: 68 - RNA, 100 nt, Artificial Sequence; identical to SEQ ID NO: 36
  source: /note=“Description of Artificial Sequence: Synthetic polynucleotide”

SEQ ID NO: 69 - RNA, 100 nt, Artificial Sequence; identical to SEQ ID NO: 37
  source: /note=“Description of Artificial Sequence: Synthetic polynucleotide”

SEQ ID NO: 70 - RNA, 100 nt, Artificial Sequence; identical to SEQ ID NO: 38
  source: /note=“Description of Artificial Sequence: Synthetic polynucleotide”

SEQ ID NO: 71 - RNA, 100 nt, Artificial Sequence; identical to SEQ ID NO: 39
  source: /note=“Description of Artificial Sequence: Synthetic polynucleotide”

SEQ ID NO: 72 - RNA, 100 nt, Artificial Sequence; identical to SEQ ID NO: 40
  source: /note=“Description of Artificial Sequence: Synthetic polynucleotide”

SEQ ID NO: 73 - RNA, 100 nt, Artificial Sequence; identical to SEQ ID NO: 41
  source: /note=“Description of Artificial Sequence: Synthetic polynucleotide”

SEQ ID NO: 74 - RNA, 100 nt, Artificial Sequence; identical to SEQ ID NO: 42
  source: /note=“Description of Artificial Sequence: Synthetic polynucleotide”

SEQ ID NO: 75 - RNA, 100 nt, Artificial Sequence; identical to SEQ ID NO: 43
  source: /note=“Description of Artificial Sequence: Synthetic polynucleotide”

SEQ ID NO: 76 - RNA, 100 nt, Artificial Sequence; identical to SEQ ID NO: 44
  source: /note=“Description of Artificial Sequence: Synthetic polynucleotide”

SEQ ID NO: 77 - RNA, 100 nt, Artificial Sequence; identical to SEQ ID NO: 45
  source: /note=“Description of Artificial Sequence: Synthetic polynucleotide”

SEQ ID NO: 78 - RNA, 100 nt, Artificial Sequence; identical to SEQ ID NO: 46
  source: /note=“Description of Artificial Sequence: Synthetic polynucleotide”

SEQ ID NO: 79 - RNA, 100 nt, Artificial Sequence; identical to SEQ ID NO: 47
  source: /note=“Description of Artificial Sequence: Synthetic polynucleotide”

SEQ ID NO: 80 - RNA, 100 nt, Artificial Sequence; identical to SEQ ID NO: 48
  source: /note=“Description of Artificial Sequence: Synthetic polynucleotide”

SEQ ID NO: 81 - RNA, 100 nt, Artificial Sequence; identical to SEQ ID NO: 49
  source: /note=“Description of Artificial Sequence: Synthetic polynucleotide”

SEQ ID NO: 82 - RNA, 100 nt, Artificial Sequence; identical to SEQ ID NO: 50
  source: /note=“Description of Artificial Sequence: Synthetic polynucleotide”

SEQ ID NO: 83 - RNA, 100 nt, Artificial Sequence; identical to SEQ ID NO: 51
  source: /note=“Description of Artificial Sequence: Synthetic polynucleotide”

SEQ ID NO: 84 - RNA, 100 nt, Artificial Sequence; identical to SEQ ID NO: 52
  source: /note=“Description of Artificial Sequence: Synthetic polynucleotide”

SEQ ID NO: 85 - RNA, 100 nt, Artificial Sequence; identical to SEQ ID NO: 53
  source: /note=“Description of Artificial Sequence: Synthetic polynucleotide”

SEQ ID NO: 86 - RNA, 100 nt, Artificial Sequence; identical to SEQ ID NO: 54
  source: /note=“Description of Artificial Sequence: Synthetic polynucleotide”

SEQ ID NO: 87 - RNA, 100 nt, Artificial Sequence; identical to SEQ ID NO: 55
  source: /note=“Description of Artificial Sequence: Synthetic polynucleotide”

SEQ ID NO: 88 - RNA, 100 nt, Artificial Sequence; identical to SEQ ID NO: 56
  source: /note=“Description of Artificial Sequence: Synthetic polynucleotide”

SEQ ID NO: 89 - RNA, 100 nt, Artificial Sequence; identical to SEQ ID NO: 57
  source: /note=“Description of Artificial Sequence: Synthetic polynucleotide”

SEQ ID NO: 90 - RNA, 100 nt, Artificial Sequence; identical to SEQ ID NO: 58
  source: /note=“Description of Artificial Sequence: Synthetic polynucleotide”

SEQ ID NO: 91 - RNA, 100 nt, Artificial Sequence; identical to SEQ ID NO: 59
  source: /note=“Description of Artificial Sequence: Synthetic polynucleotide”

SEQ ID NO: 92 - RNA, 100 nt, Artificial Sequence; identical to SEQ ID NO: 60
  source: /note=“Description of Artificial Sequence: Synthetic polynucleotide”

SEQ ID NO: 93 - RNA, 100 nt, Artificial Sequence; identical to SEQ ID NO: 61
  source: /note=“Description of Artificial Sequence: Synthetic polynucleotide”

SEQ ID NO: 94 - RNA, 100 nt, Artificial Sequence; identical to SEQ ID NO: 62
  source: /note=“Description of Artificial Sequence: Synthetic polynucleotide”

SEQ ID NO: 95 - RNA, 100 nt, Artificial Sequence; identical to SEQ ID NO: 63
  source: /note=“Description of Artificial Sequence: Synthetic polynucleotide”

SEQ ID NO: 96 - RNA, 100 nt, Artificial Sequence; identical to SEQ ID NO: 64
  source: /note=“Description of Artificial Sequence: Synthetic polynucleotide”

SEQ ID NO: 97 - RNA, 100 nt, Artificial Sequence; identical to SEQ ID NO: 65
  source: /note=“Description of Artificial Sequence: Synthetic polynucleotide”

SEQ ID NO: 98 - RNA, 20 nt, Artificial Sequence
  source: /note=“Description of Artificial Sequence: Synthetic oligonucleotide”
auuugcaucugagaacccuu

SEQ ID NO: 99 - RNA, 20 nt, Artificial Sequence
  source: /note=“Description of Artificial Sequence: Synthetic oligonucleotide”
aucgggaacuggcaucuuca

SEQ ID NO: 100 - RNA, 20 nt, Artificial Sequence
  source: /note=“Description of Artificial Sequence: Synthetic oligonucleotide”
guuacaggaaaaucugaagg

SEQ ID NO: 101 - RNA, 20 nt, Artificial Sequence
  source: /note=“Description of Artificial Sequence: Synthetic oligonucleotide”
gaucgggaacuggcaucuuc

SEQ ID NO: 102 - RNA, 20 nt, Artificial Sequence
  source: /note=“Description of Artificial Sequence: Synthetic oligonucleotide”
ugcaucugagaacccuuagg

SEQ ID NO: 103 - RNA, 20 nt, Artificial Sequence
  source: /note=“Description of Artificial Sequence: Synthetic oligonucleotide”
cacucuugucuguggaaaca

SEQ ID NO: 104 - RNA, 20 nt, Artificial Sequence
  source: /note=“Description of Artificial Sequence: Synthetic oligonucleotide”
aucguuacaggaaaaucuga

SEQ ID NO: 105 - RNA, 20 nt, Artificial Sequence
  source: /note=“Description of Artificial Sequence: Synthetic oligonucleotide”
gcaucuucagggaguagcuu

SEQ ID NO: 106 - RNA, 20 nt, Artificial Sequence
  source: /note=“Description of Artificial Sequence: Synthetic oligonucleotide”
caaucuuuaaauauguugug

SEQ ID NO: 107 - RNA, 20 nt, Artificial Sequence
  source: /note=“Description of Artificial Sequence: Synthetic oligonucleotide”
ucacucuugucuguggaaac

SEQ ID NO: 108 - RNA, 20 nt, Artificial Sequence
  source: /note=“Description of Artificial Sequence: Synthetic oligonucleotide”
ugcuuguauuuuucuaguaa

SEQ ID NO: 109 - RNA, 20 nt, Artificial Sequence
  source: /note=“Description of Artificial Sequence: Synthetic oligonucleotide”
guaaauaucuacuaagacaa

SEQ ID NO: 110 - RNA, 20 nt, Artificial Sequence
  source: /note=“Description of Artificial Sequence: Synthetic oligonucleotide”
uuuuucuaguaauggaagcc

SEQ ID NO: 111 - RNA, 20 nt, Artificial Sequence
  source: /note=“Description of Artificial Sequence: Synthetic oligonucleotide”
uuauauuauugauauauuuu

SEQ ID NO: 112 - RNA, 20 nt, Artificial Sequence
  source: /note=“Description of Artificial Sequence: Synthetic oligonucleotide”
gcacagauauaaacacuuaa

SEQ ID NO: 113 - RNA, 20 nt, Artificial Sequence
  source: /note=“Description of Artificial Sequence: Synthetic oligonucleotide”
cacagauauaaacacuuaac

SEQ ID NO: 114 - RNA, 20 nt, Artificial Sequence
  source: /note=“Description of Artificial Sequence: Synthetic oligonucleotide”
gguuuuaaaaauaauaaugu

SEQ ID NO: 115 - RNA, 20 nt, Artificial Sequence
  source: /note=“Description of Artificial Sequence: Synthetic oligonucleotide”
ucagauuuuccuguaacgau

SEQ ID NO: 116 - RNA, 20 nt, Artificial Sequence
  source: /note=“Description of Artificial Sequence: Synthetic oligonucleotide”
cagauuuuccuguaacgauc

SEQ ID NO: 117 - RNA, 20 nt, Artificial Sequence
  source: /note=“Description of Artificial Sequence: Synthetic oligonucleotide”
caaugguaaauaagaaauaa

SEQ ID NO: 118 - RNA, 20 nt, Artificial Sequence
  source: /note=“Description of Artificial Sequence: Synthetic oligonucleotide”
ggaaaaucugaagguggcaa

SEQ ID NO: 119 - RNA, 20 nt, Artificial Sequence
  source: /note=“Description of Artificial Sequence: Synthetic oligonucleotide”
ggcgaucucacucuugucug

SEQ ID NO: 120 - RNA, 100 nt, Artificial Sequence
  source: /note=“Description of Artificial Sequence: Synthetic polynucleotide”
auuugcaucugagaacccuuguuuuagagcuagaaauagcaaguuaaaauaaggcuaguccguuaucaacuugaaaaaguggcaccgagucggugcuuuu

SEQ ID NO: 121 - RNA, 100 nt, Artificial Sequence
  source: /note=“Description of Artificial Sequence: Synthetic polynucleotide”
aucgggaacuggcaucuucaguuuuagagcuagaaauagcaaguuaaaauaaggcuaguccguuaucaacuugaaaaaguggcaccgagucggugcuuuu

SEQ ID NO: 122 - RNA, 100 nt, Artificial Sequence
  source: /note=“Description of Artificial Sequence: Synthetic polynucleotide”
guuacaggaaaaucugaaggguuuuagagcuagaaauagcaaguuaaaauaaggcuaguccguuaucaacuugaaaaaguggcaccgagucggugcuuuu

SEQ ID NO: 123 - RNA, 100 nt, Artificial Sequence
  source: /note=“Description of Artificial Sequence: Synthetic polynucleotide”
gaucgggaacuggcaucuucguuuuagagcuagaaauagcaaguuaaaauaaggcuaguccguuaucaacuugaaaaaguggcaccgagucggugcuuuu

SEQ ID NO: 124 - RNA, 100 nt, Artificial Sequence
  source: /note=“Description of Artificial Sequence: Synthetic polynucleotide”
ugcaucugagaacccuuaggguuuuagagcuagaaauagcaaguuaaaauaaggcuaguccguuaucaacuugaaaaaguggcaccgagucggugcuuuu

SEQ ID NO: 125 - RNA, 100 nt, Artificial Sequence
  source: /note=“Description of Artificial Sequence: Synthetic polynucleotide”
cacucuugucuguggaaacaguuuuagagcuagaaauagcaaguuaaaauaaggcuaguccguuaucaacuugaaaaaguggcaccgagucggugcuuuu

SEQ ID NO: 126 - RNA, 100 nt, Artificial Sequence
  source: /note=“Description of Artificial Sequence: Synthetic polynucleotide”
aucguuacaggaaaaucugaguuuuagagcuagaaauagcaaguuaaaauaaggcuaguccguuaucaacuugaaaaaguggcaccgagucggugcuuuu

SEQ ID NO: 127 - RNA, 100 nt, Artificial Sequence
  source: /note=“Description of Artificial Sequence: Synthetic polynucleotide”
gcaucuucagggaguagcuuguuuuagagcuagaaauagcaaguuaaaauaaggcuaguccguuaucaacuugaaaaaguggcaccgagucggugcuuuu

SEQ ID NO: 128 - RNA, 100 nt, Artificial Sequence
  source: /note=“Description of Artificial Sequence: Synthetic polynucleotide”
caaucuuuaaauauguugugguuuuagagcuagaaauagcaaguuaaaauaaggcuaguccguuaucaacuugaaaaaguggcaccgagucggugcuuuu

SEQ ID NO: 129 - RNA, 100 nt, Artificial Sequence
  source: /note=“Description of Artificial Sequence: Synthetic polynucleotide”
ucacucuugucuguggaaacguuuuagagcuagaaauagcaaguuaaaauaaggcuaguccguuaucaacuugaaaaaguggcaccgagucggugcuuuu

SEQ ID NO: 130 - RNA, 100 nt, Artificial Sequence
  source: /note=“Description of Artificial Sequence: Synthetic polynucleotide”
ugcuuguauuuuucuaguaaguuuuagagcuagaaauagcaaguuaaaauaaggcuaguccguuaucaacuugaaaaaguggcaccgagucggugcuuuu

SEQ ID NO: 131 - RNA, 100 nt, Artificial Sequence
  source: /note=“Description of Artificial Sequence: Synthetic polynucleotide”
guaaauaucuacuaagacaaguuuuagagcuagaaauagcaaguuaaaauaaggcuaguccguuaucaacuugaaaaaguggcaccgagucggugcuuuu

SEQ ID NO: 132 - RNA, 100 nt, Artificial Sequence
  source: /note=“Description of Artificial Sequence: Synthetic polynucleotide”
uuuuucuaguaauggaagccguuuuagagcuagaaauagcaaguuaaaauaaggcuaguccguuaucaacuugaaaaaguggcaccgagucggugcuuuu

SEQ ID NO: 133 - RNA, 100 nt, Artificial Sequence
  source: /note=“Description of Artificial Sequence: Synthetic polynucleotide”
uuauauuauugauauauuuuguuuuagagcuagaaauagcaaguuaaaauaaggcuaguccguuaucaacuugaaaaaguggcaccgagucggugcuuuu

SEQ ID NO: 134 - RNA, 100 nt, Artificial Sequence
  source: /note=“Description of Artificial Sequence: Synthetic polynucleotide”
gcacagauauaaacacuuaaguuuuagagcuagaaauagcaaguuaaaauaaggcuaguccguuaucaacuugaaaaaguggcaccgagucggugcuuuu

SEQ ID NO: 135 - RNA, 100 nt, Artificial Sequence
  source: /note=“Description of Artificial Sequence: Synthetic polynucleotide”
cacagauauaaacacuuaacguuuuagagcuagaaauagcaaguuaaaauaaggcuaguccguuaucaacuugaaaaaguggcaccgagucggugcuuuu

SEQ ID NO: 136 - RNA, 100 nt, Artificial Sequence
  source: /note=“Description of Artificial Sequence: Synthetic polynucleotide”
gguuuuaaaaauaauaauguguuuuagagcuagaaauagcaaguuaaaauaaggcuaguccguuaucaacuugaaaaaguggcaccgagucggugcuuuu

SEQ ID NO: 137 - RNA, 100 nt, Artificial Sequence
  source: /note=“Description of Artificial Sequence: Synthetic polynucleotide”
ucagauuuuccuguaacgauguuuuagagcuagaaauagcaaguuaaaauaaggcuaguccguuaucaacuugaaaaaguggcaccgagucggugcuuuu

SEQ ID NO: 138 - RNA, 100 nt, Artificial Sequence
  source: /note=“Description of Artificial Sequence: Synthetic polynucleotide”
cagauuuuccuguaacgaucguuuuagagcuagaaauagcaaguuaaaauaaggcuaguccguuaucaacuugaaaaaguggcaccgagucggugcuuuu

SEQ ID NO: 139 - RNA, 100 nt, Artificial Sequence
  source: /note=“Description of Artificial Sequence: Synthetic polynucleotide”
caaugguaaauaagaaauaaguuuuagagcuagaaauagcaaguuaaaauaaggcuaguccguuaucaacuugaaaaaguggcaccgagucggugcuuuu

SEQ ID NO: 140 - RNA, 100 nt, Artificial Sequence
  source: /note=“Description of Artificial Sequence: Synthetic polynucleotide”
ggaaaaucugaagguggcaaguuuuagagcuagaaauagcaaguuaaaauaaggcuaguccguuaucaacuugaaaaaguggcaccgagucggugcuuuu

SEQ ID NO: 141 - RNA, 100 nt, Artificial Sequence
  source: /note=“Description of Artificial Sequence: Synthetic polynucleotide”
ggcgaucucacucuugucugguuuuagagcuagaaauagcaaguuaaaauaaggcuaguccguuaucaacuugaaaaaguggcaccgagucggugcuuuu

SEQ ID NO: 142 - RNA, 100 nt, Artificial Sequence; identical to SEQ ID NO: 120
  source: /note=“Description of Artificial Sequence: Synthetic polynucleotide”

SEQ ID NO: 143 - RNA, 100 nt, Artificial Sequence; identical to SEQ ID NO: 121
  source: /note=“Description of Artificial Sequence: Synthetic polynucleotide”

SEQ ID NO: 144 - RNA, 100 nt, Artificial Sequence; identical to SEQ ID NO: 122
  source: /note=“Description of Artificial Sequence: Synthetic polynucleotide”

SEQ ID NO: 145 - RNA, 100 nt, Artificial Sequence; identical to SEQ ID NO: 123
  source: /note=“Description of Artificial Sequence: Synthetic polynucleotide”

SEQ ID NO: 146 - RNA, 100 nt, Artificial Sequence; identical to SEQ ID NO: 124
  source: /note=“Description of Artificial Sequence: Synthetic polynucleotide”

SEQ ID NO: 147 - RNA, 100 nt, Artificial Sequence; identical to SEQ ID NO: 125
  source: /note=“Description of Artificial Sequence: Synthetic polynucleotide”

SEQ ID NO: 148 - RNA, 100 nt, Artificial Sequence; identical to SEQ ID NO: 126
  source: /note=“Description of Artificial Sequence: Synthetic polynucleotide”

SEQ ID NO: 149 - RNA, 100 nt, Artificial Sequence; identical to SEQ ID NO: 127
  source: /note=“Description of Artificial Sequence: Synthetic polynucleotide”

SEQ ID NO: 150 - RNA, 100 nt, Artificial Sequence; identical to SEQ ID NO: 128
  source: /note=“Description of Artificial Sequence: Synthetic polynucleotide”

SEQ ID NO: 151 - RNA, 100 nt, Artificial Sequence; identical to SEQ ID NO: 129
  source: /note=“Description of Artificial Sequence: Synthetic polynucleotide”

SEQ ID NO: 152 - RNA, 100 nt, Artificial Sequence; identical to SEQ ID NO: 130
  source: /note=“Description of Artificial Sequence: Synthetic polynucleotide”

SEQ ID NO: 153 - RNA, 100 nt, Artificial Sequence; identical to SEQ ID NO: 131
  source: /note=“Description of Artificial Sequence: Synthetic polynucleotide”

SEQ ID NO: 154 - RNA, 100 nt, Artificial Sequence; identical to SEQ ID NO: 132
  source: /note=“Description of Artificial Sequence: Synthetic polynucleotide”

SEQ ID NO: 155 - RNA, 100 nt, Artificial Sequence; identical to SEQ ID NO: 133
  source: /note=“Description of Artificial Sequence: Synthetic polynucleotide”

SEQ ID NO: 156 - RNA, 100 nt, Artificial Sequence; identical to SEQ ID NO: 134
  source: /note=“Description of Artificial Sequence: Synthetic polynucleotide”

SEQ ID NO: 157 - RNA, 100 nt, Artificial Sequence; identical to SEQ ID NO: 135
  source: /note=“Description of Artificial Sequence: Synthetic polynucleotide”

SEQ ID NO: 158 - RNA, 100 nt, Artificial Sequence; identical to SEQ ID NO: 136
  source: /note=“Description of Artificial Sequence: Synthetic polynucleotide”

SEQ ID NO: 159 - RNA, 100 nt, Artificial Sequence; identical to SEQ ID NO: 137
  source: /note=“Description of Artificial Sequence: Synthetic polynucleotide”

SEQ ID NO: 160 - RNA, 100 nt, Artificial Sequence; identical to SEQ ID NO: 138
  source: /note=“Description of Artificial Sequence: Synthetic polynucleotide”

SEQ ID NO: 161 - RNA, 100 nt, Artificial Sequence; identical to SEQ ID NO: 139
  source: /note=“Description of Artificial Sequence: Synthetic polynucleotide”

SEQ ID NO: 162 - RNA, 100 nt, Artificial Sequence; identical to SEQ ID NO: 140
  source: /note=“Description of Artificial Sequence: Synthetic polynucleotide”

SEQ ID NO: 163 - RNA, 100 nt, Artificial Sequence; identical to SEQ ID NO: 141
  source: /note=“Description of Artificial Sequence: Synthetic polynucleotide”

SEQ ID NO: 164 - RNA, 20 nt, Artificial Sequence
  source: /note=“Description of Artificial Sequence: Synthetic oligonucleotide”
gagcaaccucacucuugucu

SEQ ID NO: 165 - RNA, 20 nt, Artificial Sequence
  source: /note=“Description of Artificial Sequence: Synthetic oligonucleotide”
agcaaccucacucuugucug

SEQ ID NO: 166 - RNA, 20 nt, Artificial Sequence
  source: /note=“Description of Artificial Sequence: Synthetic oligonucleotide”
accucacucuugucugggga

SEQ ID NO: 167 - RNA, 20 nt, Artificial Sequence
  source: /note=“Description of Artificial Sequence: Synthetic oligonucleotide”
ccucacucuugucuggggaa

SEQ ID NO: 168 - RNA, 20 nt, Artificial Sequence
  source: /note=“Description of Artificial Sequence: Synthetic oligonucleotide”
cucacucuugucuggggaag

SEQ ID NO: 169 - RNA, 20 nt, Artificial Sequence
  source: /note=“Description of Artificial Sequence: Synthetic oligonucleotide”
ggggaaggggagaaaaaaaa

SEQ ID NO: 170 - RNA, 20 nt, Artificial Sequence
  source: /note=“Description of Artificial Sequence: Synthetic oligonucleotide”
gggaaggggagaaaaaaaaa

SEQ ID NO: 171 - RNA, 20 nt, Artificial Sequence
  source: /note=“Description of Artificial Sequence: Synthetic oligonucleotide”
augcauuuguuucaaaauau

SEQ ID NO: 172 - RNA, 20 nt, Artificial Sequence
  source: /note=“Description of Artificial Sequence: Synthetic oligonucleotide”
ugcauuuguuucaaaauauu

SEQ ID NO: 173 - RNA, 20 nt, Artificial Sequence
  source: /note=“Description of Artificial Sequence: Synthetic oligonucleotide”
ugauuccuacagaaaaaguc

SEQ ID NO: 174 - RNA, 20 nt, Artificial Sequence
  source: /note=“Description of Artificial Sequence: Synthetic oligonucleotide”
uacagaaaaagucaggauaa

SEQ ID NO: 175 - RNA, 20 nt, Artificial Sequence
  source: /note=“Description of Artificial Sequence: Synthetic oligonucleotide”
uuucuucugccuuuaaacag

SEQ ID NO: 176 - RNA, 20 nt, Artificial Sequence
  source: /note=“Description of Artificial Sequence: Synthetic oligonucleotide”
uuauaguuuuauauucaaac

SEQ ID NO: 177 - RNA, 20 nt, Artificial Sequence
  source: /note=“Description of Artificial Sequence: Synthetic oligonucleotide”
auuuaugagaucaacagcac

SEQ ID NO: 178 - RNA, 20 nt, Artificial Sequence
  source: /note=“Description of Artificial Sequence: Synthetic oligonucleotide”
gaucaacagcacagguuuug

SEQ ID NO: 179 - RNA, 20 nt, Artificial Sequence
  source: /note=“Description of Artificial Sequence: Synthetic oligonucleotide”
uuaaauaaagcauagugcaa

SEQ ID NO: 180 - RNA, 20 nt, Artificial Sequence
  source: /note=“Description of Artificial Sequence: Synthetic oligonucleotide”
uaaagcauagugcaauggau

SEQ ID NO: 181 - RNA, 20 nt, Artificial Sequence
  source: /note=“Description of Artificial Sequence: Synthetic oligonucleotide”
uagugcaauggauaggucuu

SEQ ID NO: 182 - RNA, 20 nt, Artificial Sequence
  source: /note=“Description of Artificial Sequence: Synthetic oligonucleotide”
agugcaauggauaggucuua

SEQ ID NO: 183 - RNA, 20 nt, Artificial Sequence
  source: /note=“Description of Artificial Sequence: Synthetic oligonucleotide”
uuacuuugcacuuuccuuag

SEQ ID NO: 184 - RNA, 20 nt, Artificial Sequence
  source: /note=“Description of Artificial Sequence: Synthetic oligonucleotide”
uacuuugcacuuuccuuagu

SEQ ID NO: 185 - RNA, 20 nt, Artificial Sequence
  source: /note=“Description of Artificial Sequence: Synthetic oligonucleotide”
ucugaccuuuuauuuuaccu

SEQ ID NO: 186 - RNA, 20 nt, Artificial Sequence
  source: /note=“Description of Artificial Sequence: Synthetic oligonucleotide”
uacuaaaacuuuauuuuacu

SEQ ID NO: 187 - RNA, 20 nt, Artificial Sequence
  source: /note=“Description of Artificial Sequence: Synthetic oligonucleotide”
aaaguugaacaauagaaaaa

SEQ ID NO: 188 - RNA, 20 nt, Artificial Sequence
  source: /note=“Description of Artificial Sequence: Synthetic oligonucleotide”
aaugcauaaucuaagucaaa

SEQ ID NO: 189 - RNA, 20 nt, Artificial Sequence
  source: /note=“Description of Artificial Sequence: Synthetic oligonucleotide”
auuauccugacuuuuucugu

SEQ ID NO: 190 - RNA, 20 nt, Artificial Sequence
  source: /note=“Description of Artificial Sequence: Synthetic oligonucleotide”
ugaauuauuccucuguuuaa

SEQ ID NO: 191 - RNA, 20 nt, Artificial Sequence
  source: /note=“Description of Artificial Sequence: Synthetic oligonucleotide”
uaauuuucuuuugcccacua

SEQ ID NO: 192 - RNA, 20 nt, Artificial Sequence
  source: /note=“Description of Artificial Sequence: Synthetic oligonucleotide”
aaaaggucagaauuguuuag

SEQ ID NO: 193 - RNA, 20 nt, Artificial Sequence
  source: /note=“Description of Artificial Sequence: Synthetic oligonucleotide”
aacauccuagguaaaauaaa

SEQ ID NO: 194 - RNA, 20 nt, Artificial Sequence
  source: /note=“Description of Artificial Sequence: Synthetic oligonucleotide”
uaauaaaauucaaacauccu

SEQ ID NO: 195 - RNA, 20 nt, Artificial Sequence
  source: /note=“Description of Artificial Sequence: Synthetic oligonucleotide”
uugucauguauuucuaaaau

SEQ ID NO: 196 - RNA, 20 nt, Artificial Sequence
  source: /note=“Description of Artificial Sequence: Synthetic oligonucleotide”
uuugucauguauuucuaaaa

SEQ ID NO: 197 - RNA, 100 nt, Artificial Sequence; identical to SEQ ID NO: 34
  source: /note=“Description of Artificial Sequence: Synthetic polynucleotide”

SEQ ID NO: 198 - RNA, 100 nt, Artificial Sequence
  source: /note=“Description of Artificial Sequence: Synthetic polynucleotide”
agcaaccucacucuugucugguuuuagagcuagaaauagcaaguuaaaauaaggcuaguccguuaucaacuugaaaaaguggcaccgagucggugcuuuu

SEQ ID NO: 199 - RNA, 100 nt, Artificial Sequence
  source: /note=“Description of Artificial Sequence: Synthetic polynucleotide”
accucacucuugucuggggaguuuuagagcuagaaauagcaaguuaaaauaaggcuaguccguuaucaacuugaaaaaguggcaccgagucggugcuuuu

SEQ ID NO: 200 - RNA, 100 nt, Artificial Sequence
  source: /note=“Description of Artificial Sequence: Synthetic polynucleotide”
ccucacucuugucuggggaaguuuuagagcuagaaauagcaaguuaaaauaaggcuaguccguuaucaacuugaaaaaguggcaccgagucggugcuuuu

SEQ ID NO: 201 - RNA, 100 nt, Artificial Sequence
  source: /note=“Description of Artificial Sequence: Synthetic polynucleotide”
cucacucuugucuggggaagguuuuagagcuagaaauagcaaguuaaaauaaggcuaguccguuaucaacuugaaaaaguggcaccgagucggugcuuuu

SEQ ID NO: 202 - RNA, 100 nt, Artificial Sequence
  source: /note=“Description of Artificial Sequence: Synthetic polynucleotide”
ggggaaggggagaaaaaaaaguuuuagagcuagaaauagcaaguuaaaauaaggcuaguccguuaucaacuugaaaaaguggcaccgagucggugcuuuu

SEQ ID NO: 203 - RNA, 100 nt, Artificial Sequence
  source: /note=“Description of Artificial Sequence: Synthetic polynucleotide”
gggaaggggagaaaaaaaaaguuuuagagcuagaaauagcaaguuaaaauaaggcuaguccguuaucaacuugaaaaaguggcaccgagucggugcuuuu

SEQ ID NO: 204 - RNA, 100 nt, Artificial Sequence; identical to SEQ ID NO: 35
  source: /note=“Description of Artificial Sequence: Synthetic polynucleotide”

SEQ ID NO: 205 - RNA, 100 nt, Artificial Sequence; identical to SEQ ID NO: 36
  source: /note=“Description of Artificial Sequence: Synthetic polynucleotide”

SEQ ID NO: 206 - RNA, 100 nt, Artificial Sequence
  source: /note=“Description of Artificial Sequence: Synthetic polynucleotide”
ugauuccuacagaaaaagucguuuuagagcuagaaauagcaaguuaaaauaaggcuaguccguuaucaacuugaaaaaguggcaccgagucggugcuuuu

SEQ ID NO: 207 - RNA, 100 nt, Artificial Sequence
  source: /note=“Description of Artificial Sequence: Synthetic polynucleotide”
uacagaaaaagucaggauaaguuuuagagcuagaaauagcaaguuaaaauaaggcuaguccguuaucaacuugaaaaaguggcaccgagucggugcuuuu

SEQ ID NO: 208 - RNA, 100 nt, Artificial Sequence
  source: /note=“Description of Artificial Sequence: Synthetic polynucleotide”
uuucuucugccuuuaaacagguuuuagagcuagaaauagcaaguuaaaauaaggcuaguccguuaucaacuugaaaaaguggcaccgagucggugcuuuu

SEQ ID NO: 209 - RNA, 100 nt, Artificial Sequence
  source: /note=“Description of Artificial Sequence: Synthetic polynucleotide”
uuauaguuuuauauucaaacguuuuagagcuagaaauagcaaguuaaaauaaggcuaguccguuaucaacuugaaaaaguggcaccgagucggugcuuuu

SEQ ID NO: 210 - RNA, 100 nt, Artificial Sequence; identical to SEQ ID NO: 37
  source: /note=“Description of Artificial Sequence: Synthetic polynucleotide”

SEQ ID NO: 211 - RNA, 100 nt, Artificial Sequence; identical to SEQ ID NO: 38
  source: /note=“Description of Artificial Sequence: Synthetic polynucleotide”

SEQ ID NO: 212 - RNA, 100 nt, Artificial Sequence; identical to SEQ ID NO: 39
  source: /note=“Description of Artificial Sequence: Synthetic polynucleotide”

SEQ ID NO: 213 - RNA, 100 nt, Artificial Sequence; identical to SEQ ID NO: 40
  source: /note=“Description of Artificial Sequence: Synthetic polynucleotide”

SEQ ID NO: 214 - RNA, 100 nt, Artificial Sequence; identical to SEQ ID NO: 41
  source: /note=“Description of Artificial Sequence: Synthetic polynucleotide”

SEQ ID NO: 215 - RNA, 100 nt, Artificial Sequence
  source: /note=“Description of Artificial Sequence: Synthetic polynucleotide”
agugcaauggauaggucuuaguuuuagagcuagaaauagcaaguuaaaauaaggcuaguccguuaucaacuugaaaaaguggcaccgagucggugcuuuu

SEQ ID NO: 216 - RNA, 100 nt, Artificial Sequence
  source: /note=“Description of Artificial Sequence: Synthetic polynucleotide”
uuacuuugcacuuuccuuagguuuuagagcuagaaauagcaaguuaaaauaaggcuaguccguuaucaacuugaaaaaguggcaccgagucggugcuuuu

SEQ ID NO: 217 - RNA, 100 nt, Artificial Sequence
  source: /note=“Description of Artificial Sequence: Synthetic polynucleotide”
uacuuugcacuuuccuuaguguuuuagagcuagaaauagcaaguuaaaauaaggcuaguccguuaucaacuugaaaaaguggcaccgagucggugcuuuu

SEQ ID NO: 218 - RNA, 100 nt, Artificial Sequence
  source: /note=“Description of Artificial Sequence: Synthetic polynucleotide”
ucugaccuuuuauuuuaccuguuuuagagcuagaaauagcaaguuaaaauaaggcuaguccguuaucaacuugaaaaaguggcaccgagucggugcuuuu

SEQ ID NO: 219 - RNA, 100 nt, Artificial Sequence; identical to SEQ ID NO: 42
  source: /note=“Description of Artificial Sequence: Synthetic polynucleotide”

SEQ ID NO: 220 - RNA, 100 nt, Artificial Sequence; identical to SEQ ID NO: 43
  source: /note=“Description of Artificial Sequence: Synthetic polynucleotide”

SEQ ID NO: 221 - RNA, 100 nt, Artificial Sequence; identical to SEQ ID NO: 44
  source: /note=“Description of Artificial Sequence: Synthetic polynucleotide”

SEQ ID NO: 222 - RNA, 100 nt, Artificial Sequence
  source: /note=“Description of Artificial Sequence: Synthetic polynucleotide”
auuauccugacuuuuucuguguuuuagagcuagaaauagcaaguuaaaauaaggcuaguccguuaucaacuugaaaaaguggcaccgagucggugcuuuu

SEQ ID NO: 223 - RNA, 100 nt, Artificial Sequence
  source: /note=“Description of Artificial Sequence: Synthetic polynucleotide”
ugaauuauuccucuguuuaaguuuuagagcuagaaauagcaaguuaaaauaaggcuaguccguuaucaacuugaaaaaguggcaccgagucggugcuuuu

SEQ ID NO: 224 - RNA, 100 nt, Artificial Sequence
  source: /note=“Description of Artificial Sequence: Synthetic polynucleotide”
uaauuuucuuuugcccacuaguuuuagagcuagaaauagcaaguuaaaauaaggcuaguccguuaucaacuugaaaaaguggcaccgagucggugcuuuu

SEQ ID NO: 225 - RNA, 100 nt, Artificial Sequence
  source: /note=“Description of Artificial Sequence: Synthetic polynucleotide”
aaaaggucagaauuguuuagguuuuagagcuagaaauagcaaguuaaaauaaggcuaguccguuaucaacuugaaaaaguggcaccgagucggugcuuuu

SEQ ID NO: 226 - RNA, 100 nt, Artificial Sequence
  source: /note=“Description of Artificial Sequence: Synthetic polynucleotide”
aacauccuagguaaaauaaaguuuuagagcuagaaauagcaaguuaaaauaaggcuaguccguuaucaacuugaaaaaguggcaccgagucggugcuuuu

SEQ ID NO: 227 - RNA, 100 nt, Artificial Sequence; identical to SEQ ID NO: 45
  source: /note=“Description of Artificial Sequence: Synthetic polynucleotide”

SEQ ID NO: 228 - RNA, 100 nt, Artificial Sequence
  source: /note=“Description of Artificial Sequence: Synthetic polynucleotide”
uugucauguauuucuaaaauguuuuagagcuagaaauagcaaguuaaaauaaggcuaguccguuaucaacuugaaaaaguggcaccgagucggugcuuuu

SEQ ID NO: 229 - RNA, 100 nt, Artificial Sequence
  source: /note=“Description of Artificial Sequence: Synthetic polynucleotide”
uuugucauguauuucuaaaaguuuuagagcuagaaauagcaaguuaaaauaaggcuaguccguuaucaacuugaaaaaguggcaccgagucggugcuuuu

SEQ ID NO: 230 - RNA, 100 nt, Artificial Sequence; identical to SEQ ID NO: 34
  source: /note=“Description of Artificial Sequence: Synthetic polynucleotide”

SEQ ID NO: 231 - RNA, 100 nt, Artificial Sequence; identical to SEQ ID NO: 198
  source: /note=“Description of Artificial Sequence: Synthetic polynucleotide”

SEQ ID NO: 232 - RNA, 100 nt, Artificial Sequence; identical to SEQ ID NO: 199
  source: /note=“Description of Artificial Sequence: Synthetic polynucleotide”

SEQ ID NO: 233 - RNA, 100 nt, Artificial Sequence; identical to SEQ ID NO: 200
  source: /note=“Description of Artificial Sequence: Synthetic polynucleotide”

SEQ ID NO: 234 - RNA, 100 nt, Artificial Sequence; identical to SEQ ID NO: 201
  source: /note=“Description of Artificial Sequence: Synthetic polynucleotide”

SEQ ID NO: 235 - RNA, 100 nt, Artificial Sequence; identical to SEQ ID NO: 202
  source: /note=“Description of Artificial Sequence: Synthetic polynucleotide”

SEQ ID NO: 236 - RNA, 100 nt, Artificial Sequence; identical to SEQ ID NO: 203
  source: /note=“Description of Artificial Sequence: Synthetic polynucleotide”

SEQ ID NO: 237 - RNA, 100 nt, Artificial Sequence; identical to SEQ ID NO: 35
  source: /note=“Description of Artificial Sequence: Synthetic polynucleotide”

SEQ ID NO: 238 - RNA, 100 nt, Artificial Sequence; identical to SEQ ID NO: 36
  source: /note=“Description of Artificial Sequence: Synthetic polynucleotide”

SEQ ID NO: 239 - RNA, 100 nt, Artificial Sequence; identical to SEQ ID NO: 206
  source: /note=“Description of Artificial Sequence: Synthetic polynucleotide”

SEQ ID NO: 240 - RNA, 100 nt, Artificial Sequence; identical to SEQ ID NO: 207
  source: /note=“Description of Artificial Sequence: Synthetic polynucleotide”

SEQ ID NO: 241 - RNA, 100 nt, Artificial Sequence; identical to SEQ ID NO: 208
  source: /note=“Description of Artificial Sequence: Synthetic polynucleotide”

SEQ ID NO: 242 - RNA, 100 nt, Artificial Sequence; identical to SEQ ID NO: 209
  source: /note=“Description of Artificial Sequence: Synthetic polynucleotide”

SEQ ID NO: 243 - RNA, 100 nt, Artificial Sequence; identical to SEQ ID NO: 37
  source: /note=“Description of Artificial Sequence: Synthetic polynucleotide”

SEQ ID NO: 244 - RNA, 100 nt, Artificial Sequence; identical to SEQ ID NO: 38
  source: /note=“Description of Artificial Sequence: Synthetic polynucleotide”

SEQ ID NO: 245 - RNA, 100 nt, Artificial Sequence; identical to SEQ ID NO: 39
  source: /note=“Description of Artificial Sequence: Synthetic polynucleotide”

SEQ ID NO: 246 - RNA, 100 nt, Artificial Sequence; identical to SEQ ID NO: 40
  source: /note=“Description of Artificial Sequence: Synthetic polynucleotide”

SEQ ID NO: 247 - RNA, 100 nt, Artificial Sequence; identical to SEQ ID NO: 41
  source: /note=“Description of Artificial Sequence: Synthetic polynucleotide”

SEQ ID NO: 248 - RNA, 100 nt, Artificial Sequence; identical to SEQ ID NO: 215
  source: /note=“Description of Artificial Sequence: Synthetic polynucleotide”

SEQ ID NO: 249 - RNA, 100 nt, Artificial Sequence; identical to SEQ ID NO: 216
  source: /note=“Description of Artificial Sequence: Synthetic polynucleotide”

SEQ ID NO: 250 - RNA, 100 nt, Artificial Sequence; identical to SEQ ID NO: 217
  source: /note=“Description of Artificial Sequence: Synthetic polynucleotide”

SEQ ID NO: 251 - RNA, 100 nt, Artificial Sequence; identical to SEQ ID NO: 218
  source: /note=“Description of Artificial Sequence: Synthetic polynucleotide”

SEQ ID NO: 252 - RNA, 100 nt, Artificial Sequence; identical to SEQ ID NO: 42
  source: /note=“Description of Artificial Sequence: Synthetic polynucleotide”

SEQ ID NO: 253 - RNA, 100 nt, Artificial Sequence; identical to SEQ ID NO: 43
  source: /note=“Description of Artificial Sequence: Synthetic polynucleotide”

SEQ ID NO: 254 - RNA, 100 nt, Artificial Sequence; identical to SEQ ID NO: 44
  source: /note=“Description of Artificial Sequence: Synthetic polynucleotide”

SEQ ID NO: 255 - RNA, 100 nt, Artificial Sequence; identical to SEQ ID NO: 222
  source: /note=“Description of Artificial Sequence: Synthetic polynucleotide”

SEQ ID NO: 256 - RNA, 100 nt, Artificial Sequence; identical to SEQ ID NO: 223
  source: /note=“Description of Artificial Sequence: Synthetic polynucleotide”

SEQ ID NO: 257 - RNA, 100 nt, Artificial Sequence; identical to SEQ ID NO: 224
  source: /note=“Description of Artificial Sequence: Synthetic polynucleotide”

SEQ ID NO: 258 - RNA, 100 nt, Artificial Sequence; identical to SEQ ID NO: 225
  source: /note=“Description of Artificial Sequence: Synthetic polynucleotide”

SEQ ID NO: 259 - RNA, 100 nt, Artificial Sequence; identical to SEQ ID NO: 226
  source: /note=“Description of Artificial Sequence: Synthetic polynucleotide”

SEQ ID NO: 260 - RNA, 100 nt, Artificial Sequence; identical to SEQ ID NO: 45
  source: /note=“Description of Artificial Sequence: Synthetic polynucleotide”

SEQ ID NO: 261 - RNA, 100 nt, Artificial Sequence; identical to SEQ ID NO: 228
  source: /note=“Description of Artificial Sequence: Synthetic polynucleotide”

SEQ ID NO: 262 - RNA, 100 nt, Artificial Sequence; identical to SEQ ID NO: 229
  source: /note=“Description of Artificial Sequence: Synthetic polynucleotide”

SEQ ID NO: 263 - DNA, 145 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 264 - DNA, 100 bp, Artificial Sequence
  source: /note=“Description of Artificial Sequence: Synthetic polynucleotide”
taggtcagtgaagagaagaacaaaaagcagcatattacagttagttgtcttcatcaatctttaaatatgttgtgtggtttttctctccctgtttccacag

SEQ ID NO: 265 - DNA, 1296 bp, Artificial Sequence
  source: /note=“Description of Artificial Sequence: Synthetic polynucleotide”
tttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 266 - DNA, 276 bp, Artificial Sequence
  source: /note=“Description of Artificial Sequence: Synthetic polynucleotide”
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccc

SEQ ID NO: 267 - DNA, 192 bp, Artificial Sequence
  source: /note=“Description of Artificial Sequence: Synthetic polynucleotide”
aaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 268 - DNA, 1296 bp, Artificial Sequence
  source: /note=“Description of Artificial Sequence: Synthetic polynucleotide”
ttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaa

SEQ ID NO: 269 - DNA, 100 bp, Artificial Sequence
  source: /note=“Description of Artificial Sequence: Synthetic polynucleotide”
ctgtggaaacagggagagaaaaaccacacaacatatttaaagattgatgaagacaactaactgtaatatgctgctttttgttcttctcttcactgaccta

SEQ ID NO: 270 - DNA, 145 bp, Artificial Sequence
  source: /note=“Description of Artificial Sequence: Synthetic polynucleotide”
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 271 - DNA, 100 bp, Artificial Sequence
  source: /note=“Description of Artificial Sequence: Synthetic polynucleotide”
gattatttggattaaaaacaaagactttcttaagagatgtaaaattttcatgatgttttcttttttgctaaaactaaagaattattcttttacatttcag

SEQ ID NO: 272 - DNA, 1329 bp, Artificial Sequence
  source: /note=“Description of Artificial Sequence: Synthetic polynucleotide”
tttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtctcccggtatgtcaactggattaaggaaaaaacaaagctcactgtcagcggatggagactgttcaagaagatcagctaa

SEQ ID NO: 273 - DNA, 1329 bp, Artificial Sequence
  source: /note=“Description of Artificial Sequence: Synthetic polynucleotide”
ttaggaaatcttcttaaacagccgccagccgctcacggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaa

SEQ ID NO: 274 - DNA, 100 bp, Artificial Sequence
  source: /note=“Description of Artificial Sequence: Synthetic polynucleotide”
ctgaaatgtaaaagaataattctttagttttagcaaaaaagaaaacatcatgaaaattttacatctcttaagaaagtctttgtttttaatccaaataatc

SEQ ID NO: 275 - DNA, 1446 bp, Artificial Sequence
  source: /note=“Description of Artificial Sequence: Synthetic polynucleotide”
tttcttgatcatgaaaacgccaacaaaattctgaatcggccaaagaggtataattcaggtaaattggaagagtttgttcaagggaaccttgagagagaatgtatggaagaaaagtgtagttttgaagaagcagtattcactttggaggactttgtcggtgactggaggcaaaccgctggttataatctcgaccaagtactggaacagggcggggtaagttccctctttcagaatttgggtgtaagcgtcacaccaatccagcggattgtgttgtctggagagaacggactcaaaattgacatccatgttatcattccatatgaaggtctcagtggagaccaaatggggcagatcgagaagattttcaaggtagtttacccagtcgacgatcaccacttcaaagtcattctccactatggcacacttgttatcgacggagtaactcctaatatgattgattactttggtcgcccgtatgagggcatcgcagtgtttgatggcaaaaagatcaccgtaacaggaacgttgtggaatgggaacaagataatcgacgagagattgataaatccagacgggtcactcctgttcagggttacaattaacggcgtcacaggatggagactctgtgaacgaatactggccacaaatttttcactcctgaagcaggccggagacgtggaggaaaacccagggcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagcccgaagaagaagagaaaggtctaa

SEQ ID NO: 276 - DNA, 1446 bp, Artificial Sequence
  source: /note=“Description of Artificial Sequence: Synthetic polynucleotide”
ttacaccttcctcttcttcttggggctgccgccgcccttgtacagctcgtccatgcccagggtgatgccggcggcggtcacgaactccagcagcaccatgtggtccctcttctcgttggggtccttgctcagggcgctctgggtgctcaggtagtggttgtcgggcagcagcacggggccgtcgccgatgggggtgttctgctggtagtggtcggccagctgcacgctgccgtcctcgatgttgtgcctgatcttgaagttcaccttgatgccgttcttctgcttgtcggccatgatgtacacgttgtggctgttgtagttgtactccagcttgtggcccaggatgttgccgtcctccttgaagtcgatgcccttcagctcgatcctgttcaccagggtgtcgccctcgaacttcacctcggccctggtcttgtagttgccgtcgtccttgaagaagatggtcctctcctgcacgtagccctcgggcatggcgctcttgaagaagtcgtgctgcttcatgtggtcggggtacctgctgaagcactgcacgccgtaggtcagggtggtcaccagggtgggccagggcacgggcagcttgccggtggtgcagatgaacttcagggtcagcttgccgtaggtggcgtcgccctcgccctcgccgctcacgctgaacttgtggccgttcacgtcgccgtccagctccaccaggatgggcaccacgccggtgaacagctcctcgcccttgctcacggggccggggttctcctccacgtcgccggcctgcttcagcaggctgaagttggtggccaggatcctctcgcacagcctccagccggtcacgccgttgatggtcaccctgaacagcaggctgccgtcggggttgatcagcctctcgtcgatgatcttgttgccgttccacagggtgccggtcacggtgatcttcttgccgtcgaacacggcgatgccctcgtagggcctgccgaagtagtcgatcatgttgggggtcacgccgtcgatcaccagggtgccgtagtgcaggatcaccttgaagtggtggtcgtccacggggtacaccaccttgaaaatcttctcgatctggcccatctggtcgccgctcaggccctcgtaggggatgatcacgtggatgtcgatcttcaggccgttctcgccgctcagcacgatcctctggatgggggtcacgctcacgcccaggttctggaacaggctgctcacgccgccctgctccagcacctggtccaggttgtagccggcggtctgcctccagtcgcccacgaagtcctccagggtgaacacggcctcctcgaagctgcacttctcctccatgcactccctctccaggttgccctgcacgaactcctccagcttgccgctgttgtacctcttgggcctgttcaggatcttgttggcgttctcgtggtccaggaa

SEQ ID NO: 277 - DNA, 3570 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 278 - DNA, 3636 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctctgattatttggattaaaaacaaagactttcttaagagatgtaaaattttcatgatgttttcttttttgctaaaactaaagaattattcttttacatttcagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtctcccggtatgtcaactggattaaggaaaaaacaaagctcactgtcagcggatggagactgttcaagaagatcagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggaaatcttcttaaacagccgccagccgctcacggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgaaatgtaaaagaataattctttagttttagcaaaaaagaaaacatcatgaaaattttacatctcttaagaaagtctttgtttttaatccaaataatcagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 279 - DNA, 3615 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcagtattcactttggaggactttgtcggtgactggaggcaaaccgctggttataatctcgaccaagtactggaacagggcggggtaagttccctctttcagaatttgggtgtaagcgtcacaccaatccagcggattgtgttgtctggagagaacggactcaaaattgacatccatgttatcattccatatgaaggtctcagtggagaccaaatggggcagatcgagaagattttcaaggtagtttacccagtcgacgatcaccacttcaaagtcattctccactatggcacacttgttatcgacggagtaactcctaatatgattgattactttggtcgcccgtatgagggcatcgcagtgtttgatggcaaaaagatcaccgtaacaggaacgttgtggaatgggaacaagataatcgacgagagattgataaatccagacgggtcactcctgttcagggttacaattaacggcgtcacaggatggagactctgtgaacgaatactggccacaaatttttcactcctgaagcaggccggagacgtggaggaaaacccagggcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagcccgaagaagaagagaaaggtctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacaccttcctcttcttcttggggctgccgccgcccttgtacagctcgtccatgcccagggtgatgccggcggcggtcacgaactccagcagcaccatgtggtccctcttctcgttggggtccttgctcagggcgctctgggtgctcaggtagtggttgtcgggcagcagcacggggccgtcgccgatgggggtgttctgctggtagtggtcggccagctgcacgctgccgtcctcgatgttgtgcctgatcttgaagttcaccttgatgccgttcttctgcttgtcggccatgatgtacacgttgtggctgttgtagttgtactccagcttgtggcccaggatgttgccgtcctccttgaagtcgatgcccttcagctcgatcctgttcaccagggtgtcgccctcgaacttcacctcggccctggtcttgtagttgccgtcgtccttgaagaagatggtcctctcctgcacgtagccctcgggcatggcgctcttgaagaagtcgtgctgcttcatgtggtcggggtacctgctgaagcactgcacgccgtaggtcagggtggtcaccagggtgggccagggcacgggcagcttgccggtggtgcagatgaacttcagggtcagcttgccgtaggtggcgtcgccctcgccctcgccgctcacgctgaacttgtggccgttcacgtcgccgtccagctccaccaggatgggcaccacgccggtgaacagctcctcgcccttgctcacggggccggggttctcctccacgtcgccggcctgcttcagcaggctgaagttggtggccaggatcctctcgcacagcctccagccggtcacgccgttgatggtcaccctgaacagcaggctgccgtcggggttgatcagcctctcgtcgatgatcttgttgccgttccacagggtgccggtcacggtgatcttcttgccgtcgaacacggcgatgccctcgtagggcctgccgaagtagtcgatcatgttgggggtcacgccgtcgatcaccagggtgccgtagtgcaggatcaccttgaagtggtggtcgtccacggggtacaccaccttgaaaatcttctcgatctggcccatctggtcgccgctcaggccctcgtaggggatgatcacgtggatgtcgatcttcaggccgttctcgccgctcagcacgatcctctggatgggggtcacgctcacgcccaggttctggaacaggctgctcacgccgccctgctccagcacctggtccaggttgtagccggcggtctgcctccagtcgcccacgaagtcctccagggtgaacacggcctcctcgaagctgcacttctcctccatgcactccctctccaggttgccctgcacgaactcctccagcttgccgctgttgtacctcttgggcctgttcaggatcttgttggcgttctcgtggtccaggaa

SEQ ID NO: 280 - DNA, 3636 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtctcccggtatgtcaactggattaaggaaaaaacaaagctcactgtcagcggatggagactgttcaagaagatcagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggaaatcttcttaaacagccgccagccgctcacggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 281 - DNA, 1954 bp, Artificial Sequence
  source: /note=“Description of Artificial Sequence: Synthetic polynucleotide”
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgataggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 282 - DNA, 2359 bp, Artificial Sequence
  source: /note=“Description of Artificial Sequence: Synthetic polynucleotide”
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggtcgtctccggctctgctttttccaggggtgtgtttcgccgagaagcacgtaagagttttatgttttttcatctctgcttgtatttttctagtaatggaagcctggtattttaaaatagttaaattttcctttagtgctgatttctagattattattactgttgttgttgttattattgtcattatttgcatctgagaactaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 283 - DNA, 2925 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgtaacgatcgggaactggcatcttcagggagtagcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccgtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagaatctcatggtatcttgagaacattattttgtctctttgtagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 284 - DNA, 3477 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttagcctctggcaaaatgaagtgggtaacctttctcctcctcctcttcgtctccggctctgctttttccaggggtgtgtttcgccgagaagcacgtaagagttttatgttttttcatctctgcttgtatttttctagtaatggaagcctggtattttaaaatagttaaattttcctttagtgctgatttctagattattattactgttgttgttgttattattgtcattatttgcatctgagaacccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgtaacgatcgggaactggcatcttcagggagtagcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccgtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagaatctcatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 285 - DNA, 2476 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaagtatattagagcgagtctttctgcacacagatcacctttcctatcaaccccactagcctctggcaaaatgaagtgggtaacctttctcctcctcctcttcgtctccggctctgctttttccaggggtgtgtttcgccgagaagcacgtaagagttttatgttttttcatctctgcttgtatttttctagtaatggaagcctggtattttaaaatagttaaattttcctttagtgctgatttctagattattattactgttgttgttgttattattgtcattatttgcatctgagaacctttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 286 - DNA, 4175 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaagtatattagagcgagtctttctgcacacagatcacctttcctatcaaccccactagcctctggcaaaatgaagtgggtaacctttctcctcctcctcttcgtctccggctctgctttttccaggggtgtgtttcgccgagaagcacgtaagagttttatgttttttcatctctgcttgtatttttctagtaatggaagcctggtattttaaaatagttaaattttcctttagtgctgatttctagattattattactgttgttgttgttattattgtcattatttgcatctgagaacctttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgtaacgatcgggaactggcatcttcagggagtagcttaggtcagtgaagagaagaacaaaaagcagcatattacagttagttgtcttcatcaatctttaaatatgttgtgtggtttttctctccctgtttccacagacaagagtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagatccatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcctttgcttggaaaggcaggtgttcccattacgcctcagagaatagctgacgggaagaggctttctagatagttgtatgaaagatatacaaaatctcgcaggtatacacaggcatgatttgctggttgggagagccacttgcctcatactgaggtttttgtgtctgcttttcagagtcctgattgccttttcccagtatctccagaaatgctcatacgatgagcatgccaaattagtgcaggaagtaacagactttgcaaagacgtgtgttgccgatgagtctgccgccaactgtgacaaatcccttgtgagtaccttctgattttgtggatctactttcctgctttctggaactctgtttcaaagccaatcatgactccatcacttaaggccccgggaacactgtggcagagggcagcagagagattgataaagccagggtgatgggaattttctgtgggactccatttcatagtaattgcagaagctacaatacactcaaaaagtctcaccacatgactgcccaaatgggagcttgacagtgacagtgacagtagatatgccaaagtggatgagggaaagaccacaagagctaaaccctgtaaaaagaactgtaggcaactaaggaatgcagagagaaagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 287 - DNA, 3675 bp, Artificial Sequence
  source: /note=“Description of Artificial Sequence: Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaagtatattagagcgagtctttctgcacacagatcacctttcctatcaaccccactagcctctggcaaaatgaagtgggtaacctttctcctcctcctcttcgtctccggctctgctttttccaggggtgtgtttcgccgagaagcacgtaagagttttatgttttttcatctctgcttgtatttttctagtaatggaagcctggtattttaaaatagttaaattttcctttagtgctgatttctagattattattactgttgttgttgttattattgtcattatttgcatctgagaacctttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgtaacgatcgggaactggcatcttcagggagtagcttaggtcagtgaagagaagaacaaaaagcagcatattacagttagttgtcttcatcaatctttaaatatgttgtgtggtttttctctccctgtttccacagacaagagtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagatccatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcctttgcttggaaaggcaggtgttcccattacgcctcagagaatagctgacgggaagaggctttctagatagttgtatgaaagatatacaaaatctcgcaggtatacacaggcatgatttgctggttgggagagccacttagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 288-299 - skipped (12 entries)

SEQ ID NO: 300 - RNA, 22 nt, Artificial Sequence
  source: /note=“Description of Artificial Sequence: Synthetic oligonucleotide”
guuuuagagcuaugcuguuuug

SEQ ID NO: 301 - RNA, 80 nt, Artificial Sequence
  source: /note=“Description of Artificial Sequence: Synthetic oligonucleotide”
guuuuagagcuagaaauagcaaguuaaaauaaggcuaguccguuaucaacuugaaaaaguggcaccgagucggugcuuuu

SEQ ID NO: 302 - RNA, 76 nt, Artificial Sequence
  source: /note=“Description of Artificial Sequence: Synthetic oligonucleotide”
guuuuagagcuagaaauagcaaguuaaaauaaggcuaguccguuaucaacuugaaaaaguggcaccgagucggugc

SEQ ID NO: 303-349 - skipped (47 entries)

SEQ ID NO: 350 - RNA, 100 nt, Artificial Sequence
  source: /note=“Description of Artificial Sequence: Synthetic polynucleotide”
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 351-499 - skipped (149 entries)

SEQ ID NO: 500 - DNA, 9 bp, Artificial Sequence
  source: /note=“Description of Artificial Sequence: Synthetic oligonucleotide”
ctggaccga

SEQ ID NO: 501 - DNA, 9 bp, Artificial Sequence
  source: /note=“Description of Artificial Sequence: Synthetic oligonucleotide”
tcggtccag

SEQ ID NO: 502 - DNA, 9 bp, Artificial Sequence
  source: /note=“Description of Artificial Sequence: Synthetic oligonucleotide”
tcggtccag

SEQ ID NO: 503-599 - skipped (97 entries)

SEQ ID NO: 600 - protein, 7 aa, Simian virus 40
PKKKRKV

SEQ ID NO: 601 - protein, 7 aa, Simian virus 40
PKKKRRV

SEQ ID NO: 602 - protein, 16 aa, Unknown
  source: /note=“Description of Unknown: Nucleoplasmin bipartite NLS sequence”
KRPAATKKAGQAKKKK

SEQ ID NO: 603-702 - skipped (100 entries)

SEQ ID NO: 703 - DNA, 4104 bp, Artificial Sequence
  source: /note=“Description of Artificial Sequence: Synthetic polynucleotide”
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgat

SEQ ID NO: 704 - DNA, 4140 bp, Artificial Sequence
  source: /note=“Description of Artificial Sequence: Synthetic polynucleotide”
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 705 - RNA, 4501 nt, Artificial Sequence
  source: /note=“Description of Artificial Sequence: Synthetic polynucleotide”
gggucccgcagucggcguccagcggcucugcuuguucgugugugugucguugcaggccuuauucggauccgccaccauggacaagaaguacagcaucggacuggacaucggaacaaacagcgucggaugggcagucaucacagacgaauacaaggucccgagcaagaaguucaagguccugggaaacacagacagacacagcaucaagaagaaccugaucggagcacugcuguucgacagcggagaaacagcagaagcaacaagacugaagagaacagcaagaagaagauacacaagaagaaagaacagaaucugcuaccugcaggaaaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguacccgacaaucuaccaccugagaaagaagcuggucgacagcacagacaaggcagaccugagacugaucuaccuggcacuggcacacaugaucaaguucagaggacacuuccugaucgaaggagaccugaacccggacaacagcgacgucgacaagcuguucauccagcugguccagacauacaaccagcuguucgaagaaaacccgaucaacgcaagcggagucgacgcaaaggcaauccugagcgcaagacugagcaagagcagaagacuggaaaaccugaucgcacagcugccgggagaaaagaagaacggacuguucggaaaccugaucgcacugagccugggacugacaccgaacuucaagagcaacuucgaccuggcagaagacgcaaagcugcagcugagcaaggacacauacgacgacgaccuggacaaccugcuggcacagaucggagaccaguacgcagaccuguuccuggcagcaaagaaccugagcgacgcaauccugcugagcgacauccugagagucaacacagaaaucacaaaggcaccgcugagcgcaagcaugaucaagagauacgacgaacaccaccaggaccugacacugcugaaggcacuggucagacagcagcugccggaaaaguacaaggaaaucuucuucgaccagagcaagaacggauacgcaggauacaucgacggaggagcaagccaggaagaauucuacaaguucaucaagccgauccuggaaaagauggacggaacagaagaacugcuggucaagcugaacagagaagaccugcugagaaagcagagaacauucgacaacggaagcaucccgcaccagauccaccugggagaacugcacgcaauccugagaagacaggaagacuucuacccguuccugaaggacaacagagaaaagaucgaaaagauccugacauucagaaucccguacuacgucggaccgcuggcaagaggaaacagcagauucgcauggaugacaagaaagagcgaagaaacaaucacaccguggaacuucgaagaagucgucgacaagggagcaagcgcacagagcuucaucgaaagaaugacaaacuucgacaagaaccugccgaacgaaaagguccugccgaagcacagccugcuguacgaauacuucacagucuacaacgaacugacaaaggucaaguacgucacagaaggaaugagaaagccggcauuccugagcggagaacagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaacauaccacgaccugcugaagaucaucaaggacaaggacuuccuggacaacgaagaaaacgaagacauccuggaagacaucguccugacacugacacuguucgaagacagagaaaugaucgaagaaagacugaagacauacgcacaccuguucgacgacaaggucaugaagcagcugaagagaagaagauacacaggauggggaagacugagcagaaagcugaucaacggaaucagagacaagcagagcggaaagacaauccuggacuuccugaagagcgacggauucgcaaacagaaacuucaugcagcugauccacgacgacagccugacauucaaggaagacauccagaaggcacaggucagcggacagggagacagccugcacgaacacaucgcaaaccuggcaggaagcccggcaaucaagaagggaauccugcagacagucaaggucgucgacgaacuggucaaggucaugggaagacacaagccggaaaacaucgucaucgaaauggcaagagaaaaccagacaacacagaagggacagaagaacagcagagaaagaaugaagagaaucgaagaaggaaucaaggaacugggaagccagauccugaaggaacacccggucgaaaacacacagcugcagaacgaaaagcuguaccuguacuaccugcagaacggaagagacauguacgucgaccaggaacuggacaucaacagacugagcgacuacgacgucgaccacaucgucccgcagagcuuccugaaggacgacagcaucgacaacaagguccugacaagaagcgacaagaacagaggaaagagcgacaacgucccgagcgaagaagucgucaagaagaugaagaacuacuggagacagcugcugaacgcaaagcugaucacacagagaaaguucgacaaccugacaaaggcagagagaggaggacugagcgaacuggacaaggcaggauucaucaagagacagcuggucgaaacaagacagaucacaaagcacgucgcacagauccuggacagcagaaugaacacaaaguacgacgaaaacgacaagcugaucagagaagucaaggucaucacacugaagagcaagcuggucagcgacuucagaaaggacuuccaguucuacaaggucagagaaaucaacaacuaccaccacgcacacgacgcauaccugaacgcagucgucggaacagcacugaucaagaaguacccgaagcuggaaagcgaauucgucuacggagacuacaaggucuacgacgucagaaagaugaucgcaaagagcgaacaggaaaucggaaaggcaacagcaaaguacuucuucuacagcaacaucaugaacuucuucaagacagaaaucacacuggcaaacggagaaaucagaaagagaccgcugaucgaaacaaacggagaaacaggagaaaucgucugggacaagggaagagacuucgcaacagucagaaagguccugagcaugccgcaggucaacaucgucaagaagacagaaguccagacaggaggauucagcaaggaaagcauccugccgaagagaaacagcgacaagcugaucgcaagaaagaaggacugggacccgaagaaguacggaggauucgacagcccgacagucgcauacagcguccuggucgucgcaaaggucgaaaagggaaagagcaagaagcugaagagcgucaaggaacugcugggaaucacaaucauggaaagaagcagcuucgaaaagaacccgaucgacuuccuggaagcaaagggauacaaggaagucaagaaggaccugaucaucaagcugccgaaguacagccuguucgaacuggaaaacggaagaaagagaaugcuggcaagcgcaggagaacugcagaagggaaacgaacuggcacugccgagcaaguacgucaacuuccuguaccuggcaagccacuacgaaaagcugaagggaagcccggaagacaacgaacagaagcagcuguucgucgaacagcacaagcacuaccuggacgaaaucaucgaacagaucagcgaauucagcaagagagucauccuggcagacgcaaaccuggacaagguccugagcgcauacaacaagcacagagacaagccgaucagagaacaggcagaaaacaucauccaccuguucacacugacaaaccugggagcaccggcagcauucaaguacuucgacacaacaaucgacagaaagagauacacaagcacaaaggaaguccuggacgcaacacugauccaccagagcaucacaggacuguacgaaacaagaaucgaccugagccagcugggaggagacggaggaggaagcccgaagaagaagagaaaggucuagcuagccaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 706-799 - skipped (94 entries)

SEQ ID NO: 800 - RNA, 6 nt, Artificial Sequence
  source: /note=“Description of Artificial Sequence: Synthetic oligonucleotide”
aauaaa

SEQ ID NO: 801 - RNA, 6 nt, Artificial Sequence
  source: /note=“Description of Artificial Sequence: Synthetic oligonucleotide”
uauaaa

SEQ ID NO: 802 - RNA, 6 nt, Artificial Sequence
  source: /note=“Description of Artificial Sequence: Synthetic oligonucleotide”
akuaaa

SEQ ID NO: 803-1128 - skipped (326 entries)

SEQ ID NO: 1129 - RNA, 20 nt, Artificial Sequence
  source: /note=“Description of Artificial Sequence: Synthetic oligonucleotide”
gaguccgagcagaagaagaa

SEQ ID NO: 1130 - RNA, 20 nt, Artificial Sequence
  source: /note=“Description of Artificial Sequence: Synthetic oligonucleotide”
gacccccuccaccccgccuc

SEQ ID NO: 1131 - protein, 18 aa, Artificial Sequence
  source: /note=“Description of Artificial Sequence: Synthetic peptide”
EGRGSLLTCGDVEENPGP

SEQ ID NO: 1132 - protein, 19 aa, Artificial Sequence
  source: /note=“Description of Artificial Sequence: Synthetic peptide”
ATNFSLLKQAGDVEENPGP

SEQ ID NO: 1133 - protein, 20 aa, Artificial Sequence
  source: /note=“Description of Artificial Sequence: Synthetic peptide”
QCTNYALLKLAGDVESNPGP

SEQ ID NO: 1134 - protein, 22 aa, Artificial Sequence
  source: /note=“Description of Artificial Sequence: Synthetic peptide”
VKQTLNFDLLKLAGDVESNPGP

SEQ ID NO: 1135 - protein, 21 aa, Artificial Sequence
  source: /note=“Description of Artificial Sequence: Synthetic peptide”
GSGEGRGSLLTCGDVEENPGP

SEQ ID NO: 1136 - protein, 22 aa, Artificial Sequence
  source: /note=“Description of Artificial Sequence: Synthetic peptide”
GSGATNFSLLKQAGDVEENPGP

SEQ ID NO: 1137 - protein, 23 aa, Artificial Sequence
  source: /note=“Description of Artificial Sequence: Synthetic peptide”
GSGQCTNYALLKLAGDVESNPGP

SEQ ID NO: 1138 - protein, 25 aa, Artificial Sequence
  source: /note=“Description of Artificial Sequence: Synthetic peptide”
GSGVKQTLNFDLLKLAGDVESNPGP